SEQUENCE LISTING (67 sequences)

SEQ ID NO: 1 - protein, 916 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRGSGSGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNG

SEQ ID NO: 2 - protein, 916 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRGSGSGSGRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNG

SEQ ID NO: 3 - protein, 1147 aa, synthetic construct
MFVFLVLLPLVSSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSGSGSGMQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRGSGSGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNG

SEQ ID NO: 4 - protein, 1147 aa, synthetic construct
MFVFLVLLPLVSSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSGSGSGMQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRGSGSGSGRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNG

SEQ ID NO: 5 - protein, 1124 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICGSGSGSRVSPSTEVVRFPNITNLCPFGQVFNASNFPSVYAWERLRISDCVADYAVLYNSSSSFSTFKCYGVSPTKLNDLCFSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTELVKNKCVNFGSGSGSRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTDLIKNQCVNF

SEQ ID NO: 6 - protein, 1124 aa, synthetic construct; identical to SEQ ID NO: 5

SEQ ID NO: 7 - protein, 1136 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRGSGSGSRVSPSLDVVRFPNMTNICPFDQVFNKTQFPSVYAWERVRISDCVSDYTVLYNSSASFSTFKCYGVSPTKLNDLCFSGVYADYFVVKGDHVHQIAPGQTGVIADYNYKLPSEFVGCILAWNTRTIDSKRGFYYRLFRHGNIRPYERDTSNVPYNAAGGTCNQPGTHNCYEPLQDYGFTSTSGVGYQPFRVVVLSFELLNAPATVCGPKQSTDLVKNKCVNFGSGSGSRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTDLIKNQCVNF

SEQ ID NO: 8 - protein, 1139 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRGSGSGSRVQPTISIVRFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGGNYGYLYRLFRKSKLKPFERDISTEIYQAGSTPCNGQVGLNCYYPLERYGFHPTTGVNYQPFRVVVLSFELLNGPATVCGPKLSTTLVKDKCVNFGSGSGSRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTDLIKNQCVNF

SEQ ID NO: 9 - protein, 1364 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRGSGSGSRVSPSLDVVRFPNMTNICPFDQVFNKTQFPSVYAWERVRISDCVSDYTVLYNSSASFSTFKCYGVSPTKLNDLCFSGVYADYFVVKGDHVHQIAPGQTGVIADYNYKLPSEFVGCILAWNTRTIDSKRGFYYRLFRHGNIRPYERDTSNVPYNAAGGTCNQPGTHNCYEPLQDYGFTSTSGVGYQPFRVVVLSFELLNAPATVCGPKQSTDLVKNKCVNFGSGSGSRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTDLIKNQCVNFGSGSGSRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTDLIKNQCVNF

SEQ ID NO: 10 - protein, 1364 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRGSGSGSRVSPSLDVVRFPNMTNICPFDQVFNKTQFPSVYAWERVRISDCVSDYTVLYNSSASFSTFKCYGVSPTKLNDLCFSGVYADYFVVKGDHVHQIAPGQTGVIADYNYKLPSEFVGCILAWNTRTIDSKRGFYYRLFRHGNIRPYERDTSNVPYNAAGGTCNQPGTHNCYEPLQDYGFTSTSGVGYQPFRVVVLSFELLNAPATVCGPKQSTDLVKNKCVNFGSGSGSRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTDLIKNQCVNFGSGSGSRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 11 - protein, 1367 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRGSGSGSRVQPTDSIVRFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKSTNLVKNKCVNFGSGSGSRVSPSTEVVRFPNITNLCPFGQVFNASNFPSVYAWERLRISDCVADYAVLYNSSSSFSTFKCYGVSPTKLNDLCFSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTELVKNKCVNFGSGSGSRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTDLIKNQCVNF

SEQ ID NO: 12 - protein, 668 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGIC

SEQ ID NO: 13 - protein, 682 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPR

SEQ ID NO: 14 - protein, 222 aa, synthetic construct
RVSPSTEVVRFPNITNLCPFGQVFNASNFPSVYAWERLRISDCVADYAVLYNSSSSFSTFKCYGVSPTKLNDLCFSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTELVKNKCVNF

SEQ ID NO: 15 - protein, 222 aa, synthetic construct
RVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTDLIKNQCVNF

SEQ ID NO: 16 - protein, 205 aa, synthetic construct
RVSPTQEVIRFPNITNRCPFDKVFNATRFPNVYAWERTKISDCVADYTVLYNSTSFSTFKCYGVSPSKLIDLCFTSVYADTFLIRSSEVRQVAPGETGVIADYNYKLPDDFTGCVIAWNTAKHDTGNYYYRSHRKTKLKPFERDLSSDDGNGVYTLSTYDFNPNVPVAYQATRVVVLSFELLNAPATVCGPKLSTELVKNQCVNF

SEQ ID NO: 17 - protein, 222 aa, synthetic construct
RVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTDLIKNQCVNF

SEQ ID NO: 18 - protein, 220 aa, synthetic construct
RVSPSLDVVRFPNMTNICPFDQVFNKTQFPSVYAWERVRISDCVSDYTVLYNSSASFSTFKCYGVSPTKLNDLCFSGVYADYFVVKGDHVHQIAPGQTGVIADYNYKLPSEFVGCILAWNTRTIDSKRGFYYRLFRHGNIRPYERDTSNVPYNAAGGTCNQPGTHNCYEPLQDYGFTSTSGVGYQPFRVVVLSFELLNAPATVCGPKQSTDLVKNKCVNF

SEQ ID NO: 19 - protein, 223 aa, synthetic construct
RVQPTISIVRFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGGNYGYLYRLFRKSKLKPFERDISTEIYQAGSTPCNGQVGLNCYYPLERYGFHPTTGVNYQPFRVVVLSFELLNGPATVCGPKLSTTLVKDKCVNF

SEQ ID NO: 20 - protein, 222 aa, synthetic construct
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 21 - protein, 223 aa, synthetic construct
RVQPTDSIVRFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKSTNLVKNKCVNF

SEQ ID NO: 22 - protein, 227 aa, synthetic construct
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNG

SEQ ID NO: 23 - protein, 236 aa, synthetic construct
MFVFLVLLPLVSSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 24 - protein, 227 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNG

SEQ ID NO: 25 - DNA, 2751 bp, synthetic construct
  source 1..2751: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagaggatccggcagtgggtcaggaagggttcagcccacagagtccatagtacggttccctaatattaccaacctgtgtccgttcggtgaagtcttcaatgccacaagattcgccagcgtttatgcctggaaccgaaagagaattagtaattgtgttgcggattattctgtcctttacaatagtgcaagcttctccaccttcaagtgttacggggtttccccgacgaaactcaacgacctttgttttacaaatgtgtatgcagattcatttgttatccgaggagatgaggtacggcaaatagcccccggacaaacaggaaagatagccgattacaactataaactgcctgatgacttcactgggtgcgtcatcgcatggaatagcaacaaccttgattccaaagtagggggcaattataattaccgctatcgacttttccgcaagtccaacctcaaacccttcgagcgcgatatcagcacggaaatttaccaagcgggcagcaaaccatgcaacggagtcgaaggatttaattgttacttcccgttgcaatcctatggctttcaaccaaccaacggtgttggttaccaaccttatagagttgtcgttctcagttttgagctcctccatgccccggccaccgtatgcggcccaaagaaatcaacaaacctcgttaagaacaaatgtgtcaactttaatttcaatgggtag

SEQ ID NO: 26 - DNA, 2751 bp, synthetic construct
  source 1..2751: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagaggatccggcagtgggtcaggacgagtccaaccaacagagtcaatcgtaagattccccaatatcaccaacctctgcccctttgacgaagttttcaacgctaccagatttgcaagtgtgtacgcctggaataggaagcggatcagtaactgcgtcgccgactactctgtactttataacctcgcgcccttctttacattcaagtgctatggcgtctcccccacgaaacttaatgacctctgtttcacgaacgtctacgccgattcctttgtgataagaggggatgaggtaagacaaatagcacctggtcagacaggcaacatagcagactataactataagttgcccgacgactttacaggatgtgtaattgcttggaacagtaacaagctggattcaaaggtttccggcaattataactacctgtacagactgttcagaaaaagtaatctcaaacccttcgagagagacatttcaaccgaaatctatcaggccgggaacaaaccatgcaacggcgtcgctggattcaactgttacttccccctccgatcatactcctttaggcccacatatggagtgggtcatcaaccttaccgagttgttgtcctgtctttcgagctcttgcacgcgccggcaactgtgtgtggacctaaaaagagtacgaacttggtgaagaataagtgtgtaaactttaactttaatggttag

SEQ ID NO: 27 - DNA, 3444 bp, synthetic construct
  source 1..3444: mol_type = other DNA
atgttcgtgtttctggtgctgctgcctctggtgtccagccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactatagtgtactgtataactcagcatccttttccacgtttaagtgctacggggtcagtcctacaaagttgaatgacttgtgctttacaaacgtatatgctgattcctttgtcatacgaggcgatgaggtcagacagattgctccgggacaaaccggaaatattgccgactacaactacaaacttcccgacgattttactgggtgtgtaattgcctggaacagtaacaatcttgactcaaaagtggggggaaattacaattacctttatagactcttccgcaagagcaatctcaagccttttgaacgcgatatatctaccgaaatataccaggccggttctactccatgcaatggagttaagggatttaattgttactttcctctccaatcttatgggttccagccaacttatggagtcggttatcagccatatcgagtcgttgtgctcagcttcgaactgcttcatgccccagctactgtttgtgggccgaaaaagtccactaatctcgtgaagaataagtgcgtaaactttggcagtgggtcaggatccggaatgcagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagaggatccggcagtgggtcaggaagggttcagcccacagagtccatagtacggttccctaatattaccaacctgtgtccgttcggtgaagtcttcaatgccacaagattcgccagcgtttatgcctggaaccgaaagagaattagtaattgtgttgcggattattctgtcctttacaatagtgcaagcttctccaccttcaagtgttacggggtttccccgacgaaactcaacgacctttgttttacaaatgtgtatgcagattcatttgttatccgaggagatgaggtacggcaaatagcccccggacaaacaggaaagatagccgattacaactataaactgcctgatgacttcactgggtgcgtcatcgcatggaatagcaacaaccttgattccaaagtagggggcaattataattaccgctatcgacttttccgcaagtccaacctcaaacccttcgagcgcgatatcagcacggaaatttaccaagcgggcagcaaaccatgcaacggagtcgaaggatttaattgttacttcccgttgcaatcctatggctttcaaccaaccaacggtgttggttaccaaccttatagagttgtcgttctcagttttgagctcctccatgccccggccaccgtatgcggcccaaagaaatcaacaaacctcgttaagaacaaatgtgtcaactttaatttcaatgggtag

SEQ ID NO: 28 - DNA, 3444 bp, synthetic construct
  source 1..3444: mol_type = other DNA
atgttcgtgtttctggtgctgctgcctctggtgtccagccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactatagtgtactgtataactcagcatccttttccacgtttaagtgctacggggtcagtcctacaaagttgaatgacttgtgctttacaaacgtatatgctgattcctttgtcatacgaggcgatgaggtcagacagattgctccgggacaaaccggaaatattgccgactacaactacaaacttcccgacgattttactgggtgtgtaattgcctggaacagtaacaatcttgactcaaaagtggggggaaattacaattacctttatagactcttccgcaagagcaatctcaagccttttgaacgcgatatatctaccgaaatataccaggccggttctactccatgcaatggagttaagggatttaattgttactttcctctccaatcttatgggttccagccaacttatggagtcggttatcagccatatcgagtcgttgtgctcagcttcgaactgcttcatgccccagctactgtttgtgggccgaaaaagtccactaatctcgtgaagaataagtgcgtaaactttggcagtgggtcaggatccggaatgcagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagaggatccggcagtgggtcaggacgagtccaaccaacagagtcaatcgtaagattccccaatatcaccaacctctgcccctttgacgaagttttcaacgctaccagatttgcaagtgtgtacgcctggaataggaagcggatcagtaactgcgtcgccgactactctgtactttataacctcgcgcccttctttacattcaagtgctatggcgtctcccccacgaaacttaatgacctctgtttcacgaacgtctacgccgattcctttgtgataagaggggatgaggtaagacaaatagcacctggtcagacaggcaacatagcagactataactataagttgcccgacgactttacaggatgtgtaattgcttggaacagtaacaagctggattcaaaggtttccggcaattataactacctgtacagactgttcagaaaaagtaatctcaaacccttcgagagagacatttcaaccgaaatctatcaggccgggaacaaaccatgcaacggcgtcgctggattcaactgttacttccccctccgatcatactcctttaggcccacatatggagtgggtcatcaaccttaccgagttgttgtcctgtctttcgagctcttgcacgcgccggcaactgtgtgtggacctaaaaagagtacgaacttggtgaagaataagtgtgtaaactttaactttaatggttag

SEQ ID NO: 29 - DNA, 3381 bp, synthetic construct
  source 1..3381: mol_type = other DNA
atgtttgtgtttctagtgctactaccactagtgtcctctcagtgcgtgaacctgatcacacggacccagtcatacaccaactccttcaccagaggagtgtactaccctgacaaggtgttcagatctagcgtgctgcattctacccaggacctgtttctgcctttcttcagcaatgtgacctggttccacgccatccacgtgtctggcacaaacggcaccaagagattcgacaaccctgtgctgcctttcaacgatggcgtgtacttcgcctctacagagaagtccaacatcatccggggctggatcttcggcaccacactggactcgaagacacaatctctgctgatcgtgaacaacgccaccaacgttgtcatcaaggtgtgcgagttccagttctgcaacgaccctttcttggacgtgtactaccacaagaacaacaagtcctggatggaatccgaattcagagtgtactccagcgccaacaactgtacattcgagtacgtcagccagccctttctgatggacctggaaggcaagcagggaaatttcaagaacctgagagagttcgtgttcaagaacatcgacggctacttcaagatctacagcaagcacacccctattaacctgggcagagatctgccccagggcttttccgctctggagcccctggtggacctgcctatcggcatcaacatcacccggtttcagaccctcctagccctgcacagatcctatctgacccctggcgacagcagttccggctggaccgccggggctgctgcctactacgtggggtacctgcagcctagaaccttcctgctgaagtacaacgagaacggcaccatcaccgacgccgtggactgtgctctggatccactgtccgagaccaagtgcactctcaagagcttcaccgtggaaaagggaatctaccagacctccaactttagagtgcagcccaccgagtcaatcgtgagattccccaacatcaccaacctgtgcccttttgacgaggtgttcaacgctaccagatttgcttctgtgtacgcctggaaccggaagagaatctctaactgcgtggccgattactccgtgctgtacaacttcgctcctttcttcgccttcaagtgttacggcgtgtcccctaccaagctgaacgacctgtgtttcactaacgtgtacgccgactctttcgtgatccggggcaacgaggtgtcccagatcgccccaggccagacaggcaacatcgctgactacaactacaagctgcctgacgatttcaccggctgtgtgatcgcctggaactccaacaagctggattccaaagtgggcggcaactacaactatctgtaccggctgttcagaaagtccaatctgaagcccttcgaacgcgacatctccaccgagatctatcaggccggcaacaagccttgtaacggcgtcgctggcttcaactgctacttccctttgcgatcttatggcttccggcctacctacggcgtgggccaccagccttaccgggtggtggtgctgagctttgagctgctgcacgctcctgctacagtgtgcggccctaagaagtccaccaatctggtgaaaaataagtgcgttaacttcaacttcaacggcctgaccggcaccggcgtgctgacagagtcaaataagaagttcctgcctttccagcagtttggcagggacatcgccgacaccacggacgctgtgcgggaccctcagaccctggaaatcctggatatcaccccttgctccttcggcggcgtgagcgtgattacccctggaaccaacacctctaatcaagtggccgtgctgtaccaaggcgtgaactgcaccgaagtgcccgtggctatccacgccgatcagctgacccctacatggcgggtatacagtaccggctctaacgtgttccagacccgggctggctgcctgatcggcgccgagtacgtgaacaactcctacgagtgcgacatccccatcggcgctggcatttgcggatctggctctggatccagagtctctccaagcaccgaggtggtgcggtttcctaatatcaccaacctgtgccctttcggacaagtgtttaacgcctccaacttcccatcggtgtacgcctgggagagactgcgcatctccgactgcgtcgccgactatgccgtgctgtacaattcttcctcctctttctccacctttaagtgctacggcgtgagccctacaaagctgaacgacctgtgcttctcctctgtgtacgccgactacttcgtggtgaaaggcgatgatgtgagacagatcgcccctgcccagaccggcgtgatcgccgattacaactacaagctccccgacgacttcacaggctgcgtgctggcctggaataccaactccgtagattccaaatccggaaacaacttctactaccggctgttcagacacggcaagatcaagccctacgaaagagacatctccaacgtgctgtacaactccgctggcggcacctgttcctccatctctcagctgggctgctacgagcccctgaaatcctatgggttcacccccaccgtgggggtgggctaccagccttatagggtcgtggtgttatccttcgaactgctgaacgctccagctaccgtgtgcggccccaagaaatcaaccgagctggtcaagaacaagtgcgtgaacttcggctctggcagcggctctagagtggcaccctctaaagaagtggtcagattccccaatatcaccaatctgtgtccttttggcgaggtcttcaacgccaccaccttcccttctgtgtacgcctgggaacggaaacggatctctaactgcgtcgccgactactctgttctctacaactccacctctttctccacctttaaatgttacggcgtctctgctacaaaactgaacgatctgtgcttctctaacgtgtacgctgactcttttgtggtaaagggcgacgacgtgagacagatcgcccctggccaaaccggagtcatcgctgattataactacaaactgcctgatgacttcctgggatgtgtgctggcttggaacaccaatagcaaggacagcagcacatctggcaactacaactacctgtacagatgggtgaggagaagcaagctcaacccgtatgaacgggacctgtctaacgacatctactctccaggcggacagtcctgctccgctgtgggccctaactgctacaatcctctgagaccctacggcttcttcaccaccgctggcgtgggacatcagccttacagagttgttgtgctgagcttcgagctgctgaacgcccctgccaccgtgtgtggccctaagctgtctactgatctgatcaaaaaccagtgcgtgaatttctgataataa

SEQ ID NO: 30 - DNA, 3381 bp, synthetic construct; identical to SEQ ID NO: 29
  source 1..3381: mol_type = other DNA

SEQ ID NO: 31 - DNA, 3417 bp, synthetic construct
  source 1..3417: mol_type = other DNA
atgtttgtgtttctagtgctactaccactagtgtcttcccagtgcgtcaatctgaccaccagaacccaactgccccctgcttacactaactctttcaccagaggcgtgtactaccctgacaaggtgttccggtcgtccgtgctgcattctacccaggatctcttcttgcctttcttctctaacgtgacctggttccacgccatccatgtgtctggcacaaacggcaccaagagattcgacaaccctgtgctgcctttcaacgacggcgtgtatttcgcctctaccgagaagtctaacatcatccggggctggatcttcggcaccacactggacagcaagacccagtcgctgctgatcgtgaacaacgccaccaacgtggtcatcaaggtctgcgaattccagttctgcaacgatcctttcctgggcgtatactaccacaagaacaacaagtcctggatggaatccgaattcagagtatactcctccgccaataactgcacattcgagtacgtgtctcagcctttcctgatggacctggaaggcaagcagggcaacttcaaaaacctgagagagttcgtgttcaagaacatcgacggctacttcaagatctacagcaagcacacccctatcaacctggtgcgggatctgccacagggcttttccgctctggaacctctggtggacctgcccatcggcatcaatatcacccgctttcaaaccctgctggccctgcacagatcttacctgacaccaggcgactcctcctccggctggaccgccggcgctgccgcctactacgtgggctacctccagcctcggacctttctgctcaagtacaacgagaacggcacaatcaccgacgccgtggactgcgccctggaccccctgtccgagacaaagtgtaccctgaagtccttcaccgtggaaaaaggaatctatcaaacctccaactttcgtgtgcaacctactgaatctatcgtgagatttcctaatattaccaatctgtgtccttttggcgaggtgttcaacgccactcggttcgcttccgtgtacgcctggaacagaaagcggatcagcaactgcgttgctgactactccgtgctgtacaactccgcttccttcagcaccttcaagtgctatggggtctcccctaccaagctgaacgatctgtgcttcaccaacgtgtatgcagactctttcgtgatcagaggcgacgaggtcagacagatcgcccccggccagaccggaaagatcgctgactacaactacaagctgcccgacgacttcacaggctgtgtgatcgcgtggaactctaacaacctagactctaaggtaggcggcaactataactacctgtacagactgtttcggaagagcaacctgaagcctttcgagcgggacatctcgaccgagatctaccaggctggcagcacaccctgcaacggggtggaaggatttaactgctacttcccactgcagtcttatggcttccagcccacaaacggcgtgggctaccaaccttaccgggtggtggtgctgtccttcgagctgctgcacgctcctgctaccgtgtgtggcccaaagaaatctaccaacctggtgaagaacaagtgtgtcaacttcaatttcaacggcctgaccggcaccggcgtcctgaccgagtctaataagaagttcctgccctttcagcagttcggcagagatatcgccgataccaccgacgctgtgcgggacccccagacgctggaaatcctggatatcacgccttgctctttcggcggcgtgtccgtgatcacacccggcaccaacaccagcaaccaagtggctgtgttgtatcaggacgtgaactgcaccgaagtgcctgtggccatccacgccgatcagctgacccctacctggagagtgtactcaaccggcagcaacgtgttccagaccagagcgggctgcctgatcggcgccgagcacgtgaacaactcctacgagtgcgacatccctatcggagccggcatctgcgccagctaccaaacacagaccaactcccctcggggatctggctctggttccagagtgtctccttctctggacgtggtgaggtttccaaacatgaccaacatctgccctttcgaccaggtgttcaacaagacccagttcccttccgtctacgcttgggaacgggtgagaatctccgactgtgtgtccgactacaccgtgctgtacaacagctctgcttctttctccaccttcaagtgttacggcgtgtctcctaccaagctgaacgacctgtgcttctcaggcgtgtacgccgactacttcgtggtcaaaggcgatcacgtgcaccagatcgcccctggccagaccggcgtgatcgccgactacaattacaaactgccttccgagttcgtaggctgcatcctggcttggaacacaagaaccatcgactccaagagaggattctactaccggctgttcagacacggcaatatccggccgtacgagagagacacctccaatgtgccctacaatgctgctggaggcacctgcaaccagcctggcacccacaactgctacgagcctctgcaggactacggcttcacatccacatcgggagtgggctaccagccattccgcgtggtggtcttatcctttgagctgctgaacgcccctgccaccgtgtgtggccccaagcagagcaccgatctagtgaaaaacaagtgcgtcaatttcggctccggaagcggctccagagtggctccctctaaagaggtggtgagattccccaacattaccaacctgtgcccttttggagaggtcttcaatgctaccaccttccctagcgtatacgcttgggagcggaagcggatctccaactgcgtggccgactactctgtgctgtacaactccaccagcttttctaccttcaaatgctacggcgtgtccgccaccaagctgaacgatctgtgcttttccaacgtgtacgccgattccttcgtggttaagggcgacgatgtgagacagatcgcccctggccagacaggtgtaatcgctgattacaactacaagctgcctgacgacttcaccggctgtgtgctggcctggaacaccagaaacatcgatgctacacagaccggcaactacaactacaagtaccggagcctgcggcatggcaagctgagacctttcgagagagatatctccaacgtgccattctctcccgatggcaaaccttgtaccccacctgctttcaactgctactggcccctgaacgactacggcttctatatcactaacggcatcggctaccagccatacagagtggtggttctgagtttcgaactgctcaatgctccagccaccgtgtgcggacctaagctttccaccgacctgataaaaaaccagtgtgtgaacttctgataataa

SEQ ID NO: 32 - DNA, 3426 bp, synthetic construct
  source 1..3426: mol_type = other DNA
atgtttgtgtttctagtgctactaccactagtgtcctcccagtgtgtgaatctgacaaccagaacccagctgcctccagcctacaccaattccttcaccagaggagtctactaccctgacaaagtgttcagatcctctgtgctccactcaacacaggatctgtttctgcccttcttctccaacgtcacttggttccacgctatccatgtgtccggaacaaacggcacaaagagattcgacaaccccgtgttgcctttcaacgacggcgtgtacttcgcctctaccgagaagtccaacatcatcagaggctggatcttcggcaccacactcgattctaagacccagtctctgctgatcgttaacaacgccaccaacgtggtcatcaaggtgtgcgagttccagttctgcaacgaccccttcctgggcgtctactaccacaagaacaacaagtcttggatggaatccgagttccgtgtttacagctccgccaacaactgtaccttcgagtacgtctctcagcccttcctaatggacctggaaggtaaacagggcaatttcaagaacctgagagagttcgtgttcaagaacatcgacggatattttaagatctactctaagcacacccctatcaacctggtgcgggacctgcctcaaggcttctccgctctggagcctctagtggacctgcctattggcatcaacatcaccagattccagaccctactggctctgcacagatcttacctgactcctggcgacagctccagcggctggaccgccggcgctgccgcttactacgtgggctacctgcagcctcggaccttcctgctgaagtacaacgagaacggaaccatcaccgacgctgtggactgcgccctggatcctctgtctgaaactaagtgcaccctgaagtcgtttaccgtggaaaagggcatctaccagacctccaacttcagagtgcagccaaccgaatctatcgtgcgcttccccaacatcacaaacctgtgccccttcggcgaagtgttcaacgctacacggttcgcctccgtatacgcttggaacagaaaaagaatctccaactgtgtggctgattactccgtgctgtataacagcgcctctttttccaccttcaagtgctacggggtgtctcctaccaaactgaacgacctgtgctttaccaacgtgtacgccgactcctttgtgatccggggcgatgaggtgcggcagatcgcccctggccagaccggcaagatcgccgactataattacaagctgcctgacgacttcaccggctgcgtgatcgcatggaactccaacaatctggactccaaggtgggcggcaactacaactatctgtatagactgttcagaaagtccaacctgaaacctttcgagagagacatcagcaccgagatctaccaggccggctctaccccttgcaacggcgtagagggcttcaactgctacttccccctgcagtcctacggctttcaacctaccaacggagtgggctaccaaccttaccgggtggtggtgctgtccttcgagctgctgcacgcccctgctactgtgtgcggacctaagaaatctaccaatctggtgaaaaacaagtgcgtgaacttcaacttcaacggcctgacaggcactggcgtcctgaccgagtccaacaagaagttcctgcctttccagcagttcggcagagacatcgccgacaccaccgacgccgttagagatcctcagaccctggagatcctggacatcaccccttgctcctttggcggcgtgtccgtgatcacccctggcaccaacaccagtaatcaggtggccgtgctgtaccaggacgtgaactgcaccgaagtgcctgtggccatccacgccgaccagctgacacctacatggcgggtttactccacaggatccaacgtgttccagacccgggctggctgcctgatcggcgccgagcacgtgaacaactcctacgagtgcgacatccctatcggcgctggcatctgcgcttcttaccaaacccagaccaactccccccgcggctctggctccggcagtcgggtgcaacctaccatctccatcgtgcggtttcctaacatcaccaacctgtgccctttcggcgaggtcttcaacgccagcaagttcgcttccgtgtatgcctggaacagaaagagaatctccaactgcgtcgccgactactccgtgctgtacaattctacaagtttctccaccttcaagtgctacggcgtgtcccctaccaagctgaacgacctgtgcttcaccaacgtctacgccgactccttcgtggtgaagggcgatgaagtgagacagatcgctccaggacagacaggcgtaattgccgactataattataaactccccgatgacttcaccggctgcgtgattgcctggaactctgtcaagcaggacgctttgaccggcggaaactacggctacctgtaccggctgttcagaaagtccaagctgaaaccttttgaacgggacatctctaccgaaatctaccaggctgggagcaccccttgtaacggacaagtgggcctgaactgttactaccccctggaaagatacggcttccatcctaccaccggcgtgaattaccagcctttccgggttgtggttctgagctttgagctgctgaacggcccagctacggtgtgcggccccaagctctctaccacccttgtgaaggacaagtgcgtcaatttcggctctggctctgggtccagagtggctccttccaaagaggtggtgagatttcctaatatcaccaacctgtgtcctttcggcgaagtgttcaacgccaccaccttcccatctgtgtacgcctgggagagaaagcggatcagcaactgtgtggctgattattctgtgctgtacaactccacctccttctccacctttaagtgttacggcgtgtctgctaccaagctgaacgatctctgttttagtaacgtgtacgccgactccttcgtggtgaagggcgacgatgtgcggcagatcgcccctggccagaccggcgtgatcgctgattacaactacaagctgcctgacgactttacaggctgtgtgctggcttggaacacccggaacatcgatgccacccagaccggcaactacaactacaagtaccgctctctgcggcacggcaagctgaggccctttgagcgggatatctctaacgtgcctttcagccctgatggcaagccctgcacccctccagccttcaattgttactggcctctgaacgactacggcttctacatcaccaacggaatcggctaccagccctatagagtggtggtgctgtcgttcgagctgctgaatgctcccgccaccgtgtgtggcccaaagctgtccaccgacctgatcaagaaccagtgcgtgaacttctgataataa

SEQ ID NO: 33 - DNA, 4101 bp, synthetic construct
  source 1..4101: mol_type = other DNA
atgtttgtgtttctagtgctactaccactagtgtcctcccagtgcgtgaacctgaccacaagaacccagctgcctcctgcgtacaccaactccttcaccagaggcgtgtactaccctgacaaggtgtttcgttcctccgtcttgcactccacacaggatctgtttctgcctttcttctctaacgtgacgtggttccacgccatccacgtgtccggtaccaacggcacaaagagattcgacaaccccgtgctgcctttcaacgacggcgtttacttcgcctccactgagaagtctaacatcatcagaggatggatcttcggtaccaccctcgacagcaagacccaatccctgctgatagtgaacaacgctacaaacgtggtgatcaaggtgtgcgagttccagttctgcaacgatccttttctgggagtgtactaccacaagaataacaagtcctggatggaaagtgaattcagagtgtactctagcgccaacaactgtacctttgagtacgtgtcccaacctttcctgatggacctggaaggaaagcagggcaacttcaagaacctgagagagttcgtgttcaagaacatcgatggctacttcaagatctactccaagcacacacctatcaacttggttagagacctcccgcagggcttctccgctctggaacctctggtcgacctgcccattggcatcaacatcacacggttccagaccctgctggctctgcacagatcctacctgaccccaggagattcctcttctggctggaccgccggcgctgccgcttactacgtgggctacctgcagcctcggacattcctgctgaagtacaacgagaacggcaccatcaccgacgctgtggactgtgccctggatcccctgagcgagacaaagtgtaccctgaaatcttttaccgtggaaaagggcatttaccagacctccaacttcagggtgcagcctactgagtccatcgtgcggttccctaacatcaccaacctgtgccccttcggcgaagtgtttaacgccaccagatttgcttctgtgtacgcttggaacagaaagagaatcagcaactgcgtggctgattactccgtcctgtacaattcagcaagcttctccaccttcaaatgctacggagtgtctccaaccaagctgaacgacctgtgtttcaccaacgtgtacgccgactctttcgtgatccggggcgacgaagtccggcaaatcgcccccggccagaccggcaagatcgccgactacaactacaagctgcctgacgatttcaccggatgtgtcatcgcttggaactccaacaacctggatagtaaagtgggcggcaactataattatttgtaccggctgttccggaagtctaatctgaagcctttcgagagagacatctctaccgagatctaccaggccggctctacaccttgcaacggcgtagagggcttcaactgctacttccccctgcagagctacggcttccagcctactaacggcgttggctaccagccttacagagtggtggtgctgtctttcgagctgctgcatgctccagccacagtgtgcggccctaagaagtccacgaacctggtgaagaacaaatgcgtgaacttcaacttcaatggcctgaccggcaccggcgtgctgacagagtctaacaagaagttcctgccattccagcagttcggcagagatattgctgacaccactgatgccgtacgggaccctcagaccctggaaatcctggatatcaccccttgttccttcggcggcgtctccgtgatcactcctggaaccaacacctccaaccaggtggccgtgctgtaccaggacgtgaactgcaccgaggtgcctgtggccatccatgccgaccagctgacgcccacctggcgggtgtactccaccggatctaatgtgttccaaacccgggctggctgcctgatcggcgccgagcacgtgaataactcctacgagtgcgacatccctatcggggccggaatctgcgcgtcataccagacccagacaaactctcctagaggctctggctctggcagcagagtttcgccttctctggacgtggtcagattccctaacatgaccaacatctgccctttcgaccaggtgttcaacaaaacccagttcccctccgtgtacgcctgggaacgggtgagaatttccgactgcgtctcagattacaccgtgctctataacagctctgcctctttctctacctttaagtgctacggcgtgagccctaccaagttaaatgacctgtgctttagcggcgtgtacgccgattatttcgtggtaaagggtgaccacgtgcaccagatagcccccggccagaccggcgtgatcgctgactacaattataagctgcccagcgagttcgtgggctgcatcctggcctggaacactagaacaatcgactccaagagaggcttctactaccggctgttccggcatggcaacatcagaccttacgagcgcgatacctccaatgtgccctacaacgctgctggcgggacctgcaaccagcctggcacccacaactgctacgagcctctgcaggactacggcttcacctccacatctggcgtcggctaccaaccattcagagtggtggtgctgtctttcgagctgctgaacgctccagctaccgtgtgtggccctaagcaatccacagatctggtgaagaacaagtgtgtgaatttcggttccggctccggctctagagtggctccttctaaagaggttgtgcggtttcctaacatcaccaacctgtgtcctttcggcgaggtgttcaacgccaccacgttcccctctgtgtacgcctgggagagaaagaggatctccaactgtgttgctgactactcggtgctgtataactccacctctttttccaccttcaagtgctacggcgtctccgccaccaagctcaatgacctgtgcttctcgaacgtgtacgcagactccttcgtggtcaagggcgacgacgtgagacagatcgcccctggccagactggcgtgatcgccgactataattacaaactgccggacgactttctgggctgcgtgctggcttggaacaccaactctaaggactcttccacctctggcaactacaactacctgtatagatgggtgcgtaggagtaaactcaacccttacgagcgggacctgtccaacgatatctattcccctggcggccagtcctgctccgctgtgggccccaattgctacaaccccctgcggccttacggcttcttcacaaccgctggagtgggccaccagccttaccgggtggtggtgctgtcttttgaactgctgaatgctcctgctaccgtgtgcggccccaagctgtccaccgacctgataaaaaaccagtgtgtgaacttcggctctggcagcggttctagagtcgcccctagtaaagaagtggtgagattccccaacatcaccaacctgtgccctttcggcgaagtgttcaacgccaccaccttcccttctgtgtacgcttgggagcggaagcggatctccaactgtgtggccgactatagcgtgttatacaactctacctctttttccaccttcaagtgctatggcgtgtctgccaccaaactgaacgacctgtgcttctcgaacgtgtacgctgatagctttgtggtgaaaggcgacgacgtgagacagatcgcacctggacagaccggagtgatcgccgactataactacaagcttccagatgacttcaccggctgtgtgctggcttggaacacccggaacatcgacgccacccagacaggaaactacaactacaagtaccgctccctgagacacggcaagctgagacccttcgagcgggacatctccaacgtgcctttctctcctgatggcaagccctgcacccctccagccttcaactgttactggcctctgaacgactacggcttttacatcaccaatggcatcggatatcagccttacagagtggtggtcctgtcctttgagctgctgaacgcccctgccaccgtgtgcggccctaagctgtccaccgacctgatcaagaaccagtgcgtgaacttttgataataa

SEQ ID NO: 34 - DNA, 4101 bp, synthetic construct
  source 1..4101: mol_type = other DNA
atgtttgtgtttctagtgctactaccactagtctcttcccagtgtgtgaacctgaccaccagaacccagctgcctcctgcctacacaaattcttttacacgcggagtgtactaccctgacaaggtgttcagatcctctgtactgcatagtacacaggacctgtttctgcccttcttcagcaacgtgacctggttccacgctattcatgtttctggcaccaatggaaccaagagattcgacaatcctgtgctgccattcaacgacggcgtgtacttcgcctccacggagaagtccaacatcatcaggggctggatcttcggcaccaccctggattcaaagacccagtctctgctgatcgtgaataatgctaccaacgtggtcatcaaagtatgcgagttccagttctgcaacgacccttttctgggcgtgtactaccacaagaacaataagtcctggatggaatccgagttcagagtatactcttctgccaacaactgtaccttcgagtacgtctctcagcctttcctgatggaccttgagggcaagcagggcaacttcaaaaacctgagagagttcgtgttcaaaaacatcgacggctacttcaagatctactctaagcacacccctatcaacctggtgcgcgacctgccccaaggcttttccgctctggaacctctggtggatctgcctatcggcatcaacatcacccgcttccaaaccctgctcgctctgcaccgctcctacctgacccctggcgattcctcctccggctggaccgccggcgctgccgcctactacgtaggatatctgcagcctcggaccttcctgctgaagtacaatgagaacggcaccatcaccgacgccgtggactgcgccctggaccccctgtccgagacaaagtgcaccctaaagtccttcacagtggaaaagggaatctaccagacatccaacttcagagtgcagcctaccgagtccatcgtgagattccctaacatcaccaacctgtgtcctttcggcgaggtgttcaacgccactagattcgcctccgtgtacgcttggaaccggaagcggatcagcaattgtgttgccgactactccgtcctgtacaactccgcgagtttctccactttcaagtgctacggcgtgtcacctaccaagttgaacgatctgtgcttcactaacgtctatgccgacagctttgtgatcagaggcgacgaggtgcggcagattgcacctggccagaccggcaagatcgccgattacaactacaagctgcccgatgattttaccggctgtgtgatcgcctggaactccaacaacctggactctaaggtcggaggcaactacaactacctgtacagactgtttagaaagtctaacctgaagcccttcgagcgggacatctccactgagatctaccaggccggatccacaccttgtaacggcgtggaaggcttcaactgctacttccctctgcagtcttatggcttccagcctaccaacggcgtcggctaccagccttacagagtggtcgtcctctccttcgagctgctgcacgccccagccaccgtgtgtgggcctaagaaatcgacaaacctggtgaagaacaagtgcgtgaacttcaacttcaacggtctgaccggaaccggagttcttaccgaatccaacaagaagttcctgcctttccagcagtttggcagagacatcgccgacaccaccgatgctgtgagagatcctcagacactggaaatcctggatatcaccccatgctccttcggcggcgtgagtgtgatcacccctggcaccaacacatccaaccaagtggccgtgctgtaccaggacgtgaactgtaccgaggttcccgtggccatccacgccgaccagctgacccctacctggcgggtgtactccaccggctccaacgtgttccaaactagagccggctgcctgatcggggccgagcacgtgaacaactcttacgagtgcgacatccccatcggcgctggcatctgcgcttcctaccagacacagaccaactctcctagaggctccggctccggctctagagtgtctccctccctcgacgtggttcgattccccaacatgaccaacatctgccctttcgaccaggtgttcaacaaaacccagtttccaagcgtgtacgcctgggagagagtcagaatctctgactgtgtcagcgactacaccgtgctgtacaacagttctgcctccttctccacatttaagtgctacggcgtgtcccctacaaagctgaacgacctgtgcttctctggcgtctacgccgactacttcgtcgtgaagggcgaccacgtgcaccagatcgctccaggccagaccggcgttattgccgactacaactacaagctgccttccgagttcgtgggatgcatcctggcctggaacactcggaccatcgactccaagcggggcttctactaccggctgttccggcacggtaacatccgcccctacgagagagacacctctaatgtaccctacaatgccgccggcggcacctgtaaccagcccggtacccacaactgctacgagcctctgcaggactacggcttcacctctaccagcggagtgggctaccaaccattcagagtggtggtgctgagttttgaactgctgaacgctcctgctacagtgtgcggccctaagcagtcaaccgatctggtgaagaacaagtgcgtgaatttcggctccggatctggatctagagtggccccctctaaagaggtcgtgaggttccctaacatcaccaatctgtgccctttcggcgaagtgttcaacgctacaacgttcccctccgtttatgcctgggagagaaagcggatctccaactgcgtggctgattattccgtgctgtacaattcgacatctttctccacctttaagtgttacggcgtgtccgccaccaaactgaacgacctttgcttttccaacgtgtatgctgacagtttcgtggtgaagggcgacgatgtgcgacagatagcgcccggccagaccggcgtgatcgctgactacaactacaagctgcctgatgacttcctcggctgcgtgctggcctggaataccaactccaaggactcctcaacctctggcaactataattacctgtacagatgggtgagacggagcaaactgaacccgtacgaacgggatctttctaatgacatctactctcccggtggacagtcctgctccgccgtgggcccaaattgttacaacccactgcggccttacggcttcttcaccaccgctggcgtgggccatcagccttacagagtggtggtgctgtcctttgagctgctgaacgctcctgctaccgtgtgcggccctaaattgtctaccgacctgatcaagaaccagtgcgtgaatttcggcagcggctctggatccagagtggtgccttccggcgacgtcgttagatttccgaacatcaccaatctgtgtcctttcggcgaagtgttcaacgccaccaagttcccttctgtgtacgcttgggaacggaagaaaatctccaactgtgtggccgactactctgtgttgtataactctaccttcttttccaccttcaagtgctacggcgtgtctgccaccaagctgaacgatctctgcttctccaacgtgtacgcagactccttcgtggtgaaaggcgacgacgtcagacagatcgctcccggacagaccggcgtgatcgccgattacaactacaagctgcccgacgacttcatgggctgtgtgctggcttggaataccagaaacatcgatgccacctctactggcaactacaactacaagtaccgctacctgagacacggcaaactgcggccttttgaacgggacatctccaacgtgccattcagccctgatggcaagccttgcacgccccctgctctgaactgctactggcctctgaacgactacggattctacacaaccaccggcatcggatatcagccttaccgggtggtagtgctgagcttcgagctgctgaacgccccagctaccgtgtgcggccccaagctgtctaccgacctcatcaagaaccagtgcgtgaatttttgataataa

SEQ ID NO: 35 - DNA, 4110 bp, synthetic construct
  source 1..4110: mol_type = other DNA
atgtttgtgtttctagtgctactaccactagtcagttctcagtgcgtgaacctgacaacgagaacccagctgcctccagcctacaccaactcattcacaagaggcgtgtactaccccgacaaggtgttcagaagctctgtgctgcactccacccaggatctgttcctgcccttcttctccaacgtgacctggtttcacgccatccacgtctccggcaccaatggcaccaagcggttcgacaacccagtgctcccattcaacgacggcgtgtacttcgcctccaccgagaagtctaacatcatcagaggttggatcttcggcaccaccctggactccaagacccaatccctgctgatcgtgaacaacgccactaatgtggtcatcaaagtgtgcgagttccagttctgcaacgaccccttcctgggtgtgtactaccacaagaacaacaagtcttggatggaaagcgagttcagagtgtactcatctgctaacaattgcaccttcgagtacgtgagtcagccttttctgatggacctggaaggaaagcagggcaacttcaaaaacctgagagagttcgtgttcaaaaatatcgacggctacttcaagatctactctaagcacacccctatcaacctggtccgggatctgcctcagggctttagcgccctcgagcctctggtggacctgcccatcggcatcaacatcacccggttccagactctgttagctctgcacagatcctacctcacccccggcgattcctcttctggctggaccgctggcgctgctgcctactacgtgggctacctgcagcctcgaacgtttctgctgaagtacaacgagaacggcaccatcacagacgccgtagactgtgccctggatcctctgtctgagacaaagtgcacgctgaagtccttcaccgtggaaaagggcatctaccagacatccaacttccgggtgcagcctaccgagtctatcgtgcggttccccaacataaccaacctgtgtcctttcggcgaggtatttaacgctaccagattcgcctccgtgtacgcctggaacagaaaacggatctctaactgcgttgccgactacagtgtgctgtacaactccgcttcattcagcaccttcaagtgctacggcgtgtcccctaccaagctgaatgatctgtgcttcaccaacgtgtacgctgactctttcgtgatccggggcgacgaagtgagacagatcgcccctggccagacaggcaagatcgccgactataactacaagctgcctgacgacttcaccggatgcgtgattgcctggaactccaataacctggattctaaggtgggcggcaactacaactacctgtatcgtctgttccggaagtccaatctgaagccattcgaaagagatatttccaccgagatctaccaggctggaagcaccccttgcaacggcgtcgaaggcttcaactgctacttccccctgcagtcttacggctttcagcctaccaacggcgttggataccaaccttacagagtggtcgtgctgtcttttgagctgctgcatgctccagcaaccgtgtgcggacccaagaagtccaccaatctggtgaagaacaagtgcgtgaacttcaacttcaacggcctgacgggtaccggcgtactgacagagtccaacaaaaagtttctgcctttccagcagttcggcagagatatcgccgacaccaccgacgccgtgagagatcctcagaccctggagatcctggatatcaccccttgtagcttcggcggagtgtccgtgatcacacccggcacaaacacctctaaccaggtggccgtcctgtaccaggacgtgaactgcaccgaagtgcctgtggcaatccacgccgaccagctgacccccacctggcgggtttactctaccggcagcaacgtgtttcagaccagagctggctgcctgattggcgccgagcacgtgaacaactcgtacgagtgcgacatccctatcggagccggcatctgcgcctcctaccagacccagaccaactctcctagaggctccggatccggctctcgggtgcagcccaccgactccatcgtcagattccctaacatcaccaacctctgcccttttggagaagtgttcaacgccaccacgttcgcctctgtgtacgcttggaacagaaagcggatctccaactgcgtggcggactactctgtcctgtacaattccacctccttttccaccttcaagtgctacggcgtgtcccctacaaagctgaacgatctgtgcttcaccaatgtgtacgccgatagcttcgttatcaccggcgacgaggtgcgccagatcgctcctggccagaccggcaagatcgccgactacaactacaagttgcccgatgacttcaccggctgtgtgatcgcttggaacagcaagcacatcgatgccaaggaaggtggcaacttcaactacctgtacagactgtttagaaaggccaaccttaagccatttgagagagacatctccacagaaatctaccaggctggctccaaaccttgcaatggccagacaggcctgaactgctactatcctctgtaccgctacggcttctaccctaccgatggcgtgggccaccagccttatagagtcgtggtgctgtctttcgaactgctgaacgcccctgccaccgtgtgtggccctaagaagtctacaaacctggtgaaaaataagtgcgtgaacttcggctctggctctggctctcgggtgtccccaagcacggaggtggtccggttcccaaacatcaccaacctgtgcccttttggccaagtgttcaatgcttcaaactttccttctgtgtacgcttgggagcggctgaggatctccgactgcgtggctgattacgccgtgctctacaactctagcagctctttctctaccttcaagtgctacggcgtgtctccaaccaagctgaacgacctgtgtttctcctccgtgtacgccgactacttcgtggtgaagggcgacgacgtgagacagatcgctcctgctcagaccggagtgatcgccgattacaactacaagctgcctgatgacttcaccggctgcgtgctggcctggaacaccaactccgtggactccaagagtgggaacaacttctactacagactgtttagacatggcaagatcaagccttacgagcgcgacatctccaacgtgctgtacaactctgccggcggcacctgctcctccattagccagctgggatgttacgagcccctgaaaagctacggctttacccctactgtaggcgtcggctatcagccttaccgggttgtggtgctgtccttcgaactgctgaacgcccctgctaccgtgtgtggccctaagaaatccaccgagctggtgaagaacaagtgcgtgaacttcggctccggctccggctccagagtggcaccgtctaaagaggtggtgcggttcccaaacatcaccaacctgtgtcctttcggcgaagtcttcaacgccaccacgttccctagcgtttacgcctgggaacggaagagaatctctaactgtgtggccgactacagcgtgctgtacaactccacctcgttttccaccttcaagtgttatggcgtgtccgccaccaagttgaacgacctgtgcttcagcaatgtgtatgccgactccttcgtggtcaaaggcgacgacgtgcggcagatcgcccctggacaaactggcgtgatcgccgactataactacaaactgcctgacgatttcaccggctgtgtgctggcttggaatacccggaacatcgatgctacacagaccggcaactacaactacaagtacaggagcctgagacacggcaagcttaggcctttcgagagagacatttccaacgtgcccttctcccctgacggcaagccctgcacaccccctgcgttcaactgttactggcctctgaacgactatggcttttacatcaccaacgggatcggctatcaaccgtaccgggtggtggtgctgtcttttgagctgctgaacgcgcctgccacagtgtgcggccccaagctctccaccgacttgatcaagaaccaatgtgtgaacttctgataataa

SEQ ID NO: 36 - DNA, 2004 bp, synthetic construct
  source 1..2004: mol_type = other DNA
atgtttgtgtttctagtgctactaccactagtgtcctctcagtgcgtgaacctgatcacacggacccagtcatacaccaactccttcaccagaggagtgtactaccctgacaaggtgttcagatctagcgtgctgcattctacccaggacctgtttctgcctttcttcagcaatgtgacctggttccacgccatccacgtgtctggcacaaacggcaccaagagattcgacaaccctgtgctgcctttcaacgatggcgtgtacttcgcctctacagagaagtccaacatcatccggggctggatcttcggcaccacactggactcgaagacacaatctctgctgatcgtgaacaacgccaccaacgttgtcatcaaggtgtgcgagttccagttctgcaacgaccctttcttggacgtgtactaccacaagaacaacaagtcctggatggaatccgaattcagagtgtactccagcgccaacaactgtacattcgagtacgtcagccagccctttctgatggacctggaaggcaagcagggaaatttcaagaacctgagagagttcgtgttcaagaacatcgacggctacttcaagatctacagcaagcacacccctattaacctgggcagagatctgccccagggcttttccgctctggagcccctggtggacctgcctatcggcatcaacatcacccggtttcagaccctcctagccctgcacagatcctatctgacccctggcgacagcagttccggctggaccgccggggctgctgcctactacgtggggtacctgcagcctagaaccttcctgctgaagtacaacgagaacggcaccatcaccgacgccgtggactgtgctctggatccactgtccgagaccaagtgcactctcaagagcttcaccgtggaaaagggaatctaccagacctccaactttagagtgcagcccaccgagtcaatcgtgagattccccaacatcaccaacctgtgcccttttgacgaggtgttcaacgctaccagatttgcttctgtgtacgcctggaaccggaagagaatctctaactgcgtggccgattactccgtgctgtacaacttcgctcctttcttcgccttcaagtgttacggcgtgtcccctaccaagctgaacgacctgtgtttcactaacgtgtacgccgactctttcgtgatccggggcaacgaggtgtcccagatcgccccaggccagacaggcaacatcgctgactacaactacaagctgcctgacgatttcaccggctgtgtgatcgcctggaactccaacaagctggattccaaagtgggcggcaactacaactatctgtaccggctgttcagaaagtccaatctgaagcccttcgaacgcgacatctccaccgagatctatcaggccggcaacaagccttgtaacggcgtcgctggcttcaactgctacttccctttgcgatcttatggcttccggcctacctacggcgtgggccaccagccttaccgggtggtggtgctgagctttgagctgctgcacgctcctgctacagtgtgcggccctaagaagtccaccaatctggtgaaaaataagtgcgttaacttcaacttcaacggcctgaccggcaccggcgtgctgacagagtcaaataagaagttcctgcctttccagcagtttggcagggacatcgccgacaccacggacgctgtgcgggaccctcagaccctggaaatcctggatatcaccccttgctccttcggcggcgtgagcgtgattacccctggaaccaacacctctaatcaagtggccgtgctgtaccaaggcgtgaactgcaccgaagtgcccgtggctatccacgccgatcagctgacccctacatggcgggtatacagtaccggctctaacgtgttccagacccgggctggctgcctgatcggcgccgagtacgtgaacaactcctacgagtgcgacatccccatcggcgctggcatttgc

SEQ ID NO: 37 - DNA, 2004 bp, synthetic construct
  source 1..2004: mol_type = other DNA
atgtttgtgtttctagtgctactaccactagtgtccagccagtgcgtcaatctcatcacccgcacacaatcctacacaaactccttcaccagaggcgtgtactaccctgataaggtgttcagatcctccgtgctgcactccacccaggacctgttcctgccgttcttcagcaacgtgacctggttccacgccatccacgtgtcaggaacaaacggcaccaagagattcgacaaccccgtcctgcctttcaacgacggcgtctacttcgcctccacagagaagtctaatatcatcagaggctggatcttcggcactaccctggactccaagacacagtctctgctcatcgtgaacaacgccaccaacgtcgtcatcaaggtgtgcgagttccagttctgcaacgacccatttctggacgtgtactaccacaagaacaacaagtcctggatggaatccgagttcagagtgtactcttccgccaacaactgcaccttcgagtacgtgtctcaacctttcctgatggacctggaaggcaagcagggtaacttcaagaatctgagagagtttgtgttcaagaacatcgatggctactttaagatctacagcaagcacacccctatcaacctgggcagagatttaccccagggattctccgctctagaacccctggtggacctgcccatcggcatcaacatcacaagattccaaaccctgctggctctgcacagatcttatctgacccccggcgactcctctagcggctggaccgctggtgctgccgcctactacgtgggctacctccagcctagaaccttcctgctgaagtacaacgagaacggcaccatcaccgatgctgtggactgcgccctggatcctctgtccgaaaccaaatgtaccctgaagagcttcaccgtggaaaagggcatctaccagacaagcaactttcgggtgcagcccaccgaatccatcgtgcggttcccaaatatcaccaatctgtgtcctttcgacgaggtgttcaatgctaccagattcgcctctgtgtacgcctggaaccggaagagaatcagcaactgcgtggccgattactctgtactgtataatttcgctcccttcttcgccttcaagtgctacggagtgtcgcctacaaaacttaacgatctgtgcttcaccaacgtgtacgccgacagcttcgtgatccggggcaatgaagtgtctcagatcgccccaggacagaccggcaacatcgcggactacaactacaaactgccagacgactttaccggctgcgtgatcgcttggaactccaacaaactggacagcaaagtgggcggcaactacaactacctgtacagactgtttagaaagtctaacctgaagccctttgaaagagatatctctacagagatctaccaggctggcaacaagccttgtaatggcgttgctggcttcaactgctacttccctctgcggtcttacggcttccggcctacctacggcgtgggccaccagccttaccgggtagtcgtactcagcttcgagctgctacatgctcccgctacagtgtgcggccccaagaagtccacgaacctggtcaagaacaagtgtgtgaactttaacttcaacggcctgaccggcacgggcgtcctgaccgagtccaataaaaagttcctacccttccagcagttcggcagagatatcgctgacaccaccgacgccgtgcgggaccctcagaccctggaaatcctggacatcaccccttgctccttcggtggcgtttccgtgattacccctggcaccaacacctccaatcaagtggccgtgctgtaccagggcgtgaactgtaccgaagtgcctgtggcgatccacgccgaccagctgaccccaacctggagagtgtattctaccggatccaacgtgtttcagaccagagctggatgcctgatcggagccgagtacgtgaacaacagctacgagtgcgacattcccatcggcgccggcatctgc

SEQ ID NO: 38 - DNA, 2046 bp, synthetic construct
  source 1..2046: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccaga

SEQ ID NO: 39 - DNA, 2046 bp, synthetic construct; identical to SEQ ID NO: 38
  source 1..2046: mol_type = other DNA

SEQ ID NO: 40 - DNA, 2010 bp, synthetic construct
  source 1..2010: mol_type = other DNA
atgcagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccaga

SEQ ID NO: 41 - DNA, 2010 bp, synthetic construct; identical to SEQ ID NO: 40
  source 1..2010: mol_type = other DNA

SEQ ID NO: 42 - DNA, 2046 bp, synthetic construct
  source 1..2046: mol_type = other DNA
atgtttgtgtttctagtgctactaccactagtgtcttcccagtgcgtcaatctgaccaccagaacccaactgccccctgcttacactaactctttcaccagaggcgtgtactaccctgacaaggtgttccggtcgtccgtgctgcattctacccaggatctcttcttgcctttcttctctaacgtgacctggttccacgccatccatgtgtctggcacaaacggcaccaagagattcgacaaccctgtgctgcctttcaacgacggcgtgtatttcgcctctaccgagaagtctaacatcatccggggctggatcttcggcaccacactggacagcaagacccagtcgctgctgatcgtgaacaacgccaccaacgtggtcatcaaggtctgcgaattccagttctgcaacgatcctttcctgggcgtatactaccacaagaacaacaagtcctggatggaatccgaattcagagtatactcctccgccaataactgcacattcgagtacgtgtctcagcctttcctgatggacctggaaggcaagcagggcaacttcaaaaacctgagagagttcgtgttcaagaacatcgacggctacttcaagatctacagcaagcacacccctatcaacctggtgcgggatctgccacagggcttttccgctctggaacctctggtggacctgcccatcggcatcaatatcacccgctttcaaaccctgctggccctgcacagatcttacctgacaccaggcgactcctcctccggctggaccgccggcgctgccgcctactacgtgggctacctccagcctcggacctttctgctcaagtacaacgagaacggcacaatcaccgacgccgtggactgcgccctggaccccctgtccgagacaaagtgtaccctgaagtccttcaccgtggaaaaaggaatctatcaaacctccaactttcgtgtgcaacctactgaatctatcgtgagatttcctaatattaccaatctgtgtccttttggcgaggtgttcaacgccactcggttcgcttccgtgtacgcctggaacagaaagcggatcagcaactgcgttgctgactactccgtgctgtacaactccgcttccttcagcaccttcaagtgctatggggtctcccctaccaagctgaacgatctgtgcttcaccaacgtgtatgcagactctttcgtgatcagaggcgacgaggtcagacagatcgcccccggccagaccggaaagatcgctgactacaactacaagctgcccgacgacttcacaggctgtgtgatcgcgtggaactctaacaacctagactctaaggtaggcggcaactataactacctgtacagactgtttcggaagagcaacctgaagcctttcgagcgggacatctcgaccgagatctaccaggctggcagcacaccctgcaacggggtggaaggatttaactgctacttcccactgcagtcttatggcttccagcccacaaacggcgtgggctaccaaccttaccgggtggtggtgctgtccttcgagctgctgcacgctcctgctaccgtgtgtggcccaaagaaatctaccaacctggtgaagaacaagtgtgtcaacttcaatttcaacggcctgaccggcaccggcgtcctgaccgagtctaataagaagttcctgccctttcagcagttcggcagagatatcgccgataccaccgacgctgtgcgggacccccagacgctggaaatcctggatatcacgccttgctctttcggcggcgtgtccgtgatcacacccggcaccaacaccagcaaccaagtggctgtgttgtatcaggacgtgaactgcaccgaagtgcctgtggccatccacgccgatcagctgacccctacctggagagtgtactcaaccggcagcaacgtgttccagaccagagcgggctgcctgatcggcgccgagcacgtgaacaactcctacgagtgcgacatccctatcggagccggcatctgcgccagctaccaaacacagaccaactcccctcgg

SEQ ID NO: 43 - DNA, 2046 bp, synthetic construct
  source 1..2046: mol_type = other DNA
atgtttgtgtttctagtgctactaccactagtgtcctcccagtgtgtgaatctgacaaccagaacccagctgcctccagcctacaccaattccttcaccagaggagtctactaccctgacaaagtgttcagatcctctgtgctccactcaacacaggatctgtttctgcccttcttctccaacgtcacttggttccacgctatccatgtgtccggaacaaacggcacaaagagattcgacaaccccgtgttgcctttcaacgacggcgtgtacttcgcctctaccgagaagtccaacatcatcagaggctggatcttcggcaccacactcgattctaagacccagtctctgctgatcgttaacaacgccaccaacgtggtcatcaaggtgtgcgagttccagttctgcaacgaccccttcctgggcgtctactaccacaagaacaacaagtcttggatggaatccgagttccgtgtttacagctccgccaacaactgtaccttcgagtacgtctctcagcccttcctaatggacctggaaggtaaacagggcaatttcaagaacctgagagagttcgtgttcaagaacatcgacggatattttaagatctactctaagcacacccctatcaacctggtgcgggacctgcctcaaggcttctccgctctggagcctctagtggacctgcctattggcatcaacatcaccagattccagaccctactggctctgcacagatcttacctgactcctggcgacagctccagcggctggaccgccggcgctgccgcttactacgtgggctacctgcagcctcggaccttcctgctgaagtacaacgagaacggaaccatcaccgacgctgtggactgcgccctggatcctctgtctgaaactaagtgcaccctgaagtcgtttaccgtggaaaagggcatctaccagacctccaacttcagagtgcagccaaccgaatctatcgtgcgcttccccaacatcacaaacctgtgccccttcggcgaagtgttcaacgctacacggttcgcctccgtatacgcttggaacagaaaaagaatctccaactgtgtggctgattactccgtgctgtataacagcgcctctttttccaccttcaagtgctacggggtgtctcctaccaaactgaacgacctgtgctttaccaacgtgtacgccgactcctttgtgatccggggcgatgaggtgcggcagatcgcccctggccagaccggcaagatcgccgactataattacaagctgcctgacgacttcaccggctgcgtgatcgcatggaactccaacaatctggactccaaggtgggcggcaactacaactatctgtatagactgttcagaaagtccaacctgaaacctttcgagagagacatcagcaccgagatctaccaggccggctctaccccttgcaacggcgtagagggcttcaactgctacttccccctgcagtcctacggctttcaacctaccaacggagtgggctaccaaccttaccgggtggtggtgctgtccttcgagctgctgcacgcccctgctactgtgtgcggacctaagaaatctaccaatctggtgaaaaacaagtgcgtgaacttcaacttcaacggcctgacaggcactggcgtcctgaccgagtccaacaagaagttcctgcctttccagcagttcggcagagacatcgccgacaccaccgacgccgttagagatcctcagaccctggagatcctggacatcaccccttgctcctttggcggcgtgtccgtgatcacccctggcaccaacaccagtaatcaggtggccgtgctgtaccaggacgtgaactgcaccgaagtgcctgtggccatccacgccgaccagctgacacctacatggcgggtttactccacaggatccaacgtgttccagacccgggctggctgcctgatcggcgccgagcacgtgaacaactcctacgagtgcgacatccctatcggcgctggcatctgcgcttcttaccaaacccagaccaactccccccgc

SEQ ID NO: 44 - DNA, 2046 bp, synthetic construct
  source 1..2046: mol_type = other DNA
atgtttgtgtttctagtgctactaccactagtgtcctcccagtgcgtgaacctgaccacaagaacccagctgcctcctgcgtacaccaactccttcaccagaggcgtgtactaccctgacaaggtgtttcgttcctccgtcttgcactccacacaggatctgtttctgcctttcttctctaacgtgacgtggttccacgccatccacgtgtccggtaccaacggcacaaagagattcgacaaccccgtgctgcctttcaacgacggcgtttacttcgcctccactgagaagtctaacatcatcagaggatggatcttcggtaccaccctcgacagcaagacccaatccctgctgatagtgaacaacgctacaaacgtggtgatcaaggtgtgcgagttccagttctgcaacgatccttttctgggagtgtactaccacaagaataacaagtcctggatggaaagtgaattcagagtgtactctagcgccaacaactgtacctttgagtacgtgtcccaacctttcctgatggacctggaaggaaagcagggcaacttcaagaacctgagagagttcgtgttcaagaacatcgatggctacttcaagatctactccaagcacacacctatcaacttggttagagacctcccgcagggcttctccgctctggaacctctggtcgacctgcccattggcatcaacatcacacggttccagaccctgctggctctgcacagatcctacctgaccccaggagattcctcttctggctggaccgccggcgctgccgcttactacgtgggctacctgcagcctcggacattcctgctgaagtacaacgagaacggcaccatcaccgacgctgtggactgtgccctggatcccctgagcgagacaaagtgtaccctgaaatcttttaccgtggaaaagggcatttaccagacctccaacttcagggtgcagcctactgagtccatcgtgcggttccctaacatcaccaacctgtgccccttcggcgaagtgtttaacgccaccagatttgcttctgtgtacgcttggaacagaaagagaatcagcaactgcgtggctgattactccgtcctgtacaattcagcaagcttctccaccttcaaatgctacggagtgtctccaaccaagctgaacgacctgtgtttcaccaacgtgtacgccgactctttcgtgatccggggcgacgaagtccggcaaatcgcccccggccagaccggcaagatcgccgactacaactacaagctgcctgacgatttcaccggatgtgtcatcgcttggaactccaacaacctggatagtaaagtgggcggcaactataattatttgtaccggctgttccggaagtctaatctgaagcctttcgagagagacatctctaccgagatctaccaggccggctctacaccttgcaacggcgtagagggcttcaactgctacttccccctgcagagctacggcttccagcctactaacggcgttggctaccagccttacagagtggtggtgctgtctttcgagctgctgcatgctccagccacagtgtgcggccctaagaagtccacgaacctggtgaagaacaaatgcgtgaacttcaacttcaatggcctgaccggcaccggcgtgctgacagagtctaacaagaagttcctgccattccagcagttcggcagagatattgctgacaccactgatgccgtacgggaccctcagaccctggaaatcctggatatcaccccttgttccttcggcggcgtctccgtgatcactcctggaaccaacacctccaaccaggtggccgtgctgtaccaggacgtgaactgcaccgaggtgcctgtggccatccatgccgaccagctgacgcccacctggcgggtgtactccaccggatctaatgtgttccaaacccgggctggctgcctgatcggcgccgagcacgtgaataactcctacgagtgcgacatccctatcggggccggaatctgcgcgtcataccagacccagacaaactctcctaga

SEQ ID NO: 45 - DNA, 2046 bp, synthetic construct
  source 1..2046: mol_type = other DNA
atgtttgtgtttctagtgctactaccactagtctcttcccagtgtgtgaacctgaccaccagaacccagctgcctcctgcctacacaaattcttttacacgcggagtgtactaccctgacaaggtgttcagatcctctgtactgcatagtacacaggacctgtttctgcccttcttcagcaacgtgacctggttccacgctattcatgtttctggcaccaatggaaccaagagattcgacaatcctgtgctgccattcaacgacggcgtgtacttcgcctccacggagaagtccaacatcatcaggggctggatcttcggcaccaccctggattcaaagacccagtctctgctgatcgtgaataatgctaccaacgtggtcatcaaagtatgcgagttccagttctgcaacgacccttttctgggcgtgtactaccacaagaacaataagtcctggatggaatccgagttcagagtatactcttctgccaacaactgtaccttcgagtacgtctctcagcctttcctgatggaccttgagggcaagcagggcaacttcaaaaacctgagagagttcgtgttcaaaaacatcgacggctacttcaagatctactctaagcacacccctatcaacctggtgcgcgacctgccccaaggcttttccgctctggaacctctggtggatctgcctatcggcatcaacatcacccgcttccaaaccctgctcgctctgcaccgctcctacctgacccctggcgattcctcctccggctggaccgccggcgctgccgcctactacgtaggatatctgcagcctcggaccttcctgctgaagtacaatgagaacggcaccatcaccgacgccgtggactgcgccctggaccccctgtccgagacaaagtgcaccctaaagtccttcacagtggaaaagggaatctaccagacatccaacttcagagtgcagcctaccgagtccatcgtgagattccctaacatcaccaacctgtgtcctttcggcgaggtgttcaacgccactagattcgcctccgtgtacgcttggaaccggaagcggatcagcaattgtgttgccgactactccgtcctgtacaactccgcgagtttctccactttcaagtgctacggcgtgtcacctaccaagttgaacgatctgtgcttcactaacgtctatgccgacagctttgtgatcagaggcgacgaggtgcggcagattgcacctggccagaccggcaagatcgccgattacaactacaagctgcccgatgattttaccggctgtgtgatcgcctggaactccaacaacctggactctaaggtcggaggcaactacaactacctgtacagactgtttagaaagtctaacctgaagcccttcgagcgggacatctccactgagatctaccaggccggatccacaccttgtaacggcgtggaaggcttcaactgctacttccctctgcagtcttatggcttccagcctaccaacggcgtcggctaccagccttacagagtggtcgtcctctccttcgagctgctgcacgccccagccaccgtgtgtgggcctaagaaatcgacaaacctggtgaagaacaagtgcgtgaacttcaacttcaacggtctgaccggaaccggagttcttaccgaatccaacaagaagttcctgcctttccagcagtttggcagagacatcgccgacaccaccgatgctgtgagagatcctcagacactggaaatcctggatatcaccccatgctccttcggcggcgtgagtgtgatcacccctggcaccaacacatccaaccaagtggccgtgctgtaccaggacgtgaactgtaccgaggttcccgtggccatccacgccgaccagctgacccctacctggcgggtgtactccaccggctccaacgtgttccaaactagagccggctgcctgatcggggccgagcacgtgaacaactcttacgagtgcgacatccccatcggcgctggcatctgcgcttcctaccagacacagaccaactctcctaga

SEQ ID NO: 46 - DNA, 2046 bp, synthetic construct
  source 1..2046: mol_type = other DNA
atgtttgtgtttctagtgctactaccactagtcagttctcagtgcgtgaacctgacaacgagaacccagctgcctccagcctacaccaactcattcacaagaggcgtgtactaccccgacaaggtgttcagaagctctgtgctgcactccacccaggatctgttcctgcccttcttctccaacgtgacctggtttcacgccatccacgtctccggcaccaatggcaccaagcggttcgacaacccagtgctcccattcaacgacggcgtgtacttcgcctccaccgagaagtctaacatcatcagaggttggatcttcggcaccaccctggactccaagacccaatccctgctgatcgtgaacaacgccactaatgtggtcatcaaagtgtgcgagttccagttctgcaacgaccccttcctgggtgtgtactaccacaagaacaacaagtcttggatggaaagcgagttcagagtgtactcatctgctaacaattgcaccttcgagtacgtgagtcagccttttctgatggacctggaaggaaagcagggcaacttcaaaaacctgagagagttcgtgttcaaaaatatcgacggctacttcaagatctactctaagcacacccctatcaacctggtccgggatctgcctcagggctttagcgccctcgagcctctggtggacctgcccatcggcatcaacatcacccggttccagactctgttagctctgcacagatcctacctcacccccggcgattcctcttctggctggaccgctggcgctgctgcctactacgtgggctacctgcagcctcgaacgtttctgctgaagtacaacgagaacggcaccatcacagacgccgtagactgtgccctggatcctctgtctgagacaaagtgcacgctgaagtccttcaccgtggaaaagggcatctaccagacatccaacttccgggtgcagcctaccgagtctatcgtgcggttccccaacataaccaacctgtgtcctttcggcgaggtatttaacgctaccagattcgcctccgtgtacgcctggaacagaaaacggatctctaactgcgttgccgactacagtgtgctgtacaactccgcttcattcagcaccttcaagtgctacggcgtgtcccctaccaagctgaatgatctgtgcttcaccaacgtgtacgctgactctttcgtgatccggggcgacgaagtgagacagatcgcccctggccagacaggcaagatcgccgactataactacaagctgcctgacgacttcaccggatgcgtgattgcctggaactccaataacctggattctaaggtgggcggcaactacaactacctgtatcgtctgttccggaagtccaatctgaagccattcgaaagagatatttccaccgagatctaccaggctggaagcaccccttgcaacggcgtcgaaggcttcaactgctacttccccctgcagtcttacggctttcagcctaccaacggcgttggataccaaccttacagagtggtcgtgctgtcttttgagctgctgcatgctccagcaaccgtgtgcggacccaagaagtccaccaatctggtgaagaacaagtgcgtgaacttcaacttcaacggcctgacgggtaccggcgtactgacagagtccaacaaaaagtttctgcctttccagcagttcggcagagatatcgccgacaccaccgacgccgtgagagatcctcagaccctggagatcctggatatcaccccttgtagcttcggcggagtgtccgtgatcacacccggcacaaacacctctaaccaggtggccgtcctgtaccaggacgtgaactgcaccgaagtgcctgtggcaatccacgccgaccagctgacccccacctggcgggtttactctaccggcagcaacgtgtttcagaccagagctggctgcctgattggcgccgagcacgtgaacaactcgtacgagtgcgacatccctatcggagccggcatctgcgcctcctaccagacccagaccaactctcctaga

SEQ ID NO: 47 - DNA, 666 bp, synthetic construct
  source 1..666: mol_type = other DNA
agagtctctccaagcaccgaggtggtgcggtttcctaatatcaccaacctgtgccctttcggacaagtgtttaacgcctccaacttcccatcggtgtacgcctgggagagactgcgcatctccgactgcgtcgccgactatgccgtgctgtacaattcttcctcctctttctccacctttaagtgctacggcgtgagccctacaaagctgaacgacctgtgcttctcctctgtgtacgccgactacttcgtggtgaaaggcgatgatgtgagacagatcgcccctgcccagaccggcgtgatcgccgattacaactacaagctccccgacgacttcacaggctgcgtgctggcctggaataccaactccgtagattccaaatccggaaacaacttctactaccggctgttcagacacggcaagatcaagccctacgaaagagacatctccaacgtgctgtacaactccgctggcggcacctgttcctccatctctcagctgggctgctacgagcccctgaaatcctatgggttcacccccaccgtgggggtgggctaccagccttatagggtcgtggtgttatccttcgaactgctgaacgctccagctaccgtgtgcggccccaagaaatcaaccgagctggtcaagaacaagtgcgtgaacttc

SEQ ID NO: 48 - DNA, 666 bp, synthetic construct
  source 1..666: mol_type = other DNA
agagtggcaccctctaaagaagtggtcagattccccaatatcaccaatctgtgtccttttggcgaggtcttcaacgccaccaccttcccttctgtgtacgcctgggaacggaaacggatctctaactgcgtcgccgactactctgttctctacaactccacctctttctccacctttaaatgttacggcgtctctgctacaaaactgaacgatctgtgcttctctaacgtgtacgctgactcttttgtggtaaagggcgacgacgtgagacagatcgcccctggccaaaccggagtcatcgctgattataactacaaactgcctgatgacttcctgggatgtgtgctggcttggaacaccaatagcaaggacagcagcacatctggcaactacaactacctgtacagatgggtgaggagaagcaagctcaacccgtatgaacgggacctgtctaacgacatctactctccaggcggacagtcctgctccgctgtgggccctaactgctacaatcctctgagaccctacggcttcttcaccaccgctggcgtgggacatcagccttacagagttgttgtgctgagcttcgagctgctgaacgcccctgccaccgtgtgtggccctaagctgtctactgatctgatcaaaaaccagtgcgtgaatttc

SEQ ID NO: 49 - DNA, 615 bp, synthetic construct
  source 1..615: mol_type = other DNA
cgcgtgtcccctacccaagaggtgattcgttttcctaacatcaccaacagatgtcctttcgacaaggtgttcaatgccacccggttccctaacgtgtacgcctgggaacggacaaaaatctccgactgcgtggccgactacaccgtgctgtacaattctacatctttttctactttcaaatgctacggcgtgtccccatctaagctgatcgacctgtgttttacctctgtgtatgccgatactttcctgatccggtcctcggaggtgagacaggtcgcccctggcgagacaggggtgatcgccgactacaactacaagctgcctgacgattttaccggctgcgtgatcgcctggaataccgccaagcacgacacgggcaattactactatagatcccaccggaagaccaagctgaaacctttcgagcgcgatctgagcagcgacgacggcaacggcgtttacaccctgtccacctatgacttcaaccctaacgtacccgtggcctaccaggccacccgggtggtggtgctgtctttcgagctgctgaacgcccctgccaccgtgtgtggccctaagctgtctaccgaactggtgaagaaccagtgcgtgaacttc

SEQ ID NO: 50 - DNA, 666 bp, synthetic construct
  source 1..666: mol_type = other DNA
agagtttccccttccaccgaggtggtcagattccccaacatcacaaacctgtgcccattcggccaggtgttcaatgcttccaacttcccatctgtgtacgcctgggagcggctgaggatctctgactgcgtggccgactacgccgtgctgtacaactcttcctccagcttctctaccttcaagtgttacggcgtgtctcctaccaagctgaatgatctgtgcttcagctctgtgtacgccgactacttcgtggtgaagggtgatgatgtacggcaaatcgcccctgctcagaccggcgtgattgccgactacaattacaagctgccagacgacttcaccggctgtgtcctggcttggaacaccaactccgtggacagcaagtccggaaacaacttctactacagactgttcagacacggcaagatcaagccttacgagcgggacatctcgaacgtgctgtacaactccgccggcggaacctgtagctctatctcccagctgggctgctacgagcctctgaagagctacggcttcactcccaccgtgggcgtcggctatcagccttacagggtggttgtgctgtcctttgagctgctgaacgcccctgctaccgtgtgcggccccaagaaatccacagagctggtgaagaacaagtgcgtgaacttc

SEQ ID NO: 51 - DNA, 666 bp, synthetic construct
  source 1..666: mol_type = other DNA
cgggtggctccttctaaagaggtggtgagattccctaacatcaccaacctgtgcccttttggcgaggtcttcaacgctaccaccttcccatccgtgtacgcttgggagagaaaacggatcagcaattgcgtggctgattattccgtgctgtacaactctacctctttttccaccttcaagtgctacggcgtatctgctactaagctgaacgacctgtgcttctccaacgtgtatgctgactctttcgtggtcaagggcgacgacgtgcggcagatcgctcctggccagaccggcgtgatcgccgactataactacaagctgcctgatgacttcacaggctgtgttctggcctggaacaccagaaacatcgacgccacccagaccggcaattacaactacaagtacagatctctgcggcatggaaagctgaggcctttcgagcgggatatctccaacgtccccttttcccctgatggcaagccttgcacccctcctgctttcaactgctactggcctctgaacgactacggcttctacatcaccaacggcatcggctaccagccttatagagtggtggtgctgtctttcgagctgctgaacgctcctgccaccgtgtgtggacctaagctgtcaaccgacctgatcaagaaccagtgcgtgaacttc

SEQ ID NO: 52 - DNA, 660 bp, synthetic construct
  source 1..660: mol_type = other DNA
agagtgtctccttctctggacgtggtgaggtttccaaacatgaccaacatctgccctttcgaccaggtgttcaacaagacccagttcccttccgtctacgcttgggaacgggtgagaatctccgactgtgtgtccgactacaccgtgctgtacaacagctctgcttctttctccaccttcaagtgttacggcgtgtctcctaccaagctgaacgacctgtgcttctcaggcgtgtacgccgactacttcgtggtcaaaggcgatcacgtgcaccagatcgcccctggccagaccggcgtgatcgccgactacaattacaaactgccttccgagttcgtaggctgcatcctggcttggaacacaagaaccatcgactccaagagaggattctactaccggctgttcagacacggcaatatccggccgtacgagagagacacctccaatgtgccctacaatgctgctggaggcacctgcaaccagcctggcacccacaactgctacgagcctctgcaggactacggcttcacatccacatcgggagtgggctaccagccattccgcgtggtggtcttatcctttgagctgctgaacgcccctgccaccgtgtgtggccccaagcagagcaccgatctagtgaaaaacaagtgcgtcaatttc

SEQ ID NO: 53 - DNA, 666 bp, synthetic construct
  source 1..666: mol_type = other DNA
agagtggctccctctaaagaggtggtgagattccccaacattaccaacctgtgcccttttggagaggtcttcaatgctaccaccttccctagcgtatacgcttgggagcggaagcggatctccaactgcgtggccgactactctgtgctgtacaactccaccagcttttctaccttcaaatgctacggcgtgtccgccaccaagctgaacgatctgtgcttttccaacgtgtacgccgattccttcgtggttaagggcgacgatgtgagacagatcgcccctggccagacaggtgtaatcgctgattacaactacaagctgcctgacgacttcaccggctgtgtgctggcctggaacaccagaaacatcgatgctacacagaccggcaactacaactacaagtaccggagcctgcggcatggcaagctgagacctttcgagagagatatctccaacgtgccattctctcccgatggcaaaccttgtaccccacctgctttcaactgctactggcccctgaacgactacggcttctatatcactaacggcatcggctaccagccatacagagtggtggttctgagtttcgaactgctcaatgctccagccaccgtgtgcggacctaagctttccaccgacctgataaaaaaccagtgtgtgaacttc

SEQ ID NO: 54 - DNA, 669 bp, synthetic construct
  source 1..669: mol_type = other DNA
cgggtgcaacctaccatctccatcgtgcggtttcctaacatcaccaacctgtgccctttcggcgaggtcttcaacgccagcaagttcgcttccgtgtatgcctggaacagaaagagaatctccaactgcgtcgccgactactccgtgctgtacaattctacaagtttctccaccttcaagtgctacggcgtgtcccctaccaagctgaacgacctgtgcttcaccaacgtctacgccgactccttcgtggtgaagggcgatgaagtgagacagatcgctccaggacagacaggcgtaattgccgactataattataaactccccgatgacttcaccggctgcgtgattgcctggaactctgtcaagcaggacgctttgaccggcggaaactacggctacctgtaccggctgttcagaaagtccaagctgaaaccttttgaacgggacatctctaccgaaatctaccaggctgggagcaccccttgtaacggacaagtgggcctgaactgttactaccccctggaaagatacggcttccatcctaccaccggcgtgaattaccagcctttccgggttgtggttctgagctttgagctgctgaacggcccagctacggtgtgcggccccaagctctctaccacccttgtgaaggacaagtgcgtcaatttc

SEQ ID NO: 55 - DNA, 666 bp, synthetic construct
  source 1..666: mol_type = other DNA
agagtggctccttccaaagaggtggtgagatttcctaatatcaccaacctgtgtcctttcggcgaagtgttcaacgccaccaccttcccatctgtgtacgcctgggagagaaagcggatcagcaactgtgtggctgattattctgtgctgtacaactccacctccttctccacctttaagtgttacggcgtgtctgctaccaagctgaacgatctctgttttagtaacgtgtacgccgactccttcgtggtgaagggcgacgatgtgcggcagatcgcccctggccagaccggcgtgatcgctgattacaactacaagctgcctgacgactttacaggctgtgtgctggcttggaacacccggaacatcgatgccacccagaccggcaactacaactacaagtaccgctctctgcggcacggcaagctgaggccctttgagcgggatatctctaacgtgcctttcagccctgatggcaagccctgcacccctccagccttcaattgttactggcctctgaacgactacggcttctacatcaccaacggaatcggctaccagccctatagagtggtggtgctgtcgttcgagctgctgaatgctcccgccaccgtgtgtggcccaaagctgtccaccgacctgatcaagaaccagtgcgtgaacttc

SEQ ID NO: 56 - DNA, 660 bp, synthetic construct
  source 1..660: mol_type = other DNA
agagtttcgccttctctggacgtggtcagattccctaacatgaccaacatctgccctttcgaccaggtgttcaacaaaacccagttcccctccgtgtacgcctgggaacgggtgagaatttccgactgcgtctcagattacaccgtgctctataacagctctgcctctttctctacctttaagtgctacggcgtgagccctaccaagttaaatgacctgtgctttagcggcgtgtacgccgattatttcgtggtaaagggtgaccacgtgcaccagatagcccccggccagaccggcgtgatcgctgactacaattataagctgcccagcgagttcgtgggctgcatcctggcctggaacactagaacaatcgactccaagagaggcttctactaccggctgttccggcatggcaacatcagaccttacgagcgcgatacctccaatgtgccctacaacgctgctggcgggacctgcaaccagcctggcacccacaactgctacgagcctctgcaggactacggcttcacctccacatctggcgtcggctaccaaccattcagagtggtggtgctgtctttcgagctgctgaacgctccagctaccgtgtgtggccctaagcaatccacagatctggtgaagaacaagtgtgtgaatttc

SEQ ID NO: 57 - DNA, 666 bp, synthetic construct
  source 1..666: mol_type = other DNA
agagtggctccttctaaagaggttgtgcggtttcctaacatcaccaacctgtgtcctttcggcgaggtgttcaacgccaccacgttcccctctgtgtacgcctgggagagaaagaggatctccaactgtgttgctgactactcggtgctgtataactccacctctttttccaccttcaagtgctacggcgtctccgccaccaagctcaatgacctgtgcttctcgaacgtgtacgcagactccttcgtggtcaagggcgacgacgtgagacagatcgcccctggccagactggcgtgatcgccgactataattacaaactgccggacgactttctgggctgcgtgctggcttggaacaccaactctaaggactcttccacctctggcaactacaactacctgtatagatgggtgcgtaggagtaaactcaacccttacgagcgggacctgtccaacgatatctattcccctggcggccagtcctgctccgctgtgggccccaattgctacaaccccctgcggccttacggcttcttcacaaccgctggagtgggccaccagccttaccgggtggtggtgctgtcttttgaactgctgaatgctcctgctaccgtgtgcggccccaagctgtccaccgacctgataaaaaaccagtgtgtgaacttc

SEQ ID NO: 58 - DNA, 666 bp, synthetic construct
  source 1..666: mol_type = other DNA
agagtcgcccctagtaaagaagtggtgagattccccaacatcaccaacctgtgccctttcggcgaagtgttcaacgccaccaccttcccttctgtgtacgcttgggagcggaagcggatctccaactgtgtggccgactatagcgtgttatacaactctacctctttttccaccttcaagtgctatggcgtgtctgccaccaaactgaacgacctgtgcttctcgaacgtgtacgctgatagctttgtggtgaaaggcgacgacgtgagacagatcgcacctggacagaccggagtgatcgccgactataactacaagcttccagatgacttcaccggctgtgtgctggcttggaacacccggaacatcgacgccacccagacaggaaactacaactacaagtaccgctccctgagacacggcaagctgagacccttcgagcgggacatctccaacgtgcctttctctcctgatggcaagccctgcacccctccagccttcaactgttactggcctctgaacgactacggcttttacatcaccaatggcatcggatatcagccttacagagtggtggtcctgtcctttgagctgctgaacgcccctgccaccgtgtgcggccctaagctgtccaccgacctgatcaagaaccagtgcgtgaacttt

SEQ ID NO: 59 - DNA, 660 bp, synthetic construct
  source 1..660: mol_type = other DNA
agagtgtctccctccctcgacgtggttcgattccccaacatgaccaacatctgccctttcgaccaggtgttcaacaaaacccagtttccaagcgtgtacgcctgggagagagtcagaatctctgactgtgtcagcgactacaccgtgctgtacaacagttctgcctccttctccacatttaagtgctacggcgtgtcccctacaaagctgaacgacctgtgcttctctggcgtctacgccgactacttcgtcgtgaagggcgaccacgtgcaccagatcgctccaggccagaccggcgttattgccgactacaactacaagctgccttccgagttcgtgggatgcatcctggcctggaacactcggaccatcgactccaagcggggcttctactaccggctgttccggcacggtaacatccgcccctacgagagagacacctctaatgtaccctacaatgccgccggcggcacctgtaaccagcccggtacccacaactgctacgagcctctgcaggactacggcttcacctctaccagcggagtgggctaccaaccattcagagtggtggtgctgagttttgaactgctgaacgctcctgctacagtgtgcggccctaagcagtcaaccgatctggtgaagaacaagtgcgtgaatttc

SEQ ID NO: 60 - DNA, 666 bp, synthetic construct
  source 1..666: mol_type = other DNA
agagtggccccctctaaagaggtcgtgaggttccctaacatcaccaatctgtgccctttcggcgaagtgttcaacgctacaacgttcccctccgtttatgcctgggagagaaagcggatctccaactgcgtggctgattattccgtgctgtacaattcgacatctttctccacctttaagtgttacggcgtgtccgccaccaaactgaacgacctttgcttttccaacgtgtatgctgacagtttcgtggtgaagggcgacgatgtgcgacagatagcgcccggccagaccggcgtgatcgctgactacaactacaagctgcctgatgacttcctcggctgcgtgctggcctggaataccaactccaaggactcctcaacctctggcaactataattacctgtacagatgggtgagacggagcaaactgaacccgtacgaacgggatctttctaatgacatctactctcccggtggacagtcctgctccgccgtgggcccaaattgttacaacccactgcggccttacggcttcttcaccaccgctggcgtgggccatcagccttacagagtggtggtgctgtcctttgagctgctgaacgctcctgctaccgtgtgcggccctaaattgtctaccgacctgatcaagaaccagtgcgtgaatttc

SEQ ID NO: 61 - DNA, 669 bp, synthetic construct
  source 1..669: mol_type = other DNA
agagtggtgccttccggcgacgtcgttagatttccgaacatcaccaatctgtgtcctttcggcgaagtgttcaacgccaccaagttcccttctgtgtacgcttgggaacggaagaaaatctccaactgtgtggccgactactctgtgttgtataactctaccttcttttccaccttcaagtgctacggcgtgtctgccaccaagctgaacgatctctgcttctccaacgtgtacgcagactccttcgtggtgaaaggcgacgacgtcagacagatcgctcccggacagaccggcgtgatcgccgattacaactacaagctgcccgacgacttcatgggctgtgtgctggcttggaataccagaaacatcgatgccacctctactggcaactacaactacaagtaccgctacctgagacacggcaaactgcggccttttgaacgggacatctccaacgtgccattcagccctgatggcaagccttgcacgccccctgctctgaactgctactggcctctgaacgactacggattctacacaaccaccggcatcggatatcagccttaccgggtggtagtgctgagcttcgagctgctgaacgccccagctaccgtgtgcggccccaagctgtctaccgacctcatcaagaaccagtgcgtgaatttttga

SEQ ID NO: 62 - DNA, 669 bp, synthetic construct
  source 1..669: mol_type = other DNA
cgggtgcagcccaccgactccatcgtcagattccctaacatcaccaacctctgcccttttggagaagtgttcaacgccaccacgttcgcctctgtgtacgcttggaacagaaagcggatctccaactgcgtggcggactactctgtcctgtacaattccacctccttttccaccttcaagtgctacggcgtgtcccctacaaagctgaacgatctgtgcttcaccaatgtgtacgccgatagcttcgttatcaccggcgacgaggtgcgccagatcgctcctggccagaccggcaagatcgccgactacaactacaagttgcccgatgacttcaccggctgtgtgatcgcttggaacagcaagcacatcgatgccaaggaaggtggcaacttcaactacctgtacagactgtttagaaaggccaaccttaagccatttgagagagacatctccacagaaatctaccaggctggctccaaaccttgcaatggccagacaggcctgaactgctactatcctctgtaccgctacggcttctaccctaccgatggcgtgggccaccagccttatagagtcgtggtgctgtctttcgaactgctgaacgcccctgccaccgtgtgtggccctaagaagtctacaaacctggtgaaaaataagtgcgtgaacttc

SEQ ID NO: 63 - DNA, 666 bp, synthetic construct
  source 1..666: mol_type = other DNA
cgggtgtccccaagcacggaggtggtccggttcccaaacatcaccaacctgtgcccttttggccaagtgttcaatgcttcaaactttccttctgtgtacgcttgggagcggctgaggatctccgactgcgtggctgattacgccgtgctctacaactctagcagctctttctctaccttcaagtgctacggcgtgtctccaaccaagctgaacgacctgtgtttctcctccgtgtacgccgactacttcgtggtgaagggcgacgacgtgagacagatcgctcctgctcagaccggagtgatcgccgattacaactacaagctgcctgatgacttcaccggctgcgtgctggcctggaacaccaactccgtggactccaagagtgggaacaacttctactacagactgtttagacatggcaagatcaagccttacgagcgcgacatctccaacgtgctgtacaactctgccggcggcacctgctcctccattagccagctgggatgttacgagcccctgaaaagctacggctttacccctactgtaggcgtcggctatcagccttaccgggttgtggtgctgtccttcgaactgctgaacgcccctgctaccgtgtgtggccctaagaaatccaccgagctggtgaagaacaagtgcgtgaacttc

SEQ ID NO: 64 - DNA, 666 bp, synthetic construct
  source 1..666: mol_type = other DNA
agagtggcaccgtctaaagaggtggtgcggttcccaaacatcaccaacctgtgtcctttcggcgaagtcttcaacgccaccacgttccctagcgtttacgcctgggaacggaagagaatctctaactgtgtggccgactacagcgtgctgtacaactccacctcgttttccaccttcaagtgttatggcgtgtccgccaccaagttgaacgacctgtgcttcagcaatgtgtatgccgactccttcgtggtcaaaggcgacgacgtgcggcagatcgcccctggacaaactggcgtgatcgccgactataactacaaactgcctgacgatttcaccggctgtgtgctggcttggaatacccggaacatcgatgctacacagaccggcaactacaactacaagtacaggagcctgagacacggcaagcttaggcctttcgagagagacatttccaacgtgcccttctcccctgacggcaagccctgcacaccccctgcgttcaactgttactggcctctgaacgactatggcttttacatcaccaacgggatcggctatcaaccgtaccgggtggtggtgctgtcttttgagctgctgaacgcgcctgccacagtgtgcggccccaagctctccaccgacttgatcaagaaccaatgtgtgaacttc

SEQ ID NO: 65 - DNA, 681 bp, synthetic construct
  source 1..681: mol_type = other DNA
cgagtccaaccaacagagtcaatcgtaagattccccaatatcaccaacctctgcccctttgacgaagttttcaacgctaccagatttgcaagtgtgtacgcctggaataggaagcggatcagtaactgcgtcgccgactactctgtactttataacctcgcgcccttctttacattcaagtgctatggcgtctcccccacgaaacttaatgacctctgtttcacgaacgtctacgccgattcctttgtgataagaggggatgaggtaagacaaatagcacctggtcagacaggcaacatagcagactataactataagttgcccgacgactttacaggatgtgtaattgcttggaacagtaacaagctggattcaaaggtttccggcaattataactacctgtacagactgttcagaaaaagtaatctcaaacccttcgagagagacatttcaaccgaaatctatcaggccgggaacaaaccatgcaacggcgtcgctggattcaactgttacttccccctccgatcatactcctttaggcccacatatggagtgggtcatcaaccttaccgagttgttgtcctgtctttcgagctcttgcacgcgccggcaactgtgtgtggacctaaaaagagtacgaacttggtgaagaataagtgtgtaaactttaactttaatggt

SEQ ID NO: 66 - DNA, 708 bp, synthetic construct
  source 1..708: mol_type = other DNA
atgttcgtgtttctggtgctgctgcctctggtgtccagccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactatagtgtactgtataactcagcatccttttccacgtttaagtgctacggggtcagtcctacaaagttgaatgacttgtgctttacaaacgtatatgctgattcctttgtcatacgaggcgatgaggtcagacagattgctccgggacaaaccggaaatattgccgactacaactacaaacttcccgacgattttactgggtgtgtaattgcctggaacagtaacaatcttgactcaaaagtggggggaaattacaattacctttatagactcttccgcaagagcaatctcaagccttttgaacgcgatatatctaccgaaatataccaggccggttctactccatgcaatggagttaagggatttaattgttactttcctctccaatcttatgggttccagccaacttatggagtcggttatcagccatatcgagtcgttgtgctcagcttcgaactgcttcatgccccagctactgtttgtgggccgaaaaagtccactaatctcgtgaagaataagtgcgtaaacttt

SEQ ID NO: 67 - DNA, 681 bp, synthetic construct
  source 1..681: mol_type = other DNA
agggttcagcccacagagtccatagtacggttccctaatattaccaacctgtgtccgttcggtgaagtcttcaatgccacaagattcgccagcgtttatgcctggaaccgaaagagaattagtaattgtgttgcggattattctgtcctttacaatagtgcaagcttctccaccttcaagtgttacggggtttccccgacgaaactcaacgacctttgttttacaaatgtgtatgcagattcatttgttatccgaggagatgaggtacggcaaatagcccccggacaaacaggaaagatagccgattacaactataaactgcctgatgacttcactgggtgcgtcatcgcatggaatagcaacaaccttgattccaaagtagggggcaattataattaccgctatcgacttttccgcaagtccaacctcaaacccttcgagcgcgatatcagcacggaaatttaccaagcgggcagcaaaccatgcaacggagtcgaaggatttaattgttacttcccgttgcaatcctatggctttcaaccaaccaacggtgttggttaccaaccttatagagttgtcgttctcagttttgagctcctccatgccccggccaccgtatgcggcccaaagaaatcaacaaacctcgttaagaacaaatgtgtcaactttaatttcaatggg

(19) United States
(12) Patent Application Publication
(10) Pub. No.: US 2026/0240986 A1
(43) Pub. Date: Aug. 20, 2026

(54) FUSION POLYPEPTIDES, IMMUNOGENIC COMPOSITIONS, METHODS AND USES THEREOF

(71) Applicant: UNIVERSITY OF SASKATCHEWAN, Saskatoon (CA)

(72) Inventors: Arinjay Banerjee, Saskatchewan (CA); Darryl Falzarano; Qiang Liu; Alyson Ann Kelvin

(21) Appl. No.: 19/164,334

(22) PCT Filed: Mar. 18, 2024
(86) PCT No.: PCT/CA2024/050325

§ 371 Date: Sep. 11, 2025

Related U.S. Application Data
(60) Provisional application No. 63/452,586, filed on Mar. 16, 2023.

(51) Int. Cl. A61K 39/215; A61K 39/00; A61K 39/295; A61K 39/39; A61P 31/14; C07K 14/165; C07K 14/37; C12N 1/16; C12N 1/20; C12N 5/04; C12N 5/07; C12N 5/071; C12N 15/62

(52) U.S. Cl. CPC A61K 39/215; A61K 39/295; A61K 39/39; A61P 31/14; C07K 14/165; C07K 14/37; C12N 1/16; C12N 1/20; C12N 5/04; C12N 5/0601; C12N 5/0602; C12N 15/62; A61K 2039/55566; C07K 2319/03; C12N 2510/00

(57) ABSTRACT

Fusion polypeptides, and their use in subunit vaccine compositions to elicit immune responses against two or more pathogens are described, as well as polynucleotides encoding therefor. Also described are methods for treating and preventing infection by two or more pathogens.

### TECHNICAL FIELD

The present invention generally relates to pathogens. In particular, the invention pertains to fusion polypeptides, immunogenic compositions comprising fusion polypeptides and methods of simultaneously treating and/or preventing one or more pathogen-related condition or pathogen infection.

### BACKGROUND

The emergence of new pathogens and new variants of existing pathogens with enhanced transmission or with the potential to circumvent vaccine elicited immunity are significant barriers to ending pandemics, such as that of SARS-CoV-2, as well as the prevention of future outbreaks. Further concerning is the significant potential for new pathogens, such as for example, Coronaviridae, to spill-over from an animal reservoir and initiating an entirely new pandemic. Broadly protective or pan-pathogen vaccines that can protect against several bacterial, fungal, and/or viral species, such as Betacoronaviruses, at once offers a solution to the continual emergence of new pathogens and/or new pathogen variants.

Antigen design and the time needed for vaccine production are two constraints for the development and implementation of vaccines for the treatment and/or prevention of infectious disease. Current vaccine strategies for the treatment and prevention of viruses, for example, require providing a vaccine to match circulating strains to keep up with new and potentially detrimental emerging variants. This strategy is limited in that it only targets a single viral variant, which may result in diminished or a lack of protection across a broad range of viral variants and/or species. Furthermore, the significant time required for production of many current vaccine formulations precludes the ability to rapidly produce new formulations targeting any new or potential variants. Additionally, each vaccine is produced separately which represents a significant time and financial burden on the manufacturer and/or developer. Additionally, delays in vaccine procurement in low- and middle-income countries may be further exacerbated with conventional approaches, contributing to an increased vaccine disparity.

Subunit vaccines, which are vaccine compositions that include one or more selected antigen but not all antigens, derived from or homologous to, an antigen from a pathogen of interest, provides a system amenable to more rapid and cost effective vaccine manufacturing, however, the use of fewer potentially antigenic proteins has limited the efficacy of these subunit vaccines in comparison to their traditional vaccine counterpart.

Multivalent vaccines are those which illicit an immune response to two or more antigens, however, current multivalent vaccines are limited in their efficacy in the neutralization of two or more antigens to prevent illness, as well as their amenability to rapid production and/or modification.

Current multivalent vaccines such as those for the treatment and/or prevention of Diphtheria, Tetanus, and/or Pertussis comprise separately prepared pathogenic components, such as inactivated toxins, which are combined to produce a final immunogenic composition.

Bivalent vaccines targeting COVID-19 inducing SARS-CoV-2 virus and variants thereof comprise separately prepared mRNA directed against ancestral SARS-CoV-2 virus and against the BA.4/BA.5 variants. The use of said bivalent vaccines for the prevention of COVID-19 resulted in no significant differences in the ability to neutralize any SARS-CoV-2 variant from monovalent vaccines targeting the ancestral strain in which the variants were derived.

The ability to provide two or more antigens and a delivery system thereof that facilitate the neutralization of multiple pathogens and/or pathogen variants in a single composition is required. Specifically, there is a need to develop broadly protective, rapidly editable, low cost, and temperature stable vaccines that will protect against current and emerging viruses, such as coronaviruses, and a platform for the preparation thereof.

### SUMMARY OF THE INVENTION

In certain embodiments herein there is provided a fusion polypeptide that may comprise an amino acid sequence encoding, in sequence, a first antigen, a linker, and at least one additional antigen.

In certain embodiments, the at least one additional antigen may comprise a plurality of antigens and each antigen of the plurality of antigens may be linked to the preceding antigen with a linking peptide comprising the linker.

In certain embodiments, the sequence of the first antigen may comprise a greater number of amino acids than the sequence of at least one of the at least one or more antigen.

In certain embodiments, the first antigen may comprise an antigenic peptide or fragment thereof from a first pathogen.

In certain embodiments, the first antigen may comprise a first prokaryotic antigen, a first viral antigen, or a first fungal antigen.

In certain embodiments, the first viral antigen may comprise a component of a surface glycoprotein from a virus, a lipoprotein or an extracellular domain of a transmembrane protein.

In certain embodiments, the first viral antigen may comprise a first coronavirus antigen comprising a MERS-CoV antigen, a SARS-CoV-1 antigen, a SARS-CoV-2 antigen (for example but not limited to a Wuhan coronavirus antigen, a BA.1 coronavirus antigen, a BA.2 coronavirus antigen, or a B.1.351 coronavirus antigen), a HCoV-HKU1 antigen, a HCoV-OC43 antigen, a BtCoV-RaTG13 antigen, a Bt SL-CoV-WIV1 antigen, a HKU4-CoV antigen, a HKU5-CoV antigen, a PDF-2108 CoV antigen, a SHC014-CoV antigen, a BISCoV-RsSHC014 antigen, a BISCoV-Rs3367 antigen, a BANAL-52 antigen, a BANAL-236 antigen, a Pangolin-CoV GX/P2V antigen, a Bat coronavirus CDPHE15 antigen, a Bat coronavirus HKU10 antigen, a Rhinolophus ferrumequinum alphacoronavirus HuB-2013 antigen, a Human coronavirus 229E antigen, a Lucheng Rn rat coronavirus antigen, a Mink coronavirus 1 antigen, a Ferret coronavirus antigen, a Miniopterus bat coronavirus 1 antigen, a Miniopterus bat coronavirus HKU8 antigen, a Myotis ricketti alphacoronavirus Sax-2011 antigen, a Nyctalus velutinus alphacoronavirus SC-2013 antigen, a Pipistrellus kuhlii coronavirus 3398 antigen, a Porcine epidemic diarrhea virus antigen, a Scotophilus bat coronavirus 512 antigen, a Rhinolophus bat coronavirus HKU2 antigen, a Human coronavirus NL63 antigen, a NL63-related bat coronavirus strain BtKYNL63-9b antigen, a Sorex araneus coronavirus T14 antigen, a Suncus murinus coronavirus X74 antigen, a Alphacoronavirus 1 antigen, a Canine coronavirus antigen, a Feline coronavirus antigen, a Human CCoV-HuPn-2018 antigen, a Porcine transmissible gastroenteritis coronavirus antigen, a Swine acute diarrhea syndrome coronavirus (SADS-CoV) antigen, a HCoV-NL63 antigen, a Khosta2 sarbecovirus antigen, a Kenya bat coronavirus BtKY72 antigen, a bat SARS coronavirus HKU3 antigen, a NL63-related bat coronavirus strain BtKYNL63-9b antigen, a HCoV-229E antigen or any combination thereof.

In certain embodiments, the first coronavirus antigen may comprise a spike (S) protein, or fragment thereof. In certain embodiments, the first coronavirus antigen may comprise a SARS-CoV-2 spike protein or a protein encoded by a nucleotide sequence with at least 70% identity to the SARS-CoV-2 spike protein. In certain embodiments, the first coronavirus antigen may comprise an S1 protein, or fragment thereof. In certain embodiments, the first coronavirus antigen may comprise a SARS-CoV-2 S1 protein or a protein encoded by a nucleotide sequence with at least 70% identity to the SARS-CoV-2 S1 protein.

In certain embodiments, the linker sequence may comprise 6 to 24 amino acids. In certain embodiments, the linker sequence may comprise glycine (G), serine(S), alanine (A), or any combination thereof. In certain embodiments, an amino acid sequence of the linker may comprise GSGSGS or GAAASG.

In certain embodiments, the at least one additional antigen may comprise at least one additional antigenic peptide or fragment thereof from at least one additional pathogen.

In certain embodiments, the at least one additional antigen may comprise an amino acid sequence of at least 8 amino acids.

In certain embodiments, the at least one additional antigen protein may comprise at least one additional prokaryotic antigen, at least one additional viral antigen, at least one additional fungal antigen, or any combination thereof.

In certain embodiments, the at least one additional viral antigen may comprise at least one coronavirus antigen, wherein the at least one coronavirus antigen is a Coronaviridae antigen.

In certain embodiments, the at least one coronavirus antigen may comprise a MERS-CoV antigen, a SARS-CoV-1 antigen, a SARS-CoV-2 antigen (for example but not limited to a Wuhan coronavirus antigen, a BA. 1 coronavirus antigen, a BA.2 coronavirus antigen, or a B.1.351 coronavirus antigen), a HCoV-HKU1 antigen, a HCoV-OC43 antigen, a BtCoV-RaTG13 antigen, a Bt SL-CoV-WIV1 antigen, a HKU4-CoV antigen, a HKU5-CoV antigen, a PDF-2108 CoV antigen, a SHC014-CoV antigen, a BtSCoV-RsSHC014 antigen, a BISCoV-Rs3367 antigen, a BANAL-52 antigen, a BANAL-236 antigen, a Pangolin-CoV GX/P2V antigen, a Bat coronavirus CDPHE15 antigen, a Bat coronavirus HKU10 antigen, a Rhinolophus ferrumequinum alphacoronavirus HuB-2013 antigen, a Human coronavirus 229E antigen, a Lucheng Rn rat coronavirus antigen, a Mink coronavirus 1 antigen, a Ferret coronavirus antigen, a Miniopterus bat coronavirus 1 antigen, a Miniopterus bat coronavirus HKU8 antigen, a Myotis ricketti alphacoronavirus Sax-2011 antigen, a Nyctalus velutinus alphacoronavirus SC-2013 antigen, a Pipistrellus kuhlii coronavirus 3398 antigen, a Porcine epidemic diarrhea virus antigen, a Scotophilus bat coronavirus 512 antigen, a Rhinolophus bat coronavirus HKU2 antigen, a Human coronavirus NL63 antigen, a NL63-related bat coronavirus strain BtKYNL63-9b antigen, a Sorex araneus coronavirus T14 antigen, a Suncus murinus coronavirus X74 antigen, a Alphacoronavirus 1 antigen, a Canine coronavirus antigen, a Feline coronavirus antigen, a Human CCoV-HuPn-2018 antigen, a Porcine transmissible gastroenteritis coronavirus antigen, a Swine acute diarrhea syndrome coronavirus (SADS-CoV) antigen, a HCoV-NL63 antigen. a NL63-related bat coronavirus strain BtKYNL63-9b antigen, a HCoV-229E antigen, a Khosta2 sarbecovirus antigen, a Kenya bat coronavirus BtKY72 antigen, a bat SARS coronavirus HKU3 antigen or any combination thereof.

In certain embodiments, the at least one coronavirus antigen may comprise a receptor-binding domain, fragment thereof, or a protein encoded by a nucleotide with at least 70% identity to a Coronaviridae receptor binding domain.

In certain embodiments, the first antigen and the at least one additional antigen may each independently comprise a prokaryotic antigen, a viral antigen, or a fungal antigen.

In certain embodiments the fusion polypeptide may further comprise, in sequence, a third antigen and the linker sequence upstream of the first antigen.

In certain embodiments, the plurality of antigens may comprise prokaryotic antigens, viral antigens, fungal antigens, or any combination thereof.

In certain embodiments, the viral antigens may comprise coronavirus antigens.

In certain embodiments, the coronavirus antigens may comprise MERS-CoV antigens, SARS-CoV-1 antigens, SARS-CoV-2 antigens (for example but not limited to Wuhan coronavirus antigens, BA. 1 coronavirus antigens, BA.2 coronavirus antigens, or B.1.351 coronavirus antigens), HCoV-HKU1 antigens, HCoV-OC43 antigens, BtCoV-RaTG13 antigens, Bt SL-CoV-WIV1 antigens, HKU4-CoV antigens, HKU5-CoV antigens, PDF-2108 CoV antigens, SHC014-CoV antigens, BtSCoV-RsSHC014 antigens, BtSCoV-Rs3367 antigens, BANAL-52 antigens, BANAL-236 antigens, Pangolin-CoV GX/P2V antigens, Bat coronavirus CDPHE15 antigens, Bat coronavirus HKU10 antigens, Rhinolophus ferrumequinum alphacoronavirus HuB-2013 antigens, Human coronavirus 229E antigens, Lucheng Rn rat coronavirus antigens, Mink coronavirus 1 antigens, Ferret coronavirus antigens, Miniopterus bat coronavirus 1 antigens, Miniopterus bat coronavirus HKU8 antigens, Myotis ricketti alphacoronavirus Sax-2011 antigens, Nyctalus velutinus alphacoronavirus SC-2013 antigens, Pipistrellus kuhlii coronavirus 3398 antigens, Porcine epidemic diarrhea virus antigens, Scotophilus bat coronavirus 512 antigens, Rhinolophus bat coronavirus HKU2 antigens, Human coronavirus NL63 antigens, NL63-related bat coronavirus strain BtKYNL63-9b antigens, Sorex araneus coronavirus T14 antigens, Suncus murinus coronavirus X74 antigens, Alphacoronavirus 1 antigens, Canine coronavirus antigens, Feline coronavirus antigens, Human CCOV-HuPn-2018 antigens, Porcine transmissible gastroenteritis coronavirus antigens, Swine acute diarrhea syndrome coronavirus (SADS-CoV) antigens, HCoV-NL63 antigens. NL63-related bat coronavirus strain BtKYNL63-9b antigens, HCoV-229E antigens, Khosta2 sarbecovirus antigens, Kenya bat coronavirus BtKY72 antigens, bat SARS coronavirus HKU3 antigens, or any combination thereof.

In certain embodiments, the coronavirus antigen may comprise a receptor-binding domain, fragment thereof, or a protein encoded by a nucleotide with at least 70% identity to a Coronaviridae receptor binding domain.

In certain embodiments, the first antigen, and the at least one additional antigen, each may independently comprise a prokaryotic antigen, a viral antigen, or a fungal antigen.

In certain embodiments there is provided a fusion polypeptide that may comprise an amino acid sequence encoding a plurality of antigens in series, wherein each of the plurality of antigens is linked with a linker sequence to the preceding antigen.

In certain embodiments, the plurality of antigens may comprise two or more antigens.

In certain embodiments, each of the plurality of antigens may independently comprise a prokaryotic antigen, a viral antigen, or a fungal antigen.

In certain embodiments, the linker sequence may comprise 6 to 24 amino acids. In certain embodiments, the linker sequence may comprise glycine (G), serine(S), alanine (D), or any combination thereof.

In certain embodiments, the fusion polypeptide may comprise a Wuhan coronavirus antigen and a B.1.617.2 coronavirus antigen.

In certain embodiments, the fusion polypeptide may comprise a Wuhan coronavirus antigen and a BA.2 coronavirus antigen.

In certain embodiments, the fusion polypeptide may comprise a Wuhan coronavirus antigen and a BA. 1 coronavirus antigen.

In certain embodiments, the fusion polypeptide may comprise a B.1.351 coronavirus antigen, a Wuhan coronavirus antigen and a BA.2 coronavirus antigen.

In certain embodiments, the fusion polypeptide may comprise a B.1.351 coronavirus antigen, a Wuhan coronavirus antigen and a BA. 1 coronavirus antigen.

In certain embodiments, the fusion polypeptide may comprise a Wuhan coronavirus antigen, a Khosta2 sarbecovirus antigen and a bat SARS-like coronavirus WIV1 antigen.

In certain embodiments, the fusion polypeptide may comprise a BA.2 coronavirus antigen, a Kenya bat coronavirus BtKY72 antigen and a BtSCoV-RsSHC014 antigen.

In certain embodiments, the fusion polypeptide may comprise a Wuhan coronavirus antigen, a pangolin coronavirus Pang17 antigen and a bat SARS-like coronavirus WIV1 antigen.

In certain embodiments, the fusion polypeptide may comprise a Wuhan coronavirus antigen, a Khosta2 sarbecovirus antigen, a BtSCoV-RsSHC014 antigen and a bat SARS-like coronavirus WIV1 antigen.

In certain embodiments, the fusion polypeptide may comprise a BA.2 coronavirus antigen, a bat SARS coronavirus HKU3 antigen, a Kenya bat coronavirus BtKY72 antigen and a bat SARS-like coronavirus WIV1 antigen

In certain embodiments, the fusion polypeptide may comprise a Wuhan coronavirus antigen, a Khosta2 sarbecovirus antigen, a BISCoV-RsSHC014 antigen and a SARS coronavirus Urbani antigen.

In certain embodiments, the fusion polypeptide may comprise a Wuhan coronavirus antigen, a bat coronavirus RaTG13 antigen, a Kenya bat coronavirus BtKY72 antigen and a bat SARS-like coronavirus WIV1 antigen.

In certain embodiments, the antigen may comprise a receptor binding domain or an S1 domain.

In certain embodiments, the antigen may comprise a sequence selected from the group consisting of SEQ ID NOs: 12-24.

In certain embodiments herein there is provided a fusion polypeptide of SEQ ID NO: 1 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 1.

In certain embodiments herein there is provided a fusion polypeptide of SEQ ID NO: 2 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 2.

In certain embodiments herein there is provided a fusion polypeptide of SEQ ID NO: 3 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 3.

In certain embodiments herein there is provided a fusion polypeptide of SEQ ID NO: 4 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 4.

In certain embodiments herein there is provided a fusion polypeptide of SEQ ID NO: 5 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 5.

In certain embodiments herein there is provided a fusion polypeptide of SEQ ID NO: 6 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 6.

In certain embodiments herein there is provided a fusion polypeptide of SEQ ID NO: 7 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 7.

In certain embodiments herein there is provided a fusion polypeptide of SEQ ID NO: 8 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 8.

In certain embodiments herein there is provided a fusion polypeptide of SEQ ID NO: 9 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 9.

In certain embodiments herein there is provided a fusion polypeptide of SEQ ID NO: 10 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 10.

In certain embodiments herein there is provided a fusion polypeptide of SEQ ID NO: 11 or a sequence with at least 90% identity, at least 91% identity, at least 92% identity, at least 93% identity, at least 94% identity, at least 95% identity, at least 96% identity, at least 97% identity, at least 98% identity, at least 99% identity to SEQ ID NO: 11.

In certain embodiments there is provided an immunogenic composition comprising a fusion polypeptide as described herein, and a pharmaceutically acceptable salt, carrier, or adjuvant.

In certain embodiments there is provided a use of a fusion polypeptide or immunogenic composition as described herein in the manufacture of a medicament for the treatment or prevention of a pathogenic infection in a subject in need thereof.

In certain embodiments there is provided a use of a fusion polypeptide or immunogenic composition as described herein for the treatment or prevention of a pathogenic infection in a subject in need thereof.

In certain embodiments there is provided a method of treating or preventing a pathogenic infection comprising administering an effective amount of a fusion polypeptide or an immunogenic composition as described herein to a subject in need thereof. In certain embodiments, the pathogenic infection may comprise a viral infection, a bacterial infection, or a fungal infection.

In certain embodiments of a use or method, the pathogenic infection may comprise a viral infection. In certain embodiments of a use or method, the viral infection may comprise a Coronaviridae virus.

In certain embodiments of a use or method, the coronavirus may comprise MERS-CoV, SARS-CoV-1, SARS-CoV-2, HCoV-HKU1, HCoV-OC43, BtCoV-RaTG13, Bt SL-CoV-WIV1, HKU4-CoV, HKU5-CoV, PDF-2108 CoV, SHC014-CoV, BISCoV-RsSHC014, BISCoV-Rs3367, BANAL-52, BANAL-236, Pangolin-CoV GX/P2V, Bat coronavirus CDPHE15, Bat coronavirus HKU10, Rhinolophus ferrumequinum alphacoronavirus HuB-2013, Human coronavirus 229E, Lucheng Rn rat coronavirus, Mink coronavirus 1, Ferret coronavirus, Miniopterus bat coronavirus 1, Miniopterus bat coronavirus HKU8, Myotis ricketti alphacoronavirus Sax-2011, Nyctalus velutinus alphacoronavirus SC-2013, Pipistrellus kuhlii coronavirus 3398, Porcine epidemic diarrhea virus, Scotophilus bat coronavirus 512, Rhinolophus bat coronavirus HKU2, Human coronavirus NL63, NL63-related bat coronavirus strain BtKYNL63-9b, Sorex araneus coronavirus T14, Suncus murinus coronavirus X74, Alphacoronavirus 1, Canine coronavirus, Feline coronavirus, Human CCoV-HuPn-2018, Porcine transmissible gastroenteritis coronavirus, Swine acute diarrhea syndrome coronavirus (SADS-CoV), HCoV-NL63. NL63-related bat coronavirus strain BtKYNL63-9b, HCoV-229E, Khosta2 sarbecovirus, bat SARS-like coronavirus WIV1, Kenya bat coronavirus BtKY72, bat coronavirus SHC014, pangolin coronavirus Pang17, bat SARS coronavirus HKU3, SARS coronavirus Urbani, bat coronavirus RaTG13, Wuhan coronavirus, BA. 1 coronavirus, BA.2 coronavirus, B.1.351 coronavirus or any combination thereof; or a virus with at least 70% nucleotide identity with MERS-CoV, SARS-CoV-1, SARS-CoV-2, HCoV-HKU1, HCoV-OC43, BtCoV-RaTG13, Bt SL-CoV-WIV1, HKU4-CoV, HKU5-CoV, PDF-2108 COV, SHC014-CoV, BtSCoV-RsSHC014, BtSCoV-Rs3367, BANAL-52, BANAL-236, Pangolin-CoV GX/P2V, Bat coronavirus CDPHE15, Bat coronavirus HKU10, Rhinolophus ferrumequinum alphacoronavirus HuB-2013, Human coronavirus 229E, Lucheng Rn rat coronavirus, Mink coronavirus 1, Ferret coronavirus, Miniopterus bat coronavirus 1, Miniopterus bat coronavirus HKU8, Myotis ricketti alphacoronavirus Sax-2011, Nyctalus velutinus alphacoronavirus SC-2013, Pipistrellus kuhlii coronavirus 3398, Porcine epidemic diarrhea virus, Scotophilus bat coronavirus 512, Rhinolophus bat coronavirus HKU2, Human coronavirus NL63, NL63-related bat coronavirus strain BtKYNL63-9b, Sorex araneus coronavirus T14, Suncus murinus coronavirus X74, Alphacoronavirus 1, Canine coronavirus, Feline coronavirus, Human CCoV-HuPn-2018, Porcine transmissible gastroenteritis coronavirus, Swine acute diarrhea syndrome coronavirus (SADS-CoV), HCoV-NL63. NL63-related bat coronavirus strain BtKYNL63-9b, HCoV-229E, Khosta2 sarbecovirus, bat SARS-like coronavirus WIV1, Kenya bat coronavirus BtKY72, bat coronavirus SHC014, pangolin coronavirus Pang17, bat SARS coronavirus HKU3, SARS coronavirus Urbani, bat coronavirus RaTG13, Wuhan coronavirus, BA.1 coronavirus, BA.2 coronavirus, B.1.351 coronavirus, or any combination thereof.

In certain embodiments of a use or method, the fusion polypeptide or the immunogenic composition is for administration one or more times.

In certain embodiments of a use or method, the fusion polypeptide or the immunogenic composition neutralizes two or more different pathogens, two or more different antigens, or both.

In certain embodiments of a use or method, the two or more different pathogens comprise two or more protein variants derived from a same parental pathogen.

In certain embodiments of a use or method, the two or more pathogens comprise two or more Coronaviridae, two or more Coronaviridae variants, or any combination thereof.

In certain embodiments of a use or method, the fusion polypeptide or the immunogenic composition may reduce the quantity of a pathogen in the subject.

In certain embodiments there is provided a nucleic acid encoding a fusion polypeptide as described herein or encoding the fusion polypeptide with at least 90% identity, at least 91% identity, at least 92% identity, at least 93% identity, at least 94% identity, at least 95% identity, at least 96% identity, at least 97% identity, at least 98% identity, at least 99% identity to a fusion protein as described herein; or a nucleic acid encoding a fusion polypeptide comprising a sequence selected from the group consisting of: SEQ ID NO: 25-67 or encoding a fusion polypeptide with at least 90% identity, at least 91% identity, at least 92% identity, at least 93% identity, at least 94% identity, at least 95% identity, at least 96% identity, at least 97% identity, at least 98% identity, at least 99% identity to any one of SEQ ID NOs: 25-67.

In certain embodiments there is provided a cell transformed with a nucleic acid described herein or expressing a fusion polypeptide as described herein. In certain embodiments, the cell may comprise a mammalian cell, a yeast cell, a plant cell, a bacterial cell, or an insect/arthropod cell. In certain embodiments, the mammalian cell may comprise a Chinese Hamster Ovary (CHO) cell.

### BRIEF DESCRIPTION OF THE DRAWINGS

These and other features of the invention will become more apparent from the following description in which reference is made to the appended drawings wherein:

FIG. 1 is a schematic of exemplary fusion polypeptides as described herein. FIG. 1A provides a fusion polypeptide comprising, in sequence, a first antigen, a linker and one additional antigen. FIG. 1B provides a fusion polypeptide comprising, in sequence, a first antigen, a linker and one additional antigen, wherein the one additional antigen is a plurality of antigens, wherein n is an integer corresponding to the number of antigens comprising the “at least one additional” antigen in the fusion polypeptide and each antigen is linked to the preceding antigen by a linker sequence.

FIG. 2 is a schematic of exemplary fusion polypeptides as described herein. FIG. 2A provides a fusion polypeptide comprising, in sequence, a third antigen, a linker, a first antigen, a linker and one additional antigen. FIG. 2B provides a fusion polypeptide comprising, in sequence, a third antigen, a linker, a first antigen, a linker and one additional antigen, wherein the one additional antigen is a plurality of antigens, wherein n is an integer corresponding to the number of antigens comprising the “at least one additional” antigen in the fusion polypeptide and each antigen is linked to the preceding antigen by a linker sequence.

FIG. 3 is a schematic of an exemplary fusion polypeptide as described herein. FIG. 3 provides a fusion polypeptide comprising a plurality of antigens, wherein n is an integer corresponding to the number of antigens in the fusion polypeptide and each antigen is linked to the preceding antigen by a linker sequence.

FIG. 4 is a schematic of exemplary vaccines and study design. Vaccine candidates and challenge virus were altered between studies, but the overall study design and timeline remained the same. FIG. 4A provides exemplary vaccines comprising fusion polypeptides as described herein.

FIG. 4B provides an exemplary study design for the treatment and/or prevention of a pathogen infection with fusion polypeptides as described herein.

FIG. 5 provides neutralizing titers for 3 pathogen variants (SARS-CoV-2: Beta, Delta and Omicron) treated with COVAC-2 (control, unlinked WuhanS1), 4296 (WuhanS1-dRBD), or 4304 (WuhanS1-oRBD). Virus Neutralizing (VN) titres of PBS control or COVAC-2 (row 1), or S1Wu-dRBD (row 2), or S1Wu-oRBD (row 3) vaccinated hamsters challenged with variant of concern Beta (column 1), Delta (column 2) or Omicron (column 3). RBD: receptor binding domain; dRBD: SARS-CoV-2 delta variant RBD; oRBD: SARS-CoV-2 omicron variant RBD; S1Wu: SARS-CoV-2 Wuhan S1.

FIG. 6 provides weight loss data following a two dose vaccination with COVAC-2 (WuhanS1), 4304 (WuhanS1-oRBD), or 4296 (WuhanS1-dRBD), followed by challenge with Beta, Delta, or Omicron SARS-CoV-2 variants of concern (VOCs). RBD: receptor binding domain. dRBD: SARS-CoV-2 Delta variant RBD; oRBD: SARS-CoV-2 omicron variant RBD; S1Wu: SARS-CoV-2 Wuhan S1.

FIG. 7 provides infectious virus levels in the nasal cavity following a two dose vaccination with COVAC-2 (WuhanS1), 4304 (WuhanS1-oRBD), or 4296 (WuhanS1-dRBD), followed by challenge with beta, delta, or omicron SARS-CoV-2 variants of concern (VOCs). RBD: receptor binding domain; dRBD: SARS-CoV-2 delta variant RBD; oRBD: SARS-CoV-2 omicron variant RBD; S1Wu: SARS-CoV-2 Wuhan S1.

FIG. 8 provides infectious virus levels in the lung following a two dose vaccination with COVAC-2 (WuhanS1), 4304 (WuhanS1-omicronRBD), or 4296 (WuhanS1-deltaRBD), followed by challenge with Beta, Delta, or Omicron SARS-CoV-2 variants of concern (VOCs). RBD: receptor binding domain; deltaRBD: SARS-CoV-2 delta variant RBD; oRBD: SARS-CoV-2 omicron variant RBD; S1Wu: SARS-CoV-2 Wuhan S1.

FIG. 9 provides exemplary vaccines comprising three fused (linked) or three unlinked antigens, wherein the linked and unlinked vaccines differ only in that the antigens are fused in the linked.

FIG. 9A provides anti-S1 titers following a two dose vaccination for three antigens provided as a fusion (linked) or unlinked. FIG. 9B provides virus neutralization titers for the Delta SARS-CoV-2 variant following a two dose vaccination with three antigens provided as a fusion (linked) or unlinked.

FIG. 9C provides body weights of animals challenged with the Delta SARS-CoV-2 variant of concern following a two dose vaccination with three antigens provided as a fusion (linked) or unlinked. Ag: Antigen; RBD: receptor binding domain. omiRBD: SARS-CoV-2 omicron variant RBD; betaRBD: SARS-CoV-2 beta variant RBD; WhS1: SARS-CoV-2 Wuhan S1.

FIG. 10 provides an exemplary bioinformatics pipeline used to select viral sequences for candidate antigen design in the fusion polypeptide subunit platform.

FIG. 11 is a schematic of exemplary vaccines comprising fusion polypeptides as described herein and their phylogenetic relation in clades 1a, 1b, 2 and 3.

FIG. 12 provides an exemplary study design for vaccines and immunogenicity evaluation and the results thereof. FIG. 12A provides an exemplary study design for immunogenicity evaluation from animals administered with fusion polypeptides as described herein. FIG. 12B provides an exemplary serum binding ELISA from animals administered with fusion polypeptides as described herein. Significant differences from PBS are marked in blue (above the value), while significant differences from Pfizer are marked in red (below the value). Pfizer: Pfizer-BioNTech Comiranty®Original and Omicron BA.4/.5, bivalent COVID-19 vaccine, 4499: WhS1-Khosta2-WIV1, 4500: WhS1-Pang17-WIV1, 4501: WhS1-Khosta2-RsSHCO14-WIV1, 4502: WhS1-Khosta2-RsSHCO14-Urbani, 4503: WhS1-RatG13-BtKY72-WIV1.

FIG. 13 provides an exemplary IFNg/IL-5 ELISPOT from animals administered with fusion polypeptides as described herein with a dose of 5 μg. All groups were stimulated with S1 from the ancestral SARS-CoV-2 strain and Urbani RBD. Additionally, candidate vaccines were stimulated with their own antigen. Significant differences from PBS are marked in blue, while significant differences from Pfizer are marked in red. Pfizer: Pfizer-BioNTech Comiranty®Original and Omicron BA.4/.5, bivalent COVID-19 vaccine, 4499: WhS1-Khosta2-WIV1, 4500: WhS1-Pang17-WIV1, 4501: WhS1-Khosta2-RsSHCO14-WIV1, 4502: WhS1-Khosta2-RsSHCO14-Urbani, 4503: WhS1-RatG13-BtKY72-WIV1.

FIG. 14 is a schematic of exemplary study design vaccines and immunogenicity evaluation. Hamsters were vaccinated on Day 0 and Day 28 with respective antigens. Hamsters were challenged intranasally on Day 48 with the Delta variant of SARS-CoV-2 (1×10⁵ TCID₅₀). Serum was collected for ELISA and viral neutralization assays on Days 0, 28 and 48. Nasal wash samples were collected on Days 1, 3, 5, 7 and 10 post challenge. Animals were euthanized on Days 5 and 10 post challenge for respiratory tissue collection.

FIG. 15 provides an exemplary percent body weight change analysis from animals administered with fusion polypeptides as described herein. Significant differences from PBS are marked in blue (above the value), while significant differences from Pfizer are marked in red (below the value). Pfizer: Pfizer-BioNTech Comiranty®Original and Omicron BA.4/.5, bivalent COVID-19 vaccine, 4499: WhS1-Khosta2-WIV1, 4500: WhS1-Pang17-WIV1, 4501: WhS1-Khosta2-RsSHCO14-WIV1, 4502: WhS1-Khosta2-RsSHCO14-Urbani, 4503: WhS1-RatG13-BtKY72-WIV1.

FIG. 16 provides an exemplary ELISA from animals administered with fusion polypeptides as described herein. Significant differences from PBS are marked in blue (above the value), while significant differences from Pfizer are marked in red (below the value). Pfizer: Pfizer-BioNTech Comiranty®Original and Omicron BA.4/.5, bivalent COVID-19 vaccine, 4499: WhS1-Khosta2-WIV1, 4500: WhS1-Pang17-WIV1, 4501: WhS1-Khosta2-RsSHCO14-WIV1, 4502: WhS1-Khosta2-RsSHCO14-Urbani, 4503: WhS1-RatG13-BtKY72-WIV1.

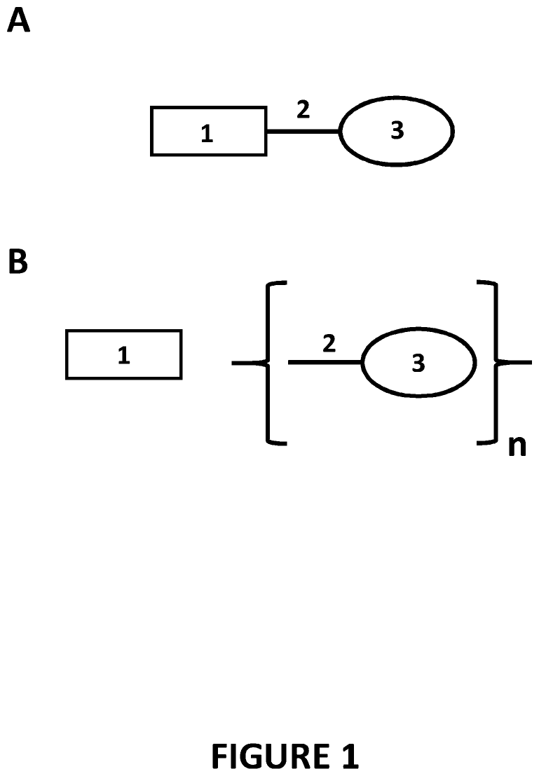

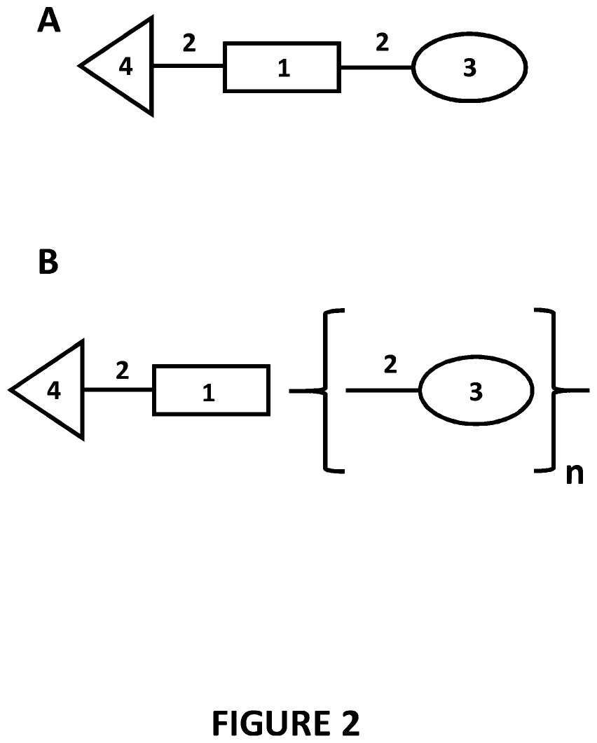

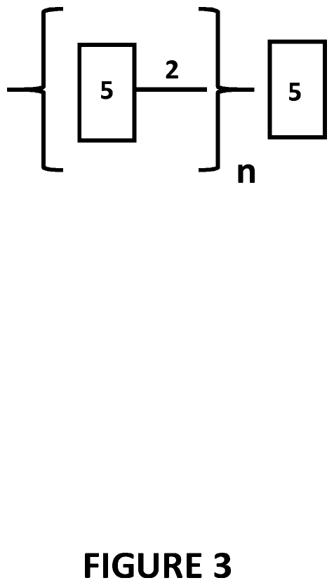

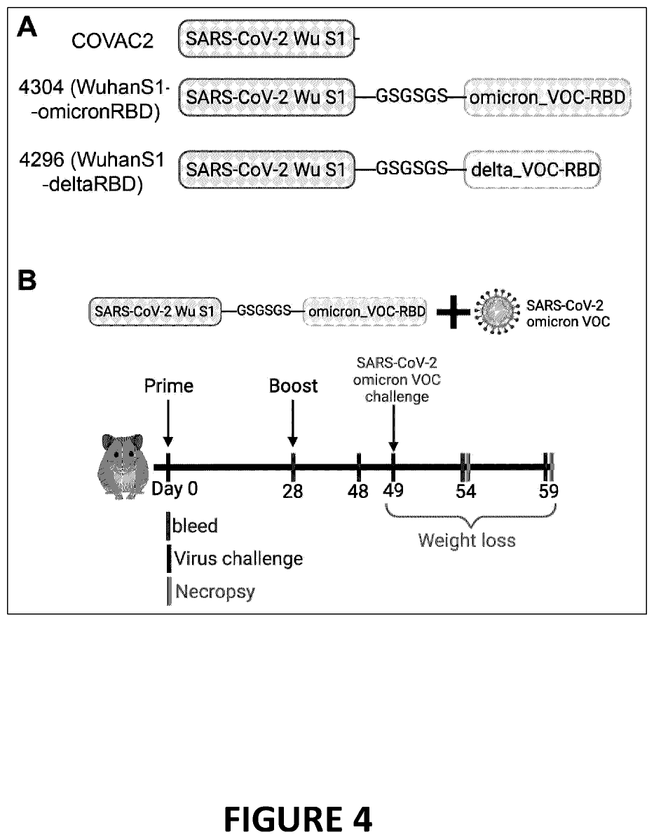

### DETAILED DESCRIPTION

Described herein are fusion polypeptides, immunogenic compositions comprising fusion polypeptides, and methods and uses thereof. It will be appreciated that embodiments and examples are provided herein for illustrative purposes intended for those skilled in the art, and are not meant to be limiting in any way.

The practice of the present invention will employ, unless otherwise indicated, conventional methods of microbiology, virology, chemistry, biochemistry, recombinant DNA techniques and immunology, within the skill of the art. Such techniques are explained fully in the literature. See, e.g., Fundamental Virology, Current Edition, vol. I & II (B. N. Fields and D. M. Knipe, eds.); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company); A. L. Lehninger, Biochemistry (Worth Publishers, Inc., current edition); Sambrook, et al., Molecular Cloning: A Laboratory Manual (current edition); Methods In Enzymology (S. Colowick and N. Kaplan eds., Academic Press, Inc.).

All publications, patents and patent applications cited herein, whether supra or infra, are hereby incorporated by reference in their entireties.

It must be noted that, as used in this specification and the appended claims, the singular forms “a”, “an” and “the” include plural referents unless the content clearly dictates otherwise. Thus, for example, reference to “an antigen” may include a mixture of two or more such antigens, and the like.

As used herein, the term “coronavirus” may correspond to any virus of the Coronaviridae family or an antigen derived therefrom. As would be known to the person of skill, common names for coronavirus family members, such as for example “Omicron”, may be referred to as such or by alternative names that would be known to the skilled person, such as for example “BA. 1” or “BA.2”. It is also contemplated that sub-lineages of coronavirus family members, such as for example BA.4, BA.5, XBB.1.5 or any other sub-lineages known to the person of skill may be provided in fusion proteins as described herein. As would be known to the person of skill, a sublineage may comprise the same and/or different antigens than the parental strain or other sublineages thereof.

In certain embodiments there is provided herein a fusion polypeptide comprising an amino acid sequence encoding, in sequence, a first antigen (1), a linker (2), and at least one additional antigen (3).

The terms “polypeptide” and “protein” as used herein refer to a polymer of amino acid residues and are not limited to a minimum length, and peptides, oligopeptides, dimers, multimers, and the like, may be included within the definition. Both full-length proteins and fragments thereof are encompassed by the definition. The terms also may include post-translational modifications of the polypeptide or protein, such as for example, glycosylation, acetylation, phosphorylation and the like. Furthermore, for purposes of the present invention, a “polypeptide” may refer to a protein which includes modifications, such as deletions, additions and substitutions, to the native sequence, as long as the protein maintains the desired activity, such as for example, recognition by a B-cell. These modifications may be deliberate, as through site-directed mutagenesis, or may be unintended, such as through mutations in a host genome which produce the proteins or errors due to PCR amplification.

The term “peptide” as used herein refers to a fragment of a polypeptide. Thus, a peptide can include a C-terminal deletion, an N-terminal deletion and/or an internal deletion of the native polypeptide, so long as the entire protein sequence is not present. A peptide will generally include at least about 3-10 contiguous amino acid residues of the full-length molecule, and can include at least about 15-25 contiguous amino acid residues of the full-length molecule, or at least about 20-50 or more contiguous amino acid residues of the full-length molecule, or any integer between 3 amino acids and the number of amino acids in the full-length sequence, provided that the peptide in question retains the ability to elicit the desired biological response.

As used herein the phrase a “fusion polypeptide” may refer to a single polypeptide encoded by two or more different amino acid sequences, wherein the different amino acid sequences may be derived from and comprise:
i) different polypeptides from a pathogen, such as, by way of non-limiting example, a SARS-CoV-2 spike protein and a SARS-CoV-2 nucleocapsid protein;
ii) different antigens from a pathogen, such as, by way of non-limiting example, an antigenic fragment of a SARS-CoV-2 spike protein and an antigenic fragment of a SARS-CoV-2 nucleocapsid protein;
iii) different variants of a polypeptide from a pathogen, such as, by way of non-limiting example, a receptor binding domain of a SARS-CoV-2 comprising delta variant mutations, a receptor binding domain of a SARS-CoV-2 comprising omicron variant mutations, and/or a receptor binding domain of a SARS-CoV-2 comprising beta variant mutations;
iv) polypeptides from different pathogens, such as, by way of non-limiting example, a Coronaviridae virus, an Orthomyxoviridae virus, and/or an Pneumoviridae virus, or it may comprise, for example, a Betacoronavirus and an Alphacoronavirus;
v) an amino acid sequence from a different genus, lineage, species, variant, serotype, and/or strain than the ancestral or parent pathogen or microorganism; or any combination thereof;

As used herein, an “antigen” refers to a molecule, such as a protein, polypeptide, or fragment thereof, containing one or more epitopes (either linear, conformational or both) that will stimulate a host's immune-system to make a humoral and/or a cellular antigen-specific response. The term may be used interchangeably with the term “immunogen”. Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide which expresses an antigen or antigenic determinant in vivo, such as in nucleic acid immunization applications, is also included in the definition of antigen herein. As used herein, a “plurality of antigens” may refer to one or more antigens, wherein the one or more antigens may be different antigens, different variants of an antigen, antigens from different pathogenic organisms, or any combination thereof.

As used herein, a “linker” or a “linker sequence” may refer to a peptide or polypeptide containing one or more amino acid residues (e.g, 1, 2, 3, 4, 5, 6, 7, 8, 9, 10, 11, 12, 13, 14, 15, 16, 17, 18, 19, 20, 21, 22, 23, 24 or more amino acid residues) joined by a peptide bond(s). Linkers may provide rotational freedom that allows each component of the fusion protein to interact with its intended target, such as for example, a B-cell, without hindrance. These linkers may be mixtures of glycine (G), alanine (A) and serine(S), such as (GSGSGS) n or (GAAASG) n, where n is 1, 2, 3, 4, 5, 6 or greater. In certain embodiments wherein the fusion polypeptide comprises two or more linkers, each linker may independently be a different linker sequence or the same linker sequence.

In certain embodiments, it is contemplated that the first antigen and the one or more additional antigens are different antigens, wherein the different antigens may comprise sequences derived from a different organism, protein, family, genus, lineage, species, variant, serotype, strain or any combination thereof. In certain embodiments, the first antigen and the one or more additional antigens may comprise antigens from different organisms, such as a virus and a prokaryote, or they may comprise antigens from the same organism, such as two different viral antigens. In certain embodiments, the first antigen and the one or more additional antigens may comprise antigens from the same family of viruses, such as Coronaviridae, or they may comprise antigens from different families of viruses, such as Coronaviridae and Orthomyxoviridae. In certain embodiments, the first antigen and the one or more additional antigens may comprise antigens from different genus, such as a Betacoronavirus and an Alphacoronavirus. In certain embodiments, the first antigen and the one or more additional antigens may comprise antigens from different proteins, such as a SARS-CoV-2 spike protein and a SARS-CoV-2 nucleocapsid protein. In certain embodiments, the first antigen and the one or more additional antigens may comprise different variants, such as a SARS-CoV-2 delta variant or a SARS-CoV-2 omicron variant.

As used herein, a “fungus” may comprise any organism in the kingdom Fungi, and a “fungal antigen” may comprise any antigenic sequence therefrom. In certain embodiments, the fungal antigen may be derived from a fungal organism that contributes to or is causative in a disease or condition in a vertebrate subject, such as for example, Candida, Cryptococcus, or Aspergillus.

As used herein, a “prokaryote” may comprise any prokaryotic organism, and a “prokaryotic antigen” may comprise any antigenic sequence therefrom. In certain embodiments, the prokaryotic antigen may be derived from a prokaryotic organism that contributes to or is causative in a disease or condition in a vertebrate subject, such as for example, Pneumococcus, Corynebacterium, or Clostridium.

As used herein, a “virus” may comprise any submicroscopic organism or agent that replicates only inside the living cells of an organism, and a “viral antigen” may comprise any antigenic sequence therefrom. In certain embodiments, the viral antigen may be derived from a virus that contributes to or is causative in a disease or condition in a vertebrate subject, such as for example, Coronaviridae, Orthomyxoviridae, or Pneumoviridae.

In certain embodiments, the at least one additional antigen may comprise a plurality of antigens and each antigen of the plurality of antigens may be linked to the preceding antigen with a linking peptide comprising the linker. In certain embodiments, the plurality of antigens may comprise two or more antigens, such as 2, 3, 4, 5, 6, 7, 8, 9 or 10 antigens. In certain embodiments, the plurality of antigens may each independently comprise a different antigen, wherein the different antigen may comprise an antigen derived from a different organism, phyla, genus, family, lineage, species, variant, serotype, and strain than an ancestral pathogen, a parent pathogen, microorganism or antigenic sequence.

As used herein, the terms “preceding” and “upstream” in reference to a polypeptide, an amino acid sequence or nucleotide sequence may refer to an element, such as an amino acid sequence encoding, for example, an antigen, antigenic fragment or linker, that is in the N-terminal position of the element to which it is “preceding” or is “upstream” of in the polypeptide or in the resulting polypeptide encoded by the nucleotide sequence. As used herein, the terms “proceeding” and “downstream” in reference to a polypeptide, an amino acid sequence or a nucleotide sequence may refer to an element, such as an antigenic amino acid sequence, that is in the C-terminal position of the element to which is “proceeding” or is “downstream” of.

In certain embodiments, the amino acid sequence of the first antigen may comprise a greater number of amino acids than the amino acid sequence of at least one of the at least one or more antigen. In certain embodiments, a greater number of amino acids may consist of 1 or more additional amino acids in the first antigen in comparison to the at least one of the at least one or more antigen, such as, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 or more amino acids.

In certain embodiments, the first antigen may comprise an antigenic peptide or fragment thereof from a first pathogen. In certain embodiments, the first pathogen may comprise a vertebrate pathogen comprising a viral pathogen, a prokaryotic pathogen, or a eukaryotic pathogen, such as a fungal pathogen.

In certain embodiments, the first viral pathogen may comprise: Adeno-associated virus, Aichi virus, Australian bat lyssavirus, Banna virus, Barmah forest virus, BK polyomavirus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern chimpanzee simian foamy virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis delta virus Hepatitis E virus, Horsepox virus, Human adenovirus, Human astrovirus, Human coronavirus, Human cytomegalovirus. Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 16,18 Human papillomavirus 2, Human parainfluenza, Human parvovirus B19, Human respiratory syncytial virus, Human rhinovirus, Human SARS coronavirus, Human T-lymphotropic virus, Human torovirus, Influenza A virus, Influenza B virus, Influenza C virus, Isfahan virus, Japanese encephalitis virus, JC polyomavirus, Junin arenavirus, KI Polyomavirus, Lagos bat virus, Lake Victoria marburgvirus, Langat virus, Lassa virus, Louping ill virus, Lymphocytic choriomeningitis virus. Machupo virus, Mammalian orthorubulavirus 5 (Simian virus 5), Mayaro virus, Measles virus, Merkel cell polyomavirus, MERS coronavirus, Mokola virus, Molluscum contagiosum virus, Monkeypox virus. Mumps virus, Murray valley encephalitis virus, New York virus, Nipah virus, Norwalk virus. O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever Naples phlebovirus (Toscana virus), Sandfly fever sicilian virus, Sapporo virus, SARS coronavirus, SARS coronavirus 2, Semliki forest virus, Seoul virus, Simian foamy virus, Sindbis virus, Southampton virus, St. louis encephalitis virus, Tick-borne powassan virus, Torque teno virus, Uukuniemi virus, Vaccinia virus, Varicella-zoster virus, Variola virus, Venezuelan equine encephalitis virus, Vesicular stomatitis virus, West Nile virus, Western equine encephalitis virus, WU polyomavirus, Yaba monkey tumor virus, Yaba-like disease virus, Yellow fever virus, Zika virus, Khosta2 sarbecovirus, bat SARS-like WIV1 coronavirus, Kenya bat coronavirus BtKY72, pangolin Pang17 coronavirus, bat SARS HKU3 coronavirus, SARS Urbani coronavirus, bat RaTG13 coronavirus, Wuhan coronavirus, BA. 1 coronavirus, BA.2 coronavirus, B.1.351 coronavirus, any serotype thereof, any variant thereof, or any combination thereof.

In certain embodiments, the first antigen may comprise a first viral antigen. In certain embodiments, the first viral antigen may be derived from a viral pathogen. In certain embodiments, the first viral antigen may comprise any antigen derived from a first viral pathogen.

In certain embodiments, the first prokaryotic pathogen may comprise: Actinomyces israelii, Bacillus anthracis, Bacteroides fragilis, Bordetella pertussis, Borrelia, Brucella, Campylobacter jejuni, Chlamydia, Chlamydophila psittaci, Clostridium, Corynebacterium diphtheriae, Ehrlichia, Enterococcus, Escherichia, Francisella tularensis, Haemophilus influenzae, Helicobacter pylori, Klebsiella pneumoniae, Legionella pneumophila, Leptospira, Listeria monocytogenes, Mycobacterium, Mycoplasma pneumoniae, Neisseria, Pseudomonas aeruginosa, Nocardia asteroides, Rickettsia rickettsii, Salmonella, Shigella, Staphylococcus, Streptococcus, Treponema pallidum, Vibrio cholerae, Yersinia pestis, or any combination thereof.

In certain embodiments, the first antigen may comprise a first prokaryotic antigen. In certain embodiments, the first prokaryotic antigen may be derived from a prokaryotic pathogen. In certain embodiments, the first prokaryotic antigen may comprise any antigen derived from a first prokaryotic pathogen.

In certain embodiments, the first eukaryotic pathogen may comprise: Trypanosoma brucei, Entamoeba histolytica, Babesia bovis, Babesia divergens, Candida albicans, Trypanosoma cruzi, Giardia lamblia, Acanthamoeba culbertsoni, Necator americanus, Leishmania major, Bruga malayi, Plasmodium falciparum, Plasmodium vivax, Toxoplasma gondii, Trichomonas vaginalis, Aedes aegypti, or any combination thereof.

In certain embodiments, the first antigen may comprise a first eukaryotic antigen. In certain embodiments, the first eukaryotic antigen may be derived from a eukaryotic pathogen. In certain embodiments, the first eukaryotic antigen may comprise any antigen derived from the first eukaryotic pathogen.

In certain embodiments, the first viral antigen may comprise a component of a surface glycoprotein from a virus, a lipoprotein or an extracellular domain of a transmembrane protein. As used herein, a “surface glycoprotein” may refer to a molecule comprising a polypeptide and a carbohydrate chain. A “component of a surface glycoprotein” may comprise a full-length glycoprotein or a fragment thereof. In certain embodiments, the component of the surface glycoprotein, the lipoprotein or the extracellular domain of the transmembrane protein may be an antigenic component.

In certain embodiments, the first viral antigen may comprise a first coronavirus antigen. In certain embodiments, the first coronavirus antigen may comprise an antigen from a Coronaviridae virus. In certain embodiments, the first coronavirus antigen may comprise an antigen from a Betacoronavirus, a Gammacoronavirus, an Alphacoronavirus, a Deltacoronavirus or any combination thereof. In certain embodiments, the first coronavirus antigen may comprise a MERS-CoV antigen, a SARS-CoV-1 antigen, a SARS-CoV-2 antigen (for example but not limited to Wuhan coronavirus antigens, BA. 1 coronavirus antigens, BA.2 coronavirus antigens, or B.1.351 coronavirus antigens), a HCoV-HKU1 antigen, a HCoV-OC43 antigen, a BtCoV-RaTG13 antigen, a Bt SL-CoV-WIV1 antigen, a HKU4-CoV antigen, a HKU5-CoV antigen, a PDF-2108 CoV antigen, a SHC014-CoV antigen, a BtSCoV-RsSHC014 antigen, a BISCoV-Rs3367 antigen, a BANAL-52 antigen, a BANAL-236 antigen, a Pangolin-CoV GX/P2V antigen, a Bat coronavirus CDPHE15 antigen, a Bat coronavirus HKU10 antigen, a Rhinolophus ferrumequinum alphacoronavirus HuB-2013 antigen, a Human coronavirus 229E antigen, a Lucheng Rn rat coronavirus antigen, a Mink coronavirus 1 antigen, a Ferret coronavirus antigen, a Miniopterus bat coronavirus 1 antigen, a Miniopterus bat coronavirus HKU8 antigen, a Myotis ricketti alphacoronavirus Sax-2011 antigen, a Nyctalus velutinus alphacoronavirus SC-2013 antigen, a Pipistrellus kuhlii coronavirus 3398 antigen, a Porcine epidemic diarrhea virus antigen, a Scotophilus bat coronavirus 512 antigen, a Rhinolophus bat coronavirus HKU2 antigen, a Human coronavirus NL63 antigen, a NL63-related bat coronavirus strain BtKYNL63-9b antigen, a Sorex araneus coronavirus T14 antigen, a Suncus murinus coronavirus X74 antigen, a Alphacoronavirus 1 antigen, a Canine coronavirus antigen, a Feline coronavirus antigen, a Human CCOV-HuPn-2018 antigen, a Porcine transmissible gastroenteritis coronavirus antigen, a Swine acute diarrhea syndrome coronavirus (SADS-CoV) antigen, a HCoV-NL63 antigen, a NL63-related bat coronavirus strain BtKYNL63-9b antigen, a HCoV-229E antigen, a Khosta2 sarbecovirus antigen, a Kenya bat coronavirus BtKY72 antigen, a bat SARS coronavirus HKU3 antigen, or any combination thereof. In certain embodiments, the first coronavirus antigen may comprise an antigen derived from a Coronaviridae spike protein, S1 protein, receptor binding domain (RBD) and/or any fragment thereof. In certain embodiments, the first coronavirus antigen may comprise a sequence selected from SEQ ID NOs: 12 to 24 or a nucleic acid selected from SEQ ID NOs: 25-67 encoding the antigen. In certain embodiments the antigen sequence may have at least 90% identity, at least 91% identity, at least 92% identity, at least 93% identity, at least 94% identity, at least 95% identity, at least 96% identity, at least 97% identity, at least 98% identity, at least 99% identity to a sequence selected from SEQ ID NOs: 12 to 24 or the nucleic acid encoding the fusion polypeptide selected from SEQ ID NOs: 25-67.

In certain embodiments, the first coronavirus antigen may comprise a spike (S) protein, or fragment thereof. As used herein, a coronavirus spike (S) protein may comprise a spike protein or fragment thereof from a Betacoronavirus, a Gammacoronavirus, an Alphacoronavirus, a Deltacoronavirus or any combination thereof. In certain embodiments, the spike protein may comprise any Coronaviridae spike protein or a fragment thereof, or a protein encoded by a nucleotide sequence with at least 70% identity to any Coronaviridae spike protein. In certain embodiments, the spike (S) protein, or fragment thereof may be a receptor binding domain (RBD) from a Betacoronavirus, a Gammacoronavirus, an Alphacoronavirus, a Deltacoronavirus or any combination thereof. In certain embodiments, the RBD may comprise any Coronaviridae RBD or a fragment thereof, or a protein encoded by a nucleotide sequence with at least 70% identity to any Coronaviridae RBD. In certain embodiments, the spike (S) protein, or fragment thereof may be an S1 protein from a Betacoronavirus, a Gammacoronavirus, an Alphacoronavirus, a Deltacoronavirus or any combination thereof. In certain embodiments, the spike protein may comprise any Coronaviridae S1 protein or a fragment thereof, or a protein encoded by a nucleotide sequence with at least 70% identity to any Coronaviridae S1 protein.

In certain embodiments, the linker may comprise a sequence of 6 or more amino acids in length. In certain embodiments, the linker may comprise a sequence of 6 to 24 amino acids. In certain embodiments, the linker may comprise a sequence consisting of one or more of glycine, serine and alanine. In certain embodiments, the linker may comprise a sequence, that when operatively linked to the preceding antigen, does not impair the ability of any of the antigens in the fusion polypeptide to interact with their intended target, such as for example, a B-cell. In certain embodiments, the linker may comprise 1 or more repeating unit of GSGSGS or GAAASG, such as for example 1, 2, 3, 4, 5, or 6 repeating units. In certain embodiments, the linker may comprise GSGSGS, GSGSGS GSGSGS, GSGSGS GSGSGS GSGSGS, GSGSGS GSGSGS GSGSGS GSGSGS, GSGSGS GSGSGS GSGSGS GSGSGS GSGSGS, GSGSGS GSGSGS GSGSGS GSGSGS GSGSGS, GAAASG, GAAASG GAAASG, GAAASG GAAASG GAAASG, GAAASG GAAASG GAAASG GAAASG, GAAASG GAAASG GAAASG GAAASG GAAASG, or GAAASG GAAASG GAAASG GAAASG GAAASG GAAASG. In certain embodiments wherein the fusion polypeptide may comprise more than one linker, each linker may independently comprise the same or a different linker sequence.

In certain embodiments, the at least one additional antigen comprises at least one additional antigenic peptide or fragment thereof from at least one additional pathogen. In certain embodiments, the first pathogen may comprise a vertebrate pathogen comprising a viral pathogen, a prokaryotic pathogen, or a eukaryotic pathogen, such as a fungal pathogen. In certain embodiments, the at least one additional antigen may comprise an antigen as disclosed for the first antigen, wherein the at least one additional antigen is different than the first antigen.

In certain embodiments, the at least one additional antigen may comprise an amino acid sequence of at least 8 amino acids.

In certain embodiments, the at least one additional antigen may comprise a vertebrate pathogen comprising at least one additional prokaryotic antigen, at least one additional viral antigen, at least one additional fungal antigen, or any combination thereof.

In certain embodiments, the at least one viral pathogen may comprise: Adeno-associated virus, Aichi virus, Australian bat lyssavirus, Banna virus, Barmah forest virus, BK polyomavirus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern chimpanzee simian foamy virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis delta virus E virus, Horsepox virus, Human adenovirus, Human astrovirus, Human coronavirus, Human cytomegalovirus. Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 16,18 Human papillomavirus 2, Human parainfluenza, Human parvovirus B19, Human respiratory syncytial virus, Human rhinovirus, Human SARS coronavirus, Human T-lymphotropic virus, Human torovirus, Influenza A virus, Influenza B virus, Influenza C virus, Isfahan virus, Japanese encephalitis virus, JC polyomavirus, Junin arenavirus, KI Polyomavirus, Lagos bat virus, Lake Victoria marburgvirus, Langat virus, Lassa virus, Louping ill virus, Lymphocytic choriomeningitis virus. Machupo virus, Mammalian orthorubulavirus 5 (Simian virus 5), Mayaro virus, Measles virus, Merkel cell polyomavirus, MERS coronavirus, Mokola virus, Molluscum contagiosum virus, Monkeypox virus. Mumps virus, Murray valley encephalitis virus, New York virus, Nipah virus, Norwalk virus. O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever Naples phlebovirus (Toscana virus), Sandfly fever sicilian virus, Sapporo virus, SARS coronavirus, SARS coronavirus 2, Semliki forest virus, Seoul virus, Simian foamy virus, Sindbis virus, Southampton virus, St. louis encephalitis virus, Tick-borne powassan virus, Torque teno virus, Uukuniemi virus, Vaccinia virus, Varicella-zoster virus, Variola virus, Venezuelan equine encephalitis virus, Vesicular stomatitis virus, West Nile virus, Western equine encephalitis virus, WU polyomavirus, Yaba monkey tumor virus, Yaba-like disease virus, Yellow fever virus, Zika virus, Khosta2 sarbecovirus, bat SARS-like WIV1 coronavirus, Kenya bat coronavirus BtKY72, pangolin Pang17 coronavirus, bat SARS HKU3 coronavirus, SARS Urbani coronavirus, bat RaTG13 coronavirus, Wuhan coronavirus, BA. 1 coronavirus, BA.2 coronavirus, B.1.351 coronavirus, any serotype thereof, any variant thereof, or any combination thereof.

In certain embodiments, the at least one antigen may comprise at least one viral antigen. In certain embodiments, the at least one viral antigen may be derived from a viral pathogen. In certain embodiments, the at least one viral antigen may comprise any antigen derived from a viral pathogen described herein.

In certain embodiments, the at least one prokaryotic pathogen may comprise: Actinomyces israelii, Bacillus anthracis, Bacteroides fragilis, Bordetella pertussis, Borrelia, Brucella, Campylobacter jejuni, Chlamydia, Chlamydophila psittaci, Clostridium, Corynebacterium diphtheriae, Ehrlichia, Enterococcus, Escherichia, Francisella tularensis, Haemophilus influenzae, Helicobacter pylori, Klebsiella pneumoniae, Legionella pneumophila, Leptospira, Listeria monocytogenes, Mycobacterium, Mycoplasma pneumoniae, Neisseria, Pseudomonas aeruginosa, Nocardia asteroides, Rickettsia rickettsii, Salmonella, Shigella, Staphylococcus, Streptococcus, Treponema pallidum, Vibrio cholerae, Yersinia pestis, or any combination thereof.

In certain embodiments, the at least one antigen may comprise at least one prokaryotic antigen. In certain embodiments, the at least one prokaryotic antigen may be derived from a prokaryotic pathogen. In certain embodiments, the at least one prokaryotic antigen may comprise any antigen derived from the at least one prokaryotic pathogen.

In certain embodiments, the at least one eukaryotic pathogen may comprise Trypanosoma brucei, Entamoeba histolytica, Babesia bovis, Babesia divergens, Candida albicans, Trypanosoma cruzi, Giardia lamblia, Acanthamoeba culbertsoni, Necator americanus, Leishmania major, Bruga malayi, Plasmodium falciparum, Plasmodium vivax, Toxoplasma gondii, Trichomonas vaginalis, Aedes aegypti, or any combination thereof.

In certain embodiments, the at least one antigen may comprise at least one eukaryotic antigen. In certain embodiments, the at least one eukaryotic antigen may be derived from a eukaryotic pathogen. In certain embodiments, the at least one eukaryotic antigen may comprise any antigen derived from the at least one eukaryotic pathogen.

In certain embodiments, the at least one viral antigen may comprise at least one coronavirus antigen. In certain embodiments, the at least one coronavirus antigen may comprise an antigen from a Coronaviridae virus. In certain embodiments, the at least one coronavirus antigen may comprise a MERS-CoV antigen, a SARS-CoV-1 antigen, a SARS-CoV-2 antigen (for example but not limited to Wuhan coronavirus antigens, BA.1 coronavirus antigens, BA.2 coronavirus antigens, or B.1.351 coronavirus antigens), a HCoV-HKU1 antigen, a HCoV-OC43 antigen, a BtCoV-RaTG13 antigen, a Bt SL-CoV-WIV1 antigen, a HKU4-CoV antigen, a HKU5-CoV antigen, a PDF-2108 CoV antigen, a SHC014-CoV antigen, a BISCoV-RsSHC014 antigen, a BISCoV-Rs3367 antigen, a BANAL-52 antigen, a BANAL-236 antigen, a Pangolin-CoV GX/P2V antigen, a Bat coronavirus CDPHE15 antigen, a Bat coronavirus HKU10 antigen, a Rhinolophus ferrumequinum alphacoronavirus HuB-2013 antigen, a Human coronavirus 229E antigen, a Lucheng Rn rat coronavirus antigen, a Mink coronavirus 1 antigen, a Ferret coronavirus antigen, a Miniopterus bat coronavirus 1 antigen, a Miniopterus bat coronavirus HKU8 antigen, a Myotis ricketti alphacoronavirus Sax-2011 antigen, a Nyctalus velutinus alphacoronavirus SC-2013 antigen, a Pipistrellus kuhlii coronavirus 3398 antigen, a Porcine epidemic diarrhea virus antigen, a Scotophilus bat coronavirus 512 antigen, a Rhinolophus bat coronavirus HKU2 antigen, a Human coronavirus NL63 antigen, a NL63-related bat coronavirus strain BtKYNL63-9b antigen, a Sorex araneus coronavirus T14 antigen, a Suncus murinus coronavirus X74 antigen, a Alphacoronavirus 1 antigen, a Canine coronavirus antigen, a Feline coronavirus antigen, a Human CCoV-HuPn-2018 antigen, a Porcine transmissible gastroenteritis coronavirus antigen, a Swine acute diarrhea syndrome coronavirus (SADS-CoV) antigen, a HCoV-NL63 antigen, a NL63-related bat coronavirus strain BtKYNL63-9b antigen, a HCoV-229E antigen, a Khosta2 sarbecovirus antigen, a Kenya bat coronavirus BtKY72 antigen, a bat SARS coronavirus HKU3 antigen, or any combination thereof. In certain embodiments, the at least one viral antigen may comprise a sequence selected from SEQ ID NOs: 12 to 24 or a nucleic acid selected from SEQ ID NOs: 25-67 encoding the antigen. In certain embodiments, the at least one antigen may comprise an antigen derived from a Coronaviridae spike protein, S1 protein, receptor binding domain (RBD) and/or any fragment thereof. In certain embodiments the antigen sequence may have at least 90% identity, at least 91% identity, at least 92% identity, at least 93% identity, at least 94% identity, at least 95% identity, at least 96% identity, at least 97% identity, at least 98% identity, at least 99% identity to a sequence selected from SEQ ID NOs: 12 to 24 or the nucleic acid encoding the fusion polypeptide selected from SEQ ID NOs: 25-67.

In certain embodiments, the at least one coronavirus antigen may comprise a spike (S) protein, or fragment thereof. As used herein, a coronavirus spike (S) protein may comprise a spike protein or fragment thereof from a Betacoronavirus, a Gammacoronavirus, an Alphacoronavirus, a Deltacoronavirus or any combination thereof. In certain embodiments, the spike protein may comprise any Coronaviridae spike protein or a fragment thereof, or a protein encoded by a nucleotide sequence with at least 70% identity to any Coronaviridae spike protein. In certain embodiments, the spike (S) protein, or fragment thereof may be a receptor binding domain (RBD) from a Betacoronavirus, a Gammacoronavirus, an Alphacoronavirus, a Deltacoronavirus or any combination thereof. In certain embodiments, the RBD may comprise any Coronaviridae RBD or a fragment thereof, or a protein encoded by a nucleotide sequence with at least 70% identity to any Coronaviridae RBD. In certain embodiments, the spike (S) protein, or fragment thereof may be an S1 protein from a Betacoronavirus, a Gammacoronavirus, an Alphacoronavirus, a Deltacoronavirus or any combination thereof. In certain embodiments, the spike protein may comprise any Coronaviridae S1 protein or a fragment thereof, or a protein encoded by a nucleotide sequence with at least 70% identity to any Coronaviridae S1 protein.

In certain embodiments, the first antigen and the at least one additional antigen each independently comprise a prokaryotic antigen, a viral antigen, or a fungal antigen. In certain embodiments, the first antigen may comprise a viral antigen and the at least one additional antigen may comprise a prokaryotic antigen. In certain embodiments, the first antigen may comprise a viral antigen and the at least one additional antigen may comprise a viral antigen, such as for example, the first antigen may comprise a SARS-CoV-2 S1 protein and the at least one additional antigen may comprise a SARS-CoV-2 receptor binding domain. In certain embodiments, the first antigen may comprise a delta SARS-CoV-2 variant and the at least one additional antigen may comprise an omicron SARS-CoV-2 variant.

In certain embodiments, the fusion polypeptide may further comprise, in sequence, a third antigen (4) and the linker sequence (2) upstream of the first antigen protein.

In certain embodiments, the third antigen may comprise a vertebrate pathogen comprising a third prokaryotic antigen, a third viral antigen, a third fungal antigen, or any combination thereof.

In certain embodiments, the third viral pathogen may comprise: Adeno-associated virus, Aichi virus, Australian bat lyssavirus, Banna virus, Barmah forest virus, BK polyomavirus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern chimpanzee simian foamy virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis delta virus Hepatitis E virus, Horsepox virus, Human adenovirus, Human astrovirus, Human coronavirus, Human cytomegalovirus. Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 16,18 Human papillomavirus 2, Human parainfluenza, Human parvovirus B19, Human respiratory syncytial virus, Human rhinovirus, Human SARS coronavirus, Human T-lymphotropic virus, Human torovirus, Influenza A virus, Influenza B virus, Influenza C virus, Isfahan virus, Japanese encephalitis virus, JC polyomavirus, Junin arenavirus, KI Polyomavirus, Lagos bat virus, Lake Victoria marburgvirus, Langat virus, Lassa virus, Louping ill virus, Lymphocytic choriomeningitis virus. Machupo virus, Mammalian orthorubulavirus 5 (Simian virus 5), Mayaro virus, Measles virus, Merkel cell polyomavirus, MERS coronavirus, Mokola virus, Molluscum contagiosum virus, Monkeypox virus. Mumps virus, Murray valley encephalitis virus, New York virus, Nipah virus, Norwalk virus. O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever Naples phlebovirus (Toscana virus), Sandfly fever sicilian virus, Sapporo virus, SARS coronavirus, SARS coronavirus 2, Semliki forest virus, Seoul virus, Simian foamy virus, Sindbis virus, Southampton virus, St. louis encephalitis virus, Tick-borne powassan virus, Torque teno virus, Uukuniemi virus, Vaccinia virus, Varicella-zoster virus, Variola virus, Venezuelan equine encephalitis virus, Vesicular stomatitis virus, West Nile virus, Western equine encephalitis virus, WU polyomavirus, Yaba monkey tumor virus, Yaba-like disease virus, Yellow fever virus, Zika virus, Khosta2 sarbecovirus, bat SARS-like WIV1 coronavirus, Kenya bat coronavirus BtKY72, pangolin Pang17 coronavirus, bat SARS HKU3 coronavirus, SARS Urbani coronavirus, bat RaTG13 coronavirus, Wuhan coronavirus, BA. 1 coronavirus, BA.2 coronavirus, B.1.351 coronavirus, any serotype thereof, any variant thereof, or any combination thereof.

In certain embodiments, the third antigen may comprise a third viral antigen. In certain embodiments, the third viral antigen may be derived from a viral pathogen. In certain embodiments, the third viral antigen may comprise any antigen derived from the third viral pathogen.

In certain embodiments, the third prokaryotic pathogen may comprise: Actinomyces israelii, Bacillus anthracis, Bacteroides fragilis, Bordetella pertussis, Borrelia, Brucella, Campylobacter jejuni, Chlamydia, Chlamydophila psittaci, Clostridium, Corynebacterium diphtheriae, Ehrlichia, Enterococcus, Escherichia, Francisella tularensis, Haemophilus influenzae, Helicobacter pylori, Klebsiella pneumoniae, Legionella pneumophila, Leptospira, Listeria monocytogenes, Mycobacterium, Mycoplasma pneumoniae, Neisseria, Pseudomonas aeruginosa, Nocardia asteroides, Rickettsia rickettsii, Salmonella, Shigella, Staphylococcus, Streptococcus, Treponema pallidum, Vibrio cholerae, Yersinia pestis, or any combination thereof.

In certain embodiments, the third antigen may comprise a third prokaryotic antigen. In certain embodiments, the third prokaryotic antigen may be derived from a prokaryotic pathogen. In certain embodiments, the third prokaryotic antigen may comprise any antigen derived from a third prokaryotic pathogen.

In certain embodiments, the third eukaryotic pathogen may comprise: Trypanosoma brucei, Entamoeba histolytica, Babesia bovis, Babesia divergens, Candida albicans, Trypanosoma cruzi, Giardia lamblia, Acanthamoeba culbertsoni, Necator americanus, Leishmania major, Bruga malayi, Plasmodium falciparum, Plasmodium vivax, Toxoplasma gondii, Trichomonas vaginalis, Aedes aegypti, or any combination thereof.

In certain embodiments, the third antigen may comprise a third eukaryotic antigen. In certain embodiments, the third eukaryotic antigen may be derived from a eukaryotic pathogen. In certain embodiments, the third eukaryotic antigen may comprise any antigen derived from a third eukaryotic pathogen.

In certain embodiments, the third viral antigen may comprise a third coronavirus antigen. In certain embodiments, the third coronavirus antigen may comprise an antigen from a Coronaviridae virus. In certain embodiments, the third coronavirus antigen may comprise a MERS-CoV antigen, a SARS-CoV-1 antigen, a SARS-CoV-2 antigen (for example but not limited to Wuhan coronavirus antigens, BA. 1 coronavirus antigens, BA.2 coronavirus antigens, or B.1.351 coronavirus antigens), a HCoV-HKU1 antigen, a HCoV-OC43 antigen, a BtCoV-RaTG13 antigen, a Bt SL-CoV-WIV1 antigen, a HKU4-CoV antigen, a HKU5-CoV antigen, a PDF-2108 CoV antigen, a SHC014-CoV antigen, a BtSCoV-RsSHC014 antigen, a BISCoV-Rs3367 antigen, a BANAL-52 antigen, a BANAL-236 antigen, a Pangolin-CoV GX/P2V antigen, a Bat coronavirus CDPHE15 antigen, a Bat coronavirus HKU10 antigen, a Rhinolophus ferrumequinum alphacoronavirus HuB-2013 antigen, a Human coronavirus 229E antigen, a Lucheng Rn rat coronavirus antigen, a Mink coronavirus 1 antigen, a Ferret coronavirus antigen, a Miniopterus bat coronavirus 1 antigen, a Miniopterus bat coronavirus HKU8 antigen, a Myotis ricketti alphacoronavirus Sax-2011 antigen, a Nyctalus velutinus alphacoronavirus SC-2013 antigen, a Pipistrellus kuhlii coronavirus 3398 antigen, a Porcine epidemic diarrhea virus antigen, a Scotophilus bat coronavirus 512 antigen, a Rhinolophus bat coronavirus HKU2 antigen, a Human coronavirus NL63 antigen, a NL63-related bat coronavirus strain BtKYNL63-9b antigen, a Sorex araneus coronavirus T14 antigen, a Suncus murinus coronavirus X74 antigen, an Alphacoronavirus 1 antigen, a Canine coronavirus antigen, a Feline coronavirus antigen, a Human CCoV-HuPn-2018 antigen, a Porcine transmissible gastroenteritis coronavirus antigen, a Swine acute diarrhea syndrome coronavirus (SADS-CoV) antigen, a HCoV-NL63 antigen, a NL63-related bat coronavirus strain BtKYNL63-9b antigen, a HCoV-229E antigen, a Khosta2 sarbecovirus antigen, a Kenya bat coronavirus BtKY72 antigen, a bat SARS coronavirus HKU3 antigen, or any combination thereof. In certain embodiments, the third viral antigen may comprise a sequence selected from SEQ ID NOs: 12 to 24 or a nucleic acid selected from SEQ ID NOs: 25-67 encoding the antigen. In certain embodiments, the third viral antigen may comprise an antigen derived from a Coronaviridae spike protein, S1 protein, receptor binding domain (RBD) and/or any fragment thereof. In certain embodiments the antigen sequence may have at least 90% identity, at least 91% identity, at least 92% identity, at least 93% identity, at least 94% identity, at least 95% identity, at least 96% identity, at least 97% identity, at least 98% identity, at least 99% identity to a sequence selected from SEQ ID NOs: 12 to 24 or the nucleic acid encoding the fusion polypeptide selected from SEQ ID NOs: 25-67.

In certain embodiments, the third coronavirus antigen may comprise a spike (S) protein, or fragment thereof. As used herein, a coronavirus spike (S) protein may comprise a spike protein or fragment thereof from a Betacoronavirus, a Gammacoronavirus, an Alphacoronavirus, a Deltacoronavirus or any combination thereof. In certain embodiments, the spike protein may comprise any Coronaviridae spike protein or a fragment thereof, a protein encoded by a nucleotide sequence with at least 70% identity to any Coronaviridae spike protein. In certain embodiments, the spike (S) protein, or fragment thereof may be a receptor binding domain (RBD) from a Betacoronavirus, a Gammacoronavirus, an Alphacoronavirus, a Deltacoronavirus or any combination thereof. In certain embodiments, the RBD may comprise any Coronaviridae RBD or a fragment thereof, or a protein encoded by a nucleotide sequence with at least 70% identity to any Coronaviridae RBD. In certain embodiments, the spike (S) protein, or fragment thereof may be an S1 protein from a Betacoronavirus, a Gammacoronavirus, an Alphacoronavirus, a Deltacoronavirus or any combination thereof. In certain embodiments, the spike protein may comprise any Coronaviridae S1 protein or a fragment thereof, or a protein encoded by a nucleotide sequence with at least 70% identity to any Coronaviridae S1 protein.

In certain embodiments, the fusion polypeptide may comprise a plurality of antigens (5), wherein the plurality of antigens may comprise vertebrate pathogens comprising prokaryotic antigens, viral antigens, fungal antigens, or any combination thereof.

In certain embodiments, the plurality of antigens of viral pathogens may comprise: Adeno-associated virus, Aichi virus, Australian bat lyssavirus, Banna virus, Barmah forest virus, BK polyomavirus, Bunyamwera virus, Bunyavirus La Crosse, Bunyavirus snowshoe hare, Cercopithecine herpesvirus Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern chimpanzee simian foamy virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis delta virus, Hepatitis E virus, Horsepox virus, Human adenovirus, Human astrovirus, Human coronavirus, Human cytomegalovirus. Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 16,18, Human papillomavirus 2, Human parainfluenza, Human parvovirus B19, Human respiratory syncytial virus, Human rhinovirus, Human SARS coronavirus, Human T-lymphotropic virus, Human torovirus, Influenza A virus, Influenza B virus, Influenza C virus, Isfahan virus, Japanese encephalitis virus, JC polyomavirus, Junin arenavirus, KI Polyomavirus, Lagos bat virus, Lake Victoria marburgvirus, Langat virus, Lassa virus, Louping ill virus, Lymphocytic choriomeningitis virus. Machupo virus, Mammalian orthorubulavirus 5 (Simian virus 5), Mayaro virus, Measles virus, Merkel cell polyomavirus, MERS coronavirus, Mokola virus, Molluscum contagiosum virus, Monkeypox virus. Mumps virus, Murray valley encephalitis virus, New York virus, Nipah virus, Norwalk virus. O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever Naples phlebovirus (Toscana virus), Sandfly fever sicilian virus, Sapporo virus, SARS coronavirus, SARS coronavirus 2, Semliki forest virus, Seoul virus, Simian foamy virus, Sindbis virus, Southampton virus, St. louis encephalitis virus, Tick-borne powassan virus, Torque teno virus, Uukuniemi virus, Vaccinia virus, Varicella-zoster virus, Variola virus, Venezuelan equine encephalitis virus, Vesicular stomatitis virus, West Nile virus, Western equine encephalitis virus, WU polyomavirus, Yaba monkey tumor virus, Yaba-like disease virus, Yellow fever virus, Zika virus, Khosta2 sarbecovirus, bat SARS-like WIV1 coronavirus, Kenya bat coronavirus BtKY72, pangolin Pang17 coronavirus, bat SARS HKU3 coronavirus, SARS Urbani coronavirus, bat RaTG13 coronavirus, Wuhan coronavirus, BA. 1 coronavirus, BA.2 coronavirus, B.1.351 coronavirus, any serotype thereof, any variant thereof, or any combination thereof.

In certain embodiments, the plurality of antigens may comprise a plurality of viral antigens. In certain embodiments, the plurality of viral antigens may be derived from a viral pathogen. In certain embodiments, the plurality of viral antigens may comprise any antigen derived from the plurality of antigens of viral pathogens.

In certain embodiments, the plurality of prokaryotic pathogens may comprise: Actinomyces israelii, Bacillus anthracis, Bacteroides fragilis, Bordetella pertussis, Borrelia, Brucella, Campylobacter jejuni, Chlamydia, Chlamydophila psittaci, Clostridium, Corynebacterium diphtheriae, Ehrlichia, Enterococcus, Escherichia, Francisella tularensis, Haemophilus influenzae, Helicobacter pylori, Klebsiella pneumoniae, Legionella pneumophila, Leptospira, Listeria monocytogenes, Mycobacterium, Mycoplasma pneumoniae, Neisseria, Pseudomonas aeruginosa, Nocardia asteroides, Rickettsia rickettsii, Salmonella, Shigella, Staphylococcus, Streptococcus, Treponema pallidum, Vibrio cholerae, Yersinia pestis, or any combination thereof.

In certain embodiments, the plurality of antigens may comprise a plurality of prokaryotic antigens. In certain embodiments, the plurality of prokaryotic antigens may be derived from a prokaryotic pathogen. In certain embodiments, the plurality of prokaryotic antigens may comprise any known antigen derived from the plurality of prokaryotic pathogens.

In certain embodiments, the plurality of eukaryotic pathogens may comprise: Trypanosoma brucei, Entamoeba histolytica, Babesia bovis, Babesia divergens, Candida albicans, Trypanosoma cruzi, Giardia lamblia, Acanthamoeba culbertsoni, Necator americanus, Leishmania major, Bruga malayi, Plasmodium falciparum, Plasmodium vivax, Toxoplasma gondii, Trichomonas vaginalis, Aedes aegypti, or any combination thereof.

In certain embodiments, the plurality of antigens may comprise a plurality of eukaryotic antigens. In certain embodiments, the plurality of eukaryotic antigens may be derived from a plurality of eukaryotic pathogens. In certain embodiments, the plurality of eukaryotic antigen may comprise any antigen derived from the plurality of eukaryotic antigens.

In certain embodiments, the plurality of viral antigens may comprise a plurality of coronavirus antigens. In certain embodiments, the plurality of coronavirus antigens may comprise antigens from a Coronaviridae virus. In certain embodiments, the plurality of coronavirus antigens may comprise antigens from a Betacoronavirus, a Gammacoronavirus, an Alphacoronavirus, a Deltacoronavirus or any combination thereof. In certain embodiments, the plurality of coronavirus antigens may comprise MERS-CoV antigens, SARS-CoV-1 antigens, SARS-CoV-2 antigens (for example but not limited to Wuhan coronavirus antigens, BA. 1 coronavirus antigens, BA.2 coronavirus antigens, or B.1.351 coronavirus antigens), HCoV-HKU1 antigens, HCoV-OC43 antigens, BtCoV-RaTG13 antigens, Bt SL-CoV-WIV1 antigens, HKU4-CoV antigens, HKU5-CoV antigens, PDF-2108 CoV antigens, SHC014-CoV antigens, BtSCoV-RsSHC014 antigens, BtSCoV-Rs3367 antigens, BANAL-52 antigens, BANAL-236 antigens, Pangolin-CoV GX/P2V antigens, Bat coronavirus CDPHE15 antigens, Bat coronavirus HKU10 antigens, Rhinolophus ferrumequinum alphacoronavirus HuB-2013 antigens, Human coronavirus 229E antigens, Lucheng Rn rat coronavirus antigens, Mink coronavirus 1 antigens, Ferret coronavirus antigens, Miniopterus bat coronavirus 1 antigens, Miniopterus bat coronavirus HKU8 antigens, Myotis ricketti alphacoronavirus Sax-2011 antigens, Nyctalus velutinus alphacoronavirus SC-2013 antigens, Pipistrellus kuhlii coronavirus 3398 antigens, Porcine epidemic diarrhea virus antigens, Scotophilus bat coronavirus 512 antigens, Rhinolophus bat coronavirus HKU2 antigens, Human coronavirus NL63 antigens, NL63-related bat coronavirus strain BtKYNL63-9b antigens, Sorex araneus coronavirus T14 antigens, Suncus murinus coronavirus X74 antigens, Alphacoronavirus 1 antigens, Canine coronavirus antigens, Feline coronavirus antigens, Human CCoV-HuPn-2018 antigens, Porcine transmissible gastroenteritis coronavirus antigens, Swine acute diarrhea syndrome coronavirus (SADS-CoV) antigens, HCoV-NL63 antigens, NL63-related bat coronavirus strain BtKYNL63-9b antigens, HCoV-229E antigens, Khosta2 sarbecovirus antigen, Kenya bat coronavirus BtKY72 antigens, bat SARS coronavirus HKU3 antigens, or any combination thereof. In certain embodiments, the plurality of viral antigens may comprise a sequence selected from SEQ ID NOs: 12 to 24 or a nucleic acid selected from SEQ ID NOs: 25-67 encoding the antigen. In certain embodiments, the plurality of viral antigens may comprise an antigen derived from a Coronaviridae spike protein, S1 protein, receptor binding domain (RBD) and/or any fragment thereof. In certain embodiments the antigen sequence may have at least 90% identity, at least 91% identity, at least 92% identity, at least 93% identity, at least 94% identity, at least 95% identity, at least 96% identity, at least 97% identity, at least 98% identity, at least 99% identity to a sequence selected from SEQ ID NOs: 12 to 24 or the nucleic acid encoding the fusion polypeptide selected from SEQ ID NOs: 25-67.

In certain embodiments, the plurality of coronavirus antigens may comprise a spike (S) protein, S1 protein, receptor binding domain, or fragment thereof. As used herein, a coronavirus spike (S) protein may comprise a spike protein or fragment thereof from a Betacoronavirus, a Gammacoronavirus, an Alphacoronavirus, a Deltacoronavirus or any combination thereof. In certain embodiments, the spike protein may comprise any Coronaviridae spike protein or a fragment thereof, or a protein encoded by a nucleotide sequence with at least 70% identity to any Coronaviridae spike protein. In certain embodiments, the spike (S) protein, or fragment thereof may be a receptor binding domain (RBD) from a Betacoronavirus, a Gammacoronavirus, an Alphacoronavirus, a Deltacoronavirus or any combination thereof. In certain embodiments, the RBD may comprise any Coronaviridae RBD or a fragment thereof, or a protein encoded by a nucleotide sequence with at least 70% identity to any Coronaviridae RBD. In certain embodiments, the spike (S) protein, or fragment thereof may be an S1 protein from a Betacoronavirus, a Gammacoronavirus, an Alphacoronavirus, a Deltacoronavirus or any combination thereof. In certain embodiments, the spike protein may comprise any Coronaviridae S1 protein or a fragment thereof, or a protein encoded by a nucleotide sequence with at least 70% identity to any Coronaviridae S1 protein.

In certain embodiments, the plurality of antigens may comprise two or more antigens, such as for example 2 antigens, 3 antigens, 4 antigens, 5 antigens, 6 antigens, 7 antigens, 8 antigens, 9 antigens or 10 antigens. In certain embodiments, the plurality of antigens may comprise at least 2 antigens. In certain embodiments, the plurality of antigens may comprise 2 to 4 antigens.

In certain embodiments, the terms “first antigen” and “third antigen” may refer to the number of antigens in the fusion construct, such that the “third” antigen is one of three said antigens present in the fusion polypeptide, such as three viral antigens. In certain embodiments, the terms “first” and “third” may comprise a position of the antigen in the fusion polypeptide, such that the “third” antigen is in a third position, such as for example, preceding the first antigen, but may only be the first or second of said antigen in the fusion polypeptide.

In certain embodiments, the fusion polypeptide may comprise a sequence selected from: SEQ ID NOs 1, 2, 3, 4, 5, 6, 7, 8, 9, 10 or 11 or a nucleic acid encoding the sequence thereof. In certain embodiments, the fusion polypeptide may consist of a sequence selected from: SEQ ID NOs 1, 2, 3, 4, 5, 6, 7, 8, 9, 10 or 11 or a nucleic acid encoding the sequence thereof. In certain embodiments, the fusion polypeptide may comprise a sequence with at least 90% identity, at least 91% identity, at least 92% identity, at least 93% identity, at least 94% identity, at least 95% identity, at least 96% identity, at least 97% identity, at least 98% identity, at least 99% identity to the fusion polypeptide of any one of SEQ ID NOs: 1-11.

As used herein, an “immunogenic” composition, protein, polypeptide or peptide may be a composition or a molecule which includes one or more epitopes and can modulate an immune response. Such peptides can be identified using any number of epitope mapping techniques, well known in the art. See, e.g., Epitope Mapping Protocols in Methods in Molecular Biology (2018) (Johan Rockberg and Johan Nilvebrant, Eds.) Springer, New York. For example, linear epitopes may be determined by for example, software programs, (See., e.g., Saha et al., Structure, Function, and Bioinformatics (2006) 65:40-48); or by concurrently synthesizing large numbers of peptides on solid supports, the peptides corresponding to portions of the protein molecule, and reacting the peptides with antibodies while the peptides are still attached to the supports. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins may be identified using standard antigenicity and hydropathy plots, such as those calculated using, for example, the Omiga software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., Mol. Biol. (1982) 157: 105-132 for hydropathy plots.

Immunogenic molecules, for purposes of the present invention, will usually be at least about 5 amino acids in length, such as at least about 10 to about 15 or more amino acids in length. There is no critical upper limit to the length of the molecule, which can comprise the full-length of the protein sequence, or even a fusion protein comprising two or more epitopes, proteins, antigens, etc.

As used herein, the term “epitope” may refer to a site on an antigen which is recognized by a T-cell receptor, a B-cell and/or an antibody. Several different epitopes may be carried by a single antigenic molecule. The term “epitope” also includes modified sequences of amino acids which stimulate responses against the whole organism. The epitope may be generated from knowledge of the amino acid and corresponding DNA sequences of the polypeptide, as well as from the nature of particular amino acids, such as size, charge etc., and the codon table, without undue experimentation, as would be known from, for example, Ivan Roitt, Essential Immunology, Janis Kuby, Immunology.

An “immunological response” to an antigen or composition is the development in a subject of a humoral and/or a cellular immune response to an antigen present in the composition of interest. As used herein, a “humoral immune response” may refer to an immune response mediated by antibody molecules, while a “cellular immune response” is one mediated by T-lymphocytes and/or other white blood cells. One important aspect of cellular immunity involves an antigen-specific response by cytotoxic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells may help stimulate the function, and focus the activity, of nonspecific, effector cells against cells displaying peptide antigens in association with MHC molecules on their surface. A “cellular immune response” may also refer to the production of cytokines, such as interferons, chemokines and other such molecules, produced by activated T-cells and/or other white blood cells, including those derived from CD4+ and CD8+ T-cells. In certain embodiments, the reduction in a pathogen may comprise the reduction of the pathogen by a humoral and/or cellular immune response to the antigen comprised in a fusion polypeptide in a subject.

Thus, an immunological response as used herein may be one that stimulates the production of antibodies. The antigen of interest may also elicit production of CTLs. Hence, an immunological response may include one or more of the following effects: the production of antibodies by B-cells; and/or the activation of suppressor T-cells and/or memory/effector T-cells directed specifically to an antigen or antigens present in the fusion polypeptide, composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The innate immune system of mammals also recognizes and responds to molecular features of pathogenic organisms via activation of Toll-like receptors and similar receptor molecules on immune cells. Upon activation of the innate immune system, various non-adaptive immune response cells are activated to, e.g., produce various cytokines, lymphokines and chemokines. Cells activated by an innate immune response include immature and mature dendritic cells of the monocyte and plasmacytoid lineage (MDC, PDC), as well as gamma, delta, alpha and beta T cells and B cells and the like. Thus, the present invention also contemplates an immune response wherein the immune response involves both an innate and adaptive response.

In certain embodiments there is provided herein an immunogenic composition that may comprise a fusion polypeptide as described herein, and a pharmaceutically acceptable salt, carrier, or adjuvant. In certain embodiments, the pharmaceutically acceptable salt, carrier, or adjuvant may comprise an oil-in-water emulsion adjuvant. In certain embodiments, the pharmaceutically acceptable salt, carrier, or adjuvant may comprise an squalene-in-water emulsion adjuvant. In certain embodiments, the pharmaceutically acceptable salt, carrier, or adjuvant may comprise Sepivac SWE™ adjuvant.

An “immunogenic composition” as used herein may be a composition that comprises an immunogenic molecule where administration of the composition to a subject results in the development in the subject of a humoral and/or a cellular immune response to the molecule(s) of interest.

In certain embodiments there is provided a use of a fusion polypeptide or the immunogenic composition as described herein in the manufacture of a medicament for the treatment or prevention of a pathogenic infection in a subject in need thereof.

In certain embodiments there is provided a use of a fusion polypeptide or the immunogenic composition described herein for the treatment or prevention of a pathogenic infection in a subject in need thereof.

In certain embodiments there is provided, a method of treating or preventing a pathogenic infection comprising administering an effective amount of a fusion polypeptide or immunogenic composition as described herein to a subject in need thereof. In certain embodiments, the effective amount is an amount sufficient to treat and/or prevent the pathogenic infection.

In certain embodiments, the pathogenic infection may comprise a viral infection from a viral pathogen, a prokaryotic infection from a prokaryotic pathogen, or a eukaryotic infection from a eukaryotic pathogen.

In certain embodiments, the viral infection may comprise an infection from Adeno-associated virus, Aichi virus, Australian bat lyssavirus, Banna virus, Barmah forest virus, BK polyomavirus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern chimpanzee simian foamy virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis delta virus, Hepatitis E virus, Horsepox virus, Human adenovirus, Human astrovirus, Human coronavirus, Human cytomegalovirus. Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 16,18, Human papillomavirus 2, Human parainfluenza, Human parvovirus B19, Human respiratory syncytial virus, Human rhinovirus, Human SARS coronavirus, Human T-lymphotropic virus, Human torovirus, Influenza A virus, Influenza B virus, Influenza C virus, Isfahan virus, Japanese encephalitis virus, JC polyomavirus, Junin arenavirus, KI Polyomavirus, Lagos bat virus, Lake Victoria marburgvirus, Langat virus, Lassa virus, Louping ill virus, Lymphocytic choriomeningitis virus. Machupo virus, Mammalian orthorubulavirus 5 (Simian virus 5), Mayaro virus, Measles virus, Merkel cell polyomavirus, MERS coronavirus, Mokola virus, Molluscum contagiosum virus, Monkeypox virus. Mumps virus, Murray valley encephalitis virus, New York virus, Nipah virus, Norwalk virus. O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever Naples phlebovirus (Toscana virus), Sandfly fever sicilian virus, Sapporo virus, SARS coronavirus, SARS coronavirus 2, Semliki forest virus, Seoul virus, Simian foamy virus, Sindbis virus, Southampton virus, St. louis encephalitis virus, Tick-borne powassan virus, Torque teno virus, Uukuniemi virus, Vaccinia virus, Varicella-zoster virus, Variola virus, Venezuelan equine encephalitis virus, Vesicular stomatitis virus, West Nile virus, Western equine encephalitis virus, WU polyomavirus, Yaba monkey tumor virus, Yaba-like disease virus, Yellow fever virus, Zika virus, any serotype thereof, any variant thereof, or any combination thereof.

In certain embodiments, the prokaryotic infection may comprise Actinomyces israelii, Bacillus anthracis, Bacteroides fragilis, Bordetella pertussis, Borrelia, Brucella, Campylobacter jejuni, Chlamydia, Chlamydophila psittaci, Clostridium, Corynebacterium diphtheriae, Ehrlichia, Enterococcus, Escherichia, Francisella tularensis, Haemophilus influenzae, Helicobacter pylori, Klebsiella pneumoniae, Legionella pneumophila, Leptospira, Listeria monocytogenes, Mycobacterium, Mycoplasma pneumoniae, Neisseria, Pseudomonas aeruginosa, Nocardia asteroides, Rickettsia rickettsii, Salmonella, Shigella, Staphylococcus, Streptococcus, Treponema pallidum, Vibrio cholerae, Yersinia pestis, or any combination thereof.

In certain embodiments, the eukaryotic infection may comprise Trypanosoma brucei, Entamoeba histolytica, Babesia bovis, Babesia divergens, Candida albicans, Trypanosoma cruzi, Giardia lamblia, Acanthamoeba culbertsoni, Necator americanus, Leishmania major, Bruga malayi, Plasmodium falciparum, Plasmodium vivax, Toxoplasma gondii, Trichomonas vaginalis, Aedes aegypti, or any combination thereof.

In certain embodiments, the viral infection may comprise a coronavirus. In certain embodiments, the coronavirus may comprise a Coronaviridae virus. In certain embodiments, the coronavirus may comprise MERS-CoV, SARS-CoV-1, SARS-CoV-2, HCoV-HKU1, HCoV-OC43, BtCoV-RaTG13, Bt SL-CoV-WIV1, HKU4-CoV, HKU5-CoV, PDF-2108 COV, SHC014-CoV, BtSCoV-RsSHC014, BtSCoV-Rs3367, BANAL-52, BANAL-236, Pangolin-CoV GX/P2V, Bat coronavirus CDPHE15, Bat coronavirus HKU10, Rhinolophus ferrumequinum alphacoronavirus HuB-2013, Human coronavirus 229E, Lucheng Rn rat coronavirus, Mink coronavirus 1, Ferret coronavirus, Miniopterus bat coronavirus 1, Miniopterus bat coronavirus HKU8, Myotis ricketti alphacoronavirus Sax-2011, Nyctalus velutinus alphacoronavirus SC-2013, Pipistrellus kuhlii coronavirus 3398, Porcine epidemic diarrhea virus, Scotophilus bat coronavirus 512, Rhinolophus bat coronavirus HKU2, Human coronavirus NL63, NL63-related bat coronavirus strain BtKYNL63-9b, Sorex araneus coronavirus T14, Suncus murinus coronavirus X74, Alphacoronavirus 1, Canine coronavirus, Feline coronavirus, Human CCoV-HuPn-2018, Porcine transmissible gastroenteritis coronavirus, Swine acute diarrhea syndrome coronavirus (SADS-CoV), HCoV-NL63. NL63-related bat coronavirus strain BtKYNL63-9b, HCoV-229E, Khosta2 sarbecovirus, bat SARS-like coronavirus WIV1, Kenya bat coronavirus BtKY72, bat coronavirus SHC014, pangolin coronavirus Pang17, bat SARS coronavirus HKU3, SARS coronavirus Urbani, bat coronavirus RaTG13, Wuhan coronavirus, BA. 1 coronavirus, BA.2 coronavirus, B.1.351 coronavirus or any combination thereof.

In certain embodiments, the fusion polypeptide or the immunogenic composition is administered or is for administration one or more times, such as, for example 1, 2, 3, 4, 5, 6, 7, 8, 9 or 10 times.

In certain embodiments, the fusion polypeptide or the immunogenic composition may neutralize two or more different pathogens, two or more different antigens, or both, such as for example 1, 2, 3, 4, 5, 6, 7, 8, 9 or 10 different pathogens or different antigens. In certain embodiments, the two or more different pathogens may comprise two or more protein variants derived from a same parental pathogen, such as for example, a SARS-CoV-2 omicron variant, a SARS-CoV-2 delta variant and/or a SARS-CoV-2 beta variant.

In certain embodiments, the fusion polypeptide or the immunogenic composition may reduce the quantity of the pathogen in the subject. In certain embodiments, the reduction of the quantity of the pathogen in a subject may comprise a reduction such that the subject is treated or prevented from symptoms associated with the pathogen. In certain embodiments, the reduction of the quantity of the pathogen in a subject may comprise a reduction such that the subject has diminished or improved symptoms associated with the pathogen. In certain embodiments, the reduction of the quantity of the pathogen in a subject may comprise a complete removal of the pathogen from the subject or the complete prevention of infection in the subject.

In certain embodiments, the fusion polypeptide or the immunogenic composition may comprise: a Wuhan coronavirus antigen, a Khosta2 sarbecovirus antigen and a bat SARS-like coronavirus WIV1 antigen. In certain embodiments, the fusion polypeptide or the immunogenic composition may comprise: a BA.2 coronavirus antigen, a Kenya bat coronavirus BtKY72 antigen and a BtSCoV-RsSHC014 antigen. In certain embodiments, the fusion polypeptide or the immunogenic composition may comprise: a Wuhan coronavirus antigen, a pangolin coronavirus Pang17 antigen and a bat SARS-like coronavirus WIV1 antigen. In certain embodiments, the fusion polypeptide or the immunogenic composition may comprise: a Wuhan coronavirus antigen, a Khosta2 sarbecovirus antigen, a BtSCoV-RsSHC014 antigen and a bat SARS-like coronavirus WIV1 antigen. In certain embodiments, the fusion polypeptide or the immunogenic composition may comprise: a BA.2 coronavirus antigen, a bat SARS coronavirus HKU3 antigen, a Kenya bat coronavirus BtKY72 antigen and a bat SARS-like coronavirus WIV1 antigen. In certain embodiments, the fusion polypeptide or the immunogenic composition may comprise: a Wuhan coronavirus antigen, a Khosta2 sarbecovirus antigen, a BtSCoV-RsSHC014 antigen and a SARS coronavirus Urbani antigen. In certain embodiments, the fusion polypeptide or the immunogenic composition may comprise: a Wuhan coronavirus antigen, a bat coronavirus RaTG13 antigen, a Kenya bat coronavirus BtKY72 antigen and a bat SARS-like coronavirus WIV1 antigen. In certain embodiments, the fusion or the immunogenic composition may comprise: a Wuhan coronavirus antigen and a BA. 1 coronavirus antigen. In certain embodiments, the fusion polypeptide or the immunogenic composition may comprise: a Wuhan coronavirus antigen and a BA.2 coronavirus antigen. In certain embodiments, the fusion polypeptide or the immunogenic composition may comprise: a Wuhan coronavirus antigen and a B. 1.617.2 coronavirus antigen. In certain embodiments, the fusion polypeptide or the immunogenic composition may comprise: a B. 1.351 coronavirus antigen, a Wuhan coronavirus antigen and a BA.2 coronavirus antigen. In certain embodiments, the fusion polypeptide or the immunogenic composition may comprise: a B.1.351 coronavirus antigen, a Wuhan coronavirus antigen and a BA.1 coronavirus antigen. In certain embodiments, the fusion polypeptide or composition may comprise any combination of two or more of: a Wuhan coronavirus antigen, a Khosta2 sarbecovirus antigen, a bat SARS-like coronavirus WIV1 antigen, a BA.2 coronavirus antigen, a Kenya bat coronavirus BtKY72 antigen, a BISCoV-RsSHC014 antigen, a pangolin coronavirus Pang17 antigen, a bat SARS coronavirus HKU3 antigen, a SARS coronavirus Urbani antigen, a bat coronavirus RaTG13 antigen and a B.1.617.2 coronavirus antigen. In certain embodiments, the fusion polypeptide or composition may comprise any combination of two or more sequences selected from 12-24 or a nucleic acid selected from SEQ ID NOs: 25-67 encoding the two or more sequences. In certain embodiments the antigen sequence may have at least 90% identity, at least 91% identity, at least 92% identity, at least 93% identity, at least 94% identity, at least 95% identity, at least 96% identity, at least 97% identity, at least 98% identity, at least 99% identity to a sequence selected from SEQ ID NOs: 12 to 24 or the nucleic acid encoding the fusion polypeptide selected from SEQ ID NOs: 25-67.

In certain embodiments, there is provided a nucleic acid encoding a fusion polypeptide as described herein. In certain embodiments, the nucleic acid encoding the fusion polypeptide may be selected from SEQ ID NOs: 25 to 67.

In certain embodiments, there is provided a cell transformed with a nucleic acid described herein or expressing a fusion polypeptide as described herein. The skilled person, in light of the teachings herein, would be able to select an appropriate method, such as a lipid-based transfection reagent, to transform the cell with the nucleic acid. In certain embodiments, the cell may comprise a mammalian cell, a yeast cell, a plant cell, a bacterial cell, or an insect/arthropod cell. The skilled person, in light of the teachings herein, would be able to select an appropriate cell for a given application. In certain embodiments, the cell comprises a Chinese Hamster Ovary (CHO) cell.

As used herein a “subunit vaccine” may refer to a vaccine composition that includes one or more selected antigens but not all antigens, derived from or homologous to, an antigen from a pathogen of interest. Such a composition may be substantially free of intact pathogen cells or pathogenic particles, or the lysate of such cells or particles. Thus, a “subunit vaccine” can be prepared from at least partially purified (preferably substantially purified) immunogenic molecules from the pathogen, or analogs thereof. The skilled person, in light of the teachings herein, would be able to select a method of obtaining an antigen included in the subunit vaccine, such as standard purification techniques, recombinant production, or synthetic production.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

As used herein, “different pathogen” may comprise the same pathogen, such as for example, a coronavirus virus but the “different pathogen” may comprise different mutations or variants in the genome of the different pathogen. In certain embodiments, the different pathogen may comprise a different protein from the same pathogen, such as, for example, a SARS-CoV-2 spike protein and a SARS-CoV-2 nucleocapsid protein. In certain embodiments, the different pathogen may comprise any pathogen that is a different organism, such as, for example, a Coronaviridae and an Orthomyxoviridae. In certain embodiments, the different pathogen may comprise any pathogen that is a different variant, such as, for example, a SARS-CoV-2 omicron variant and a SARS-CoV-2 delta variant. In certain embodiments, the different pathogen may comprise differences in one or more of the following: phyla, organism, genus, lineage, species, variant, serotype, and strain.

As used herein, the term “variant” may comprise the ancestral or parental pathogen, wherein a genome of the ancestral or parental pathogen has been modified, such as for example, one or more base pair change in the genome, one or more base pair deletion in the genome and/or one or more base pair addition in the genome as compared to the genome of the ancestral or parental pathogen. In certain embodiments, the properties of the variant ancestral or variant parental pathogen may be modified when compared to the ancestral or parental pathogen. In certain embodiments, the variant may comprise a genome modification to the ancestral or parental pathogen that enhances transmission of the pathogen, worsens symptoms associated with infection by the pathogen, and/or impairs vaccine elicited immunity. In certain embodiments, the “variant” may be a “variant of concern”. As used herein, a “variant of concern” may be any variant, wherein a genomic modification comprising the variant may modify the properties of the variant in comparison to the ancestral or parent pathogen, or be predicted to modify the properties of the variant in comparison to the ancestral or parent pathogen, such that the variant of concern may be a risk to public health or have the potential for risk to public health, such as a hypothesized change in properties of the pathogen. In certain embodiments, the “variant” or the “variant of concern” may comprise a different serotype to the ancestral or parental pathogen.

The following examples are provided for illustrative purposes and are intended for the person of skill in the art. These examples are provided to demonstrate certain embodiments as described herein, and should not be seen as limiting in any way.

### EXAMPLES

### Example 1: Expression and Purification of Fusion Polypeptides

Fusion proteins were expressed by transient transfection in Expi-CHO cells as per the manufacturers instruction. In brief, cells were transfected with 0.6 μg/ml of plasmid DNA and subsequently cultured for 3-8 days until protein production reached peak levels or cell viability began to decrease. Supernatant containing the proteins was harvested then clarified by centrifugation at 4000×g for 30 min at 4° C., the addition of 300 nM NaCl and 0.05% Tween 80 and a final filtration through a 0.2 μm filter. Clarified supernatant was concentrated, diluted 1:1 in PBS pH 8 and then loaded onto Repligen NGL Covid-19 Spike protein AR 2.0 resin. Column volume and residence times was adjusted based on the expected protein in the sample and the number of receptor binding domains (RBDs) present in the construct. The columns were washed with 10 column volumes (CV) of phosphate buffered saline (PBS) pH 8 prior to elution in 8 CV of 100 mM sodium acetate pH 5.0+1 M arginine. Fractions containing S1 protein constructs were buffer exchanged into PBS and sterile filtered prior to formulation.

### Example 2: Administration of Immunogenic Compositions Comprising Fusion Polypeptides

Six to eight week old male hamsters were vaccinated twice intramuscularly (IM), 28 days apart, with COVAC-2, a non-fused antigen of the S1 subunit (WuhanS1), fusion polypeptide antigen 4304 (WuhanS1-oRBD) or fusion polypeptide antigen 4296 (WuhanS1-dRBD) all formulated with Sepivac SWE™ adjuvant. Three weeks after the second vaccination, animals were challenged intranasally (IN) with either the Beta, Delta, or Omicron SARS-CoV-2 variant. To compare the protective immunity between fusion (linked) vs unlinked antigens, hamsters were vaccinated twice IM, 28 days apart, with either fusion polypeptide antigen 4372 or an unlinked 4372 triple antigen both formulated with Sepivac SWE™ adjuvant. Three weeks after the second vaccination, animals were challenged IN with the SARS-CoV-2 Delta variant. Animal weight and clinical signs were monitored throughout the challenge. At 5- and 10-days post-challenge (dpc), half of the animals from each group were euthanized and nasal washes and lung samples were collected for virological and pathological analysis. Sera were collected for ELISA and viral neutralization assay before each immunization and challenge.

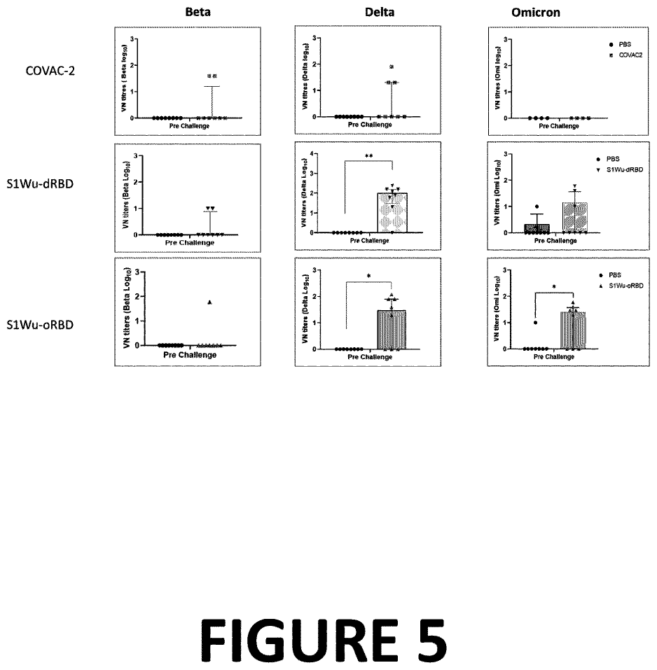

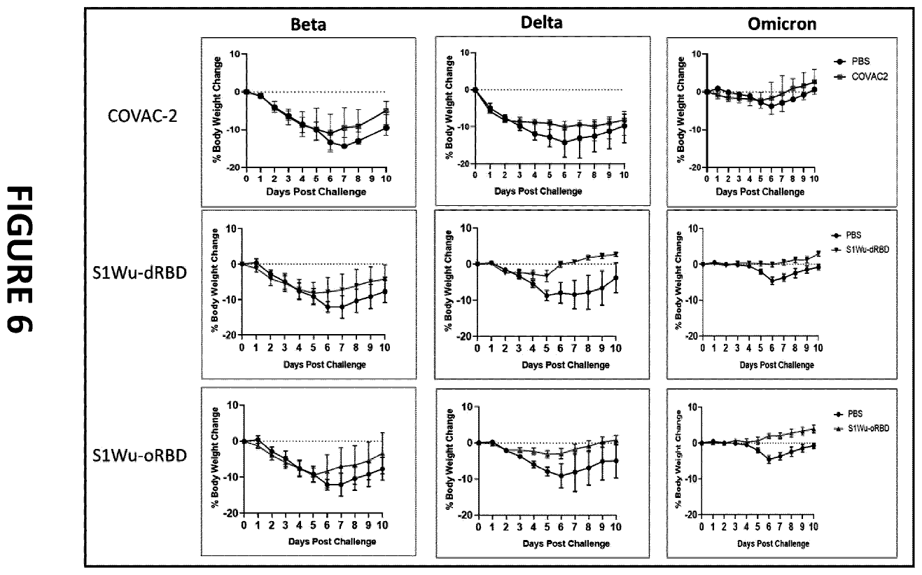

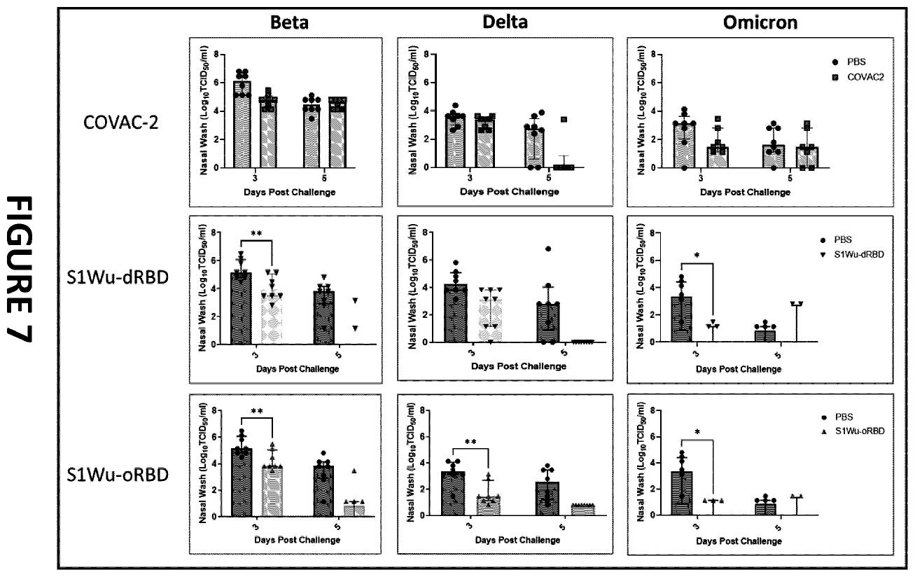

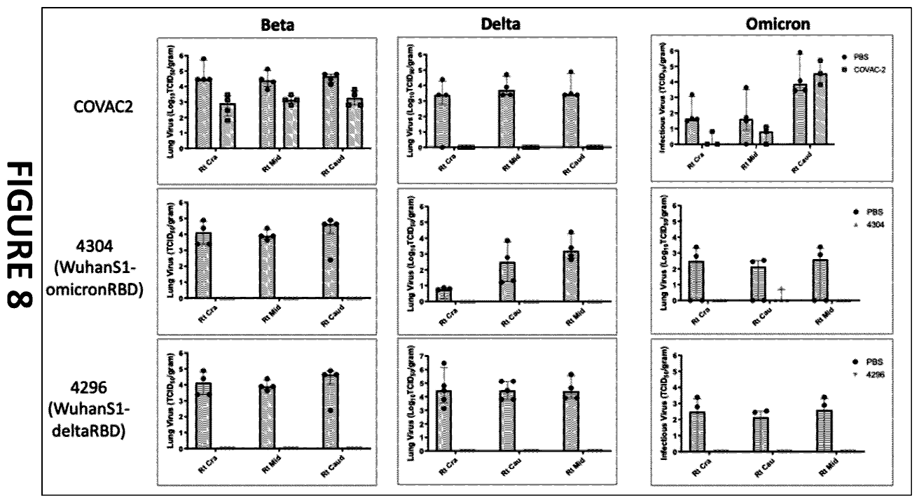

In contrast to COVAC-2, both the S1Wu-dRBD and the S1Wu-oRBD induced high virus neutralizing antibody titres in immunized animals prior to challenge (FIG. 5) that significantly prevented both weight loss (FIG. 6) and replication of the infectious virus in the upper respiratory tract (FIG. 7) against all tested variants of concern (VOCs).

COVAC-2 vaccinated Syrian hamsters lost weight when challenged with Beta and Delta VOCs, however, weight loss was slightly lower when compared to unvaccinated animals (FIG. 6). Both mock and COVAC-2 vaccinated animals gained weight starting 7 days post-infection with the Omicron VOC (FIG. 6). 4304 vaccinated hamsters lost less weight compared to mock vaccinated animals on challenge with Delta and Omicron VOCs (FIG. 6). 4296 vaccinated hamsters lost less weight compared to mock vaccinated animals on challenge with Delta and Omicron VOCs (FIG. 6).

COVAC-2 did not prevent nasal shedding of SARS-CoV-2 when challenged with Beta, Delta, and Omicron VOCs, except for reduced shedding at day 5 post challenge with Delta VOC (FIG. 7). 4304 vaccinated animals had reduced nasal shedding of virus as early as 3 days post challenge with Beta, Delta, and Omicron VOCs (FIG. 7). 4296 vaccinated animals had reduced nasal shedding of virus as early as 3 days post challenge with Beta and Omicron VOCs (FIG. 7).

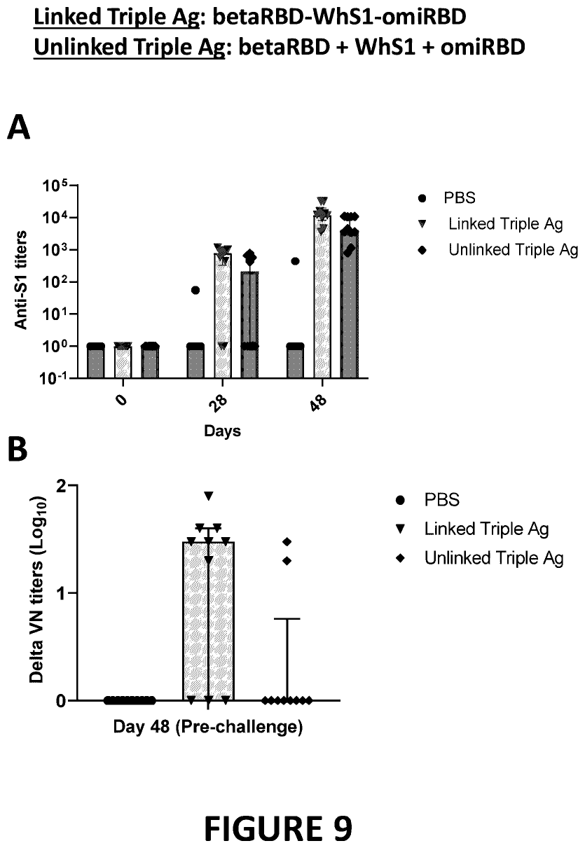

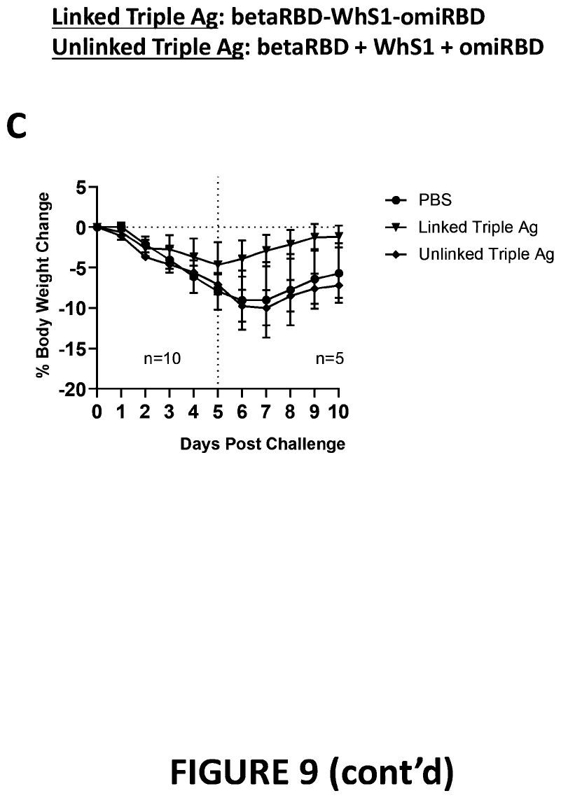

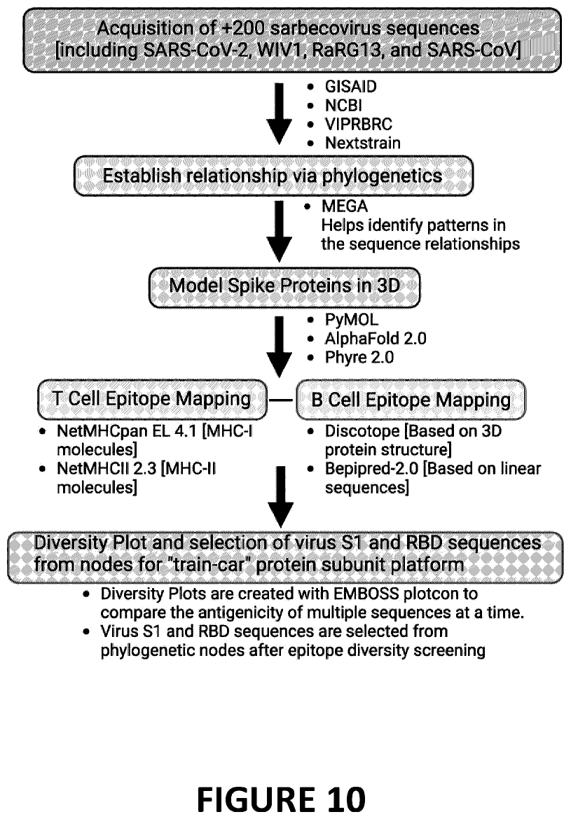

In contrast to the control group, both the linked fusion antigen (betaRBD-WhS1-omiRBD) and unlinked induced high virus S1 neutralizing antibody titres in immunized animals prior to challenge (FIG. 9A). Additionally, in contrast to both the control and unlinked triple antigen, the fusion betaRBD-WhS1-omiRBD induced high virus Delta-specific neutralizing antibody titers (FIG. 9B), in the absence of exposure to a Delta-specific antigen, that significantly prevented weight loss in response to a challenge with the Delta variant (FIG. 9C). The fusion polypeptide vaccine (linked) provided protection against heterologous strain(s) not included in the vaccine, whereas the unlinked version of the vaccine did not.

### Example 3: Pan-Sarbecovirus Candidate Antigen Design Strategy

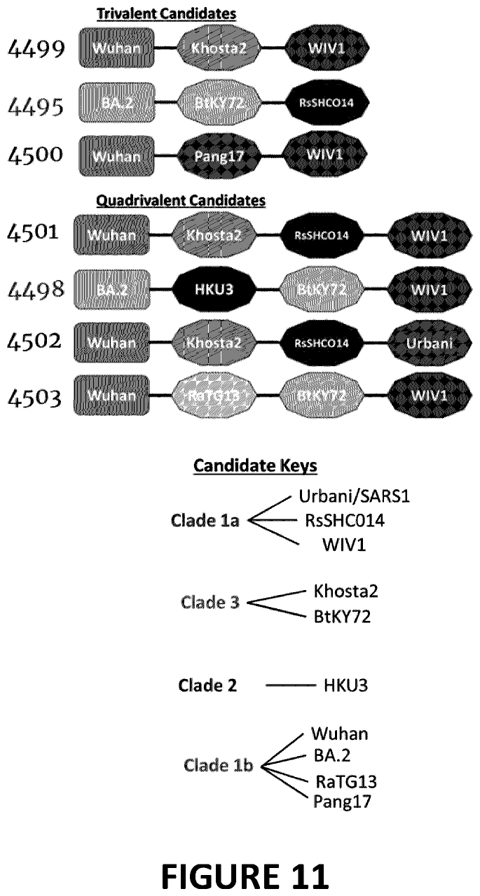

For the informatics pipeline approach, two hundred sarbecovirus spike protein sequences were acquired from GISAID and Nextstrain. Phylogenetic trees were constructed to identify relatedness among the viruses within each lineage and among the lineages using an evolutionary history approach utilizing the Maximum Likelihood method and JTT matrix-based model. Heuristic searches were used for the trees obtained automatically by applying Neighbor-Join and BioNJ algorithms to a matrix of pairwise distances estimated using the JTT model, and the topology was selected with superior log likelihood value. Evolutionary analyses were conducted in MEGA11. Rationally chosen sequences representing important nodes from each lineage on the tree at distances to cover specific clades were utilized for the vaccine candidate designs. A representative from each of the sarbecovirus clades were chosen to be included in the antigen and approximately 10 candidates were designed based on this approach (FIG. 11).

Before the candidate proteins were progressed to lead candidate selection, each candidate was screened for B cell and T cell epitopes within the chosen “engine” and “car” sequences. The diversity and similarity of each chosen component sequence in the candidates was determined. The candidates were removed from contention if the identified component sequences were not immunogenic or did not have optimal diversity to induce broadly protective responses. The B cell epitopes were predicted using Discotope and Bepipred-2.0, while T cell epitopes were predicted using NetMHCpan EL 4.1 for MHC-I molecules and NetMHCII 2.3 server for MHC-II molecules. Diversity plots were devised using EMBOSS plotcon to measure the variability within a determined length of peptide compared to the other antigens within each candidate. A BLOSUM90 scoring matrix was used to identify similar and identical sequences. Candidates that had a score reflecting a too high or too low level of similarity between the different components within the same antigen were removed, as it was hypothesized that these candidates will not be effective at eliciting a cross-reactive response. The individual epitope results were compared in RStudio using the tidyverse package (version 1.3.0) to identify epitopes shared across viruses.

Ultimately, 7 candidates were constructed (FIG. 11) and used to test their ability to be expressed and purified.

### Example 4: Candidate Evaluation in Mice

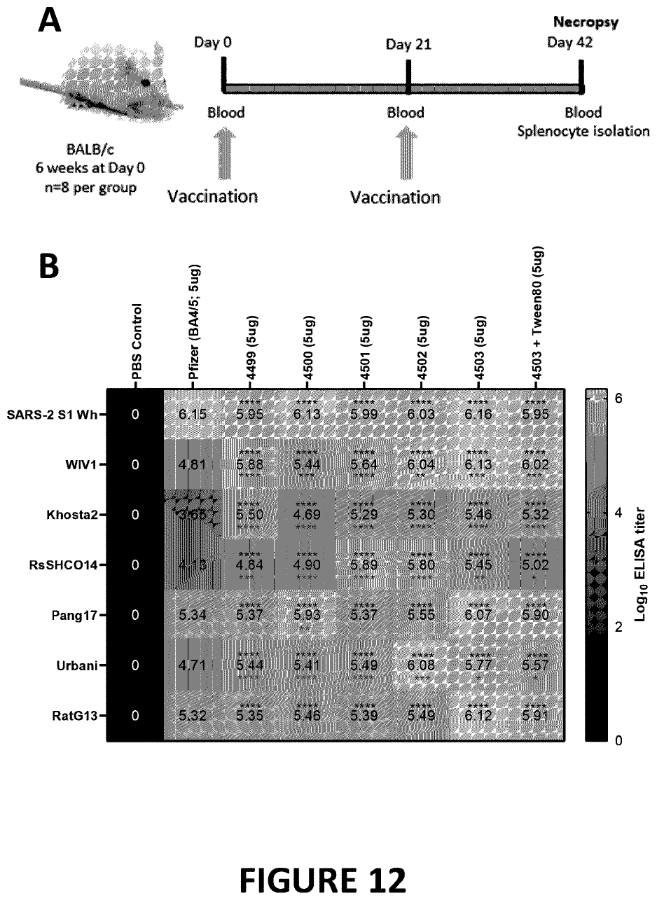

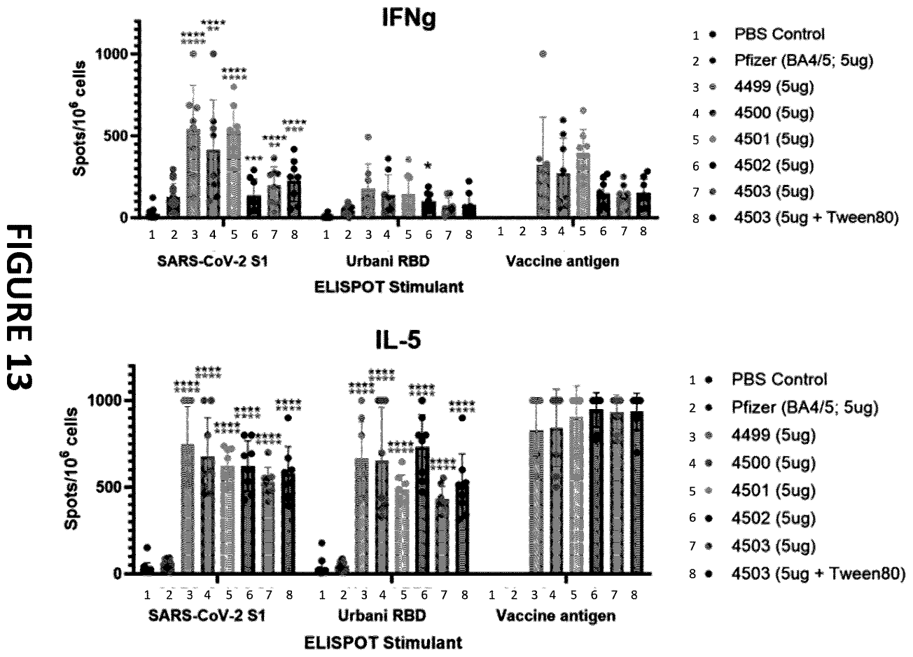

Mouse studies were carried out to evaluate the immunogenicity of all purified fusion polypeptide candidates (FIG. 12a). Animals received 5 μg of vaccine comprising a fusion polypeptide on Days 0 and 21 and were euthanized on Day 42. A Pfizer bivalent (BA4/5) comparator vaccine was included in all trials. Serum binding ELISA (FIG. 12b) and IFNg/IL-5 ELISPOT (FIG. 13) were completed. As 4503 may be primarily present as a dimer, it was evaluated both with and without Tween 80.

The serum binding ELISA (FIG. 12b) shows that the candidate antigens induce better binding antibodies (B cell response) against the panel of coronaviruses (against their respective Receptor Binding Domains (RBDs)) than the placebo group (blue stars above values), and that many also produce better binding antibodies than the Pfizer bivalent mRNA vaccine (red stars below values). Regardless of the number or positioning of antigens in the fusion polypeptide, the level of binding antibodies to SARS-CoV-2 S1 Wuhan (Wh) is similar (top row of FIG. 12b). Additionally, all the candidate antigens have demonstrated that they induce significant binding antibodies to viruses that are not represented in the candidate antigen. For example, candidate 4500, which contains Pang17 and WIV1 antigens was able to induce significant antibodies against Khosta2, RsSHC014, Urbani and RatG13.

The IFNg/IL-5 ELISPOT (FIG. 13) shows fusion polypeptides comprising the candidate antigens are also able to induce a better T cell response (IFNg and IL-5) than the Pfizer bivalent mRNA vaccine.

### Example 5: Candidate Evaluation in Hamsters

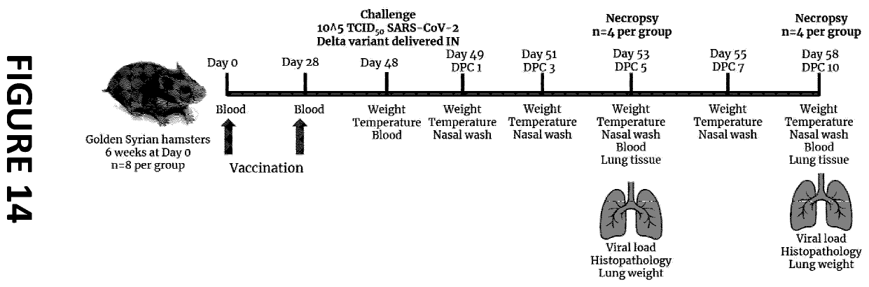

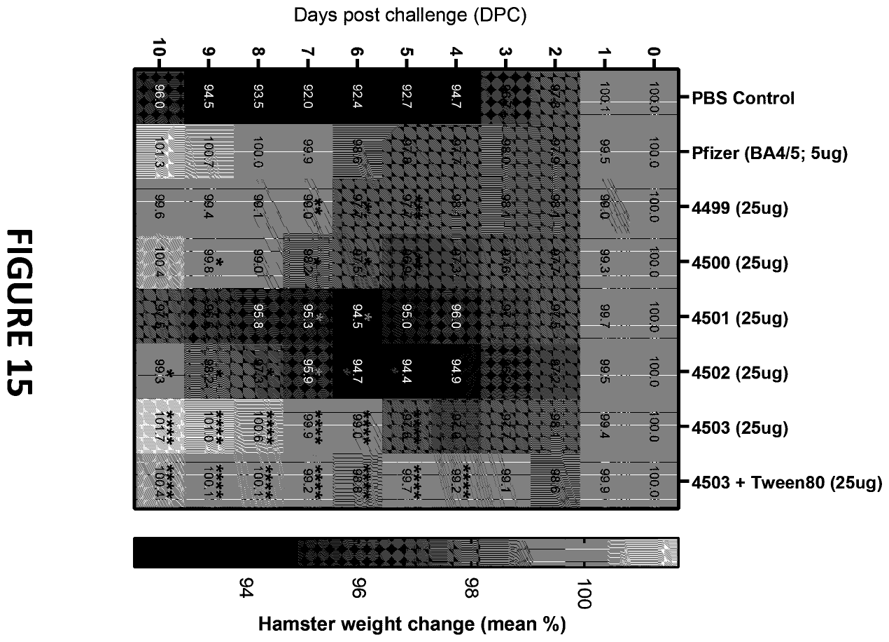

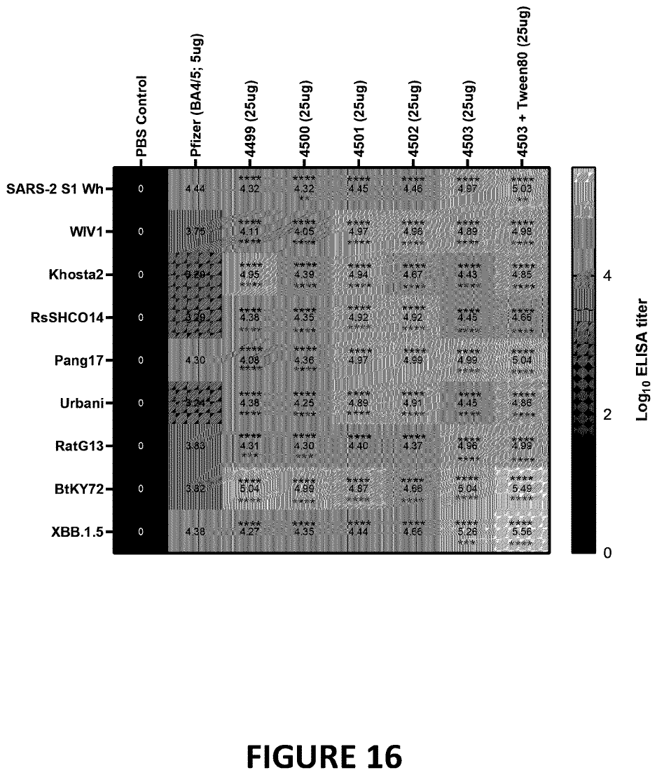

Trials were carried out to evaluate vaccine candidates comprising fusion polypeptides as described herein (FIG. 14), with each trial containing a PBS control group and a Pfizer bivalent vaccine group (BA4/5; 5 μg dose) in addition to the groups outlined in FIGS. 15 and 16. Hamsters were vaccinated on Day 0 and Day 28 with respective antigens combined with SWE adjuvant. Hamsters were challenged intranasally on Day 48 with the Delta variant of SARS-CoV-2 (1×10⁵ TCID₅₀). Serum was collected for ELISA and viral neutralization assays on Days 0, 28 and 48. Nasal wash samples were collected on Days 1, 3, 5, 7 and 10 post challenge. Animals were euthanized on Days 5 and 10 post challenge for respiratory tissue collection. As 4503 may be primarily present as a dimer, it was evaluated both with and without Tween 80.

The daily weight of hamsters was recorded and daily weight changes as a percentage of pre-challenge weight were calculated (FIG. 15). This data demonstrates that fusion polypeptides comprising candidate antigens as described herein were able to protect hamsters from weight loss after they were challenged (infected) with SARS-CoV-2 Delta variant of concern. Some candidates protected better than others, but this infection may be considered a heterologous infection because none of the vaccine candidates had a SARS-CoV-2 Delta component in them which indicates broad protection.

The ELISA (FIG. 16) shows that the candidate antigens induce better binding antibodies (B cell response) against the panel of coronaviruses (against their respective Receptor Binding Domains (RBDs)) than the placebo group (blue stars above values), and that many also produce better binding antibodies than the Pfizer bivalent mRNA vaccine (red stars below values). Regardless of the number or positioning of antigens in the fusion polypeptide, the level of binding antibodies to SARS-2 S1 Wuhan (Wh) is similar (top row of FIG. 16). Additionally, candidate 4503 produced more binding antibodies against SARS-CoV-2 XBB.1.5 variant of concern (an Omicron sublineage) than the Pfizer bivalent control vaccine did. This is significant because the Pfizer vaccine contains BA4/5 which is also an Omicron sublineage.

TABLE 1
Amino Acid sequences of exemplary fusion polypeptides
and antigens as described herein.

| Identifier | Sequence |
|---|---|
| 4296_WHS1_D | MFVFLVLLPLVSSQCVNLTTRTQLPPAYTNSFTRGVYYPDKVFRSSVLHSTQDL |
| eltaRBD (SEQ | FLPFFSNVTWFHAIHVSGTNGTKRFDNPVLPFNDGVYFASTEKSNIIRGWIFGTT |
| ID NO: 1) | LDSKTQSLLIVNNATNWVIKVCEFQFCNDPFLGVYYHKNNKSWMESEFRVYSS |
| | ANNCTFEYVSQPFLMDLEGKQGNFKNLREFVFKNIDGYFKIYSKHTPINLVRDL |
| | PQGFSALEPLVDLPIGINITRFQTLLALHRSYLTPGDSSSGWTAGAAAYYVGYL |
| | QPRTFLLKYNENGTITDAVDCALDPLSETKCTLKSFTVEKGIYQTSNFRVQPTES |
| | IVRFPNITNLCPFGEVFNATRFASVYAWNRKRISNCVADYSVLYNSASFSTFKC |
| | YGVSPTKLNDLCFTNVYADSFVIRGDEVRQIAPGQTGKIADYNYKLPDDFTGCV |
| | IAWNSNNLDSKVGGNYNYLYRLFRKSNLKPFERDISTEIYQAGSTPCNGVEGFN |
| | CYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGGV |
| | SVITPGTNTSNQVAVLYQDVNCTEVPVAIHADQLTPTWRVYSTGSNVFQTRAG |
| | CLIGAEHVNNSYECDIPIGAGICASYQTQTNSPRGSGSGSGRVQPTESIVRFPNI |
| | TNLCPFGEVFNATRFASVYAWNRKRISNCVADYSVLYNSASFSTFKCYGVSPT |
| | KLNDLCFTNVYADSFVIRGDEVRQIAPGQTGKIADYNYKLPDDFTGCVIAWNSN |
| | NLDSKVGGNYNYRYRLFRKSNLKPFERDISTEIYQAGSKPCNGVEGFNCYFPL |
| | QSYGFQPTNGVGYQPYRVVVLSFELLHAPATVCGPKKSTNLVKNKCVNFNFN |
| | G |
| 4304_WHS1_ | MFVFLVLLPLVSSQCVNLTTRTQLPPAYTNSFTRGVYYPDKVFRSSVLHSTQDL |
| OmicronRBD | FLPFFSNVTWFHAIHVSGTNGTKRFDNPVLPFNDGVYFASTEKSNIIRGWIFGTT |
| (SEQ ID NO: 2) | LDSKTQSLLIVNNATNVVIKVCEFQFCNDPFLGVYYHKNNKSWMESEFRVYSS |
| | ANNCTFEYVSQPFLMDLEGKQGNFKNLREFVFKNIDGYFKIYSKHTPINLVRDL |
| | PQGFSALEPLVDLPIGINITRFQTLLALHRSYLTPGDSSSGWTAGAAAYYVGYL |
| | QPRTFLLKYNENGTITDAVDCALDPLSETKCTLKSFTVEKGIYQTSNFRVQPTES |
| | IVRFPNITNLCPFGEVFNATRFASVYAWNRKRISNCVADYSVLYNSASFSTFKC |
| | YGVSPTKLNDLCFTNVYADSFVIRGDEVRQIAPGQTGKIADYNYKLPDDFTGCV |
| | IAWNSNNLDSKVGGNYNYLYRLFRKSNLKPFERDISTEIYQAGSTPCNGVEGEN |
| | CYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGGV |
| | SVITPGTNTSNQVAVLYQDVNCTEVPVAIHADQLTPTWRVYSTGSNVFQTRAG |
| | CLIGAEHVNNSYECDIPIGAGICASYQTQTNSPRGSGSGSGRVQPTESIVRFPNI |
| | TNLCPFDEVFNATRFASVYAWNRKRISNCVADYSVLYNLAPFFTFKCYGVSPTK |
| | LNDLCFTNVYADSFVIRGDEVRQIAPGQTGNIADYNYKLPDDFTGCVIAWNSNK |
| | LDSKVSGNYNYLYRLFRKSNLKPFERDISTEIYQAGNKPCNGVAGENCYFPLRS |
| | YSFRPTYGVGHQPYRVVVLSFELLHAPATVCGPKKSTNLVKNKCVNFNFNG |
| 4371_BetaRBD | MFVFLVLLPLVSSRVQPTESIVRFPNITNLCPFGEVFNATRFASVYAWNRKRISN |
| _WHS1_DeltaR | CVADYSVLYNSASFSTFKCYGVSPTKLNDLCFTNVYADSFVIRGDEVRQIAPGQ |
| BD (SEQ ID NO: | TGNIADYNYKLPDDFTGCVIAWNSNNLDSKVGGNYNYLYRLFRKSNLKPFERDI |
| 3) | STEIYQAGSTPCNGVKGFNCYFPLQSYGFQPTYGVGYQPYRVVVLSFELLHAP |
| | ATVCGPKKSTNLVKNKCVNFGSGSGSGMQCVNLTTRTQLPPAYTNSFTRGVY |
| | YPDKVFRSSVLHSTQDLFLPFFSNVTWFHAIHVSGTNGTKRFDNPVLPFNDGV |
| | YFASTEKSNIIRGWIFGTTLDSKTQSLLIVNNATNVVIKVCEFQFCNDPFLGVYYH |
| | KNNKSWMESEFRVYSSANNCTFEYVSQPFLMDLEGKQGNFKNLREFVFKNID |
| | GYFKIYSKHTPINLVRDLPQGFSALEPLVDLPIGINITRFQTLLALHRSYLTPGDS |
| | SSGWTAGAAAYYVGYLQPRTFLLKYNENGTITDAVDCALDPLSETKCTLKSFTV |
| | EKGIYQTSNFRVQPTESIVRFPNITNLCPFGEVFNATRFASVYAWNRKRISNCV |
| | ADYSVLYNSASFSTFKCYGVSPTKLNDLCFTNVYADSFVIRGDEVRQIAPGQTG |
| | KIADYNYKLPDDFTGCVIAWNSNNLDSKVGGNYNYLYRLFRKSNLKPFERDIST |
| | EIYQAGSTPCNGVEGFNCYFPLQSYGFQPTNGVGYQPYRVVVLSFELLHAPAT |
| | VCGPKKSTNLVKNKCVNFNFNGLTGTGVLTESNKKFLPFQQFGRDIADTTDAV |
| | RDPQTLEILDITPCSFGGVSVITPGTNTSNQVAVLYQDVNCTEVPVAIHADQLTP |
| | TWRVYSTGSNVFQTRAGCLIGAEHVNNSYECDIPIGAGICASYQTQTNSPRGS |
| | GSGSGRVQPTESIVRFPNITNLCPFGEVFNATRFASVYAWNRKRISNCVADYSV |
| | LYNSASFSTFKCYGVSPTKLNDLCFTNVYADSFVIRGDEVRQIAPGQTGKIADY |
| | NYKLPDDFTGCVIAWNSNNLDSKVGGNYNYRYRLFRKSNLKPFERDISTEIYQA |
| | GSKPCNGVEGFNCYFPLQSYGFQPTNGVGYQPYRVVVLSFELLHAPATVCGP |
| | KKSTNLVKNKCVNFNFNG |
| 4372_BetaRBD | MFVFLVLLPLVSSRVQPTESIVRFPNITNLCPFGEVFNATRFASVYAWNRKRISN |
| _WHS1_Omicron | CVADYSVLYNSASFSTFKCYGVSPTKLNDLCFTNVYADSFVIRGDEVRQIAPGQ |
| RBD (SEQ ID | TGNIADYNYKLPDDFTGCVIAWNSNNLDSKVGGNYNYLYRLFRKSNLKPFERDI |
| NO: 4) | STEIYQAGSTPCNGVKGFNCYFPLQSYGFQPTYGVGYQPYRVVVLSFELLHAP |
| | ATVCGPKKSTNLVKNKCVNFGSGSGSGMQCVNLTTRTQLPPAYTNSFTRGVY |
| | YPDKVFRSSVLHSTQDLFLPFFSNVTWFHAIHVSGTNGTKRFDNPVLPFNDGV |
| | YFASTEKSNIIRGWIFGTTLDSKTQSLLIVNNATNVVIKVCEFQFCNDPFLGVYYH |
| | KNNKSWMESEFRVYSSANNCTFEYVSQPFLMDLEGKQGNFKNLREFVFKNID |
| | GYFKIYSKHTPINLVRDLPQGFSALEPLVDLPIGINITRFQTLLALHRSYLTPGDS |
| | SSGWTAGAAAYYVGYLQPRTFLLKYNENGTITDAVDCALDPLSETKCTLKSFTV |
| | EKGIYQTSNFRVQPTESIVRFPNITNLCPFGEVFNATRFASVYAWNRKRISNCV |
| | ADYSVLYNSASFSTFKCYGVSPTKLNDLCFTNVYADSFVIRGDEVRQIAPGQTG |
| | KIADYNYKLPDDFTGCVIAWNSNNLDSKVGGNYNYLYRLFRKSNLKPFERDIST |
| | EIYQAGSTPCNGVEGFNCYFPLQSYGFQPTNGVGYQPYRVVVLSFELLHAPAT |
| | VCGPKKSTNLVKNKCVNFNFNGLTGTGVLTESNKKFLPFQQFGRDIADTTDAV |
| | RDPQTLEILDITPCSFGGVSVITPGTNTSNQVAVLYQDVNCTEVPVAIHADQLTP |
| | TWRVYSTGSNVFQTRAGCLIGAEHVNNSYECDIPIGAGICASYQTQTNSPRGS |
| | GSGSGRVQPTESIVRFPNITNLCPFDEVFNATRFASVYAWNRKRISNCVADYSV |
| | LYNLAPFFTFKCYGVSPTKLNDLCFTNVYADSFVIRGDEVRQIAPGQTGNIADY |
| | NYKLPDDFTGCVIAWNSNKLDSKVSGNYNYLYRLFRKSNLKPFERDISTEIYQA |
| | GNKPCNGVAGFNCYFPLRSYSFRPTYGVGHQPYRVVVLSFELLHAPATVCGP |
| | KKSTNLVKNKCVNFNFNG |
| 4495_[S1_ | MFVFLVLLPLVSSQCVNLITRTQSYTNSFTRGVYYPDKVFRSSVLHSTQDLFLP |
| Omicron_ | FFSNVTWFHAIHVSGTNGTKRFDNPVLPFNDGVYFASTEKSNIIRGWIFGTTLD |
| BtKY72_Rs | SKTQSLLIVNNATNVVIKVCEFQFCNDPFLDVYYHKNNKSWMESEFRVYSSAN |
| SCHC014] | NCTFEYVSQPFLMDLEGKQGNFKNLREFVFKNIDGYFKIYSKHTPINLGRDLPQ |
| (SEQ ID NO: 5) | GFSALEPLVDLPIGINITRFQTLLALHRSYLTPGDSSSGWTAGAAAYYVGYLQPR |
| | TFLLKYNENGTITDAVDCALDPLSETKCTLKSFTVEKGIYQTSNFRVQPTESIVR |
| | FPNITNLCPFDEVFNATRFASVYAWNRKRISNCVADYSVLYNFAPFFAFKCYGV |
| | SPTKLNDLCFTNVYADSFVIRGNEVSQIAPGQTGNIADYNYKLPDDFTGCVIAW |
| | NSNKLDSKVGGNYNYLYRLFRKSNLKPFERDISTEIYQAGNKPCNGVAGENCY |
| | FPLRSYGFRPTYGVGHQPYRVVVLSFELLHAPATVCGPKKSTNLVKNKCVNFN |
| | FNGLTGTGVLTESNKKFLPFQQFGRDIADTTDAVRDPQTLEILDITPCSFGGVSV |
| | ITPGTNTSNQVAVLYQGVNCTEVPVAIHADQLTPTWRVYSTGSNVFQTRAGCLI |
| | GAEYVNNSYECDIPIGAGICGSGSGSRVSPSTEVVRFPNITNLCPFGQVFNASN |
| | FPSVYAWERLRISDCVADYAVLYNSSSSFSTFKCYGVSPTKLNDLCFSSVYADY |
| | FVVKGDDVRQIAPAQTGVIADYNYKLPDDFTGCVLAWNTNSVDSKSGNNFYYR |
| | LFRHGKIKPYERDISNVLYNSAGGTCSSISQLGCYEPLKSYGFTPTVGVGYQPY |
| | RVVVLSFELLNAPATVCGPKKSTELVKNKCVNFGSGSGSRVAPSKEVVRFPNIT |
| | NLCPFGEVFNATTFPSVYAWERKRISNCVADYSVLYNSTSFSTFKCYGVSATKL |
| | NDLCFSNVYADSFVVKGDDVRQIAPGQTGVIADYNYKLPDDFLGCVLAWNTNS |
| | KDSSTSGNYNYLYRWVRRSKLNPYERDLSNDIYSPGGQSCSAVGPNCYNPLR |
| | PYGFFTTAGVGHQPYRVVVLSFELLNAPATVCGPKLSTDLIKNQCVNF |
| 4498_[S1_ | MFVFLVLLPLVSSQCVNLITRTQSYTNSFTRGVYYPDKVFRSSVLHSTQDLFLP |
| Omicron_ | FFSNVTWFHAIHVSGTNGTKRFDNPVLPFNDGVYFASTEKSNIIRGWIFGTTLD |
| HKU3_BTK | SKTQSLLIVNNATNVVIKVCEFQFCNDPFLDVYYHKNNKSWMESEFRVYSSAN |
| Y72 (SEQ ID | NCTFEYVSQPFLMDLEGKQGNFKNLREFVFKNIDGYFKIYSKHTPINLGRDLPQ |
| NO: 6) | GFSALEPLVDLPIGINITRFQTLLALHRSYLTPGDSSSGWTAGAAAYYVGYLQPR |
| | TFLLKYNENGTITDAVDCALDPLSETKCTLKSFTVEKGIYQTSNFRVQPTESIVR |
| | FPNITNLCPFDEVFNATRFASVYAWNRKRISNCVADYSVLYNFAPFFAFKCYGV |
| | SPTKLNDLCFTNVYADSFVIRGNEVSQIAPGQTGNIADYNYKLPDDFTGCVIAW |
| | NSNKLDSKVGGNYNYLYRLFRKSNLKPFERDISTEIYQAGNKPCNGVAGENCY |
| | FPLRSYGFRPTYGVGHQPYRVVVLSFELLHAPATVCGPKKSTNLVKNKCVNFN |
| | FNGLTGTGVLTESNKKFLPFQQFGRDIADTTDAVRDPQTLEILDITPCSFGGVSV |
| | ITPGTNTSNQVAVLYQGVNCTEVPVAIHADQLTPTWRVYSTGSNVFQTRAGCLI |
| | GAEYVNNSYECDIPIGAGICGSGSGSRVSPSTEVVRFPNITNLCPFGQVFNASN |
| | FPSVYAWERLRISDCVADYAVLYNSSSSFSTFKCYGVSPTKLNDLCFSSVYADY |
| | FVVKGDDVRQIAPAQTGVIADYNYKLPDDFTGCVLAWNTNSVDSKSGNNFYYR |
| | LFRHGKIKPYERDISNVLYNSAGGTCSSISQLGCYEPLKSYGFTPTVGVGYQPY |
| | RVVVLSFELLNAPATVCGPKKSTELVKNKCVNFGSGSGSRVAPSKEVVRFPNIT |
| | NLCPFGEVFNATTFPSVYAWERKRISNCVADYSVLYNSTSFSTFKCYGVSATKL |
| | NDLCFSNVYADSFVVKGDDVRQIAPGQTGVIADYNYKLPDDFLGCVLAWNTNS |
| | KDSSTSGNYNYLYRWVRRSKLNPYERDLSNDIYSPGGQSCSAVGPNCYNPLR |
| | PYGFFTTAGVGHQPYRVVVLSFELLNAPATVCGPKLSTDLIKNQCVNF |
| 4499_[WHS1_K | MFVFLVLLPLVSSQCVNLTTRTQLPPAYTNSFTRGVYYPDKVFRSSVLHSTQDL |
| HOSTA2RBD_ | FLPFFSNVTWFHAIHVSGTNGTKRFDNPVLPFNDGVYFASTEKSNIIRGWIFGTT |
| WIVIRBD] (SEQ | LDSKTQSLLIVNNATNVVIKVCEFQFCNDPFLGVYYHKNNKSWMESEFRVYSS |
| ID NO: 7) | ANNCTFEYVSQPFLMDLEGKQGNFKNLREFVFKNIDGYFKIYSKHTPINLVRDL |
| | PQGFSALEPLVDLPIGINITRFQTLLALHRSYLTPGDSSSGWTAGAAAYYVGYL |
| | QPRTFLLKYNENGTITDAVDCALDPLSETKCTLKSFTVEKGIYQTSNFRVQPTES |
| | IVRFPNITNLCPFGEVFNATRFASVYAWNRKRISNCVADYSVLYNSASFSTFKC |
| | YGVSPTKLNDLCFTNVYADSFVIRGDEVRQIAPGQTGKIADYNYKLPDDFTGCV |
| | IAWNSNNLDSKVGGNYNYLYRLFRKSNLKPFERDISTEIYQAGSTPCNGVEGFN |
| | CYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGGV |
| | SVITPGTNTSNQVAVLYQDVNCTEVPVAIHADQLTPTWRVYSTGSNVFQTRAG |
| | CLIGAEHVNNSYECDIPIGAGICASYQTQTNSPRGSGSGSRVSPSLDVVRFPNM |
| | TNICPFDQVFNKTQFPSVYAWERVRISDCVSDYTVLYNSSASFSTFKCYGVSPT |
| | KLNDLCFSGVYADYFVVKGDHVHQIAPGQTGVIADYNYKLPSEFVGCILAWNTR |
| | TIDSKRGFYYRLFRHGNIRPYERDTSNVPYNAAGGTCNQPGTHNCYEPLQDYG |
| | FTSTSGVGYQPFRVVVLSFELLNAPATVCGPKQSTDLVKNKCVNFGSGSGSRV |
| | APSKEVVRFPNITNLCPFGEVFNATTFPSVYAWERKRISNCVADYSVLYNSTSF |
| | STFKCYGVSATKLNDLCFSNVYADSFVVKGDDVRQIAPGQTGVIADYNYKLPD |
| | DFTGCVLAWNTRNIDATQTGNYNYKYRSLRHGKLRPFERDISNVPFSPDGKPC |
| | TPPAFNCYWPLNDYGFYITNGIGYQPYRVVVLSFELLNAPATVCGPKLSTDLIKN |
| | QCVNF |
| 4500_[WHS1_P | MFVFLVLLPLVSSQCVNLTTRTQLPPAYTNSFTRGVYYPDKVFRSSVLHSTQDL |
| ang17RBD_WIV | FLPFFSNVTWFHAIHVSGTNGTKRFDNPVLPFNDGVYFASTEKSNIIRGWIFGTT |
| 1RBD] (SEQ ID | LDSKTQSLLIVNNATNVVIKVCEFQFCNDPFLGVYYHKNNKSWMESEFRVYSS |
| NO: 8) | ANNCTFEYVSQPFLMDLEGKQGNFKNLREFVFKNIDGYFKIYSKHTPINLVRDL |
| | PQGFSALEPLVDLPIGINITRFQTLLALHRSYLTPGDSSSGWTAGAAAYYVGYL |
| | QPRTFLLKYNENGTITDAVDCALDPLSETKCTLKSFTVEKGIYQTSNFRVQPTES |
| | IVRFPNITNLCPFGEVFNATRFASVYAWNRKRISNCVADYSVLYNSASFSTFKC |
| | YGVSPTKLNDLCFTNVYADSFVIRGDEVRQIAPGQTGKIADYNYKLPDDFTGCV |
| | IAWNSNNLDSKVGGNYNYLYRLFRKSNLKPFERDISTEIYQAGSTPCNGVEGEN |
| | CYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGGV |
| | SVITPGTNTSNQVAVLYQDVNCTEVPVAIHADQLTPTWRVYSTGSNVFQTRAG |
| | CLIGAEHVNNSYECDIPIGAGICASYQTQTNSPRGSGSGSRVQPTISIVRFPNIT |
| | NLCPFGEVFNASKFASVYAWNRKRISNCVADYSVLYNSTSFSTFKCYGVSPTK |
| | LNDLCFTNVYADSFVVKGDEVRQIAPGQTGVIADYNYKLPDDFTGCVIAWNSVK |
| | QDALTGGNYGYLYRLFRKSKLKPFERDISTEIYQAGSTPCNGQVGLNCYYPLE |
| | RYGFHPTTGVNYQPFRVVVLSFELLNGPATVCGPKLSTTLVKDKCVNFGSGSG |
| | SRVAPSKEVVRFPNITNLCPFGEVFNATTFPSVYAWERKRISNCVADYSVLYNS |
| | TSFSTFKCYGVSATKLNDLCFSNVYADSFVVKGDDVRQIAPGQTGVIADYNYKL |
| | PDDFTGCVLAWNTRNIDATQTGNYNYKYRSLRHGKLRPFERDISNVPFSPDGK |
| | PCTPPAFNCYWPLNDYGFYITNGIGYQPYRVVVLSFELLNAPATVCGPKLSTDLI |
| | KNQCVNF |
| 4501_[WHS1_K | MFVFLVLLPLVSSQCVNLTTRTQLPPAYTNSFTRGVYYPDKVFRSSVLHSTQDL |
| hosta2RBD_Rs | FLPFFSNVTWFHAIHVSGTNGTKRFDNPVLPFNDGVYFASTEKSNIIRGWIFGTT |
| SHC014RBD | LDSKTQSLLIVNNATNWVIKVCEFQFCNDPFLGVYYHKNNKSWMESEFRVYSS |
| WIV1RBD] | ANNCTFEYVSQPFLMDLEGKQGNFKNLREFVFKNIDGYFKIYSKHTPINLVRDL |
| (SEQ ID NO: 9) | PQGFSALEPLVDLPIGINITRFQTLLALHRSYLTPGDSSSGWTAGAAAYYVGYL |
| | QPRTFLLKYNENGTITDAVDCALDPLSETKCTLKSFTVEKGIYQTSNFRVQPTES |
| | IVRFPNITNLCPFGEVFNATRFASVYAWNRKRISNCVADYSVLYNSASFSTFKC |
| | YGVSPTKLNDLCFTNVYADSFVIRGDEVRQIAPGQTGKIADYNYKLPDDFTGCV |
| | IAWNSNNLDSKVGGNYNYLYRLFRKSNLKPFERDISTEIYQAGSTPCNGVEGEN |
| | CYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGGV |
| | SVITPGTNTSNQVAVLYQDVNCTEVPVAIHADQLTPTWRVYSTGSNVFQTRAG |
| | CLIGAEHVNNSYECDIPIGAGICASYQTQTNSPRGSGSGSRVSPSLDVVRFPNM |
| | TNICPFDQVFNKTQFPSVYAWERVRISDCVSDYTVLYNSSASFSTFKCYGVSPT |
| | KLNDLCFSGVYADYFVVKGDHVHQIAPGQTGVIADYNYKLPSEFVGCILAWNTR |
| | TIDSKRGFYYRLFRHGNIRPYERDTSNVPYNAAGGTCNQPGTHNCYEPLQDYG |
| | FTSTSGVGYQPFRVVVLSFELLNAPATVCGPKQSTDLVKNKCVNFGSGSGSRV |
| | APSKEVVRFPNITNLCPFGEVFNATTFPSVYAWERKRISNCVADYSVLYNSTSF |
| | STFKCYGVSATKLNDLCFSNVYADSFVVKGDDVRQIAPGQTGVIADYNYKLPD |
| | DFLGCVLAWNTNSKDSSTSGNYNYLYRWVRRSKLNPYERDLSNDIYSPGGQS |
| | CSAVGPNCYNPLRPYGFFTTAGVGHQPYRVVVLSFELLNAPATVCGPKLSTDLI |
| | KNQCVNFGSGSGSRVAPSKEVVRFPNITNLCPFGEVFNATTFPSVYAWERKRI |
| | SNCVADYSVLYNSTSFSTFKCYGVSATKLNDLCFSNVYADSFVVKGDDVRQIA |
| | PGQTGVIADYNYKLPDDFTGCVLAWNTRNIDATQTGNYNYKYRSLRHGKLRPF |
| | ERDISNVPFSPDGKPCTPPAFNCYWPLNDYGFYITNGIGYQPYRVVVLSFELLN |
| | APATVCGPKLSTDLIKNQCVNF |
| 4502_[WHS1_K | MFVFLVLLPLVSSQCVNLTTRTQLPPAYTNSFTRGVYYPDKVFRSSVLHSTQDL |
| HOSTA2RBD | FLPFFSNVTWFHAIHVSGTNGTKRFDNPVLPFNDGVYFASTEKSNIIRGWIFGTT |
| RSCH014RBD | LDSKTQSLLIVNNATNWVIKVCEFQFCNDPFLGVYYHKNNKSWMESEFRVYSS |
| URBANIRBD] | ANNCTFEYVSQPFLMDLEGKQGNFKNLREFVFKNIDGYFKIYSKHTPINLVRDL |
| (SEQ ID NO: 10) | PQGFSALEPLVDLPIGINITRFQTLLALHRSYLTPGDSSSGWTAGAAAYYVGYL |
| | QPRTFLLKYNENGTITDAVDCALDPLSETKCTLKSFTVEKGIYQTSNFRVQPTES |
| | IVRFPNITNLCPFGEVFNATRFASVYAWNRKRISNCVADYSVLYNSASFSTFKC |
| | YGVSPTKLNDLCFTNVYADSFVIRGDEVRQIAPGQTGKIADYNYKLPDDFTGCV |
| | IAWNSNNLDSKVGGNYNYLYRLFRKSNLKPFERDISTEIYQAGSTPCNGVEGEN |
| | CYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGGV |
| | SVITPGTNTSNQVAVLYQDVNCTEVPVAIHADQLTPTWRVYSTGSNVFQTRAG |
| | CLIGAEHVNNSYECDIPIGAGICASYQTQTNSPRGSGSGSRVSPSLDVVRFPNM |
| | TNICPFDQVFNKTQFPSVYAWERVRISDCVSDYTVLYNSSASFSTFKCYGVSPT |
| | KLNDLCFSGVYADYFVVKGDHVHQIAPGQTGVIADYNYKLPSEFVGCILAWNTR |
| | TIDSKRGFYYRLFRHGNIRPYERDTSNVPYNAAGGTCNQPGTHNCYEPLQDYG |
| | FTSTSGVGYQPFRVVVLSFELLNAPATVCGPKQSTDLVKNKCVNFGSGSGSRV |
| | APSKEVVRFPNITNLCPFGEVFNATTFPSVYAWERKRISNCVADYSVLYNSTSF |
| | STFKCYGVSATKLNDLCFSNVYADSFVVKGDDVRQIAPGQTGVIADYNYKLPD |
| | DFLGCVLAWNTNSKDSSTSGNYNYLYRWVRRSKLNPYERDLSNDIYSPGGQS |
| | CSAVGPNCYNPLRPYGFFTTAGVGHQPYRVVVLSFELLNAPATVCGPKLSTDLI |
| | KNQCVNFGSGSGSRVVPSGDVVRFPNITNLCPFGEVFNATKFPSVYAWERKKI |
| | SNCVADYSVLYNSTFFSTFKCYGVSATKLNDLCFSNVYADSFVVKGDDVRQIA |
| | PGQTGVIADYNYKLPDDFMGCVLAWNTRNIDATSTGNYNYKYRYLRHGKLRPF |
| | ERDISNVPFSPDGKPCTPPALNCYWPLNDYGFYTTTGIGYQPYRVVVLSFELLN |
| | APATVCGPKLSTDLIKNQCVNF |
| 4503_[WuhanS | MFVFLVLLPLVSSQCVNLTTRTQLPPAYTNSFTRGVYYPDKVFRSSVLHSTQDL |
| 1_RATG13RBD | FLPFFSNVTWFHAIHVSGTNGTKRFDNPVLPFNDGVYFASTEKSNIIRGWIFGTT |
| _BTKY72RBD_ | LDSKTQSLLIVNNATNWVIKVCEFQFCNDPFLGVYYHKNNKSWMESEFRVYSS |
| WIV1RBD] | ANNCTFEYVSQPFLMDLEGKQGNFKNLREFVFKNIDGYFKIYSKHTPINLVRDL |
| (SEQ ID NO: 11) | PQGFSALEPLVDLPIGINITRFQTLLALHRSYLTPGDSSSGWTAGAAAYYVGYL |
| | QPRTFLLKYNENGTITDAVDCALDPLSETKCTLKSFTVEKGIYQTSNFRVQPTES |
| | IVRFPNITNLCPFGEVFNATRFASVYAWNRKRISNCVADYSVLYNSASFSTFKC |
| | YGVSPTKLNDLCFTNVYADSFVIRGDEVRQIAPGQTGKIADYNYKLPDDFTGCV |
| | IAWNSNNLDSKVGGNYNYLYRLFRKSNLKPFERDISTEIYQAGSTPCNGVEGFN |
| | CYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGGV |
| | SVITPGTNTSNQVAVLYQDVNCTEVPVAIHADQLTPTWRVYSTGSNVFQTRAG |
| | CLIGAEHVNNSYECDIPIGAGICASYQTQTNSPRGSGSGSRVQPTDSIVRFPNIT |
| | NLCPFGEVFNATTFASVYAWNRKRISNCVADYSVLYNSTSFSTFKCYGVSPTKL |
| | NDLCFTNVYADSFVITGDEVRQIAPGQTGKIADYNYKLPDDFTGCVIAWNSKHID |
| | AKEGGNFNYLYRLFRKANLKPFERDISTEIYQAGSKPCNGQTGLNCYYPLYRY |
| | GFYPTDGVGHQPYRVVVLSFELLNAPATVCGPKKSTNLVKNKCVNFGSGSGS |
| | RVSPSTEVVRFPNITNLCPFGQVFNASNFPSVYAWERLRISDCVADYAVLYNSS |
| | SSFSTFKCYGVSPTKLNDLCFSSVYADYFVVKGDDVRQIAPAQTGVIADYNYKL |
| | PDDFTGCVLAWNTNSVDSKSGNNFYYRLFRHGKIKPYERDISNVLYNSAGGTC |
| | SSISQLGCYEPLKSYGFTPTVGVGYQPYRVVVLSFELLNAPATVCGPKKSTELV |
| | KNKCVNFGSGSGSRVAPSKEVVRFPNITNLCPFGEVFNATTFPSVYAWERKRI |
| | SNCVADYSVLYNSTSFSTFKCYGVSATKLNDLCFSNVYADSFVVKGDDVRQIA |
| | PGQTGVIADYNYKLPDDFTGCVLAWNTRNIDATQTGNYNYKYRSLRHGKLRPF |
| | ERDISNVPFSPDGKPCTPPAFNCYWPLNDYGFYITNGIGYQPYRVVVLSFELLN |
| | APATVCGPKLSTDLIKNQCVNF |
| 4495_4498_BA | MFVFLVLLPLVSSQCVNLITRTQSYTNSFTRGVYYPDKVFRSSVLHSTQDLFLP |
| 2S1 | FFSNVTWFHAIHVSGTNGTKRFDNPVLPFNDGVYFASTEKSNIIRGWIFGTTLD |
| (SEQ ID NO: 12) | SKTQSLLIVNNATNVVIKVCEFQFCNDPFLDVYYHKNNKSWMESEFRVYSSAN |
| | NCTFEYVSQPFLMDLEGKQGNFKNLREFVFKNIDGYFKIYSKHTPINLGRDLPQ |
| | GFSALEPLVDLPIGINITRFQTLLALHRSYLTPGDSSSGWTAGAAAYYVGYLQPR |
| | TFLLKYNENGTITDAVDCALDPLSETKCTLKSFTVEKGIYQTSNFRVQPTESIVR |
| | FPNITNLCPFDEVFNATRFASVYAWNRKRISNCVADYSVLYNFAPFFAFKCYGV |
| | SPTKLNDLCFTNVYADSFVIRGNEVSQIAPGQTGNIADYNYKLPDDFTGCVIAW |
| | NSNKLDSKVGGNYNYLYRLFRKSNLKPFERDISTEIYQAGNKPCNGVAGENCY |
| | FPLRSYGFRPTYGVGHQPYRVVVLSFELLHAPATVCGPKKSTNLVKNKCVNFN |
| | FNGLTGTGVLTESNKKFLPFQQFGRDIADTTDAVRDPQTLEILDITPCSFGGVSV |
| | ITPGTNTSNQVAVLYQGVNCTEVPVAIHADQLTPTWRVYSTGSNVFQTRAGCLI |
| | GAEYVNNSYECDIPIGAGIC |
| 4296_4304_437 | MFVFLVLLPLVSSQCVNLTTRTQLPPAYTNSFTRGVYYPDKVFRSSVLHSTQDL |
| 1_4372_4499_4 | FLPFFSNVTWFHAIHVSGTNGTKRFDNPVLPFNDGVYFASTEKSNIIRGWIFGTT |
| 500_4501_4502 | LDSKTQSLLIVNNATNVVIKVCEFQFCNDPFLGVYYHKNNKSWMESEFRVYSS |
| _4503_WuhanS | ANNCTFEYVSQPFLMDLEGKQGNFKNLREFVFKNIDGYFKIYSKHTPINLVRDL |
| 1 | PQGFSALEPLVDLPIGINITRFQTLLALHRSYLTPGDSSSGWTAGAAAYYVGYL |
| (SEQ ID NO: 13) | QPRTFLLKYNENGTITDAVDCALDPLSETKCTLKSFTVEKGIYQTSNFRVQPTES |
| | IVRFPNITNLCPFGEVFNATRFASVYAWNRKRISNCVADYSVLYNSASFSTFKC |
| | YGVSPTKLNDLCFTNVYADSFVIRGDEVRQIAPGQTGKIADYNYKLPDDFTGCV |
| | IAWNSNNLDSKVGGNYNYLYRLFRKSNLKPFERDISTEIYQAGSTPCNGVEGEN |
| | CYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGGV |
| | SVITPGTNTSNQVAVLYQDVNCTEVPVAIHADQLTPTWRVYSTGSNVFQTRAG |
| | CLIGAEHVNNSYECDIPIGAGICASYQTQTNSPR |
| 4495_4498_450 | RVSPSTEVVRFPNITNLCPFGQVFNASNFPSVYAWERLRISDCVADYAVLYNSS |
| 3_BTKY72 | SSFSTFKCYGVSPTKLNDLCFSSVYADYFVVKGDDVRQIAPAQTGVIADYNYKL |
| (SEQ ID NO: 14) | PDDFTGCVLAWNTNSVDSKSGNNFYYRLFRHGKIKPYERDISNVLYNSAGGTC |
| | SSISQLGCYEPLKSYGFTPTVGVGYQPYRVVVLSFELLNAPATVCGPKKSTELV |
| | KNKCVNF |
| 4495_4501_450 | RVAPSKEVVRFPNITNLCPFGEVFNATTFPSVYAWERKRISNCVADYSVLYNST |
| 2_RsShC014 | SFSTFKCYGVSATKLNDLCFSNVYADSFVVKGDDVRQIAPGQTGVIADYNYKLP |
| (SEQ ID NO: 15) | DDFLGCVLAWNTNSKDSSTSGNYNYLYRWVRRSKLNPYERDLSNDIYSPGGQ |
| | SCSAVGPNCYNPLRPYGFFTTAGVGHQPYRVVVLSFELLNAPATVCGPKLSTD |
| | LIKNQCVNF |
| 4498_HKU3 | RVSPTQEVIRFPNITNRCPFDKVFNATRFPNVYAWERTKISDCVADYTVLYNST |
| (SEQ ID NO: 16) | SFSTFKCYGVSPSKLIDLCFTSVYADTFLIRSSEVRQVAPGETGVIADYNYKLPD |
| | DFTGCVIAWNTAKHDTGNYYYRSHRKTKLKPFERDLSSDDGNGVYTLSTYDFN |
| | PNVPVAYQATRVVVLSFELLNAPATVCGPKLSTELVKNQCVNF |
| 4498_4499_4500_ | RVAPSKEVVRFPNITNLCPFGEVFNATTFPSVYAWERKRISNCVADYSVLYNST |
| 4501_4503 | SFSTFKCYGVSATKLNDLCFSNVYADSFVVKGDDVRQIAPGQTGVIADYNYKLP |
| WIV1 | DDFTGCVLAWNTRNIDATQTGNYNYKYRSLRHGKLRPFERDISNVPFSPDGKPC |
| (SEQ ID NO: 17) | TPPAFNCYWPLNDYGFYITNGIGYQPYRVVVLSFELLNAPATVCGPKLSTDLIK |
| | NQCVNF |
| 4499_4501_450 | RVSPSLDVVRFPNMTNICPFDQVFNKTQFPSVYAWERVRISDCVSDYTVLYNS |
| 2_Khosta2 | SASFSTFKCYGVSPTKLNDLCFSGVYADYFVVKGDHVHQIAPGQTGVIADYNY |
| (SEQ ID NO: 18) | KLPSEFVGCILAWNTRTIDSKRGFYYRLFRHGNIRPYERDTSNVPYNAAGGTCN |
| | QPGTHNCYEPLQDYGFTSTSGVGYQPFRVVVLSFELLNAPATVCGPKQSTDLV |
| | KNKCVNF |
| 4500_Pang17 | RVQPTISIVRFPNITNLCPFGEVFNASKFASVYAWNRKRISNCVADYSVLYNSTS |
| (SEQ ID NO: 19) | FSTFKCYGVSPTKLNDLCFTNVYADSFVVKGDEVRQIAPGQTGVIADYNYKLPD |
| | DFTGCVIAWNSVKQDALTGGNYGYLYRLFRKSKLKPFERDISTEIYQAGSTPCN |
| | GQVGLNCYYPLERYGFHPTTGVNYQPFRVVVLSFELLNGPATVCGPKLSTTLV |
| | KDKCVNF |
| 4502_Urbani | RVVPSGDVVRFPNITNLCPFGEVFNATKFPSVYAWERKKISNCVADYSVLYNST |
| (SEQ ID NO: 20) | FFSTFKCYGVSATKLNDLCFSNVYADSFVVKGDDVRQIAPGQTGVIADYNYKLP |
| | DDFMGCVLAWNTRNIDATSTGNYNYKYRYLRHGKLRPFERDISNVPFSPDGKP |
| | CTPPALNCYWPLNDYGFYTTTGIGYQPYRVVVLSFELLNAPATVCGPKLSTDLI |
| | KNQCVNF |
| 4503_RatG13 | RVQPTDSIVRFPNITNLCPFGEVFNATTFASVYAWNRKRISNCVADYSVLYNST |
| (SEQ ID NO: 21) | SFSTFKCYGVSPTKLNDLCFTNVYADSFVITGDEVRQIAPGQTGKIADYNYKLP |
| | DDFTGCVIAWNSKHIDAKEGGNFNYLYRLFRKANLKPFERDISTEIYQAGSKPC |
| | NGQTGLNCYYPLYRYGFYPTDGVGHQPYRVVVLSFELLNAPATVCGPKKSTNL |
| | VKNKCVNF |
| OmicronRBD | RVQPTESIVRFPNITNLCPFDEVFNATRFASVYAWNRKRISNCVADYSVLYNLA |
| (SEQ ID NO: 22) | PFFTFKCYGVSPTKLNDLCFTNVYADSFVIRGDEVRQIAPGQTGNIADYNYKLP |
| | DDFTGCVIAWNSNKLDSKVSGNYNYLYRLFRKSNLKPFERDISTEIYQAGNKPC |
| | NGVAGFNCYFPLRSYSFRPTYGVGHQPYRVVVLSFELLHAPATVCGPKKSTNL |
| | VKNKCVNFNFNG |
| BetaRBD | MFVFLVLLPLVSSRVQPTESIVRFPNITNLCPFGEVFNATRFASVYAWNRKRISN |
| (SEQ ID NO: 23) | CVADYSVLYNSASFSTFKCYGVSPTKLNDLCFTNVYADSFVIRGDEVRQIAPGQ |
| | TGNIADYNYKLPDDFTGCVIAWNSNNLDSKVGGNYNYLYRLFRKSNLKPFERDI |
| | STEIYQAGSTPCNGVKGFNCYFPLQSYGFQPTYGVGYQPYRVVVLSFELLHAP |
| | ATVCGPKKSTNLVKNKCVNF |
| DeltaRBD | RVQPTESIVRFPNITNLCPFGEVFNATRFASVYAWNRKRISNCVADYSVLYNSA |
| (SEQ ID NO: 24) | SFSTFKCYGVSPTKLNDLCFTNVYADSFVIRGDEVRQIAPGQTGKIADYNYKLP |
| | DDFTGCVIAWNSNNLDSKVGGNYNYRYRLFRKSNLKPFERDISTEIYQAGSKP |
| | CNGVEGFNCYFPLQSYGFQPTNGVGYQPYRVVVLSFELLHAPATVCGPKKST |
| | NLVKNKCVNFNFNG |

TABLE 2
Sequences of exemplary nucleic acids encoding fusion polypeptides
and antigens as described herein.

| Identifier | |
|---|---|
| 4296_WHS1_) | ATGTTCGTGTTTCTGGTGCTGCTGCCTCTGGTGTCCAGCCAGTGTGTGAACC |
| DeltaRBD | TGACCACCAGAACACAGCTGCCTCCAGCCTACACCAACAGCTTTACCAGAG |
| (SEQ ID NO: | GCGTGTACTACCCCGACAAGGTGTTCAGATCCAGCGTGCTGCACTCTACCC |
| 25) | AGGACCTGTTCCTGCCTTTCTTCAGCAACGTGACCTGGTTCCACGCCATCCA |
| | CGTGTCCGGCACCAATGGCACCAAGAGATTCGACAACCCCGTGCTGCCCTT |
| | CAACGACGGGGTGTACTTTGCCAGCACCGAGAAGTCCAACATCATCAGAGG |
| | CTGGATCTTCGGCACCACACTGGACAGCAAGACCCAGAGCCTGCTGATCGT |
| | GAACAACGCCACCAACGTGGTCATCAAAGTGTGCGAGTTCCAGTTCTGCAA |
| | CGACCCCTTCCTGGGCGTCTACTACCACAAGAACAACAAGAGCTGGATGGA |
| | AAGCGAGTTCCGGGTGTACAGCAGCGCCAACAACTGCACCTTCGAGTACGT |
| | GTCCCAGCCTTTCCTGATGGACCTGGAAGGCAAGCAGGGCAACTTCAAGAA |
| | CCTGCGCGAGTTCGTGTTCAAGAACATCGACGGCTACTTCAAGATCTACAGC |
| | AAGCACACCCCTATCAACCTCGTGCGGGATCTGCCTCAGGGCTTCTCTGCT |
| | CTGGAACCCCTGGTGGATCTGCCCATCGGCATCAACATCACCCGGTTTCAG |
| | ACACTGCTGGCCCTGCACAGAAGCTACCTGACACCTGGCGATAGCAGCAGC |
| | GGATGGACAGCTGGTGCCGCCGCTTACTATGTGGGCTACCTGCAGCCTAGA |
| | ACCTTCCTGCTGAAGTACAACGAGAACGGCACCATCACCGACGCCGTGGAT |
| | TGTGCCCTTGATCCTCTGAGCGAGACAAAGTGCACCCTGAAGTCCTTCACC |
| | GTGGAAAAGGGCATCTACCAGACCAGCAACTTCCGGGTGCAGCCCACCGAA |
| | TCCATCGTGCGGTTCCCCAATATCACCAATCTGTGCCCCTTCGGCGAGGTGT |
| | TCAATGCCACCAGATTCGCCTCTGTGTACGCCTGGAACCGGAAGCGGATCA |
| | GCAATTGCGTGGCCGACTACTCCGTGCTGTACAACTCCGCCAGCTTCAGCA |
| | CCTTCAAGTGCTACGGCGTGTCCCCTACCAAGCTGAACGACCTGTGCTTCA |
| | CAAACGTGTACGCCGACAGCTTCGTGATCCGGGGAGATGAAGTGCGGCAGA |
| | TTGCCCCTGGACAGACAGGCAAGATCGCCGACTACAACTACAAGCTGCCCG |
| | ACGACTTCACCGGCTGTGTGATTGCCTGGAACAGCAACAACCTGGACTCCA |
| | AAGTCGGCGGCAACTACAATTACCTGTACCGGCTGTTCCGGAAGTCCAATCT |
| | GAAGCCCTTCGAGCGGGACATCTCCACCGAGATCTATCAGGCCGGCAGCAC |
| | CCCTTGTAACGGCGTGGAAGGCTTCAACTGCTACTTCCCACTGCAGTCCTAC |
| | GGCTTTCAGCCCACAAATGGCGTGGGCTATCAGCCCTACAGAGTGGTGGTG |
| | CTGAGCTTCGAACTGCTGCATGCCCCTGCCACAGTGTGCGGCCCTAAGAAA |
| | AGCACCAATCTCGTGAAGAACAAATGCGTGAACTTCAACTTCAACGGCCTGA |
| | CCGGCACCGGCGTGCTGACAGAGAGCAACAAGAAGTTCCTGCCATTCCAGC |
| | AGTTTGGCCGGGATATCGCCGATACCACAGACGCCGTTAGAGATCCCCAGA |
| | CACTGGAAATCCTGGACATCACCCCTTGCAGCTTCGGCGGAGTGTCTGTGA |
| | TCACCCCTGGCACCAACACCAGCAATCAGGTGGCAGTGCTGTACCAGGACG |
| | TGAACTGTACCGAAGTGCCCGTGGCCATTCACGCCGATCAGCTGACACCTA |
| | CATGGCGGGTGTACTCCACCGGCAGCAATGTGTTTCAGACCAGAGCCGGCT |
| | GTCTGATCGGAGCCGAGCACGTGAACAATAGCTACGAGTGCGACATCCCCA |
| | TCGGCGCTGGCATCTGTGCCAGCTACCAGACACAGACAAACAGCCCCAGAG |
| | GATCCGGCAGTGGGTCAGGAAGGGTTCAGCCCACAGAGTCCATAGTACGGT |
| | TCCCTAATATTACCAACCTGTGTCCGTTCGGTGAAGTCTTCAATGCCACAAG |
| | ATTCGCCAGCGTTTATGCCTGGAACCGAAAGAGAATTAGTAATTGTGTTGCG |
| | GATTATTCTGTCCTTTACAATAGTGCAAGCTTCTCCACCTTCAAGTGTTACGG |
| | GGTTTCCCCGACGAAACTCAACGACCTTTGTTTTACAAATGTGTATGCAGATT |
| | CATTTGTTATCCGAGGAGATGAGGTACGGCAAATAGCCCCCGGACAAACAG |
| | GAAAGATAGCCGATTACAACTATAAACTGCCTGATGACTTCACTGGGTGCGT |
| | CATCGCATGGAATAGCAACAACCTTGATTCCAAAGTAGGGGGCAATTATAAT |
| | TACCGCTATCGACTTTTCCGCAAGTCCAACCTCAAACCCTTCGAGCGCGATA |
| | TCAGCACGGAAATTTACCAAGCGGGCAGCAAACCATGCAACGGAGTCGAAG |
| | GATTTAATTGTTACTTCCCGTTGCAATCCTATGGCTTTCAACCAACCAACGGT |
| | GTTGGTTACCAACCTTATAGAGTTGTCGTTCTCAGTTTTGAGCTCCTCCATGC |
| | CCCGGCCACCGTATGCGGCCCAAAGAAATCAACAAACCTCGTTAAGAACAA |
| | ATGTGTCAACTTTAATTTCAATGGGTAG |
| 4304_WHS1_ | ATGTTCGTGTTTCTGGTGCTGCTGCCTCTGGTGTCCAGCCAGTGTGTGAACC |
| OmicronRBD | TGACCACCAGAACACAGCTGCCTCCAGCCTACACCAACAGCTTTACCAGAG |
| (SEQ ID NO: | GCGTGTACTACCCCGACAAGGTGTTCAGATCCAGCGTGCTGCACTCTACCC |
| 26) | AGGACCTGTTCCTGCCTTTCTTCAGCAACGTGACCTGGTTCCACGCCATCCA |
| | CGTGTCCGGCACCAATGGCACCAAGAGATTCGACAACCCCGTGCTGCCCTT |
| | CAACGACGGGGTGTACTTTGCCAGCACCGAGAAGTCCAACATCATCAGAGG |
| | CTGGATCTTCGGCACCACACTGGACAGCAAGACCCAGAGCCTGCTGATCGT |
| | GAACAACGCCACCAACGTGGTCATCAAAGTGTGCGAGTTCCAGTTCTGCAA |
| | CGACCCCTTCCTGGGCGTCTACTACCACAAGAACAACAAGAGCTGGATGGA |
| | AAGCGAGTTCCGGGTGTACAGCAGCGCCAACAACTGCACCTTCGAGTACGT |
| | GTCCCAGCCTTTCCTGATGGACCTGGAAGGCAAGCAGGGCAACTTCAAGAA |
| | CCTGCGCGAGTTCGTGTTCAAGAACATCGACGGCTACTTCAAGATCTACAGC |
| | AAGCACACCCCTATCAACCTCGTGCGGGATCTGCCTCAGGGCTTCTCTGCT |
| | CTGGAACCCCTGGTGGATCTGCCCATCGGCATCAACATCACCCGGTTTCAG |
| | ACACTGCTGGCCCTGCACAGAAGCTACCTGACACCTGGCGATAGCAGCAGC |
| | GGATGGACAGCTGGTGCCGCCGCTTACTATGTGGGCTACCTGCAGCCTAGA |
| | ACCTTCCTGCTGAAGTACAACGAGAACGGCACCATCACCGACGCCGTGGAT |
| | TGTGCCCTTGATCCTCTGAGCGAGACAAAGTGCACCCTGAAGTCCTTCACC |
| | GTGGAAAAGGGCATCTACCAGACCAGCAACTTCCGGGTGCAGCCCACCGAA |
| | TCCATCGTGCGGTTCCCCAATATCACCAATCTGTGCCCCTTCGGCGAGGTGT |
| | TCAATGCCACCAGATTCGCCTCTGTGTACGCCTGGAACCGGAAGCGGATCA |
| | GCAATTGCGTGGCCGACTACTCCGTGCTGTACAACTCCGCCAGCTTCAGCA |
| | CCTTCAAGTGCTACGGCGTGTCCCCTACCAAGCTGAACGACCTGTGCTTCA |
| | CAAACGTGTACGCCGACAGCTTCGTGATCCGGGGAGATGAAGTGCGGCAGA |
| | TTGCCCCTGGACAGACAGGCAAGATCGCCGACTACAACTACAAGCTGCCCG |
| | ACGACTTCACCGGCTGTGTGATTGCCTGGAACAGCAACAACCTGGACTCCA |
| | AAGTCGGCGGCAACTACAATTACCTGTACCGGCTGTTCCGGAAGTCCAATCT |
| | GAAGCCCTTCGAGCGGGACATCTCCACCGAGATCTATCAGGCCGGCAGCAC |
| | CCCTTGTAACGGCGTGGAAGGCTTCAACTGCTACTTCCCACTGCAGTCCTAC |
| | GGCTTTCAGCCCACAAATGGCGTGGGCTATCAGCCCTACAGAGTGGTGGTG |
| | CTGAGCTTCGAACTGCTGCATGCCCCTGCCACAGTGTGCGGCCCTAAGAAA |
| | AGCACCAATCTCGTGAAGAACAAATGCGTGAACTTCAACTTCAACGGCCTGA |
| | CCGGCACCGGCGTGCTGACAGAGAGCAACAAGAAGTTCCTGCCATTCCAGC |
| | AGTTTGGCCGGGATATCGCCGATACCACAGACGCCGTTAGAGATCCCCAGA |
| | CACTGGAAATCCTGGACATCACCCCTTGCAGCTTCGGCGGAGTGTCTGTGA |
| | TCACCCCTGGCACCAACACCAGCAATCAGGTGGCAGTGCTGTACCAGGACG |
| | TGAACTGTACCGAAGTGCCCGTGGCCATTCACGCCGATCAGCTGACACCTA |
| | CATGGCGGGTGTACTCCACCGGCAGCAATGTGTTTCAGACCAGAGCCGGCT |
| | GTCTGATCGGAGCCGAGCACGTGAACAATAGCTACGAGTGCGACATCCCCA |
| | TCGGCGCTGGCATCTGTGCCAGCTACCAGACACAGACAAACAGCCCCAGAG |
| | GATCCGGCAGTGGGTCAGGACGAGTCCAACCAACAGAGTCAATCGTAAGAT |
| | TCCCCAATATCACCAACCTCTGCCCCTTTGACGAAGTTTTCAACGCTACCAG |
| | ATTTGCAAGTGTGTACGCCTGGAATAGGAAGCGGATCAGTAACTGCGTCGC |
| | CGACTACTCTGTACTTTATAACCTCGCGCCCTTCTTTACATTCAAGTGCTATG |
| | GCGTCTCCCCCACGAAACTTAATGACCTCTGTTTCACGAACGTCTACGCCGA |
| | TTCCTTTGTGATAAGAGGGGATGAGGTAAGACAAATAGCACCTGGTCAGACA |
| | GGCAACATAGCAGACTATAACTATAAGTTGCCCGACGACTTTACAGGATGTG |
| | TAATTGCTTGGAACAGTAACAAGCTGGATTCAAAGGTTTCCGGCAATTATAAC |
| | TACCTGTACAGACTGTTCAGAAAAAGTAATCTCAAACCCTTCGAGAGAGACA |
| | TTTCAACCGAAATCTATCAGGCCGGGAACAAACCATGCAACGGCGTCGCTG |
| | GATTCAACTGTTACTTCCCCCTCCGATCATACTCCTTTAGGCCCACATATGGA |
| | GTGGGTCATCAACCTTACCGAGTTGTTGTCCTGTCTTTCGAGCTCTTGCACG |
| | CGCCGGCAACTGTGTGTGGACCTAAAAAGAGTACGAACTTGGTGAAGAATA |
| | AGTGTGTAAACTTTAACTTTAATGGTTAG |
| 4371_BetaRB | ATGTTCGTGTTTCTGGTGCTGCTGCCTCTGGTGTCCAGCCGGGTGCAGCCC |
| D_WHS1_ | ACCGAATCCATCGTGCGGTTCCCCAATATCACCAATCTGTGCCCCTTCGGCG |
| DeltaRBD (SEQ ID | AGGTGTTCAATGCCACCAGATTCGCCTCTGTGTACGCCTGGAACCGGAAGC |
| NO: 27) | GGATCAGCAATTGCGTGGCCGACTATAGTGTACTGTATAACTCAGCATCCTT |
| | TTCCACGTTTAAGTGCTACGGGGTCAGTCCTACAAAGTTGAATGACTTGTGC |
| | TTTACAAACGTATATGCTGATTCCTTTGTCATACGAGGCGATGAGGTCAGAC |
| | AGATTGCTCCGGGACAAACCGGAAATATTGCCGACTACAACTACAAACTTCC |
| | CGACGATTTTACTGGGTGTGTAATTGCCTGGAACAGTAACAATCTTGACTCA |
| | AAAGTGGGGGGAAATTACAATTACCTTTATAGACTCTTCCGCAAGAGCAATC |
| | TCAAGCCTTTTGAACGCGATATATCTACCGAAATATACCAGGCCGGTTCTAC |
| | TCCATGCAATGGAGTTAAGGGATTTAATTGTTACTTTCCTCTCCAATCTTATG |
| | GGTTCCAGCCAACTTATGGAGTCGGTTATCAGCCATATCGAGTCGTTGTGCT |
| | CAGCTTCGAACTGCTTCATGCCCCAGCTACTGTTTGTGGGCCGAAAAAGTCC |
| | ACTAATCTCGTGAAGAATAAGTGCGTAAACTTTGGCAGTGGGTCAGGATCCG |
| | GAATGCAGTGTGTGAACCTGACCACCAGAACACAGCTGCCTCCAGCCTACA |
| | CCAACAGCTTTACCAGAGGCGTGTACTACCCCGACAAGGTGTTCAGATCCA |
| | GCGTGCTGCACTCTACCCAGGACCTGTTCCTGCCTTTCTTCAGCAACGTGAC |
| | CTGGTTCCACGCCATCCACGTGTCCGGCACCAATGGCACCAAGAGATTCGA |
| | CAACCCCGTGCTGCCCTTCAACGACGGGGTGTACTTTGCCAGCACCGAGAA |
| | GTCCAACATCATCAGAGGCTGGATCTTCGGCACCACACTGGACAGCAAGAC |
| | CCAGAGCCTGCTGATCGTGAACAACGCCACCAACGTGGTCATCAAAGTGTG |
| | CGAGTTCCAGTTCTGCAACGACCCCTTCCTGGGCGTCTACTACCACAAGAAC |
| | AACAAGAGCTGGATGGAAAGCGAGTTCCGGGTGTACAGCAGCGCCAACAAC |
| | TGCACCTTCGAGTACGTGTCCCAGCCTTTCCTGATGGACCTGGAAGGCAAG |
| | CAGGGCAACTTCAAGAACCTGCGCGAGTTCGTGTTCAAGAACATCGACGGC |
| | TACTTCAAGATCTACAGCAAGCACACCCCTATCAACCTCGTGCGGGATCTGC |
| | CTCAGGGCTTCTCTGCTCTGGAACCCCTGGTGGATCTGCCCATCGGCATCA |
| | ACATCACCCGGTTTCAGACACTGCTGGCCCTGCACAGAAGCTACCTGACAC |
| | CTGGCGATAGCAGCAGCGGATGGACAGCTGGTGCCGCCGCTTACTATGTG |
| | GGCTACCTGCAGCCTAGAACCTTCCTGCTGAAGTACAACGAGAACGGCACC |
| | ATCACCGACGCCGTGGATTGTGCCCTTGATCCTCTGAGCGAGACAAAGTGC |
| | ACCCTGAAGTCCTTCACCGTGGAAAAGGGCATCTACCAGACCAGCAACTTC |
| | CGGGTGCAGCCCACCGAATCCATCGTGCGGTTCCCCAATATCACCAATCTG |
| | TGCCCCTTCGGCGAGGTGTTCAATGCCACCAGATTCGCCTCTGTGTACGCC |
| | TGGAACCGGAAGCGGATCAGCAATTGCGTGGCCGACTACTCCGTGCTGTAC |
| | AACTCCGCCAGCTTCAGCACCTTCAAGTGCTACGGCGTGTCCCCTACCAAG |
| | CTGAACGACCTGTGCTTCACAAACGTGTACGCCGACAGCTTCGTGATCCGG |
| | GGAGATGAAGTGCGGCAGATTGCCCCTGGACAGACAGGCAAGATCGCCGA |
| | CTACAACTACAAGCTGCCCGACGACTTCACCGGCTGTGTGATTGCCTGGAA |
| | CAGCAACAACCTGGACTCCAAAGTCGGCGGCAACTACAATTACCTGTACCG |
| | GCTGTTCCGGAAGTCCAATCTGAAGCCCTTCGAGCGGGACATCTCCACCGA |
| | GATCTATCAGGCCGGCAGCACCCCTTGTAACGGCGTGGAAGGCTTCAACTG |
| | CTACTTCCCACTGCAGTCCTACGGCTTTCAGCCCACAAATGGCGTGGGCTAT |
| | CAGCCCTACAGAGTGGTGGTGCTGAGCTTCGAACTGCTGCATGCCCCTGCC |
| | ACAGTGTGCGGCCCTAAGAAAAGCACCAATCTCGTGAAGAACAAATGCGTG |
| | AACTTCAACTTCAACGGCCTGACCGGCACCGGCGTGCTGACAGAGAGCAAC |
| | AAGAAGTTCCTGCCATTCCAGCAGTTTGGCCGGGATATCGCCGATACCACA |
| | GACGCCGTTAGAGATCCCCAGACACTGGAAATCCTGGACATCACCCCTTGC |
| | AGCTTCGGCGGAGTGTCTGTGATCACCCCTGGCACCAACACCAGCAATCAG |
| | GTGGCAGTGCTGTACCAGGACGTGAACTGTACCGAAGTGCCCGTGGCCATT |
| | CACGCCGATCAGCTGACACCTACATGGCGGGTGTACTCCACCGGCAGCAAT |
| | GTGTTTCAGACCAGAGCCGGCTGTCTGATCGGAGCCGAGCACGTGAACAAT |
| | AGCTACGAGTGCGACATCCCCATCGGCGCTGGCATCTGTGCCAGCTACCAG |
| | ACACAGACAAACAGCCCCAGAGGATCCGGCAGTGGGTCAGGAAGGGTTCA |
| | GCCCACAGAGTCCATAGTACGGTTCCCTAATATTACCAACCTGTGTCCGTTC |
| | GGTGAAGTCTTCAATGCCACAAGATTCGCCAGCGTTTATGCCTGGAACCGAA |
| | AGAGAATTAGTAATTGTGTTGCGGATTATTCTGTCCTTTACAATAGTGCAAGC |
| | TTCTCCACCTTCAAGTGTTACGGGGTTTCCCCGACGAAACTCAACGACCTTT |
| | GTTTTACAAATGTGTATGCAGATTCATTTGTTATCCGAGGAGATGAGGTACG |
| | GCAAATAGCCCCCGGACAAACAGGAAAGATAGCCGATTACAACTATAAACTG |
| | CCTGATGACTTCACTGGGTGCGTCATCGCATGGAATAGCAACAACCTTGATT |
| | CCAAAGTAGGGGGCAATTATAATTACCGCTATCGACTTTTCCGCAAGTCCAA |
| | CCTCAAACCCTTCGAGCGCGATATCAGCACGGAAATTTACCAAGCGGGCAG |
| | CAAACCATGCAACGGAGTCGAAGGATTTAATTGTTACTTCCCGTTGCAATCC |
| | TATGGCTTTCAACCAACCAACGGTGTTGGTTACCAACCTTATAGAGTTGTCG |
| | TTCTCAGTTTTGAGCTCCTCCATGCCCCGGCCACCGTATGCGGCCCAAAGA |
| | AATCAACAAACCTCGTTAAGAACAAATGTGTCAACTTTAATTTCAATGGGTAG |
| 4372_BetaRBD_ | ATGTTCGTGTTTCTGGTGCTGCTGCCTCTGGTGTCCAGCCGGGTGCAGCCC |
| WHS1_ | ACCGAATCCATCGTGCGGTTCCCCAATATCACCAATCTGTGCCCCTTCGGCG |
| OmicronRBD (SEQ | AGGTGTTCAATGCCACCAGATTCGCCTCTGTGTACGCCTGGAACCGGAAGC |
| ID NO: 28) | GGATCAGCAATTGCGTGGCCGACTATAGTGTACTGTATAACTCAGCATCCTT |
| | TTCCACGTTTAAGTGCTACGGGGTCAGTCCTACAAAGTTGAATGACTTGTGC |
| | TTTACAAACGTATATGCTGATTCCTTTGTCATACGAGGCGATGAGGTCAGAC |
| | AGATTGCTCCGGGACAAACCGGAAATATTGCCGACTACAACTACAAACTTCC |
| | CGACGATTTTACTGGGTGTGTAATTGCCTGGAACAGTAACAATCTTGACTCA |
| | AAAGTGGGGGGAAATTACAATTACCTTTATAGACTCTTCCGCAAGAGCAATC |
| | TCAAGCCTTTTGAACGCGATATATCTACCGAAATATACCAGGCCGGTTCTAC |
| | TCCATGCAATGGAGTTAAGGGATTTAATTGTTACTTTCCTCTCCAATCTTATG |
| | GGTTCCAGCCAACTTATGGAGTCGGTTATCAGCCATATCGAGTCGTTGTGCT |
| | CAGCTTCGAACTGCTTCATGCCCCAGCTACTGTTTGTGGGCCGAAAAAGTCC |
| | ACTAATCTCGTGAAGAATAAGTGCGTAAACTTTGGCAGTGGGTCAGGATCCG |
| | GAATGCAGTGTGTGAACCTGACCACCAGAACACAGCTGCCTCCAGCCTACA |
| | CCAACAGCTTTACCAGAGGCGTGTACTACCCCGACAAGGTGTTCAGATCCA |
| | GCGTGCTGCACTCTACCCAGGACCTGTTCCTGCCTTTCTTCAGCAACGTGAC |
| | CTGGTTCCACGCCATCCACGTGTCCGGCACCAATGGCACCAAGAGATTCGA |
| | CAACCCCGTGCTGCCCTTCAACGACGGGGTGTACTTTGCCAGCACCGAGAA |
| | GTCCAACATCATCAGAGGCTGGATCTTCGGCACCACACTGGACAGCAAGAC |
| | CCAGAGCCTGCTGATCGTGAACAACGCCACCAACGTGGTCATCAAAGTGTG |
| | CGAGTTCCAGTTCTGCAACGACCCCTTCCTGGGCGTCTACTACCACAAGAAC |
| | AACAAGAGCTGGATGGAAAGCGAGTTCCGGGTGTACAGCAGCGCCAACAAC |
| | TGCACCTTCGAGTACGTGTCCCAGCCTTTCCTGATGGACCTGGAAGGCAAG |
| | CAGGGCAACTTCAAGAACCTGCGCGAGTTCGTGTTCAAGAACATCGACGGC |
| | TACTTCAAGATCTACAGCAAGCACACCCCTATCAACCTCGTGCGGGATCTGC |
| | CTCAGGGCTTCTCTGCTCTGGAACCCCTGGTGGATCTGCCCATCGGCATCA |
| | ACATCACCCGGTTTCAGACACTGCTGGCCCTGCACAGAAGCTACCTGACAC |
| | CTGGCGATAGCAGCAGCGGATGGACAGCTGGTGCCGCCGCTTACTATGTG |
| | GGCTACCTGCAGCCTAGAACCTTCCTGCTGAAGTACAACGAGAACGGCACC |
| | ATCACCGACGCCGTGGATTGTGCCCTTGATCCTCTGAGCGAGACAAAGTGC |
| | ACCCTGAAGTCCTTCACCGTGGAAAAGGGCATCTACCAGACCAGCAACTTC |
| | CGGGTGCAGCCCACCGAATCCATCGTGCGGTTCCCCAATATCACCAATCTG |
| | TGCCCCTTCGGCGAGGTGTTCAATGCCACCAGATTCGCCTCTGTGTACGCC |
| | TGGAACCGGAAGCGGATCAGCAATTGCGTGGCCGACTACTCCGTGCTGTAC |
| | AACTCCGCCAGCTTCAGCACCTTCAAGTGCTACGGCGTGTCCCCTACCAAG |
| | CTGAACGACCTGTGCTTCACAAACGTGTACGCCGACAGCTTCGTGATCCGG |
| | GGAGATGAAGTGCGGCAGATTGCCCCTGGACAGACAGGCAAGATCGCCGA |
| | CTACAACTACAAGCTGCCCGACGACTTCACCGGCTGTGTGATTGCCTGGAA |
| | CAGCAACAACCTGGACTCCAAAGTCGGCGGCAACTACAATTACCTGTACCG |
| | GCTGTTCCGGAAGTCCAATCTGAAGCCCTTCGAGCGGGACATCTCCACCGA |
| | GATCTATCAGGCCGGCAGCACCCCTTGTAACGGCGTGGAAGGCTTCAACTG |
| | CTACTTCCCACTGCAGTCCTACGGCTTTCAGCCCACAAATGGCGTGGGCTAT |
| | CAGCCCTACAGAGTGGTGGTGCTGAGCTTCGAACTGCTGCATGCCCCTGCC |
| | ACAGTGTGCGGCCCTAAGAAAAGCACCAATCTCGTGAAGAACAAATGCGTG |
| | AACTTCAACTTCAACGGCCTGACCGGCACCGGCGTGCTGACAGAGAGCAAC |
| | AAGAAGTTCCTGCCATTCCAGCAGTTTGGCCGGGATATCGCCGATACCACA |
| | GACGCCGTTAGAGATCCCCAGACACTGGAAATCCTGGACATCACCCCTTGC |
| | AGCTTCGGCGGAGTGTCTGTGATCACCCCTGGCACCAACACCAGCAATCAG |
| | GTGGCAGTGCTGTACCAGGACGTGAACTGTACCGAAGTGCCCGTGGCCATT |
| | CACGCCGATCAGCTGACACCTACATGGCGGGTGTACTCCACCGGCAGCAAT |
| | GTGTTTCAGACCAGAGCCGGCTGTCTGATCGGAGCCGAGCACGTGAACAAT |
| | AGCTACGAGTGCGACATCCCCATCGGCGCTGGCATCTGTGCCAGCTACCAG |
| | ACACAGACAAACAGCCCCAGAGGATCCGGCAGTGGGTCAGGACGAGTCCA |
| | ACCAACAGAGTCAATCGTAAGATTCCCCAATATCACCAACCTCTGCCCCTTT |
| | GACGAAGTTTTCAACGCTACCAGATTTGCAAGTGTGTACGCCTGGAATAGGA |
| | AGCGGATCAGTAACTGCGTCGCCGACTACTCTGTACTTTATAACCTCGCGCC |
| | CTTCTTTACATTCAAGTGCTATGGCGTCTCCCCCACGAAACTTAATGACCTCT |
| | GTTTCACGAACGTCTACGCCGATTCCTTTGTGATAAGAGGGGATGAGGTAAG |
| | ACAAATAGCACCTGGTCAGACAGGCAACATAGCAGACTATAACTATAAGTTG |
| | CCCGACGACTTTACAGGATGTGTAATTGCTTGGAACAGTAACAAGCTGGATT |
| | CAAAGGTTTCCGGCAATTATAACTACCTGTACAGACTGTTCAGAAAAAGTAAT |
| | CTCAAACCCTTCGAGAGAGACATTTCAACCGAAATCTATCAGGCCGGGAACA |
| | AACCATGCAACGGCGTCGCTGGATTCAACTGTTACTTCCCCCTCCGATCATA |
| | CTCCTTTAGGCCCACATATGGAGTGGGTCATCAACCTTACCGAGTTGTTGTC |
| | CTGTCTTTCGAGCTCTTGCACGCGCCGGCAACTGTGTGTGGACCTAAAAAG |
| | AGTACGAACTTGGTGAAGAATAAGTGTGTAAACTTTAACTTTAATGGTTAG |
| 4495_[S1- | ATGTTTGTGTTTCTAGTGCTACTACCACTAGTGTCCTCTCAGTGCGTGAACCT |
| Omicron_ | GATCACACGGACCCAGTCATACACCAACTCCTTCACCAGAGGAGTGTACTAC |
| BtKY72_ | CCTGACAAGGTGTTCAGATCTAGCGTGCTGCATTCTACCCAGGACCTGTTTC |
| RsSCHC014] | TGCCTTTCTTCAGCAATGTGACCTGGTTCCACGCCATCCACGTGTCTGGCAC |
| (SEQ ID NO: 29) | AAACGGCACCAAGAGATTCGACAACCCTGTGCTGCCTTTCAACGATGGCGT |
| | GTACTTCGCCTCTACAGAGAAGTCCAACATCATCCGGGGCTGGATCTTCGG |
| | CACCACACTGGACTCGAAGACACAATCTCTGCTGATCGTGAACAACGCCAC |
| | CAACGTTGTCATCAAGGTGTGCGAGTTCCAGTTCTGCAACGACCCTTTCTTG |
| | GACGTGTACTACCACAAGAACAACAAGTCCTGGATGGAATCCGAATTCAGAG |
| | TGTACTCCAGCGCCAACAACTGTACATTCGAGTACGTCAGCCAGCCCTTTCT |
| | GATGGACCTGGAAGGCAAGCAGGGAAATTTCAAGAACCTGAGAGAGTTCGT |
| | GTTCAAGAACATCGACGGCTACTTCAAGATCTACAGCAAGCACACCCCTATT |
| | AACCTGGGCAGAGATCTGCCCCAGGGCTTTTCCGCTCTGGAGCCCCTGGTG |
| | GACCTGCCTATCGGCATCAACATCACCCGGTTTCAGACCCTCCTAGCCCTG |
| | CACAGATCCTATCTGACCCCTGGCGACAGCAGTTCCGGCTGGACCGCCGG |
| | GGCTGCTGCCTACTACGTGGGGTACCTGCAGCCTAGAACCTTCCTGCTGAA |
| | GTACAACGAGAACGGCACCATCACCGACGCCGTGGACTGTGCTCTGGATCC |
| | ACTGTCCGAGACCAAGTGCACTCTCAAGAGCTTCACCGTGGAAAAGGGAAT |
| | CTACCAGACCTCCAACTTTAGAGTGCAGCCCACCGAGTCAATCGTGAGATTC |
| | CCCAACATCACCAACCTGTGCCCTTTTGACGAGGTGTTCAACGCTACCAGAT |
| | TTGCTTCTGTGTACGCCTGGAACCGGAAGAGAATCTCTAACTGCGTGGCCG |
| | ATTACTCCGTGCTGTACAACTTCGCTCCTTTCTTCGCCTTCAAGTGTTACGGC |
| | GTGTCCCCTACCAAGCTGAACGACCTGTGTTTCACTAACGTGTACGCCGACT |
| | CTTTCGTGATCCGGGGCAACGAGGTGTCCCAGATCGCCCCAGGCCAGACA |
| | GGCAACATCGCTGACTACAACTACAAGCTGCCTGACGATTTCACCGGCTGT |
| | GTGATCGCCTGGAACTCCAACAAGCTGGATTCCAAAGTGGGCGGCAACTAC |
| | AACTATCTGTACCGGCTGTTCAGAAAGTCCAATCTGAAGCCCTTCGAACGCG |
| | ACATCTCCACCGAGATCTATCAGGCCGGCAACAAGCCTTGTAACGGCGTCG |
| | CTGGCTTCAACTGCTACTTCCCTTTGCGATCTTATGGCTTCCGGCCTACCTA |
| | CGGCGTGGGCCACCAGCCTTACCGGGTGGTGGTGCTGAGCTTTGAGCTGC |
| | TGCACGCTCCTGCTACAGTGTGCGGCCCTAAGAAGTCCACCAATCTGGTGA |
| | AAAATAAGTGCGTTAACTTCAACTTCAACGGCCTGACCGGCACCGGCGTGCT |
| | GACAGAGTCAAATAAGAAGTTCCTGCCTTTCCAGCAGTTTGGCAGGGACATC |
| | GCCGACACCACGGACGCTGTGCGGGACCCTCAGACCCTGGAAATCCTGGA |
| | TATCACCCCTTGCTCCTTCGGCGGCGTGAGCGTGATTACCCCTGGAACCAA |
| | CACCTCTAATCAAGTGGCCGTGCTGTACCAAGGCGTGAACTGCACCGAAGT |
| | GCCCGTGGCTATCCACGCCGATCAGCTGACCCCTACATGGCGGGTATACAG |
| | TACCGGCTCTAACGTGTTCCAGACCCGGGCTGGCTGCCTGATCGGCGCCGA |
| | GTACGTGAACAACTCCTACGAGTGCGACATCCCCATCGGCGCTGGCATTTG |
| | CGGATCTGGCTCTGGATCCAGAGTCTCTCCAAGCACCGAGGTGGTGCGGTT |
| | TCCTAATATCACCAACCTGTGCCCTTTCGGACAAGTGTTTAACGCCTCCAAC |
| | TTCCCATCGGTGTACGCCTGGGAGAGACTGCGCATCTCCGACTGCGTCGCC |
| | GACTATGCCGTGCTGTACAATTCTTCCTCCTCTTTCTCCACCTTTAAGTGCTA |
| | CGGCGTGAGCCCTACAAAGCTGAACGACCTGTGCTTCTCCTCTGTGTACGC |
| | CGACTACTTCGTGGTGAAAGGCGATGATGTGAGACAGATCGCCCCTGCCCA |
| | GACCGGCGTGATCGCCGATTACAACTACAAGCTCCCCGACGACTTCACAGG |
| | CTGCGTGCTGGCCTGGAATACCAACTCCGTAGATTCCAAATCCGGAAACAAC |
| | TTCTACTACCGGCTGTTCAGACACGGCAAGATCAAGCCCTACGAAAGAGACA |
| | TCTCCAACGTGCTGTACAACTCCGCTGGCGGCACCTGTTCCTCCATCTCTCA |
| | GCTGGGCTGCTACGAGCCCCTGAAATCCTATGGGTTCACCCCCACCGTGGG |
| | GGTGGGCTACCAGCCTTATAGGGTCGTGGTGTTATCCTTCGAACTGCTGAA |
| | CGCTCCAGCTACCGTGTGCGGCCCCAAGAAATCAACCGAGCTGGTCAAGAA |
| | CAAGTGCGTGAACTTCGGCTCTGGCAGCGGCTCTAGAGTGGCACCCTCTAA |
| | AGAAGTGGTCAGATTCCCCAATATCACCAATCTGTGTCCTTTTGGCGAGGTC |
| | TTCAACGCCACCACCTTCCCTTCTGTGTACGCCTGGGAACGGAAACGGATCT |
| | CTAACTGCGTCGCCGACTACTCTGTTCTCTACAACTCCACCTCTTTCTCCAC |
| | CTTTAAATGTTACGGCGTCTCTGCTACAAAACTGAACGATCTGTGCTTCTCTA |
| | ACGTGTACGCTGACTCTTTTGTGGTAAAGGGCGACGACGTGAGACAGATCG |
| | CCCCTGGCCAAACCGGAGTCATCGCTGATTATAACTACAAACTGCCTGATGA |
| | CTTCCTGGGATGTGTGCTGGCTTGGAACACCAATAGCAAGGACAGCAGCAC |
| | ATCTGGCAACTACAACTACCTGTACAGATGGGTGAGGAGAAGCAAGCTCAA |
| | CCCGTATGAACGGGACCTGTCTAACGACATCTACTCTCCAGGCGGACAGTC |
| | CTGCTCCGCTGTGGGCCCTAACTGCTACAATCCTCTGAGACCCTACGGCTT |
| | CTTCACCACCGCTGGCGTGGGACATCAGCCTTACAGAGTTGTTGTGCTGAG |
| | CTTCGAGCTGCTGAACGCCCCTGCCACCGTGTGTGGCCCTAAGCTGTCTAC |
| | TGATCTGATCAAAAACCAGTGCGTGAATTTCtgataataa |
| 4498_[S1_ | ATGTTTGTGTTTCTAGTGCTACTACCACTAGTGTCCTCTCAGTGCGTGAACCT |
| Omicron_ | GATCACACGGACCCAGTCATACACCAACTCCTTCACCAGAGGAGTGTACTAC |
| HKU3_BTKY72 | CCTGACAAGGTGTTCAGATCTAGCGTGCTGCATTCTACCCAGGACCTGTTTC |
| (SEQ ID NO: 30) | TGCCTTTCTTCAGCAATGTGACCTGGTTCCACGCCATCCACGTGTCTGGCAC |
| | AAACGGCACCAAGAGATTCGACAACCCTGTGCTGCCTTTCAACGATGGCGT |
| | GTACTTCGCCTCTACAGAGAAGTCCAACATCATCCGGGGCTGGATCTTCGG |
| | CACCACACTGGACTCGAAGACACAATCTCTGCTGATCGTGAACAACGCCAC |
| | CAACGTTGTCATCAAGGTGTGCGAGTTCCAGTTCTGCAACGACCCTTTCTTG |
| | GACGTGTACTACCACAAGAACAACAAGTCCTGGATGGAATCCGAATTCAGAG |
| | TGTACTCCAGCGCCAACAACTGTACATTCGAGTACGTCAGCCAGCCCTTTCT |
| | GATGGACCTGGAAGGCAAGCAGGGAAATTTCAAGAACCTGAGAGAGTTCGT |
| | GTTCAAGAACATCGACGGCTACTTCAAGATCTACAGCAAGCACACCCCTATT |
| | AACCTGGGCAGAGATCTGCCCCAGGGCTTTTCCGCTCTGGAGCCCCTGGTG |
| | GACCTGCCTATCGGCATCAACATCACCCGGTTTCAGACCCTCCTAGCCCTG |
| | CACAGATCCTATCTGACCCCTGGCGACAGCAGTTCCGGCTGGACCGCCGG |
| | GGCTGCTGCCTACTACGTGGGGTACCTGCAGCCTAGAACCTTCCTGCTGAA |
| | GTACAACGAGAACGGCACCATCACCGACGCCGTGGACTGTGCTCTGGATCC |
| | ACTGTCCGAGACCAAGTGCACTCTCAAGAGCTTCACCGTGGAAAAGGGAAT |
| | CTACCAGACCTCCAACTTTAGAGTGCAGCCCACCGAGTCAATCGTGAGATTC |
| | CCCAACATCACCAACCTGTGCCCTTTTGACGAGGTGTTCAACGCTACCAGAT |
| | TTGCTTCTGTGTACGCCTGGAACCGGAAGAGAATCTCTAACTGCGTGGCCG |
| | ATTACTCCGTGCTGTACAACTTCGCTCCTTTCTTCGCCTTCAAGTGTTACGGC |
| | GTGTCCCCTACCAAGCTGAACGACCTGTGTTTCACTAACGTGTACGCCGACT |
| | CTTTCGTGATCCGGGGCAACGAGGTGTCCCAGATCGCCCCAGGCCAGACA |
| | GGCAACATCGCTGACTACAACTACAAGCTGCCTGACGATTTCACCGGCTGT |
| | GTGATCGCCTGGAACTCCAACAAGCTGGATTCCAAAGTGGGCGGCAACTAC |
| | AACTATCTGTACCGGCTGTTCAGAAAGTCCAATCTGAAGCCCTTCGAACGCG |
| | ACATCTCCACCGAGATCTATCAGGCCGGCAACAAGCCTTGTAACGGCGTCG |
| | CTGGCTTCAACTGCTACTTCCCTTTGCGATCTTATGGCTTCCGGCCTACCTA |
| | CGGCGTGGGCCACCAGCCTTACCGGGTGGTGGTGCTGAGCTTTGAGCTGC |
| | TGCACGCTCCTGCTACAGTGTGCGGCCCTAAGAAGTCCACCAATCTGGTGA |
| | AAAATAAGTGCGTTAACTTCAACTTCAACGGCCTGACCGGCACCGGCGTGCT |
| | GACAGAGTCAAATAAGAAGTTCCTGCCTTTCCAGCAGTTTGGCAGGGACATC |
| | GCCGACACCACGGACGCTGTGCGGGACCCTCAGACCCTGGAAATCCTGGA |
| | TATCACCCCTTGCTCCTTCGGCGGCGTGAGCGTGATTACCCCTGGAACCAA |
| | CACCTCTAATCAAGTGGCCGTGCTGTACCAAGGCGTGAACTGCACCGAAGT |
| | GCCCGTGGCTATCCACGCCGATCAGCTGACCCCTACATGGCGGGTATACAG |
| | TACCGGCTCTAACGTGTTCCAGACCCGGGCTGGCTGCCTGATCGGCGCCGA |
| | GTACGTGAACAACTCCTACGAGTGCGACATCCCCATCGGCGCTGGCATTTG |
| | CGGATCTGGCTCTGGATCCAGAGTCTCTCCAAGCACCGAGGTGGTGCGGTT |
| | TCCTAATATCACCAACCTGTGCCCTTTCGGACAAGTGTTTAACGCCTCCAAC |
| | TTCCCATCGGTGTACGCCTGGGAGAGACTGCGCATCTCCGACTGCGTCGCC |
| | GACTATGCCGTGCTGTACAATTCTTCCTCCTCTTTCTCCACCTTTAAGTGCTA |
| | CGGCGTGAGCCCTACAAAGCTGAACGACCTGTGCTTCTCCTCTGTGTACGC |
| | CGACTACTTCGTGGTGAAAGGCGATGATGTGAGACAGATCGCCCCTGCCCA |
| | GACCGGCGTGATCGCCGATTACAACTACAAGCTCCCCGACGACTTCACAGG |
| | CTGCGTGCTGGCCTGGAATACCAACTCCGTAGATTCCAAATCCGGAAACAAC |
| | TTCTACTACCGGCTGTTCAGACACGGCAAGATCAAGCCCTACGAAAGAGACA |
| | TCTCCAACGTGCTGTACAACTCCGCTGGCGGCACCTGTTCCTCCATCTCTCA |
| | GCTGGGCTGCTACGAGCCCCTGAAATCCTATGGGTTCACCCCCACCGTGGG |
| | GGTGGGCTACCAGCCTTATAGGGTCGTGGTGTTATCCTTCGAACTGCTGAA |
| | CGCTCCAGCTACCGTGTGCGGCCCCAAGAAATCAACCGAGCTGGTCAAGAA |
| | CAAGTGCGTGAACTTCGGCTCTGGCAGCGGCTCTAGAGTGGCACCCTCTAA |
| | AGAAGTGGTCAGATTCCCCAATATCACCAATCTGTGTCCTTTTGGCGAGGTC |
| | TTCAACGCCACCACCTTCCCTTCTGTGTACGCCTGGGAACGGAAACGGATCT |
| | CTAACTGCGTCGCCGACTACTCTGTTCTCTACAACTCCACCTCTTTCTCCAC |
| | CTTTAAATGTTACGGCGTCTCTGCTACAAAACTGAACGATCTGTGCTTCTCTA |
| | ACGTGTACGCTGACTCTTTTGTGGTAAAGGGCGACGACGTGAGACAGATCG |
| | CCCCTGGCCAAACCGGAGTCATCGCTGATTATAACTACAAACTGCCTGATGA |
| | CTTCCTGGGATGTGTGCTGGCTTGGAACACCAATAGCAAGGACAGCAGCAC |
| | ATCTGGCAACTACAACTACCTGTACAGATGGGTGAGGAGAAGCAAGCTCAA |
| | CCCGTATGAACGGGACCTGTCTAACGACATCTACTCTCCAGGCGGACAGTC |
| | CTGCTCCGCTGTGGGCCCTAACTGCTACAATCCTCTGAGACCCTACGGCTT |
| | CTTCACCACCGCTGGCGTGGGACATCAGCCTTACAGAGTTGTTGTGCTGAG |
| | CTTCGAGCTGCTGAACGCCCCTGCCACCGTGTGTGGCCCTAAGCTGTCTAC |
| | TGATCTGATCAAAAACCAGTGCGTGAATTTCtgataataa |
| 4499_[WHS1_ | ATGTTTGTGTTTCTAGTGCTACTACCACTAGTGTCTTCCCAGTGCGTCAATCT |
| KHOSTA2RBD_ | GACCACCAGAACCCAACTGCCCCCTGCTTACACTAACTCTTTCACCAGAGGC |
| WIVIRBD] | GTGTACTACCCTGACAAGGTGTTCCGGTCGTCCGTGCTGCATTCTACCCAG |
| (SEQ ID NO: 31) | GATCTCTTCTTGCCTTTCTTCTCTAACGTGACCTGGTTCCACGCCATCCATGT |
| | GTCTGGCACAAACGGCACCAAGAGATTCGACAACCCTGTGCTGCCTTTCAA |
| | CGACGGCGTGTATTTCGCCTCTACCGAGAAGTCTAACATCATCCGGGGCTG |
| | GATCTTCGGCACCACACTGGACAGCAAGACCCAGTCGCTGCTGATCGTGAA |
| | CAACGCCACCAACGTGGTCATCAAGGTCTGCGAATTCCAGTTCTGCAACGAT |
| | CCTTTCCTGGGCGTATACTACCACAAGAACAACAAGTCCTGGATGGAATCCG |
| | AATTCAGAGTATACTCCTCCGCCAATAACTGCACATTCGAGTACGTGTCTCA |
| | GCCTTTCCTGATGGACCTGGAAGGCAAGCAGGGCAACTTCAAAAACCTGAG |
| | AGAGTTCGTGTTCAAGAACATCGACGGCTACTTCAAGATCTACAGCAAGCAC |
| | ACCCCTATCAACCTGGTGCGGGATCTGCCACAGGGCTTTTCCGCTCTGGAA |
| | CCTCTGGTGGACCTGCCCATCGGCATCAATATCACCCGCTTTCAAACCCTGC |
| | TGGCCCTGCACAGATCTTACCTGACACCAGGCGACTCCTCCTCCGGCTGGA |
| | CCGCCGGCGCTGCCGCCTACTACGTGGGCTACCTCCAGCCTCGGACCTTTC |
| | TGCTCAAGTACAACGAGAACGGCACAATCACCGACGCCGTGGACTGCGCCC |
| | TGGACCCCCTGTCCGAGACAAAGTGTACCCTGAAGTCCTTCACCGTGGAAA |
| | AAGGAATCTATCAAACCTCCAACTTTCGTGTGCAACCTACTGAATCTATCGTG |
| | AGATTTCCTAATATTACCAATCTGTGTCCTTTTGGCGAGGTGTTCAACGCCAC |
| | TCGGTTCGCTTCCGTGTACGCCTGGAACAGAAAGCGGATCAGCAACTGCGT |
| | TGCTGACTACTCCGTGCTGTACAACTCCGCTTCCTTCAGCACCTTCAAGTGC |
| | TATGGGGTCTCCCCTACCAAGCTGAACGATCTGTGCTTCACCAACGTGTATG |
| | CAGACTCTTTCGTGATCAGAGGCGACGAGGTCAGACAGATCGCCCCCGGCC |
| | AGACCGGAAAGATCGCTGACTACAACTACAAGCTGCCCGACGACTTCACAG |
| | GCTGTGTGATCGCGTGGAACTCTAACAACCTAGACTCTAAGGTAGGCGGCA |
| | ACTATAACTACCTGTACAGACTGTTTCGGAAGAGCAACCTGAAGCCTTTCGA |
| | GCGGGACATCTCGACCGAGATCTACCAGGCTGGCAGCACACCCTGCAACG |
| | GGGTGGAAGGATTTAACTGCTACTTCCCACTGCAGTCTTATGGCTTCCAGCC |
| | CACAAACGGCGTGGGCTACCAACCTTACCGGGTGGTGGTGCTGTCCTTCGA |
| | GCTGCTGCACGCTCCTGCTACCGTGTGTGGCCCAAAGAAATCTACCAACCT |
| | GGTGAAGAACAAGTGTGTCAACTTCAATTTCAACGGCCTGACCGGCACCGG |
| | CGTCCTGACCGAGTCTAATAAGAAGTTCCTGCCCTTTCAGCAGTTCGGCAGA |
| | GATATCGCCGATACCACCGACGCTGTGCGGGACCCCCAGACGCTGGAAATC |
| | CTGGATATCACGCCTTGCTCTTTCGGCGGCGTGTCCGTGATCACACCCGGC |
| | ACCAACACCAGCAACCAAGTGGCTGTGTTGTATCAGGACGTGAACTGCACC |
| | GAAGTGCCTGTGGCCATCCACGCCGATCAGCTGACCCCTACCTGGAGAGTG |
| | TACTCAACCGGCAGCAACGTGTTCCAGACCAGAGCGGGCTGCCTGATCGGC |
| | GCCGAGCACGTGAACAACTCCTACGAGTGCGACATCCCTATCGGAGCCGGC |
| | ATCTGCGCCAGCTACCAAACACAGACCAACTCCCCTCGGGGATCTGGCTCT |
| | GGTTCCAGAGTGTCTCCTTCTCTGGACGTGGTGAGGTTTCCAAACATGACCA |
| | ACATCTGCCCTTTCGACCAGGTGTTCAACAAGACCCAGTTCCCTTCCGTCTA |
| | CGCTTGGGAACGGGTGAGAATCTCCGACTGTGTGTCCGACTACACCGTGCT |
| | GTACAACAGCTCTGCTTCTTTCTCCACCTTCAAGTGTTACGGCGTGTCTCCT |
| | ACCAAGCTGAACGACCTGTGCTTCTCAGGCGTGTACGCCGACTACTTCGTG |
| | GTCAAAGGCGATCACGTGCACCAGATCGCCCCTGGCCAGACCGGCGTGAT |
| | CGCCGACTACAATTACAAACTGCCTTCCGAGTTCGTAGGCTGCATCCTGGCT |
| | TGGAACACAAGAACCATCGACTCCAAGAGAGGATTCTACTACCGGCTGTTCA |
| | GACACGGCAATATCCGGCCGTACGAGAGAGACACCTCCAATGTGCCCTACA |
| | ATGCTGCTGGAGGCACCTGCAACCAGCCTGGCACCCACAACTGCTACGAGC |
| | CTCTGCAGGACTACGGCTTCACATCCACATCGGGAGTGGGCTACCAGCCAT |
| | TCCGCGTGGTGGTCTTATCCTTTGAGCTGCTGAACGCCCCTGCCACCGTGT |
| | GTGGCCCCAAGCAGAGCACCGATCTAGTGAAAAACAAGTGCGTCAATTTCG |
| | GCTCCGGAAGCGGCTCCAGAGTGGCTCCCTCTAAAGAGGTGGTGAGATTCC |
| | CCAACATTACCAACCTGTGCCCTTTTGGAGAGGTCTTCAATGCTACCACCTT |
| | CCCTAGCGTATACGCTTGGGAGCGGAAGCGGATCTCCAACTGCGTGGCCGA |
| | CTACTCTGTGCTGTACAACTCCACCAGCTTTTCTACCTTCAAATGCTACGGC |
| | GTGTCCGCCACCAAGCTGAACGATCTGTGCTTTTCCAACGTGTACGCCGATT |
| | CCTTCGTGGTTAAGGGCGACGATGTGAGACAGATCGCCCCTGGCCAGACAG |
| | GTGTAATCGCTGATTACAACTACAAGCTGCCTGACGACTTCACCGGCTGTGT |
| | GCTGGCCTGGAACACCAGAAACATCGATGCTACACAGACCGGCAACTACAA |
| | CTACAAGTACCGGAGCCTGCGGCATGGCAAGCTGAGACCTTTCGAGAGAGA |
| | TATCTCCAACGTGCCATTCTCTCCCGATGGCAAACCTTGTACCCCACCTGCT |
| | TTCAACTGCTACTGGCCCCTGAACGACTACGGCTTCTATATCACTAACGGCA |
| | TCGGCTACCAGCCATACAGAGTGGTGGTTCTGAGTTTCGAACTGCTCAATGC |
| | TCCAGCCACCGTGTGCGGACCTAAGCTTTCCACCGACCTGATAAAAAACCA |
| | GTGTGTGAACTTCtgataataa |
| 4500_[WHS1_ | ATGTTTGTGTTTCTAGTGCTACTACCACTAGTGTCCTCCCAGTGTGTGAATCT |
| Pang17RBD_ | GACAACCAGAACCCAGCTGCCTCCAGCCTACACCAATTCCTTCACCAGAGG |
| WIV1RBD] | AGTCTACTACCCTGACAAAGTGTTCAGATCCTCTGTGCTCCACTCAACACAG |
| (SEQ ID NO: 32) | GATCTGTTTCTGCCCTTCTTCTCCAACGTCACTTGGTTCCACGCTATCCATGT |
| | GTCCGGAACAAACGGCACAAAGAGATTCGACAACCCCGTGTTGCCTTTCAA |
| | CGACGGCGTGTACTTCGCCTCTACCGAGAAGTCCAACATCATCAGAGGCTG |
| | GATCTTCGGCACCACACTCGATTCTAAGACCCAGTCTCTGCTGATCGTTAAC |
| | AACGCCACCAACGTGGTCATCAAGGTGTGCGAGTTCCAGTTCTGCAACGAC |
| | CCCTTCCTGGGCGTCTACTACCACAAGAACAACAAGTCTTGGATGGAATCCG |
| | AGTTCCGTGTTTACAGCTCCGCCAACAACTGTACCTTCGAGTACGTCTCTCA |
| | GCCCTTCCTAATGGACCTGGAAGGTAAACAGGGCAATTTCAAGAACCTGAGA |
| | GAGTTCGTGTTCAAGAACATCGACGGATATTTTAAGATCTACTCTAAGCACAC |
| | CCCTATCAACCTGGTGCGGGACCTGCCTCAAGGCTTCTCCGCTCTGGAGCC |
| | TCTAGTGGACCTGCCTATTGGCATCAACATCACCAGATTCCAGACCCTACTG |
| | GCTCTGCACAGATCTTACCTGACTCCTGGCGACAGCTCCAGCGGCTGGACC |
| | GCCGGCGCTGCCGCTTACTACGTGGGCTACCTGCAGCCTCGGACCTTCCTG |
| | CTGAAGTACAACGAGAACGGAACCATCACCGACGCTGTGGACTGCGCCCTG |
| | GATCCTCTGTCTGAAACTAAGTGCACCCTGAAGTCGTTTACCGTGGAAAAGG |
| | GCATCTACCAGACCTCCAACTTCAGAGTGCAGCCAACCGAATCTATCGTGCG |
| | CTTCCCCAACATCACAAACCTGTGCCCCTTCGGCGAAGTGTTCAACGCTACA |
| | CGGTTCGCCTCCGTATACGCTTGGAACAGAAAAAGAATCTCCAACTGTGTGG |
| | CTGATTACTCCGTGCTGTATAACAGCGCCTCTTTTTCCACCTTCAAGTGCTAC |
| | GGGGTGTCTCCTACCAAACTGAACGACCTGTGCTTTACCAACGTGTACGCC |
| | GACTCCTTTGTGATCCGGGGCGATGAGGTGCGGCAGATCGCCCCTGGCCA |
| | GACCGGCAAGATCGCCGACTATAATTACAAGCTGCCTGACGACTTCACCGG |
| | CTGCGTGATCGCATGGAACTCCAACAATCTGGACTCCAAGGTGGGCGGCAA |
| | CTACAACTATCTGTATAGACTGTTCAGAAAGTCCAACCTGAAACCTTTCGAGA |
| | GAGACATCAGCACCGAGATCTACCAGGCCGGCTCTACCCCTTGCAACGGCG |
| | TAGAGGGCTTCAACTGCTACTTCCCCCTGCAGTCCTACGGCTTTCAACCTAC |
| | CAACGGAGTGGGCTACCAACCTTACCGGGTGGTGGTGCTGTCCTTCGAGCT |
| | GCTGCACGCCCCTGCTACTGTGTGCGGACCTAAGAAATCTACCAATCTGGT |
| | GAAAAACAAGTGCGTGAACTTCAACTTCAACGGCCTGACAGGCACTGGCGT |
| | CCTGACCGAGTCCAACAAGAAGTTCCTGCCTTTCCAGCAGTTCGGCAGAGA |
| | CATCGCCGACACCACCGACGCCGTTAGAGATCCTCAGACCCTGGAGATCCT |
| | GGACATCACCCCTTGCTCCTTTGGCGGCGTGTCCGTGATCACCCCTGGCAC |
| | CAACACCAGTAATCAGGTGGCCGTGCTGTACCAGGACGTGAACTGCACCGA |
| | AGTGCCTGTGGCCATCCACGCCGACCAGCTGACACCTACATGGCGGGTTTA |
| | CTCCACAGGATCCAACGTGTTCCAGACCCGGGCTGGCTGCCTGATCGGCGC |
| | CGAGCACGTGAACAACTCCTACGAGTGCGACATCCCTATCGGCGCTGGCAT |
| | CTGCGCTTCTTACCAAACCCAGACCAACTCCCCCCGCGGCTCTGGCTCCGG |
| | CAGTCGGGTGCAACCTACCATCTCCATCGTGCGGTTTCCTAACATCACCAAC |
| | CTGTGCCCTTTCGGCGAGGTCTTCAACGCCAGCAAGTTCGCTTCCGTGTAT |
| | GCCTGGAACAGAAAGAGAATCTCCAACTGCGTCGCCGACTACTCCGTGCTG |
| | TACAATTCTACAAGTTTCTCCACCTTCAAGTGCTACGGCGTGTCCCCTACCA |
| | AGCTGAACGACCTGTGCTTCACCAACGTCTACGCCGACTCCTTCGTGGTGA |
| | AGGGCGATGAAGTGAGACAGATCGCTCCAGGACAGACAGGCGTAATTGCCG |
| | ACTATAATTATAAACTCCCCGATGACTTCACCGGCTGCGTGATTGCCTGGAA |
| | CTCTGTCAAGCAGGACGCTTTGACCGGCGGAAACTACGGCTACCTGTACCG |
| | GCTGTTCAGAAAGTCCAAGCTGAAACCTTTTGAACGGGACATCTCTACCGAA |
| | ATCTACCAGGCTGGGAGCACCCCTTGTAACGGACAAGTGGGCCTGAACTGT |
| | TACTACCCCCTGGAAAGATACGGCTTCCATCCTACCACCGGCGTGAATTACC |
| | AGCCTTTCCGGGTTGTGGTTCTGAGCTTTGAGCTGCTGAACGGCCCAGCTA |
| | CGGTGTGCGGCCCCAAGCTCTCTACCACCCTTGTGAAGGACAAGTGCGTCA |
| | ATTTCGGCTCTGGCTCTGGGTCCAGAGTGGCTCCTTCCAAAGAGGTGGTGA |
| | GATTTCCTAATATCACCAACCTGTGTCCTTTCGGCGAAGTGTTCAACGCCAC |
| | CACCTTCCCATCTGTGTACGCCTGGGAGAGAAAGCGGATCAGCAACTGTGT |
| | GGCTGATTATTCTGTGCTGTACAACTCCACCTCCTTCTCCACCTTTAAGTGTT |
| | ACGGCGTGTCTGCTACCAAGCTGAACGATCTCTGTTTTAGTAACGTGTACGC |
| | CGACTCCTTCGTGGTGAAGGGCGACGATGTGCGGCAGATCGCCCCTGGCC |
| | AGACCGGCGTGATCGCTGATTACAACTACAAGCTGCCTGACGACTTTACAG |
| | GCTGTGTGCTGGCTTGGAACACCCGGAACATCGATGCCACCCAGACCGGCA |
| | ACTACAACTACAAGTACCGCTCTCTGCGGCACGGCAAGCTGAGGCCCTTTG |
| | AGCGGGATATCTCTAACGTGCCTTTCAGCCCTGATGGCAAGCCCTGCACCC |
| | CTCCAGCCTTCAATTGTTACTGGCCTCTGAACGACTACGGCTTCTACATCAC |
| | CAACGGAATCGGCTACCAGCCCTATAGAGTGGTGGTGCTGTCGTTCGAGCT |
| | GCTGAATGCTCCCGCCACCGTGTGTGGCCCAAAGCTGTCCACCGACCTGAT |
| | CAAGAACCAGTGCGTGAACTTCtgataataa |
| 4501_[WHS1_ | ATGTTTGTGTTTCTAGTGCTACTACCACTAGTGTCCTCCCAGTGCGTGAACC |
| Khosta2RBD_ | TGACCACAAGAACCCAGCTGCCTCCTGCGTACACCAACTCCTTCACCAGAG |
| RsSHC014BD_ | GCGTGTACTACCCTGACAAGGTGTTTCGTTCCTCCGTCTTGCACTCCACACA |
| WIV1RBD] | GGATCTGTTTCTGCCTTTCTTCTCTAACGTGACGTGGTTCCACGCCATCCAC |
| (SEQ ID NO: 33) | GTGTCCGGTACCAACGGCACAAAGAGATTCGACAACCCCGTGCTGCCTTTC |
| | AACGACGGCGTTTACTTCGCCTCCACTGAGAAGTCTAACATCATCAGAGGAT |
| | GGATCTTCGGTACCACCCTCGACAGCAAGACCCAATCCCTGCTGATAGTGA |
| | ACAACGCTACAAACGTGGTGATCAAGGTGTGCGAGTTCCAGTTCTGCAACG |
| | ATCCTTTTCTGGGAGTGTACTACCACAAGAATAACAAGTCCTGGATGGAAAG |
| | TGAATTCAGAGTGTACTCTAGCGCCAACAACTGTACCTTTGAGTACGTGTCC |
| | CAACCTTTCCTGATGGACCTGGAAGGAAAGCAGGGCAACTTCAAGAACCTG |
| | AGAGAGTTCGTGTTCAAGAACATCGATGGCTACTTCAAGATCTACTCCAAGC |
| | ACACACCTATCAACTTGGTTAGAGACCTCCCGCAGGGCTTCTCCGCTCTGGA |
| | ACCTCTGGTCGACCTGCCCATTGGCATCAACATCACACGGTTCCAGACCCT |
| | GCTGGCTCTGCACAGATCCTACCTGACCCCAGGAGATTCCTCTTCTGGCTG |
| | GACCGCCGGCGCTGCCGCTTACTACGTGGGCTACCTGCAGCCTCGGACATT |
| | CCTGCTGAAGTACAACGAGAACGGCACCATCACCGACGCTGTGGACTGTGC |
| | CCTGGATCCCCTGAGCGAGACAAAGTGTACCCTGAAATCTTTTACCGTGGAA |
| | AAGGGCATTTACCAGACCTCCAACTTCAGGGTGCAGCCTACTGAGTCCATC |
| | GTGCGGTTCCCTAACATCACCAACCTGTGCCCCTTCGGCGAAGTGTTTAACG |
| | CCACCAGATTTGCTTCTGTGTACGCTTGGAACAGAAAGAGAATCAGCAACTG |
| | CGTGGCTGATTACTCCGTCCTGTACAATTCAGCAAGCTTCTCCACCTTCAAA |
| | TGCTACGGAGTGTCTCCAACCAAGCTGAACGACCTGTGTTTCACCAACGTGT |
| | ACGCCGACTCTTTCGTGATCCGGGGCGACGAAGTCCGGCAAATCGCCCCC |
| | GGCCAGACCGGCAAGATCGCCGACTACAACTACAAGCTGCCTGACGATTTC |
| | ACCGGATGTGTCATCGCTTGGAACTCCAACAACCTGGATAGTAAAGTGGGC |
| | GGCAACTATAATTATTTGTACCGGCTGTTCCGGAAGTCTAATCTGAAGCCTTT |
| | CGAGAGAGACATCTCTACCGAGATCTACCAGGCCGGCTCTACACCTTGCAA |
| | CGGCGTAGAGGGCTTCAACTGCTACTTCCCCCTGCAGAGCTACGGCTTCCA |
| | GCCTACTAACGGCGTTGGCTACCAGCCTTACAGAGTGGTGGTGCTGTCTTT |
| | CGAGCTGCTGCATGCTCCAGCCACAGTGTGCGGCCCTAAGAAGTCCACGAA |
| | CCTGGTGAAGAACAAATGCGTGAACTTCAACTTCAATGGCCTGACCGGCAC |
| | CGGCGTGCTGACAGAGTCTAACAAGAAGTTCCTGCCATTCCAGCAGTTCGG |
| | CAGAGATATTGCTGACACCACTGATGCCGTACGGGACCCTCAGACCCTGGA |
| | AATCCTGGATATCACCCCTTGTTCCTTCGGCGGCGTCTCCGTGATCACTCCT |
| | GGAACCAACACCTCCAACCAGGTGGCCGTGCTGTACCAGGACGTGAACTGC |
| | ACCGAGGTGCCTGTGGCCATCCATGCCGACCAGCTGACGCCCACCTGGCG |
| | GGTGTACTCCACCGGATCTAATGTGTTCCAAACCCGGGCTGGCTGCCTGAT |
| | CGGCGCCGAGCACGTGAATAACTCCTACGAGTGCGACATCCCTATCGGGGC |
| | CGGAATCTGCGCGTCATACCAGACCCAGACAAACTCTCCTAGAGGCTCTGG |
| | CTCTGGCAGCAGAGTTTCGCCTTCTCTGGACGTGGTCAGATTCCCTAACATG |
| | ACCAACATCTGCCCTTTCGACCAGGTGTTCAACAAAACCCAGTTCCCCTCCG |
| | TGTACGCCTGGGAACGGGTGAGAATTTCCGACTGCGTCTCAGATTACACCG |
| | TGCTCTATAACAGCTCTGCCTCTTTCTCTACCTTTAAGTGCTACGGCGTGAG |
| | CCCTACCAAGTTAAATGACCTGTGCTTTAGCGGCGTGTACGCCGATTATTTC |
| | GTGGTAAAGGGTGACCACGTGCACCAGATAGCCCCCGGCCAGACCGGCGT |
| | GATCGCTGACTACAATTATAAGCTGCCCAGCGAGTTCGTGGGCTGCATCCT |
| | GGCCTGGAACACTAGAACAATCGACTCCAAGAGAGGCTTCTACTACCGGCT |
| | GTTCCGGCATGGCAACATCAGACCTTACGAGCGCGATACCTCCAATGTGCC |
| | CTACAACGCTGCTGGCGGGACCTGCAACCAGCCTGGCACCCACAACTGCTA |
| | CGAGCCTCTGCAGGACTACGGCTTCACCTCCACATCTGGCGTCGGCTACCA |
| | ACCATTCAGAGTGGTGGTGCTGTCTTTCGAGCTGCTGAACGCTCCAGCTAC |
| | CGTGTGTGGCCCTAAGCAATCCACAGATCTGGTGAAGAACAAGTGTGTGAAT |
| | TTCGGTTCCGGCTCCGGCTCTAGAGTGGCTCCTTCTAAAGAGGTTGTGCGG |
| | TTTCCTAACATCACCAACCTGTGTCCTTTCGGCGAGGTGTTCAACGCCACCA |
| | CGTTCCCCTCTGTGTACGCCTGGGAGAGAAAGAGGATCTCCAACTGTGTTG |
| | CTGACTACTCGGTGCTGTATAACTCCACCTCTTTTTCCACCTTCAAGTGCTAC |
| | GGCGTCTCCGCCACCAAGCTCAATGACCTGTGCTTCTCGAACGTGTACGCA |
| | GACTCCTTCGTGGTCAAGGGCGACGACGTGAGACAGATCGCCCCTGGCCA |
| | GACTGGCGTGATCGCCGACTATAATTACAAACTGCCGGACGACTTTCTGGG |
| | CTGCGTGCTGGCTTGGAACACCAACTCTAAGGACTCTTCCACCTCTGGCAAC |
| | TACAACTACCTGTATAGATGGGTGCGTAGGAGTAAACTCAACCCTTACGAGC |
| | GGGACCTGTCCAACGATATCTATTCCCCTGGCGGCCAGTCCTGCTCCGCTG |
| | TGGGCCCCAATTGCTACAACCCCCTGCGGCCTTACGGCTTCTTCACAACCG |
| | CTGGAGTGGGCCACCAGCCTTACCGGGTGGTGGTGCTGTCTTTTGAACTGC |
| | TGAATGCTCCTGCTACCGTGTGCGGCCCCAAGCTGTCCACCGACCTGATAA |
| | AAAACCAGTGTGTGAACTTCGGCTCTGGCAGCGGTTCTAGAGTCGCCCCTA |
| | GTAAAGAAGTGGTGAGATTCCCCAACATCACCAACCTGTGCCCTTTCGGCGA |
| | AGTGTTCAACGCCACCACCTTCCCTTCTGTGTACGCTTGGGAGCGGAAGCG |
| | GATCTCCAACTGTGTGGCCGACTATAGCGTGTTATACAACTCTACCTCTTTTT |
| | CCACCTTCAAGTGCTATGGCGTGTCTGCCACCAAACTGAACGACCTGTGCTT |
| | CTCGAACGTGTACGCTGATAGCTTTGTGGTGAAAGGCGACGACGTGAGACA |
| | GATCGCACCTGGACAGACCGGAGTGATCGCCGACTATAACTACAAGCTTCC |
| | AGATGACTTCACCGGCTGTGTGCTGGCTTGGAACACCCGGAACATCGACGC |
| | CACCCAGACAGGAAACTACAACTACAAGTACCGCTCCCTGAGACACGGCAA |
| | GCTGAGACCCTTCGAGCGGGACATCTCCAACGTGCCTTTCTCTCCTGATGG |
| | CAAGCCCTGCACCCCTCCAGCCTTCAACTGTTACTGGCCTCTGAACGACTAC |
| | GGCTTTTACATCACCAATGGCATCGGATATCAGCCTTACAGAGTGGTGGTCC |
| | TGTCCTTTGAGCTGCTGAACGCCCCTGCCACCGTGTGCGGCCCTAAGCTGT |
| | CCACCGACCTGATCAAGAACCAGTGCGTGAACTTTtgataataa |
| 4502_[WHS1_ | ATGTTTGTGTTTCTAGTGCTACTACCACTAGTCTCTTCCCAGTGTGTGAACCT |
| KHOSTA2RBD_ | GACCACCAGAACCCAGCTGCCTCCTGCCTACACAAATTCTTTTACACGCGGA |
| RSCH014BD_ | GTGTACTACCCTGACAAGGTGTTCAGATCCTCTGTACTGCATAGTACACAGG |
| URBANIRBD] | ACCTGTTTCTGCCCTTCTTCAGCAACGTGACCTGGTTCCACGCTATTCATGTT |
| (SEQ ID NO: 34) | TCTGGCACCAATGGAACCAAGAGATTCGACAATCCTGTGCTGCCATTCAACG |
| | ACGGCGTGTACTTCGCCTCCACGGAGAAGTCCAACATCATCAGGGGCTGGA |
| | TCTTCGGCACCACCCTGGATTCAAAGACCCAGTCTCTGCTGATCGTGAATAA |
| | TGCTACCAACGTGGTCATCAAAGTATGCGAGTTCCAGTTCTGCAACGACCCT |
| | TTTCTGGGCGTGTACTACCACAAGAACAATAAGTCCTGGATGGAATCCGAGT |
| | TCAGAGTATACTCTTCTGCCAACAACTGTACCTTCGAGTACGTCTCTCAGCC |
| | TTTCCTGATGGACCTTGAGGGCAAGCAGGGCAACTTCAAAAACCTGAGAGA |
| | GTTCGTGTTCAAAAACATCGACGGCTACTTCAAGATCTACTCTAAGCACACC |
| | CCTATCAACCTGGTGCGCGACCTGCCCCAAGGCTTTTCCGCTCTGGAACCT |
| | CTGGTGGATCTGCCTATCGGCATCAACATCACCCGCTTCCAAACCCTGCTCG |
| | CTCTGCACCGCTCCTACCTGACCCCTGGCGATTCCTCCTCCGGCTGGACCG |
| | CCGGCGCTGCCGCCTACTACGTAGGATATCTGCAGCCTCGGACCTTCCTGC |
| | TGAAGTACAATGAGAACGGCACCATCACCGACGCCGTGGACTGCGCCCTGG |
| | ACCCCCTGTCCGAGACAAAGTGCACCCTAAAGTCCTTCACAGTGGAAAAGG |
| | GAATCTACCAGACATCCAACTTCAGAGTGCAGCCTACCGAGTCCATCGTGAG |
| | ATTCCCTAACATCACCAACCTGTGTCCTTTCGGCGAGGTGTTCAACGCCACT |
| | AGATTCGCCTCCGTGTACGCTTGGAACCGGAAGCGGATCAGCAATTGTGTT |
| | GCCGACTACTCCGTCCTGTACAACTCCGCGAGTTTCTCCACTTTCAAGTGCT |
| | ACGGCGTGTCACCTACCAAGTTGAACGATCTGTGCTTCACTAACGTCTATGC |
| | CGACAGCTTTGTGATCAGAGGCGACGAGGTGCGGCAGATTGCACCTGGCCA |
| | GACCGGCAAGATCGCCGATTACAACTACAAGCTGCCCGATGATTTTACCGG |
| | CTGTGTGATCGCCTGGAACTCCAACAACCTGGACTCTAAGGTCGGAGGCAA |
| | CTACAACTACCTGTACAGACTGTTTAGAAAGTCTAACCTGAAGCCCTTCGAG |
| | CGGGACATCTCCACTGAGATCTACCAGGCCGGATCCACACCTTGTAACGGC |
| | GTGGAAGGCTTCAACTGCTACTTCCCTCTGCAGTCTTATGGCTTCCAGCCTA |
| | CCAACGGCGTCGGCTACCAGCCTTACAGAGTGGTCGTCCTCTCCTTCGAGC |
| | TGCTGCACGCCCCAGCCACCGTGTGTGGGCCTAAGAAATCGACAAACCTGG |
| | TGAAGAACAAGTGCGTGAACTTCAACTTCAACGGTCTGACCGGAACCGGAG |
| | TTCTTACCGAATCCAACAAGAAGTTCCTGCCTTTCCAGCAGTTTGGCAGAGA |
| | CATCGCCGACACCACCGATGCTGTGAGAGATCCTCAGACACTGGAAATCCT |
| | GGATATCACCCCATGCTCCTTCGGCGGCGTGAGTGTGATCACCCCTGGCAC |
| | CAACACATCCAACCAAGTGGCCGTGCTGTACCAGGACGTGAACTGTACCGA |
| | GGTTCCCGTGGCCATCCACGCCGACCAGCTGACCCCTACCTGGCGGGTGT |
| | ACTCCACCGGCTCCAACGTGTTCCAAACTAGAGCCGGCTGCCTGATCGGGG |
| | CCGAGCACGTGAACAACTCTTACGAGTGCGACATCCCCATCGGCGCTGGCA |
| | TCTGCGCTTCCTACCAGACACAGACCAACTCTCCTAGAGGCTCCGGCTCCG |
| | GCTCTAGAGTGTCTCCCTCCCTCGACGTGGTTCGATTCCCCAACATGACCAA |
| | CATCTGCCCTTTCGACCAGGTGTTCAACAAAACCCAGTTTCCAAGCGTGTAC |
| | GCCTGGGAGAGAGTCAGAATCTCTGACTGTGTCAGCGACTACACCGTGCTG |
| | TACAACAGTTCTGCCTCCTTCTCCACATTTAAGTGCTACGGCGTGTCCCCTA |
| | CAAAGCTGAACGACCTGTGCTTCTCTGGCGTCTACGCCGACTACTTCGTCGT |
| | GAAGGGCGACCACGTGCACCAGATCGCTCCAGGCCAGACCGGCGTTATTG |
| | CCGACTACAACTACAAGCTGCCTTCCGAGTTCGTGGGATGCATCCTGGCCT |
| | GGAACACTCGGACCATCGACTCCAAGCGGGGCTTCTACTACCGGCTGTTCC |
| | GGCACGGTAACATCCGCCCCTACGAGAGAGACACCTCTAATGTACCCTACA |
| | ATGCCGCCGGCGGCACCTGTAACCAGCCCGGTACCCACAACTGCTACGAG |
| | CCTCTGCAGGACTACGGCTTCACCTCTACCAGCGGAGTGGGCTACCAACCA |
| | TTCAGAGTGGTGGTGCTGAGTTTTGAACTGCTGAACGCTCCTGCTACAGTGT |
| | GCGGCCCTAAGCAGTCAACCGATCTGGTGAAGAACAAGTGCGTGAATTTCG |
| | GCTCCGGATCTGGATCTAGAGTGGCCCCCTCTAAAGAGGTCGTGAGGTTCC |
| | CTAACATCACCAATCTGTGCCCTTTCGGCGAAGTGTTCAACGCTACAACGTT |
| | CCCCTCCGTTTATGCCTGGGAGAGAAAGCGGATCTCCAACTGCGTGGCTGA |
| | TTATTCCGTGCTGTACAATTCGACATCTTTCTCCACCTTTAAGTGTTACGGCG |
| | TGTCCGCCACCAAACTGAACGACCTTTGCTTTTCCAACGTGTATGCTGACAG |
| | TTTCGTGGTGAAGGGCGACGATGTGCGACAGATAGCGCCCGGCCAGACCG |
| | GCGTGATCGCTGACTACAACTACAAGCTGCCTGATGACTTCCTCGGCTGCG |
| | TGCTGGCCTGGAATACCAACTCCAAGGACTCCTCAACCTCTGGCAACTATAA |
| | TTACCTGTACAGATGGGTGAGACGGAGCAAACTGAACCCGTACGAACGGGA |
| | TCTTTCTAATGACATCTACTCTCCCGGTGGACAGTCCTGCTCCGCCGTGGGC |
| | CCAAATTGTTACAACCCACTGCGGCCTTACGGCTTCTTCACCACCGCTGGCG |
| | TGGGCCATCAGCCTTACAGAGTGGTGGTGCTGTCCTTTGAGCTGCTGAACG |
| | CTCCTGCTACCGTGTGCGGCCCTAAATTGTCTACCGACCTGATCAAGAACCA |
| | GTGCGTGAATTTCGGCAGCGGCTCTGGATCCAGAGTGGTGCCTTCCGGCGA |
| | CGTCGTTAGATTTCCGAACATCACCAATCTGTGTCCTTTCGGCGAAGTGTTC |
| | AACGCCACCAAGTTCCCTTCTGTGTACGCTTGGGAACGGAAGAAAATCTCCA |
| | ACTGTGTGGCCGACTACTCTGTGTTGTATAACTCTACCTTCTTTTCCACCTTC |
| | AAGTGCTACGGCGTGTCTGCCACCAAGCTGAACGATCTCTGCTTCTCCAAC |
| | GTGTACGCAGACTCCTTCGTGGTGAAAGGCGACGACGTCAGACAGATCGCT |
| | CCCGGACAGACCGGCGTGATCGCCGATTACAACTACAAGCTGCCCGACGAC |
| | TTCATGGGCTGTGTGCTGGCTTGGAATACCAGAAACATCGATGCCACCTCTA |
| | CTGGCAACTACAACTACAAGTACCGCTACCTGAGACACGGCAAACTGCGGC |
| | CTTTTGAACGGGACATCTCCAACGTGCCATTCAGCCCTGATGGCAAGCCTTG |
| | CACGCCCCCTGCTCTGAACTGCTACTGGCCTCTGAACGACTACGGATTCTAC |
| | ACAACCACCGGCATCGGATATCAGCCTTACCGGGTGGTAGTGCTGAGCTTC |
| | GAGCTGCTGAACGCCCCAGCTACCGTGTGCGGCCCCAAGCTGTCTACCGAC |
| | CTCATCAAGAACCAGTGCGTGAATTTTtgataataa |
| 4503_[WuhanS1_ | ATGTTTGTGTTTCTAGTGCTACTACCACTAGTCAGTTCTCAGTGCGTGAACCT |
| RATG13RBD_ | GACAACGAGAACCCAGCTGCCTCCAGCCTACACCAACTCATTCACAAGAGG |
| BTKY72RBD_ | CGTGTACTACCCCGACAAGGTGTTCAGAAGCTCTGTGCTGCACTCCACCCA |
| WIV1RBD] | GGATCTGTTCCTGCCCTTCTTCTCCAACGTGACCTGGTTTCACGCCATCCAC |
| (SEQ ID NO: 35) | GTCTCCGGCACCAATGGCACCAAGCGGTTCGACAACCCAGTGCTCCCATTC |
| | AACGACGGCGTGTACTTCGCCTCCACCGAGAAGTCTAACATCATCAGAGGTT |
| | GGATCTTCGGCACCACCCTGGACTCCAAGACCCAATCCCTGCTGATCGTGA |
| | ACAACGCCACTAATGTGGTCATCAAAGTGTGCGAGTTCCAGTTCTGCAACGA |
| | CCCCTTCCTGGGTGTGTACTACCACAAGAACAACAAGTCTTGGATGGAAAGC |
| | GAGTTCAGAGTGTACTCATCTGCTAACAATTGCACCTTCGAGTACGTGAGTC |
| | AGCCTTTTCTGATGGACCTGGAAGGAAAGCAGGGCAACTTCAAAAACCTGA |
| | GAGAGTTCGTGTTCAAAAATATCGACGGCTACTTCAAGATCTACTCTAAGCA |
| | CACCCCTATCAACCTGGTCCGGGATCTGCCTCAGGGCTTTAGCGCCCTCGA |
| | GCCTCTGGTGGACCTGCCCATCGGCATCAACATCACCCGGTTCCAGACTCT |
| | GTTAGCTCTGCACAGATCCTACCTCACCCCCGGCGATTCCTCTTCTGGCTGG |
| | ACCGCTGGCGCTGCTGCCTACTACGTGGGCTACCTGCAGCCTCGAACGTTT |
| | CTGCTGAAGTACAACGAGAACGGCACCATCACAGACGCCGTAGACTGTGCC |
| | CTGGATCCTCTGTCTGAGACAAAGTGCACGCTGAAGTCCTTCACCGTGGAAA |
| | AGGGCATCTACCAGACATCCAACTTCCGGGTGCAGCCTACCGAGTCTATCG |
| | TGCGGTTCCCCAACATAACCAACCTGTGTCCTTTCGGCGAGGTATTTAACGC |
| | TACCAGATTCGCCTCCGTGTACGCCTGGAACAGAAAACGGATCTCTAACTGC |
| | GTTGCCGACTACAGTGTGCTGTACAACTCCGCTTCATTCAGCACCTTCAAGT |
| | GCTACGGCGTGTCCCCTACCAAGCTGAATGATCTGTGCTTCACCAACGTGTA |
| | CGCTGACTCTTTCGTGATCCGGGGCGACGAAGTGAGACAGATCGCCCCTGG |
| | CCAGACAGGCAAGATCGCCGACTATAACTACAAGCTGCCTGACGACTTCAC |
| | CGGATGCGTGATTGCCTGGAACTCCAATAACCTGGATTCTAAGGTGGGCGG |
| | CAACTACAACTACCTGTATCGTCTGTTCCGGAAGTCCAATCTGAAGCCATTC |
| | GAAAGAGATATTTCCACCGAGATCTACCAGGCTGGAAGCACCCCTTGCAAC |
| | GGCGTCGAAGGCTTCAACTGCTACTTCCCCCTGCAGTCTTACGGCTTTCAGC |
| | CTACCAACGGCGTTGGATACCAACCTTACAGAGTGGTCGTGCTGTCTTTTGA |
| | GCTGCTGCATGCTCCAGCAACCGTGTGCGGACCCAAGAAGTCCACCAATCT |
| | GGTGAAGAACAAGTGCGTGAACTTCAACTTCAACGGCCTGACGGGTACCGG |
| | CGTACTGACAGAGTCCAACAAAAAGTTTCTGCCTTTCCAGCAGTTCGGCAGA |
| | GATATCGCCGACACCACCGACGCCGTGAGAGATCCTCAGACCCTGGAGATC |
| | CTGGATATCACCCCTTGTAGCTTCGGCGGAGTGTCCGTGATCACACCCGGC |
| | ACAAACACCTCTAACCAGGTGGCCGTCCTGTACCAGGACGTGAACTGCACC |
| | GAAGTGCCTGTGGCAATCCACGCCGACCAGCTGACCCCCACCTGGCGGGT |
| | TTACTCTACCGGCAGCAACGTGTTTCAGACCAGAGCTGGCTGCCTGATTGG |
| | CGCCGAGCACGTGAACAACTCGTACGAGTGCGACATCCCTATCGGAGCCGG |
| | CATCTGCGCCTCCTACCAGACCCAGACCAACTCTCCTAGAGGCTCCGGATC |
| | CGGCTCTCGGGTGCAGCCCACCGACTCCATCGTCAGATTCCCTAACATCAC |
| | CAACCTCTGCCCTTTTGGAGAAGTGTTCAACGCCACCACGTTCGCCTCTGTG |
| | TACGCTTGGAACAGAAAGCGGATCTCCAACTGCGTGGCGGACTACTCTGTC |
| | CTGTACAATTCCACCTCCTTTTCCACCTTCAAGTGCTACGGCGTGTCCCCTA |
| | CAAAGCTGAACGATCTGTGCTTCACCAATGTGTACGCCGATAGCTTCGTTAT |
| | CACCGGCGACGAGGTGCGCCAGATCGCTCCTGGCCAGACCGGCAAGATCG |
| | CCGACTACAACTACAAGTTGCCCGATGACTTCACCGGCTGTGTGATCGCTTG |
| | GAACAGCAAGCACATCGATGCCAAGGAAGGTGGCAACTTCAACTACCTGTA |
| | CAGACTGTTTAGAAAGGCCAACCTTAAGCCATTTGAGAGAGACATCTCCACA |
| | GAAATCTACCAGGCTGGCTCCAAACCTTGCAATGGCCAGACAGGCCTGAAC |
| | TGCTACTATCCTCTGTACCGCTACGGCTTCTACCCTACCGATGGCGTGGGC |
| | CACCAGCCTTATAGAGTCGTGGTGCTGTCTTTCGAACTGCTGAACGCCCCTG |
| | CCACCGTGTGTGGCCCTAAGAAGTCTACAAACCTGGTGAAAAATAAGTGCGT |
| | GAACTTCGGCTCTGGCTCTGGCTCTCGGGTGTCCCCAAGCACGGAGGTGGT |
| | CCGGTTCCCAAACATCACCAACCTGTGCCCTTTTGGCCAAGTGTTCAATGCT |
| | TCAAACTTTCCTTCTGTGTACGCTTGGGAGCGGCTGAGGATCTCCGACTGC |
| | GTGGCTGATTACGCCGTGCTCTACAACTCTAGCAGCTCTTTCTCTACCTTCA |
| | AGTGCTACGGCGTGTCTCCAACCAAGCTGAACGACCTGTGTTTCTCCTCCGT |
| | GTACGCCGACTACTTCGTGGTGAAGGGCGACGACGTGAGACAGATCGCTCC |
| | TGCTCAGACCGGAGTGATCGCCGATTACAACTACAAGCTGCCTGATGACTTC |
| | ACCGGCTGCGTGCTGGCCTGGAACACCAACTCCGTGGACTCCAAGAGTGG |
| | GAACAACTTCTACTACAGACTGTTTAGACATGGCAAGATCAAGCCTTACGAG |
| | CGCGACATCTCCAACGTGCTGTACAACTCTGCCGGCGGCACCTGCTCCTCC |
| | ATTAGCCAGCTGGGATGTTACGAGCCCCTGAAAAGCTACGGCTTTACCCCTA |
| | CTGTAGGCGTCGGCTATCAGCCTTACCGGGTTGTGGTGCTGTCCTTCGAAC |
| | TGCTGAACGCCCCTGCTACCGTGTGTGGCCCTAAGAAATCCACCGAGCTGG |
| | TGAAGAACAAGTGCGTGAACTTCGGCTCCGGCTCCGGCTCCAGAGTGGCAC |
| | CGTCTAAAGAGGTGGTGCGGTTCCCAAACATCACCAACCTGTGTCCTTTCGG |
| | CGAAGTCTTCAACGCCACCACGTTCCCTAGCGTTTACGCCTGGGAACGGAA |
| | GAGAATCTCTAACTGTGTGGCCGACTACAGCGTGCTGTACAACTCCACCTCG |
| | TTTTCCACCTTCAAGTGTTATGGCGTGTCCGCCACCAAGTTGAACGACCTGT |
| | GCTTCAGCAATGTGTATGCCGACTCCTTCGTGGTCAAAGGCGACGACGTGC |
| | GGCAGATCGCCCCTGGACAAACTGGCGTGATCGCCGACTATAACTACAAAC |
| | TGCCTGACGATTTCACCGGCTGTGTGCTGGCTTGGAATACCCGGAACATCG |
| | ATGCTACACAGACCGGCAACTACAACTACAAGTACAGGAGCCTGAGACACG |
| | GCAAGCTTAGGCCTTTCGAGAGAGACATTTCCAACGTGCCCTTCTCCCCTGA |
| | CGGCAAGCCCTGCACACCCCCTGCGTTCAACTGTTACTGGCCTCTGAACGA |
| | CTATGGCTTTTACATCACCAACGGGATCGGCTATCAACCGTACCGGGTGGT |
| | GGTGCTGTCTTTTGAGCTGCTGAACGCGCCTGCCACAGTGTGCGGCCCCAA |
| | GCTCTCCACCGACTTGATCAAGAACCAATGTGTGAACTTCtgataataa |
| 4495_BA2S1 | atgtttgtgtttctagtgctactaccactagtgtcctctcagtgcgtgaacctgatcacacggacccagtcatacacca |
| (SEQ ID NO: 36) | actccttcaccagaggagtgtactaccctgacaaggtgttcagatctagcgtgctgcattctacccaggacctgtttc |
| | tgcctttcttcagcaatgtgacctggttccacgccatccacgtgtctggcacaaacggcaccaagagattcgacaa |
| | ccctgtgctgcctttcaacgatggcgtgtacttcgcctctacagagaagtccaacatcatccggggctggatcttcg |
| | gcaccacactggactcgaagacacaatctctgctgatcgtgaacaacgccaccaacgttgtcatcaaggtgtgc |
| | gagttccagttctgcaacgaccctttcttggacgtgtactaccacaagaacaacaagtcctggatggaatccgaat |
| | tcagagtgtactccagcgccaacaactgtacattcgagtacgtcagccagccctttctgatggacctggaaggca |
| | agcagggaaatttcaagaacctgagagagttcgtgttcaagaacatcgacggctacttcaagatctacagcaag |
| | cacacccctattaacctgggcagagatctgccccagggcttttccgctctggagcccctggtggacctgcctatcg |
| | gcatcaacatcacccggtttcagaccctcctagccctgcacagatcctatctgacccctggcgacagcagttccg |
| | gctggaccgccggggctgctgcctactacgtggggtacctgcagcctagaaccttcctgctgaagtacaacgag |
| | aacggcaccatcaccgacgccgtggactgtgctctggatccactgtccgagaccaagtgcactctcaagagctt |
| | caccgtggaaaagggaatctaccagacctccaactttagagtgcagcccaccgagtcaatcgtgagattcccca |
| | acatcaccaacctgtgcccttttgacgaggtgttcaacgctaccagatttgcttctgtgtacgcctggaaccggaag |
| | agaatctctaactgcgtggccgattactccgtgctgtacaacttcgctcctttcttcgccttcaagtgttacggcgtgtc |
| | ccctaccaagctgaacgacctgtgtttcactaacgtgtacgccgactctttcgtgatccggggcaacgaggtgtcc |
| | cagatcgccccaggccagacaggcaacatcgctgactacaactacaagctgcctgacgatttcaccggctgtgt |
| | gatcgcctggaactccaacaagctggattccaaagtgggcggcaactacaactatctgtaccggctgttcagaa |
| | agtccaatctgaagcccttcgaacgcgacatctccaccgagatctatcaggccggcaacaagccttgtaacggc |
| | gtcgctggcttcaactgctacttccctttgcgatcttatggcttccggcctacctacggcgtgggccaccagccttacc |
| | gggtggtggtgctgagctttgagctgctgcacgctcctgctacagtgtgcggccctaagaagtccaccaatctggt |
| | gaaaaataagtgcgttaacttcaacttcaacggcctgaccggcaccggcgtgctgacagagtcaaataagaag |
| | ttcctgcctttccagcagtttggcagggacatcgccgacaccacggacgctgtgcgggaccctcagaccctgga |
| | aatcctggatatcaccccttgctccttcggcggcgtgagcgtgattacccctggaaccaacacctctaatcaagtg |
| | gccgtgctgtaccaaggcgtgaactgcaccgaagtgcccgtggctatccacgccgatcagctgacccctacatg |
| | gcgggtatacagtaccggctctaacgtgttccagacccgggctggctgcctgatcggcgccgagtacgtgaaca |
| | actcctacgagtgcgacatccccatcggcgctggcatttgc |
| 4498_BA2S1 | atgtttgtgtttctagtgctactaccactagtgtccagccagtgcgtcaatctcatcacccgcacacaatcctacaca |
| (SEQ ID NO: | aactccttcaccagaggcgtgtactaccctgataaggtgttcagatcctccgtgctgcactccacccaggacctgtt |
| 37) | cctgccgttcttcagcaacgtgacctggttccacgccatccacgtgtcaggaacaaacggcaccaagagattcg |
| | acaaccccgtcctgcctttcaacgacggcgtctacttcgcctccacagagaagtctaatatcatcagaggctggat |
| | cttcggcactaccctggactccaagacacagtctctgctcatcgtgaacaacgccaccaacgtcgtcatcaaggt |
| | gtgcgagttccagttctgcaacgacccatttctggacgtgtactaccacaagaacaacaagtcctggatggaatc |
| | cgagttcagagtgtactcttccgccaacaactgcaccttcgagtacgtgtctcaacctttcctgatggacctggaag |
| | gcaagcagggtaacttcaagaatctgagagagtttgtgttcaagaacatcgatggctactttaagatctacagcaa |
| | gcacacccctatcaacctgggcagagatttaccccagggattctccgctctagaacccctggtggacctgcccat |
| | cggcatcaacatcacaagattccaaaccctgctggctctgcacagatcttatctgacccccggcgactcctctagc |
| | ggctggaccgctggtgctgccgcctactacgtgggctacctccagcctagaaccttcctgctgaagtacaacgag |
| | aacggcaccatcaccgatgctgtggactgcgccctggatcctctgtccgaaaccaaatgtaccctgaagagcttc |
| | accgtggaaaagggcatctaccagacaagcaactttcgggtgcagcccaccgaatccatcgtgcggttcccaa |
| | atatcaccaatctgtgtcctttcgacgaggtgttcaatgctaccagattcgcctctgtgtacgcctggaaccggaag |
| | agaatcagcaactgcgtggccgattactctgtactgtataatttcgctcccttcttcgccttcaagtgctacggagtgt |
| | cgcctacaaaacttaacgatctgtgcttcaccaacgtgtacgccgacagcttcgtgatccggggcaatgaagtgt |
| | ctcagatcgccccaggacagaccggcaacatcgcggactacaactacaaactgccagacgactttaccggct |
| | gcgtgatcgcttggaactccaacaaactggacagcaaagtgggcggcaactacaactacctgtacagactgttt |
| | agaaagtctaacctgaagccctttgaaagagatatctctacagagatctaccaggctggcaacaagccttgtaat |
| | ggcgttgctggcttcaactgctacttccctctgcggtcttacggcttccggcctacctacggcgtgggccaccagcct |
| | taccgggtagtcgtactcagcttcgagctgctacatgctcccgctacagtgtgcggccccaagaagtccacgaac |
| | ctggtcaagaacaagtgtgtgaactttaacttcaacggcctgaccggcacgggcgtcctgaccgagtccaataa |
| | aaagttcctacccttccagcagttcggcagagatatcgctgacaccaccgacgccgtgcgggaccctcagaccc |
| | tggaaatcctggacatcaccccttgctccttcggtggcgtttccgtgattacccctggcaccaacacctccaatcaa |
| | gtggccgtgctgtaccagggcgtgaactgtaccgaagtgcctgtggcgatccacgccgaccagctgaccccaa |
| | cctggagagtgtattctaccggatccaacgtgtttcagaccagagctggatgcctgatcggagccgagtacgtga |
| | acaacagctacgagtgcgacattcccatcggcgccggcatctgc |
| 4296_WuhanS1 | atgttcgtgtttctggtgctgctgcctctggtgtccagccagtgtgtgaacctgaccaccagaacacagctgcctcca |
| (SEQ ID NO: | gcctacaccaacagctttaccagaggcgtgtactaccccgacaaggtgttcagatccagcgtgctgcactctacc |
| 38) | caggacctgttcctgcctttcttcagcaacgtgacctggttccacgccatccacgtgtccggcaccaatggcacca |
| | agagattcgacaaccccgtgctgcccttcaacgacggggtgtactttgccagcaccgagaagtccaacatcatc |
| | agaggctggatcttcggcaccacactggacagcaagacccagagcctgctgatcgtgaacaacgccaccaac |
| | gtggtcatcaaagtgtgcgagttccagttctgcaacgaccccttcctgggcgtctactaccacaagaacaacaag |
| | agctggatggaaagcgagttccgggtgtacagcagcgccaacaactgcaccttcgagtacgtgtcccagccttt |
| | cctgatggacctggaaggcaagcagggcaacttcaagaacctgcgcgagttcgtgttcaagaacatcgacggc |
| | tacttcaagatctacagcaagcacacccctatcaacctcgtgcgggatctgcctcagggcttctctgctctggaac |
| | ccctggtggatctgcccatcggcatcaacatcacccggtttcagacactgctggccctgcacagaagctacctga |
| | cacctggcgatagcagcagcggatggacagctggtgccgccgcttactatgtgggctacctgcagcctagaacc |
| | ttcctgctgaagtacaacgagaacggcaccatcaccgacgccgtggattgtgcccttgatcctctgagcgagaca |
| | aagtgcaccctgaagtccttcaccgtggaaaagggcatctaccagaccagcaacttccgggtgcagcccaccg |
| | aatccatcgtgcggttccccaatatcaccaatctgtgccccttcggcgaggtgttcaatgccaccagattcgcctct |
| | gtgtacgcctggaaccggaagcggatcagcaattgcgtggccgactactccgtgctgtacaactccgccagcttc |
| | agcaccttcaagtgctacggcgtgtcccctaccaagctgaacgacctgtgcttcacaaacgtgtacgccgacag |
| | cttcgtgatccggggagatgaagtgcggcagattgcccctggacagacaggcaagatcgccgactacaactac |
| | aagctgcccgacgacttcaccggctgtgtgattgcctggaacagcaacaacctggactccaaagtcggcggca |
| | actacaattacctgtaccggctgttccggaagtccaatctgaagcccttcgagcgggacatctccaccgagatcta |
| | tcaggccggcagcaccccttgtaacggcgtggaaggcttcaactgctacttcccactgcagtcctacggctttcag |
| | cccacaaatggcgtgggctatcagccctacagagtggtggtgctgagcttcgaactgctgcatgcccctgccaca |
| | gtgtgcggccctaagaaaagcaccaatctcgtgaagaacaaatgcgtgaacttcaacttcaacggcctgaccg |
| | gcaccggcgtgctgacagagagcaacaagaagttcctgccattccagcagtttggccgggatatcgccgatac |
| | cacagacgccgttagagatccccagacactggaaatcctggacatcaccccttgcagcttcggcggagtgtctgt |
| | gatcacccctggcaccaacaccagcaatcaggtggcagtgctgtaccaggacgtgaactgtaccgaagtgccc |
| | gtggccattcacgccgatcagctgacacctacatggcgggtgtactccaccggcagcaatgtgtttcagaccaga |
| | gccggctgtctgatcggagccgagcacgtgaacaatagctacgagtgcgacatccccatcggcgctggcatctg |
| | tgccagctaccagacacagacaaacagccccaga |
| 4304_WuhanS1 | atgttcgtgtttctggtgctgctgcctctggtgtccagccagtgtgtgaacctgaccaccagaacacagctgcctcca |
| (SEQ ID NO: | gcctacaccaacagctttaccagaggcgtgtactaccccgacaaggtgttcagatccagcgtgctgcactctacc |
| 39) | caggacctgttcctgcctttcttcagcaacgtgacctggttccacgccatccacgtgtccggcaccaatggcacca |
| | agagattcgacaaccccgtgctgcccttcaacgacggggtgtactttgccagcaccgagaagtccaacatcatc |
| | agaggctggatcttcggcaccacactggacagcaagacccagagcctgctgatcgtgaacaacgccaccaac |
| | gtggtcatcaaagtgtgcgagttccagttctgcaacgaccccttcctgggcgtctactaccacaagaacaacaag |
| | agctggatggaaagcgagttccgggtgtacagcagcgccaacaactgcaccttcgagtacgtgtcccagccttt |
| | cctgatggacctggaaggcaagcagggcaacttcaagaacctgcgcgagttcgtgttcaagaacatcgacggc |
| | tacttcaagatctacagcaagcacacccctatcaacctcgtgcgggatctgcctcagggcttctctgctctggaac |
| | ccctggtggatctgcccatcggcatcaacatcacccggtttcagacactgctggccctgcacagaagctacctga |
| | cacctggcgatagcagcagcggatggacagctggtgccgccgcttactatgtgggctacctgcagcctagaacc |
| | ttcctgctgaagtacaacgagaacggcaccatcaccgacgccgtggattgtgcccttgatcctctgagcgagaca |
| | aagtgcaccctgaagtccttcaccgtggaaaagggcatctaccagaccagcaacttccgggtgcagcccaccg |
| | aatccatcgtgcggttccccaatatcaccaatctgtgccccttcggcgaggtgttcaatgccaccagattcgcctct |
| | gtgtacgcctggaaccggaagcggatcagcaattgcgtggccgactactccgtgctgtacaactccgccagcttc |
| | agcaccttcaagtgctacggcgtgtcccctaccaagctgaacgacctgtgcttcacaaacgtgtacgccgacag |
| | cttcgtgatccggggagatgaagtgcggcagattgcccctggacagacaggcaagatcgccgactacaactac |
| | aagctgcccgacgacttcaccggctgtgtgattgcctggaacagcaacaacctggactccaaagtcggcggca |
| | actacaattacctgtaccggctgttccggaagtccaatctgaagcccttcgagcgggacatctccaccgagatcta |
| | tcaggccggcagcaccccttgtaacggcgtggaaggcttcaactgctacttcccactgcagtcctacggctttcag |
| | cccacaaatggcgtgggctatcagccctacagagtggtggtgctgagcttcgaactgctgcatgcccctgccaca |
| | gtgtgcggccctaagaaaagcaccaatctcgtgaagaacaaatgcgtgaacttcaacttcaacggcctgaccg |
| | gcaccggcgtgctgacagagagcaacaagaagttcctgccattccagcagtttggccgggatatcgccgatac |
| | cacagacgccgttagagatccccagacactggaaatcctggacatcaccccttgcagcttcggcggagtgtctgt |
| | gatcacccctggcaccaacaccagcaatcaggtggcagtgctgtaccaggacgtgaactgtaccgaagtgccc |
| | gtggccattcacgccgatcagctgacacctacatggcgggtgtactccaccggcagcaatgtgtttcagaccaga |
| | gccggctgtctgatcggagccgagcacgtgaacaatagctacgagtgcgacatccccatcggcgctggcatctg |
| | tgccagctaccagacacagacaaacagccccaga |
| 4371_Wuhan | atgcagtgtgtgaacctgaccaccagaacacagctgcctccagcctacaccaacagctttaccagaggcgtgta |
| (SEQ ID NO: | ctaccccgacaaggtgttcagatccagcgtgctgcactctacccaggacctgttcctgcctttcttcagcaacgtga |
| 40) | cctggttccacgccatccacgtgtccggcaccaatggcaccaagagattcgacaaccccgtgctgcccttcaac |
| | gacggggtgtactttgccagcaccgagaagtccaacatcatcagaggctggatcttcggcaccacactggaca |
| | gcaagacccagagcctgctgatcgtgaacaacgccaccaacgtggtcatcaaagtgtgcgagttccagttctgc |
| | aacgaccccttcctgggcgtctactaccacaagaacaacaagagctggatggaaagcgagttccgggtgtaca |
| | gcagcgccaacaactgcaccttcgagtacgtgtcccagcctttcctgatggacctggaaggcaagcagggcaa |
| | cttcaagaacctgcgcgagttcgtgttcaagaacatcgacggctacttcaagatctacagcaagcacacccctat |
| | caacctcgtgcgggatctgcctcagggcttctctgctctggaacccctggtggatctgcccatcggcatcaacatc |
| | acccggtttcagacactgctggccctgcacagaagctacctgacacctggcgatagcagcagcggatggacag |
| | ctggtgccgccgcttactatgtgggctacctgcagcctagaaccttcctgctgaagtacaacgagaacggcacca |
| | tcaccgacgccgtggattgtgcccttgatcctctgagcgagacaaagtgcaccctgaagtccttcaccgtggaaa |
| | agggcatctaccagaccagcaacttccgggtgcagcccaccgaatccatcgtgcggttccccaatatcaccaat |
| | ctgtgccccttcggcgaggtgttcaatgccaccagattcgcctctgtgtacgcctggaaccggaagcggatcagc |
| | aattgcgtggccgactactccgtgctgtacaactccgccagcttcagcaccttcaagtgctacggcgtgtcccctac |
| | caagctgaacgacctgtgcttcacaaacgtgtacgccgacagcttcgtgatccggggagatgaagtgcggcag |
| | attgcccctggacagacaggcaagatcgccgactacaactacaagctgcccgacgacttcaccggctgtgtgat |
| | tgcctggaacagcaacaacctggactccaaagtcggcggcaactacaattacctgtaccggctgttccggaagt |
| | ccaatctgaagcccttcgagcgggacatctccaccgagatctatcaggccggcagcaccccttgtaacggcgtg |
| | gaaggcttcaactgctacttcccactgcagtcctacggctttcagcccacaaatggcgtgggctatcagccctaca |
| | gagtggtggtgctgagcttcgaactgctgcatgcccctgccacagtgtgcggccctaagaaaagcaccaatctc |
| | gtgaagaacaaatgcgtgaacttcaacttcaacggcctgaccggcaccggcgtgctgacagagagcaacaag |
| | aagttcctgccattccagcagtttggccgggatatcgccgataccacagacgccgttagagatccccagacactg |
| | gaaatcctggacatcaccccttgcagcttcggcggagtgtctgtgatcacccctggcaccaacaccagcaatca |
| | ggtggcagtgctgtaccaggacgtgaactgtaccgaagtgcccgtggccattcacgccgatcagctgacaccta |
| | catggcgggtgtactccaccggcagcaatgtgtttcagaccagagccggctgtctgatcggagccgagcacgtg |
| | aacaatagctacgagtgcgacatccccatcggcgctggcatctgtgccagctaccagacacagacaaacagc |
| | cccaga |
| 4372_Wuhan | atgcagtgtgtgaacctgaccaccagaacacagctgcctccagcctacaccaacagctttaccagaggcgtgta |
| (SEQ ID NO: 41) | ctaccccgacaaggtgttcagatccagcgtgctgcactctacccaggacctgttcctgcctttcttcagcaacgtga |
| | cctggttccacgccatccacgtgtccggcaccaatggcaccaagagattcgacaaccccgtgctgcccttcaac |
| | gacggggtgtactttgccagcaccgagaagtccaacatcatcagaggctggatcttcggcaccacactggaca |
| | gcaagacccagagcctgctgatcgtgaacaacgccaccaacgtggtcatcaaagtgtgcgagttccagttctgc |
| | aacgaccccttcctgggcgtctactaccacaagaacaacaagagctggatggaaagcgagttccgggtgtaca |
| | gcagcgccaacaactgcaccttcgagtacgtgtcccagcctttcctgatggacctggaaggcaagcagggcaa |
| | cttcaagaacctgcgcgagttcgtgttcaagaacatcgacggctacttcaagatctacagcaagcacacccctat |
| | caacctcgtgcgggatctgcctcagggcttctctgctctggaacccctggtggatctgcccatcggcatcaacatc |
| | acccggtttcagacactgctggccctgcacagaagctacctgacacctggcgatagcagcagcggatggacag |
| | ctggtgccgccgcttactatgtgggctacctgcagcctagaaccttcctgctgaagtacaacgagaacggcacca |
| | tcaccgacgccgtggattgtgcccttgatcctctgagcgagacaaagtgcaccctgaagtccttcaccgtggaaa |
| | agggcatctaccagaccagcaacttccgggtgcagcccaccgaatccatcgtgcggttccccaatatcaccaat |
| | ctgtgccccttcggcgaggtgttcaatgccaccagattcgcctctgtgtacgcctggaaccggaagcggatcagc |
| | aattgcgtggccgactactccgtgctgtacaactccgccagcttcagcaccttcaagtgctacggcgtgtcccctac |
| | caagctgaacgacctgtgcttcacaaacgtgtacgccgacagcttcgtgatccggggagatgaagtgcggcag |
| | attgcccctggacagacaggcaagatcgccgactacaactacaagctgcccgacgacttcaccggctgtgtgat |
| | tgcctggaacagcaacaacctggactccaaagtcggcggcaactacaattacctgtaccggctgttccggaagt |
| | ccaatctgaagcccttcgagcgggacatctccaccgagatctatcaggccggcagcaccccttgtaacggcgtg |
| | gaaggcttcaactgctacttcccactgcagtcctacggctttcagcccacaaatggcgtgggctatcagccctaca |
| | gagtggtggtgctgagcttcgaactgctgcatgcccctgccacagtgtgcggccctaagaaaagcaccaatctc |
| | gtgaagaacaaatgcgtgaacttcaacttcaacggcctgaccggcaccggcgtgctgacagagagcaacaag |
| | aagttcctgccattccagcagtttggccgggatatcgccgataccacagacgccgttagagatccccagacactg |
| | gaaatcctggacatcaccccttgcagcttcggcggagtgtctgtgatcacccctggcaccaacaccagcaatca |
| | ggtggcagtgctgtaccaggacgtgaactgtaccgaagtgcccgtggccattcacgccgatcagctgacaccta |
| | catggcgggtgtactccaccggcagcaatgtgtttcagaccagagccggctgtctgatcggagccgagcacgtg |
| | aacaatagctacgagtgcgacatccccatcggcgctggcatctgtgccagctaccagacacagacaaacagc |
| | cccaga |
| 4499_WuhanS1 | atgtttgtgtttctagtgctactaccactagtgtcttcccagtgcgtcaatctgaccaccagaacccaactgccccctg |
| (SEQ ID NO: 42) | cttacactaactctttcaccagaggcgtgtactaccctgacaaggtgttccggtcgtccgtgctgcattctacccagg |
| | atctcttcttgcctttcttctctaacgtgacctggttccacgccatccatgtgtctggcacaaacggcaccaagagatt |
| | cgacaaccctgtgctgcctttcaacgacggcgtgtatttcgcctctaccgagaagtctaacatcatccggggctgg |
| | atcttcggcaccacactggacagcaagacccagtcgctgctgatcgtgaacaacgccaccaacgtggtcatca |
| | aggtctgcgaattccagttctgcaacgatcctttcctgggcgtatactaccacaagaacaacaagtcctggatgga |
| | atccgaattcagagtatactcctccgccaataactgcacattcgagtacgtgtctcagcctttcctgatggacctgga |
| | aggcaagcagggcaacttcaaaaacctgagagagttcgtgttcaagaacatcgacggctacttcaagatctac |
| | agcaagcacacccctatcaacctggtgcgggatctgccacagggcttttccgctctggaacctctggtggacctg |
| | cccatcggcatcaatatcacccgctttcaaaccctgctggccctgcacagatcttacctgacaccaggcgactect |
| | cctccggctggaccgccggcgctgccgcctactacgtgggctacctccagcctcggacctttctgctcaagtaca |
| | acgagaacggcacaatcaccgacgccgtggactgcgccctggaccccctgtccgagacaaagtgtaccctga |
| | agtccttcaccgtggaaaaaggaatctatcaaacctccaactttcgtgtgcaacctactgaatctatcgtgagatttc |
| | ctaatattaccaatctgtgtccttttggcgaggtgttcaacgccactcggttcgcttccgtgtacgcctggaacagaa |
| | agcggatcagcaactgcgttgctgactactccgtgctgtacaactccgcttccttcagcaccttcaagtgctatggg |
| | gtctcccctaccaagctgaacgatctgtgcttcaccaacgtgtatgcagactctttcgtgatcagaggcgacgagg |
| | tcagacagatcgcccccggccagaccggaaagatcgctgactacaactacaagctgcccgacgacttcacag |
| | gctgtgtgatcgcgtggaactctaacaacctagactctaaggtaggcggcaactataactacctgtacagactgttt |
| | cggaagagcaacctgaagcctttcgagcgggacatctcgaccgagatctaccaggctggcagcacaccctgc |
| | aacggggtggaaggatttaactgctacttcccactgcagtcttatggcttccagcccacaaacggcgtgggctac |
| | caaccttaccgggtggtggtgctgtccttcgagctgctgcacgctcctgctaccgtgtgtggcccaaagaaatctac |
| | caacctggtgaagaacaagtgtgtcaacttcaatttcaacggcctgaccggcaccggcgtcctgaccgagtcta |
| | ataagaagttcctgccctttcagcagttcggcagagatatcgccgataccaccgacgctgtgcgggacccccag |
| | acgctggaaatcctggatatcacgccttgctctttcggcggcgtgtccgtgatcacacccggcaccaacaccagc |
| | aaccaagtggctgtgttgtatcaggacgtgaactgcaccgaagtgcctgtggccatccacgccgatcagctgac |
| | ccctacctggagagtgtactcaaccggcagcaacgtgttccagaccagagcgggctgcctgatcggcgccgag |
| | cacgtgaacaactcctacgagtgcgacatccctatcggagccggcatctgcgccagctaccaaacacagacc |
| | aactcccctcgg |
| 4500_WuhanS1 | atgtttgtgtttctagtgctactaccactagtgtcctcccagtgtgtgaatctgacaaccagaacccagctgcctccag |
| (SEQ ID NO: 43) | cctacaccaattccttcaccagaggagtctactaccctgacaaagtgttcagatcctctgtgctccactcaacaca |
| | ggatctgtttctgcccttcttctccaacgtcacttggttccacgctatccatgtgtccggaacaaacggcacaaagag |
| | attcgacaaccccgtgttgcctttcaacgacggcgtgtacttcgcctctaccgagaagtccaacatcatcagaggc |
| | tggatcttcggcaccacactcgattctaagacccagtctctgctgatcgttaacaacgccaccaacgtggtcatca |
| | aggtgtgcgagttccagttctgcaacgaccccttcctgggcgtctactaccacaagaacaacaagtcttggatgg |
| | aatccgagttccgtgtttacagctccgccaacaactgtaccttcgagtacgtctctcagcccttcctaatggacctgg |
| | aaggtaaacagggcaatttcaagaacctgagagagttcgtgttcaagaacatcgacggatattttaagatctactc |
| | taagcacacccctatcaacctggtgcgggacctgcctcaaggcttctccgctctggagcctctagtggacctgcct |
| | attggcatcaacatcaccagattccagaccctactggctctgcacagatcttacctgactcctggcgacagctcca |
| | gcggctggaccgccggcgctgccgcttactacgtgggctacctgcagcctcggaccttcctgctgaagtacaac |
| | gagaacggaaccatcaccgacgctgtggactgcgccctggatcctctgtctgaaactaagtgcaccctgaagtc |
| | gtttaccgtggaaaagggcatctaccagacctccaacttcagagtgcagccaaccgaatctatcgtgcgcttccc |
| | caacatcacaaacctgtgccccttcggcgaagtgttcaacgctacacggttcgcctccgtatacgcttggaacag |
| | aaaaagaatctccaactgtgtggctgattactccgtgctgtataacagcgcctctttttccaccttcaagtgctacgg |
| | ggtgtctcctaccaaactgaacgacctgtgctttaccaacgtgtacgccgactcctttgtgatccggggcgatgag |
| | gtgcggcagatcgcccctggccagaccggcaagatcgccgactataattacaagctgcctgacgacttcaccg |
| | gctgcgtgatcgcatggaactccaacaatctggactccaaggtgggcggcaactacaactatctgtatagactgtt |
| | cagaaagtccaacctgaaacctttcgagagagacatcagcaccgagatctaccaggccggctctaccccttgc |
| | aacggcgtagagggcttcaactgctacttccccctgcagtcctacggctttcaacctaccaacggagtgggctac |
| | caaccttaccgggtggtggtgctgtccttcgagctgctgcacgcccctgctactgtgtgcggacctaagaaatctac |
| | caatctggtgaaaaacaagtgcgtgaacttcaacttcaacggcctgacaggcactggcgtcctgaccgagtcca |
| | acaagaagttcctgcctttccagcagttcggcagagacatcgccgacaccaccgacgccgttagagatcctcag |
| | accctggagatcctggacatcaccccttgctcctttggcggcgtgtccgtgatcacccctggcaccaacaccagta |
| | atcaggtggccgtgctgtaccaggacgtgaactgcaccgaagtgcctgtggccatccacgccgaccagctgac |
| | acctacatggcgggtttactccacaggatccaacgtgttccagacccgggctggctgcctgatcggcgccgagc |
| | acgtgaacaactcctacgagtgcgacatccctatcggcgctggcatctgcgcttcttaccaaacccagaccaact |
| | ccccccgc |
| 4501_WuhanS1 | atgtttgtgtttctagtgctactaccactagtgtcctcccagtgcgtgaacctgaccacaagaacccagctgcctcct |
| (SEQ ID NO: 44) | gcgtacaccaactccttcaccagaggcgtgtactaccctgacaaggtgtttcgttcctccgtcttgcactccacaca |
| | ggatctgtttctgcctttcttctctaacgtgacgtggttccacgccatccacgtgtccggtaccaacggcacaaagag |
| | attcgacaaccccgtgctgcctttcaacgacggcgtttacttcgcctccactgagaagtctaacatcatcagaggat |
| | ggatcttcggtaccaccctcgacagcaagacccaatccctgctgatagtgaacaacgctacaaacgtggtgatc |
| | aaggtgtgcgagttccagttctgcaacgatccttttctgggagtgtactaccacaagaataacaagtcctggatgg |
| | aaagtgaattcagagtgtactctagcgccaacaactgtacctttgagtacgtgtcccaacctttcctgatggacctg |
| | gaaggaaagcagggcaacttcaagaacctgagagagttcgtgttcaagaacatcgatggctacttcaagatcta |
| | ctccaagcacacacctatcaacttggttagagacctcccgcagggcttctccgctctggaacctctggtcgacctg |
| | cccattggcatcaacatcacacggttccagaccctgctggctctgcacagatcctacctgaccccaggagattcct |
| | cttctggctggaccgccggcgctgccgcttactacgtgggctacctgcagcctcggacattcctgctgaagtacaa |
| | cgagaacggcaccatcaccgacgctgtggactgtgccctggatcccctgagcgagacaaagtgtaccctgaaa |
| | tcttttaccgtggaaaagggcatttaccagacctccaacttcagggtgcagcctactgagtccatcgtgcggttccct |
| | aacatcaccaacctgtgccccttcggcgaagtgtttaacgccaccagatttgcttctgtgtacgcttggaacagaa |
| | agagaatcagcaactgcgtggctgattactccgtcctgtacaattcagcaagcttctccaccttcaaatgctacgg |
| | agtgtctccaaccaagctgaacgacctgtgtttcaccaacgtgtacgccgactctttcgtgatccggggcgacgaa |
| | gtccggcaaatcgcccccggccagaccggcaagatcgccgactacaactacaagctgcctgacgatttcaccg |
| | gatgtgtcatcgcttggaactccaacaacctggatagtaaagtgggcggcaactataattatttgtaccggctgttc |
| | cggaagtctaatctgaagcctttcgagagagacatctctaccgagatctaccaggccggctctacaccttgcaac |
| | ggcgtagagggcttcaactgctacttccccctgcagagctacggcttccagcctactaacggcgttggctaccag |
| | ccttacagagtggtggtgctgtctttcgagctgctgcatgctccagccacagtgtgcggccctaagaagtccacga |
| | acctggtgaagaacaaatgcgtgaacttcaacttcaatggcctgaccggcaccggcgtgctgacagagtctaac |
| | aagaagttcctgccattccagcagttcggcagagatattgctgacaccactgatgccgtacgggaccctcagacc |
| | ctggaaatcctggatatcaccccttgttccttcggcggcgtctccgtgatcactcctggaaccaacacctccaacca |
| | ggtggccgtgctgtaccaggacgtgaactgcaccgaggtgcctgtggccatccatgccgaccagctgacgccc |
| | acctggcgggtgtactccaccggatctaatgtgttccaaacccgggctggctgcctgatcggcgccgagcacgtg |
| | aataactcctacgagtgcgacatccctatcggggccggaatctgcgcgtcataccagacccagacaaactctcc |
| | taga |
| 4502_WuhanS1 | atgtttgtgtttctagtgctactaccactagtctcttcccagtgtgtgaacctgaccaccagaacccagctgcctcctg |
| (SEQ ID NO: 45) | cctacacaaattcttttacacgcggagtgtactaccctgacaaggtgttcagatcctctgtactgcatagtacacag |
| | gacctgtttctgcccttcttcagcaacgtgacctggttccacgctattcatgtttctggcaccaatggaaccaagagat |
| | tcgacaatcctgtgctgccattcaacgacggcgtgtacttcgcctccacggagaagtccaacatcatcaggggct |
| | ggatcttcggcaccaccctggattcaaagacccagtctctgctgatcgtgaataatgctaccaacgtggtcatcaa |
| | agtatgcgagttccagttctgcaacgacccttttctgggcgtgtactaccacaagaacaataagtcctggatggaat |
| | ccgagttcagagtatactcttctgccaacaactgtaccttcgagtacgtctctcagcctttcctgatggaccttgaggg |
| | caagcagggcaacttcaaaaacctgagagagttcgtgttcaaaaacatcgacggctacttcaagatctactctaa |
| | gcacacccctatcaacctggtgcgcgacctgccccaaggcttttccgctctggaacctctggtggatctgcctatcg |
| | gcatcaacatcacccgcttccaaaccctgctcgctctgcaccgctcctacctgacccctggcgattcctcctccgg |
| | ctggaccgccggcgctgccgcctactacgtaggatatctgcagcctcggaccttcctgctgaagtacaatgagaa |
| | cggcaccatcaccgacgccgtggactgcgccctggaccccctgtccgagacaaagtgcaccctaaagtccttc |
| | acagtggaaaagggaatctaccagacatccaacttcagagtgcagcctaccgagtccatcgtgagattccctaa |
| | catcaccaacctgtgtcctttcggcgaggtgttcaacgccactagattcgcctccgtgtacgcttggaaccggaag |
| | cggatcagcaattgtgttgccgactactccgtcctgtacaactccgcgagtttctccactttcaagtgctacggcgtgt |
| | cacctaccaagttgaacgatctgtgcttcactaacgtctatgccgacagctttgtgatcagaggcgacgaggtgcg |
| | gcagattgcacctggccagaccggcaagatcgccgattacaactacaagctgcccgatgattttaccggctgtgt |
| | gatcgcctggaactccaacaacctggactctaaggtcggaggcaactacaactacctgtacagactgtttagaa |
| | agtctaacctgaagcccttcgagcgggacatctccactgagatctaccaggccggatccacaccttgtaacggc |
| | gtggaaggcttcaactgctacttccctctgcagtcttatggcttccagcctaccaacggcgtcggctaccagcctta |
| | cagagtggtcgtcctctccttcgagctgctgcacgccccagccaccgtgtgtgggcctaagaaatcgacaaacct |
| | ggtgaagaacaagtgcgtgaacttcaacttcaacggtctgaccggaaccggagttcttaccgaatccaacaaga |
| | agttcctgcctttccagcagtttggcagagacatcgccgacaccaccgatgctgtgagagatcctcagacactgg |
| | aaatcctggatatcaccccatgctccttcggcggcgtgagtgtgatcacccctggcaccaacacatccaaccaa |
| | gtggccgtgctgtaccaggacgtgaactgtaccgaggttcccgtggccatccacgccgaccagctgacccctac |
| | ctggcgggtgtactccaccggctccaacgtgttccaaactagagccggctgcctgatcggggccgagcacgtga |
| | acaactcttacgagtgcgacatccccatcggcgctggcatctgcgcttcctaccagacacagaccaactctccta |
| | ga |
| 4503_WuhanS1 | atgtttgtgtttctagtgctactaccactagtcagttctcagtgcgtgaacctgacaacgagaacccagctgcctcca |
| (SEQ ID NO: 46) | gcctacaccaactcattcacaagaggcgtgtactaccccgacaaggtgttcagaagctctgtgctgcactccacc |
| | caggatctgttcctgcccttcttctccaacgtgacctggtttcacgccatccacgtctccggcaccaatggcaccaa |
| | gcggttcgacaacccagtgctcccattcaacgacggcgtgtacttcgcctccaccgagaagtctaacatcatcag |
| | aggttggatcttcggcaccaccctggactccaagacccaatccctgctgatcgtgaacaacgccactaatgtggt |
| | catcaaagtgtgcgagttccagttctgcaacgaccccttcctgggtgtgtactaccacaagaacaacaagtcttgg |
| | atggaaagcgagttcagagtgtactcatctgctaacaattgcaccttcgagtacgtgagtcagccttttctgatgga |
| | cctggaaggaaagcagggcaacttcaaaaacctgagagagttcgtgttcaaaaatatcgacggctacttcaag |
| | atctactctaagcacacccctatcaacctggtccgggatctgcctcagggctttagcgccctcgagcctctggtgg |
| | acctgcccatcggcatcaacatcacccggttccagactctgttagctctgcacagatcctacctcacccccggcg |
| | attcctcttctggctggaccgctggcgctgctgcctactacgtgggctacctgcagcctcgaacgtttctgctgaagt |
| | acaacgagaacggcaccatcacagacgccgtagactgtgccctggatcctctgtctgagacaaagtgcacgct |
| | gaagtccttcaccgtggaaaagggcatctaccagacatccaacttccgggtgcagcctaccgagtctatcgtgcg |
| | gttccccaacataaccaacctgtgtcctttcggcgaggtatttaacgctaccagattcgcctccgtgtacgcctgga |
| | acagaaaacggatctctaactgcgttgccgactacagtgtgctgtacaactccgcttcattcagcaccttcaagtgc |
| | tacggcgtgtcccctaccaagctgaatgatctgtgcttcaccaacgtgtacgctgactctttcgtgatccggggcga |
| | cgaagtgagacagatcgcccctggccagacaggcaagatcgccgactataactacaagctgcctgacgacttc |
| | accggatgcgtgattgcctggaactccaataacctggattctaaggtgggcggcaactacaactacctgtatcgtc |
| | tgttccggaagtccaatctgaagccattcgaaagagatatttccaccgagatctaccaggctggaagcacccctt |
| | gcaacggcgtcgaaggcttcaactgctacttccccctgcagtcttacggctttcagcctaccaacggcgttggata |
| | ccaaccttacagagtggtcgtgctgtcttttgagctgctgcatgctccagcaaccgtgtgcggacccaagaagtcc |
| | accaatctggtgaagaacaagtgcgtgaacttcaacttcaacggcctgacgggtaccggcgtactgacagagtc |
| | caacaaaaagtttctgcctttccagcagttcggcagagatatcgccgacaccaccgacgccgtgagagatcctc |
| | agaccctggagatcctggatatcaccccttgtagcttcggcggagtgtccgtgatcacacccggcacaaacacct |
| | ctaaccaggtggccgtcctgtaccaggacgtgaactgcaccgaagtgcctgtggcaatccacgccgaccagct |
| | gacccccacctggcgggtttactctaccggcagcaacgtgtttcagaccagagctggctgcctgattggcgccga |
| | gcacgtgaacaactcgtacgagtgcgacatccctatcggagccggcatctgcgcctcctaccagacccagacc |
| | aactctcctaga |
| 4495_BTKY72 | agagtctctccaagcaccgaggtggtgcggtttcctaatatcaccaacctgtgccctttcggacaagtgtttaacgc |
| (SEQ ID NO: | ctccaacttcccatcggtgtacgcctgggagagactgcgcatctccgactgcgtcgccgactatgccgtgctgtac |
| 47) | aattcttcctcctctttctccacctttaagtgctacggcgtgagccctacaaagctgaacgacctgtgcttctcctctgt |
| | gtacgccgactacttcgtggtgaaaggcgatgatgtgagacagatcgcccctgcccagaccggcgtgatcgcc |
| | gattacaactacaagctccccgacgacttcacaggctgcgtgctggcctggaataccaactccgtagattccaaa |
| | tccggaaacaacttctactaccggctgttcagacacggcaagatcaagccctacgaaagagacatctccaacgt |
| | gctgtacaactccgctggcggcacctgttcctccatctctcagctgggctgctacgagcccctgaaatcctatgggt |
| | tcacccccaccgtggggggggctaccagccttatagggtcgtggtgttatccttcgaactgctgaacgctccagct |
| | accgtgtgcggccccaagaaatcaaccgagctggtcaagaacaagtgcgtgaacttc |
| 4495_RsSHC014 | agagtggcaccctctaaagaagtggtcagattccccaatatcaccaatctgtgtccttttggcgaggtcttcaacgc |
| (SEQ ID | caccaccttcccttctgtgtacgcctgggaacggaaacggatctctaactgcgtcgccgactactctgttctctaca |
| NO: 48) | actccacctctttctccacctttaaatgttacggcgtctctgctacaaaactgaacgatctgtgcttctctaacgtgtac |
| | gctgactcttttgtggtaaagggcgacgacgtgagacagatcgcccctggccaaaccggagtcatcgctgattat |
| | aactacaaactgcctgatgacttcctgggatgtgtgctggcttggaacaccaatagcaaggacagcagcacatct |
| | ggcaactacaactacctgtacagatgggtgaggagaagcaagctcaacccgtatgaacgggacctgtctaacg |
| | acatctactctccaggcggacagtcctgctccgctgtgggccctaactgctacaatcctctgagaccctacggcttc |
| | ttcaccaccgctggcgtgggacatcagccttacagagttgttgtgctgagcttcgagctgctgaacgcccctgcca |
| | ccgtgtgtggccctaagctgtctactgatctgatcaaaaaccagtgcgtgaatttc |
| 4498_HKU3 | cgcgtgtcccctacccaagaggtgattcgttttcctaacatcaccaacagatgtcctttcgacaaggtgttcaatgcc |
| (SEQ ID NO: | acccggttccctaacgtgtacgcctgggaacggacaaaaatctccgactgcgtggccgactacaccgtgctgta |
| 49) | caattctacatctttttctactttcaaatgctacggcgtgtccccatctaagctgatcgacctgtgttttacctctgtgtatg |
| | ccgatactttcctgatccggtcctcggaggtgagacaggtcgcccctggcgagacaggggtgatcgccgactac |
| | aactacaagctgcctgacgattttaccggctgcgtgatcgcctggaataccgccaagcacgacacgggcaatta |
| | ctactatagatcccaccggaagaccaagctgaaacctttcgagcgcgatctgagcagcgacgacggcaacgg |
| | cgtttacaccctgtccacctatgacttcaaccctaacgtacccgtggcctaccaggccacccgggtggtggtgctgt |
| | ctttcgagctgctgaacgcccctgccaccgtgtgtggccctaagctgtctaccgaactggtgaagaaccagtgcgt |
| | gaacttc |
| 4498_BTKY72 | agagtttccccttccaccgaggtggtcagattccccaacatcacaaacctgtgcccattcggccaggtgttcaatg |
| (SEQ ID NO: | cttccaacttcccatctgtgtacgcctgggagcggctgaggatctctgactgcgtggccgactacgccgtgctgtac |
| 50) | aactcttcctccagcttctctaccttcaagtgttacggcgtgtctcctaccaagctgaatgatctgtgcttcagctctgtg |
| | tacgccgactacttcgtggtgaagggtgatgatgtacggcaaatcgcccctgctcagaccggcgtgattgccgac |
| | tacaattacaagctgccagacgacttcaccggctgtgtcctggcttggaacaccaactccgtggacagcaagtcc |
| | ggaaacaacttctactacagactgttcagacacggcaagatcaagccttacgagcgggacatctcgaacgtgct |
| | gtacaactccgccggcggaacctgtagctctatctcccagctgggctgctacgagcctctgaagagctacggctt |
| | cactcccaccgtgggcgtcggctatcagccttacagggtggttgtgctgtcctttgagctgctgaacgcccctgcta |
| | ccgtgtgcggccccaagaaatccacagagctggtgaagaacaagtgcgtgaacttc |
| 4498_WIV1 | cgggtggctccttctaaagaggtggtgagattccctaacatcaccaacctgtgcccttttggcgaggtcttcaacgc |
| (SEQ ID NO: | taccaccttcccatccgtgtacgcttgggagagaaaacggatcagcaattgcgtggctgattattccgtgctgtaca |
| 51) | actctacctctttttccaccttcaagtgctacggcgtatctgctactaagctgaacgacctgtgcttctccaacgtgtat |
| | gctgactctttcgtggtcaagggcgacgacgtgcggcagatcgctcctggccagaccggcgtgatcgccgactat |
| | aactacaagctgcctgatgacttcacaggctgtgttctggcctggaacaccagaaacatcgacgccacccagac |
| | cggcaattacaactacaagtacagatctctgcggcatggaaagctgaggcctttcgagcgggatatctccaacgt |
| | ccccttttcccctgatggcaagccttgcacccctcctgctttcaactgctactggcctctgaacgactacggcttctac |
| | atcaccaacggcatcggctaccagccttatagagtggtggtgctgtctttcgagctgctgaacgctcctgccaccgt |
| | gtgtggacctaagctgtcaaccgacctgatcaagaaccagtgcgtgaacttc |
| 4499_Khosta2 | agagtgtctccttctctggacgtggtgaggtttccaaacatgaccaacatctgccctttcgaccaggtgttcaacaa |
| (SEQ ID NO: | gacccagttcccttccgtctacgcttgggaacgggtgagaatctccgactgtgtgtccgactacaccgtgctgtaca |
| 52) | acagctctgcttctttctccaccttcaagtgttacggcgtgtctcctaccaagctgaacgacctgtgcttctcaggcgt |
| | gtacgccgactacttcgtggtcaaaggcgatcacgtgcaccagatcgcccctggccagaccggcgtgategcc |
| | gactacaattacaaactgccttccgagttcgtaggctgcatcctggcttggaacacaagaaccatcgactccaag |
| | agaggattctactaccggctgttcagacacggcaatatccggccgtacgagagagacacctccaatgtgcccta |
| | caatgctgctggaggcacctgcaaccagcctggcacccacaactgctacgagcctctgcaggactacggcttc |
| | acatccacatcgggagtgggctaccagccattccgcgtggtggtcttatcctttgagctgctgaacgcccctgcca |
| | ccgtgtgtggccccaagcagagcaccgatctagtgaaaaacaagtgcgtcaatttc |
| 4499_WIV1 | agagtggctccctctaaagaggtggtgagattccccaacattaccaacctgtgcccttttggagaggtcttcaatgc |
| (SEQ ID NO: | taccaccttccctagcgtatacgcttgggagcggaagcggatctccaactgcgtggccgactactctgtgctgtac |
| 53) | aactccaccagcttttctaccttcaaatgctacggcgtgtccgccaccaagctgaacgatctgtgcttttccaacgtg |
| | tacgccgattccttcgtggttaagggcgacgatgtgagacagatcgcccctggccagacaggtgtaatcgctgatt |
| | acaactacaagctgcctgacgacttcaccggctgtgtgctggcctggaacaccagaaacatcgatgctacacag |
| | accggcaactacaactacaagtaccggagcctgcggcatggcaagctgagacctttcgagagagatatctcca |
| | acgtgccattctctcccgatggcaaaccttgtaccccacctgctttcaactgctactggcccctgaacgactacggc |
| | ttctatatcactaacggcatcggctaccagccatacagagtggtggttctgagtttcgaactgctcaatgctccagc |
| | caccgtgtgcggacctaagctttccaccgacctgataaaaaaccagtgtgtgaacttc |
| 4500_Pang17 | cgggtgcaacctaccatctccatcgtgcggtttcctaacatcaccaacctgtgccctttcggcgaggtcttcaacgc |
| (SEQ ID NO: | cagcaagttcgcttccgtgtatgcctggaacagaaagagaatctccaactgcgtcgccgactactccgtgctgtac |
| 54) | aattctacaagtttctccaccttcaagtgctacggcgtgtcccctaccaagctgaacgacctgtgcttcaccaacgt |
| | ctacgccgactccttcgtggtgaagggcgatgaagtgagacagatcgctccaggacagacaggcgtaattgcc |
| | gactataattataaactccccgatgacttcaccggctgcgtgattgcctggaactctgtcaagcaggacgctttgac |
| | cggcggaaactacggctacctgtaccggctgttcagaaagtccaagctgaaaccttttgaacgggacatctctac |
| | cgaaatctaccaggctgggagcaccccttgtaacggacaagtgggcctgaactgttactaccccctggaaagat |
| | acggcttccatcctaccaccggcgtgaattaccagcctttccgggttgtggttctgagctttgagctgctgaacggcc |
| | cagctacggtgtgcggccccaagctctctaccacccttgtgaaggacaagtgcgtcaatttc |
| 4500_WIV1 | agagtggctccttccaaagaggtggtgagatttcctaatatcaccaacctgtgtcctttcggcgaagtgttcaacgc |
| (SEQ ID NO: | caccaccttcccatctgtgtacgcctgggagagaaagcggatcagcaactgtgtggctgattattctgtgctgtaca |
| 55) | actccacctccttctccacctttaagtgttacggcgtgtctgctaccaagctgaacgatctctgttttagtaacgtgtac |
| | gccgactccttcgtggtgaagggcgacgatgtgcggcagatcgcccctggccagaccggcgtgatcgctgatta |
| | caactacaagctgcctgacgactttacaggctgtgtgctggcttggaacacccggaacatcgatgccacccaga |
| | ccggcaactacaactacaagtaccgctctctgcggcacggcaagctgaggccctttgagcgggatatctctaac |
| | gtgcctttcagccctgatggcaagccctgcacccctccagccttcaattgttactggcctctgaacgactacggcttc |
| | tacatcaccaacggaatcggctaccagccctatagagtggtggtgctgtcgttcgagctgctgaatgctcccgcca |
| | ccgtgtgtggcccaaagctgtccaccgacctgatcaagaaccagtgcgtgaacttc |
| 4501_Khosta2 | agagtttcgccttctctggacgtggtcagattccctaacatgaccaacatctgccctttcgaccaggtgttcaacaaa |
| (SEQ ID NO: | acccagttcccctccgtgtacgcctgggaacgggtgagaatttccgactgcgtctcagattacaccgtgctctataa |
| 56) | cagctctgcctctttctctacctttaagtgctacggcgtgagccctaccaagttaaatgacctgtgctttagcggcgtgt |
| | acgccgattatttcgtggtaaagggtgaccacgtgcaccagatagcccccggccagaccggcgtgatcgctgac |
| | tacaattataagctgcccagcgagttcgtgggctgcatcctggcctggaacactagaacaatcgactccaagag |
| | aggcttctactaccggctgttccggcatggcaacatcagaccttacgagcgcgatacctccaatgtgccctacaa |
| | cgctgctggcgggacctgcaaccagcctggcacccacaactgctacgagcctctgcaggactacggcttcacct |
| | ccacatctggcgtcggctaccaaccattcagagtggtggtgctgtctttcgagctgctgaacgctccagctaccgtg |
| | tgtggccctaagcaatccacagatctggtgaagaacaagtgtgtgaatttc |
| 4501_RsShC014 | agagtggctccttctaaagaggttgtgcggtttcctaacatcaccaacctgtgtcctttcggcgaggtgttcaacgcc |
| (SEQ ID | accacgttcccctctgtgtacgcctgggagagaaagaggatctccaactgtgttgctgactactcggtgctgtataa |
| NO: 57) | ctccacctctttttccaccttcaagtgctacggcgtctccgccaccaagctcaatgacctgtgcttctcgaacgtgtac |
| | gcagactccttcgtggtcaagggcgacgacgtgagacagatcgcccctggccagactggcgtgatcgccgact |
| | ataattacaaactgccggacgactttctgggctgcgtgctggcttggaacaccaactctaaggactcttccacctct |
| | ggcaactacaactacctgtatagatgggtgcgtaggagtaaactcaacccttacgagcgggacctgtccaacga |
| | tatctattcccctggcggccagtcctgctccgctgtgggccccaattgctacaaccccctgcggccttacggcttctt |
| | cacaaccgctggagtgggccaccagccttaccgggtggtggtgctgtcttttgaactgctgaatgctcctgctaccg |
| | tgtgcggccccaagctgtccaccgacctgataaaaaaccagtgtgtgaacttc |
| 4501_WIV1 | agagtcgcccctagtaaagaagtggtgagattccccaacatcaccaacctgtgccctttcggcgaagtgttcaac |
| (SEQ ID NO: | gccaccaccttcccttctgtgtacgcttgggagcggaagcggatctccaactgtgtggccgactatagcgtgttata |
| 58) | caactctacctctttttccaccttcaagtgctatggcgtgtctgccaccaaactgaacgacctgtgcttctcgaacgtg |
| | tacgctgatagctttgtggtgaaaggcgacgacgtgagacagatcgcacctggacagaccggagtgatcgccg |
| | actataactacaagcttccagatgacttcaccggctgtgtgctggcttggaacacccggaacatcgacgccaccc |
| | agacaggaaactacaactacaagtaccgctccctgagacacggcaagctgagacccttcgagcgggacatct |
| | ccaacgtgcctttctctcctgatggcaagccctgcacccctccagccttcaactgttactggcctctgaacgactac |
| | ggcttttacatcaccaatggcatcggatatcagccttacagagtggtggtcctgtcctttgagctgctgaacgcccct |
| | gccaccgtgtgcggccctaagctgtccaccgacctgatcaagaaccagtgcgtgaacttt |
| 4502_Khosta2 | agagtgtctccctccctcgacgtggttcgattccccaacatgaccaacatctgccctttcgaccaggtgttcaacaa |
| (SEQ ID NO: | aacccagtttccaagcgtgtacgcctgggagagagtcagaatctctgactgtgtcagcgactacaccgtgctgta |
| 59) | caacagttctgcctccttctccacatttaagtgctacggcgtgtcccctacaaagctgaacgacctgtgcttctctgg |
| | cgtctacgccgactacttcgtcgtgaagggcgaccacgtgcaccagatcgctccaggccagaccggcgttattg |
| | ccgactacaactacaagctgccttccgagttcgtgggatgcatcctggcctggaacactcggaccatcgactcca |
| | agcggggcttctactaccggctgttccggcacggtaacatccgcccctacgagagagacacctctaatgtaccct |
| | acaatgccgccggcggcacctgtaaccagcccggtacccacaactgctacgagcctctgcaggactacggctt |
| | cacctctaccagcggagtgggctaccaaccattcagagtggtggtgctgagttttgaactgctgaacgctcctgct |
| | acagtgtgcggccctaagcagtcaaccgatctggtgaagaacaagtgcgtgaatttc |
| 4502_RsShC014 | agagtggccccctctaaagaggtcgtgaggttccctaacatcaccaatctgtgccctttcggcgaagtgttcaacg |
| (SEQ ID | ctacaacgttcccctccgtttatgcctgggagagaaagcggatctccaactgcgtggctgattattccgtgctgtac |
| NO: 60) | aattcgacatctttctccacctttaagtgttacggcgtgtccgccaccaaactgaacgacctttgcttttccaacgtgta |
| | tgctgacagtttcgtggtgaagggcgacgatgtgcgacagatagcgcccggccagaccggcgtgatcgctgact |
| | acaactacaagctgcctgatgacttcctcggctgcgtgctggcctggaataccaactccaaggactcctcaacct |
| | ctggcaactataattacctgtacagatgggtgagacggagcaaactgaacccgtacgaacgggatctttctaatg |
| | acatctactctcccggtggacagtcctgctccgccgtgggcccaaattgttacaacccactgcggccttacggcttc |
| | ttcaccaccgctggcgtgggccatcagccttacagagtggtggtgctgtcctttgagctgctgaacgctcctgctac |
| | cgtgtgcggccctaaattgtctaccgacctgatcaagaaccagtgcgtgaatttc |
| 4502_Urbani | agagtggtgccttccggcgacgtcgttagatttccgaacatcaccaatctgtgtcctttcggcgaagtgttcaacgc |
| (SEQ ID NO: | caccaagttcccttctgtgtacgcttgggaacggaagaaaatctccaactgtgtggccgactactctgtgttgtataa |
| 61) | ctctaccttcttttccaccttcaagtgctacggcgtgtctgccaccaagctgaacgatctctgcttctccaacgtgtacg |
| | cagactccttcgtggtgaaaggcgacgacgtcagacagatcgctcccggacagaccggcgtgatcgccgatta |
| | caactacaagctgcccgacgacttcatgggctgtgtgctggcttggaataccagaaacatcgatgccacctctact |
| | ggcaactacaactacaagtaccgctacctgagacacggcaaactgcggccttttgaacgggacatctccaacgt |
| | gccattcagccctgatggcaagccttgcacgccccctgctctgaactgctactggcctctgaacgactacggattct |
| | acacaaccaccggcatcggatatcagccttaccgggtggtagtgctgagcttcgagctgctgaacgccccagct |
| | accgtgtgcggccccaagctgtctaccgacctcatcaagaaccagtgcgtgaatttttga |
| 4503_RatG13 | cgggtgcagcccaccgactccatcgtcagattccctaacatcaccaacctctgcccttttggagaagtgttcaacg |
| (SEQ ID NO: | ccaccacgttcgcctctgtgtacgcttggaacagaaagcggatctccaactgcgtggcggactactctgtcctgta |
| 62) | caattccacctccttttccaccttcaagtgctacggcgtgtcccctacaaagctgaacgatctgtgcttcaccaatgt |
| | gtacgccgatagcttcgttatcaccggcgacgaggtgcgccagatcgctcctggccagaccggcaagatcgcc |
| | gactacaactacaagttgcccgatgacttcaccggctgtgtgatcgcttggaacagcaagcacatcgatgccaa |
| | ggaaggtggcaacttcaactacctgtacagactgtttagaaaggccaaccttaagccatttgagagagacatctc |
| | cacagaaatctaccaggctggctccaaaccttgcaatggccagacaggcctgaactgctactatcctctgtaccg |
| | ctacggcttctaccctaccgatggcgtgggccaccagccttatagagtcgtggtgctgtctttcgaactgctgaacg |
| | cccctgccaccgtgtgtggccctaagaagtctacaaacctggtgaaaaataagtgcgtgaacttc |
| 4503_BTKY72 | cgggtgtccccaagcacggaggtggtccggttcccaaacatcaccaacctgtgcccttttggccaagtgttcaatg |
| (SEQ ID NO: | cttcaaactttccttctgtgtacgcttgggagcggctgaggatctccgactgcgtggctgattacgccgtgctctaca |
| 63) | actctagcagctctttctctaccttcaagtgctacggcgtgtctccaaccaagctgaacgacctgtgtttctcctccgt |
| | gtacgccgactacttcgtggtgaagggcgacgacgtgagacagatcgctcctgctcagaccggagtgatcgcc |
| | gattacaactacaagctgcctgatgacttcaccggctgcgtgctggcctggaacaccaactccgtggactccaag |
| | agtgggaacaacttctactacagactgtttagacatggcaagatcaagccttacgagcgcgacatctccaacgtg |
| | ctgtacaactctgccggcggcacctgctcctccattagccagctgggatgttacgagcccctgaaaagctacggc |
| | tttacccctactgtaggcgtcggctatcagccttaccgggttgtggtgctgtccttcgaactgctgaacgcccctgcta |
| | ccgtgtgtggccctaagaaatccaccgagctggtgaagaacaagtgcgtgaacttc |
| 4503_WIV1 | agagtggcaccgtctaaagaggtggtgcggttcccaaacatcaccaacctgtgtcctttcggcgaagtcttcaac |
| (SEQ ID NO: | gccaccacgttccctagcgtttacgcctgggaacggaagagaatctctaactgtgtggccgactacagcgtgctg |
| 64) | tacaactccacctcgttttccaccttcaagtgttatggcgtgtccgccaccaagttgaacgacctgtgcttcagcaat |
| | gtgtatgccgactccttcgtggtcaaaggcgacgacgtgcggcagatcgcccctggacaaactggcgtgatcgc |
| | cgactataactacaaactgcctgacgatttcaccggctgtgtgctggcttggaatacccggaacatcgatgctaca |
| | cagaccggcaactacaactacaagtacaggagcctgagacacggcaagcttaggcctttcgagagagacattt |
| | ccaacgtgcccttctcccctgacggcaagccctgcacaccccctgcgttcaactgttactggcctctgaacgacta |
| | tggcttttacatcaccaacgggatcggctatcaaccgtaccgggtggtggtgctgtcttttgagctgctgaacgcgc |
| | ctgccacagtgtgcggccccaagctctccaccgacttgatcaagaaccaatgtgtgaacttc |
| OmicronRBD | cgagtccaaccaacagagtcaatcgtaagattccccaatatcaccaacctctgcccctttgacgaagttttcaacg |
| (SEQ ID NO: | ctaccagatttgcaagtgtgtacgcctggaataggaagcggatcagtaactgcgtcgccgactactctgtactttat |
| 65) | aacctcgcgcccttctttacattcaagtgctatggcgtctcccccacgaaacttaatgacctctgtttcacgaacgtct |
| | acgccgattcctttgtgataagaggggatgaggtaagacaaatagcacctggtcagacaggcaacatagcaga |
| | ctataactataagttgcccgacgactttacaggatgtgtaattgcttggaacagtaacaagctggattcaaaggtttc |
| | cggcaattataactacctgtacagactgttcagaaaaagtaatctcaaacccttcgagagagacatttcaaccga |
| | aatctatcaggccgggaacaaaccatgcaacggcgtcgctggattcaactgttacttccccctccgatcatactcc |
| | tttaggcccacatatggagtgggtcatcaaccttaccgagttgttgtcctgtctttcgagctcttgcacgcgccggca |
| | actgtgtgtggacctaaaaagagtacgaacttggtgaagaataagtgtgtaaactttaactttaatggt |
| BetaRBD (SEQ | atgttcgtgtttctggtgctgctgcctctggtgtccagccgggtgcagcccaccgaatccatcgtgcggttccccaat |
| ID NO: 66) | atcaccaatctgtgccccttcggcgaggtgttcaatgccaccagattcgcctctgtgtacgcctggaaccggaagc |
| | ggatcagcaattgcgtggccgactatagtgtactgtataactcagcatccttttccacgtttaagtgctacggggtca |
| | gtcctacaaagttgaatgacttgtgctttacaaacgtatatgctgattcctttgtcatacgaggcgatgaggtcagac |
| | agattgctccgggacaaaccggaaatattgccgactacaactacaaacttcccgacgattttactgggtgtgtaatt |
| | gcctggaacagtaacaatcttgactcaaaagtggggggaaattacaattacctttatagactcttccgcaagagc |
| | aatctcaagccttttgaacgcgatatatctaccgaaatataccaggccggttctactccatgcaatggagttaaggg |
| | atttaattgttactttcctctccaatcttatgggttccagccaacttatggagtcggttatcagccatatcgagtcgttgtg |
| | ctcagcttcgaactgcttcatgccccagctactgtttgtgggccgaaaaagtccactaatctcgtgaagaataagtg |
| | cgtaaacttt |
| DeltaRBD | agggttcagcccacagagtccatagtacggttccctaatattaccaacctgtgtccgttcggtgaagtcttcaatgc |
| (SEQ ID NO: | cacaagattcgccagcgtttatgcctggaaccgaaagagaattagtaattgtgttgcggattattctgtcctttacaat |
| 67) | agtgcaagcttctccaccttcaagtgttacggggtttccccgacgaaactcaacgacctttgttttacaaatgtgtatg |
| | cagattcatttgttatccgaggagatgaggtacggcaaatagcccccggacaaacaggaaagatagccgatta |
| | caactataaactgcctgatgacttcactgggtgcgtcatcgcatggaatagcaacaaccttgattccaaagtaggg |
| | ggcaattataattaccgctatcgacttttccgcaagtccaacctcaaacccttcgagcgcgatatcagcacggaaa |
| | tttaccaagcgggcagcaaaccatgcaacggagtcgaaggatttaattgttacttcccgttgcaatcctatggctttc |
| | aaccaaccaacggtgttggttaccaaccttatagagttgtcgttctcagttttgagctcctccatgccccggccaccg |
| | tatgcggcccaaagaaatcaacaaacctcgttaagaacaaatgtgtcaactttaatttcaatggg |

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

As used herein, the term “substantially” refers to an approximately +/−5% variation from a given value. If a value is not used, then substantially means almost completely, but perhaps with some variation, contamination and/or additional component. In some embodiments, “substantially” may include completely.

It should be understood that the polypeptides, immunogenic compositions, uses and methods are described in terms of “comprising,” “containing,” or “including” various components or steps, the polypeptides, immunogenic compositions, uses and methods can also “consist essentially of” or “consist of the various components and steps”. Moreover, the indefinite articles “a” or “an”, as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b” or, “from a to b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be referenced herein, the definitions that are consistent with this specification should be adopted.

Many obvious variations of the embodiments set out herein will suggest themselves to those skilled in the art in light of the present disclosure. Such obvious variations are within the full intended scope of the appended claims.

1. A fusion polypeptide comprising an amino acid sequence encoding, in sequence, a first antigen, a linker, and at least one additional antigen.

2. The fusion polypeptide of claim 1, wherein the at least one additional antigen comprises a plurality of antigens and each antigen of the plurality of antigens is linked to the preceding antigen with a linking peptide comprising the linker.

3. The fusion polypeptide of claim 1, wherein the sequence of the first antigen comprises a greater number of amino acids than the sequence of at least one of the at least one additional antigen.

4. The fusion polypeptide of claim 1, wherein the first antigen comprises an antigenic peptide or fragment thereof from a first pathogen, or
an antigenic peptide or fragment thereof from a first pathogen, and the first pathogen comprises a first prokaryotic antigen, a first viral antigen, or a first fungal antigen.

5. (canceled)

6. The fusion polypeptide of claim 4, wherein the first viral antigen comprises a component of a surface glycoprotein from a virus, a lipoprotein or an extracellular domain of a transmembrane protein;
and/or wherein the first viral antigen comprises a first coronavirus antigen comprising a MERS-CoV antigen, a SARS-CoV-1 antigen, a SARS-CoV-2 antigen (such as Wuhan coronavirus antigens, BA.1 coronavirus antigens, BA.2 coronavirus antigens, or B.1.351 coronavirus antigens), a HCoV-HKU1 antigen, a HCoV-OC43 antigen, a BtCoV-RaTG13 antigen, a Bt SL-CoV-WIV1 antigen, a HKU4-CoV antigen, a HKU5-CoV antigen, a PDF-2108 CoV antigen, a SHC014-CoV antigen, a BtSCoV-RsSHC014 antigen, a BtSCoV-Rs3367 antigen, a BANAL-52 antigen, a BANAL-236 antigen, a Pangolin-CoV GX/P2V antigen, a Bat coronavirus CDPHE15 antigen, a Bat coronavirus HKU10 antigen, a Rhinolophus ferrumequinum alphacoronavirus HuB-2013 antigen, a Human coronavirus 229E antigen, a Lucheng Rn rat coronavirus antigen, a Mink coronavirus 1 antigen, a Ferret coronavirus antigen, a Miniopterus bat coronavirus 1 antigen, a Miniopterus bat coronavirus HKU8 antigen, a Myotis ricketti alphacoronavirus Sax-2011 antigen, a Nyctalus velutinus alphacoronavirus SC-2013 antigen, a Pipistrellus kuhlii coronavirus 3398 antigen, a Porcine epidemic diarrhea virus antigen, a Scotophilus bat coronavirus 512 antigen, a Rhinolophus bat coronavirus HKU2 antigen, a Human coronavirus NL63 antigen, a NL63-related bat coronavirus strain BtKYNL63-9b antigen, a Sorex araneus coronavirus T14 antigen, a Suncus murinus coronavirus X74 antigen, an Alphacoronavirus 1 antigen, a Canine coronavirus antigen, a Feline coronavirus antigen, a Human CCoV-HuPn-2018 antigen, a Porcine transmissible gastroenteritis coronavirus antigen, a Swine acute diarrhea syndrome coronavirus (SADS-CoV) antigen, a HCoV-NL63 antigen, a NL63-related bat coronavirus strain BtKYNL63-9b antigen, a HCoV-229E antigen, a Khosta2 sarbecovirus antigen, a Kenya bat coronavirus BtKY72 antigen, a bat SARS coronavirus HKU3 antigen, or any combination thereof.

7. (canceled)

8. The fusion polypeptide of claim 6, wherein the first coronavirus antigen comprises:
a spike (S) protein, or fragment thereof, and/or wherein the first coronavirus antigen comprises a SARS-CoV-2 spike protein or a protein encoded by a nucleotide sequence with at least 70% identity to the SARS-CoV-2 spike protein.

9. (canceled)

10. The fusion polypeptide of claim 1, wherein the linker sequence comprises:
6 to 24 amino acids, and/or glycine (G), serine(S), alanine (A), or any combination thereof.

11. (canceled)

12. The fusion polypeptide of claim 10 wherein an amino acid sequence of the linker comprises GSGSGS or GAAASG.

13. The fusion polypeptide of claim 1, wherein the at least one additional antigen comprises:
at least one additional antigenic peptide or fragment thereof from at least one additional pathogen;
and/or an amino acid sequence of at least 8 amino acids; and/or
at least one additional prokaryotic antigen, at least one additional viral antigen, at least one additional fungal antigen, or any combination thereof.

14-15. (canceled)

16. The fusion polypeptide of claim 13, wherein the at least one additional viral antigen comprises at least one coronavirus antigen.

17. The fusion polypeptide of claim 16, wherein the at least one coronavirus antigen comprises a MERS-CoV antigen, a SARS-CoV-1 antigen, a SARS-CoV-2 antigen, a HCoV-HKU1 antigen, a HCoV-OC43 antigen, a BtCoV-RaTG13 antigen, a Bt SL-CoV-WIV1 antigen, a HKU4-CoV antigen, a HKU5-CoV antigen, a PDF-2108 CoV antigen, a SHC014-CoV antigen, a BtSCoV-RsSHC014 antigen, BtSCoV-Rs3367 antigen, a BANAL-52 antigen, a BANAL-236 antigen, a Pangolin-CoV GX/P2V antigen, a Bat coronavirus CDPHE15 antigen, a Bat coronavirus HKU10 antigen, a Rhinolophus ferrumequinum alphacoronavirus HuB-2013 antigen, a Human coronavirus 229E antigen, a Lucheng Rn rat coronavirus antigen, a Mink coronavirus 1 antigen, a Ferret coronavirus antigen, a Miniopterus bat coronavirus 1 antigen, a Miniopterus bat coronavirus HKU8 antigen, a Myotis ricketti alphacoronavirus Sax-2011 antigen, a Nyctalus velutinus alphacoronavirus SC-2013 antigen, a Pipistrellus kuhlii coronavirus 3398 antigen, a Porcine epidemic diarrhea virus antigen, a Scotophilus bat coronavirus 512 antigen, a Rhinolophus bat coronavirus HKU2 antigen, a Human coronavirus NL63 antigen, a NL63-related bat coronavirus strain BtKYNL63-9b antigen, a Sorex araneus coronavirus T14 antigen, a Suncus murinus coronavirus X74 antigen, an Alphacoronavirus 1 antigen, a Canine coronavirus antigen, a Feline coronavirus antigen, a Human CCoV-HuPn-2018 antigen, a Porcine transmissible gastroenteritis coronavirus antigen, a Swine acute diarrhea syndrome coronavirus (SADS-CoV) antigen, a HCoV-NL63 antigen, a NL63-related bat coronavirus strain BtKYNL63-9b antigen, a HCoV-229E antigen, a Khosta2 sarbecovirus antigen, a Kenya bat coronavirus BtKY72 antigen, a bat SARS coronavirus HKU3 antigen, a Wuhan coronavirus antigen, a BA. 1 coronavirus antigen, a BA.2 coronavirus antigen, a B.1.351 coronavirus antigen or any combination thereof; or
wherein the at least one coronavirus antigen comprises a receptor-binding domain, fragment thereof, or a protein encoded by a nucleotide sequence with at least 70% identity to a Coronaviridae receptor binding domain.

18. (canceled)

19. The fusion polypeptide of claim 1, wherein the first antigen and the at least one additional antigen each independently comprise a prokaryotic antigen, a viral antigen, or a fungal antigen.

20. The fusion polypeptide of claim 1, further comprising, in sequence, a third antigen and the linker sequence upstream of the first antigen.

21. The fusion polypeptide of claim 2, wherein the plurality of antigens comprises prokaryotic antigens, viral antigens, fungal antigens, or any combination thereof.

22. The fusion polypeptide of claim 21, wherein the viral antigens comprise coronavirus antigens.

23. The fusion polypeptide of claim 22, wherein the coronavirus antigens comprise a MERS-CoV antigens, SARS-CoV-1 antigens, SARS-CoV-2 antigens, HCoV-HKU1 antigens, HCoV-OC43 antigens, BtCoV-RaTG13 antigens, Bt SL-CoV-WIV1 antigens, HKU4-CoV antigens, HKU5-CoV antigens, PDF-2108 CoV antigens, SHC014-CoV antigens, BtSCoV-RsSHC014 antigens, BtSCoV-Rs3367 antigens, BANAL-52 antigens, BANAL-236 antigens, Pangolin-CoV GX/P2V antigens, Bat coronavirus CDPHE15 antigens, Bat coronavirus HKU10 antigens, Rhinolophus ferrumequinum alphacoronavirus HuB-2013 antigens, Human coronavirus 229E antigens, Lucheng Rn rat coronavirus antigens, Mink coronavirus 1 antigens, Ferret coronavirus antigens, Miniopterus bat coronavirus 1 antigens, Miniopterus bat coronavirus HKU8 antigens, Myotis ricketti alphacoronavirus Sax-2011 antigens, Nyctalus velutinus alphacoronavirus SC-2013 antigens, Pipistrellus kuhlii coronavirus 3398 antigens, Porcine epidemic diarrhea virus antigens, Scotophilus bat coronavirus 512 antigens, Rhinolophus bat coronavirus HKU2 antigens, Human coronavirus NL63 antigens, NL63-related bat coronavirus strain BtKYNL63-9b antigens, Sorex araneus coronavirus T14 antigens, Suncus murinus coronavirus X74 antigens, Alphacoronavirus 1 antigens, Canine coronavirus antigens, Feline coronavirus antigens, Human CCoV-HuPn-2018 antigens, Porcine transmissible gastroenteritis coronavirus antigens, Swine acute diarrhea syndrome coronavirus (SADS-CoV) antigens, HCoV-NL63 antigens, NL63-related bat coronavirus strain BtKYNL63-9b antigens, HCoV-229E antigens, Khosta2 sarbecovirus antigens, Kenya bat coronavirus BtKY72 antigens, bat SARS coronavirus HKU3 antigens, Wuhan coronavirus antigens, BA.1 coronavirus antigens, BA.2 coronavirus antigens, B.1.351 coronavirus antigens or any combination thereof; or
wherein the coronavirus antigens comprise a receptor-binding domain, fragment thereof, or a protein encoded by a nucleotide sequence with at least 70% identity to a Coronaviridae receptor binding domain.

24-25. (canceled)

26. A fusion polypeptide comprising an amino acid sequence encoding a plurality of antigens in series, wherein each of the plurality of antigens is linked with a linker sequence to the preceding antigen.

27. The fusion polypeptide of claim 26, wherein the plurality of antigens comprises three or more antigens.

28. The fusion polypeptide of claim 26, wherein each of the plurality of antigens independently comprises a prokaryotic antigen, a viral antigen, or a fungal antigen.

29. The fusion polypeptide of claim 26, wherein the linker sequence comprises:
6 to 24 amino acids; and/or glycine (G), serine(S), alanine (A), or any combination thereof.

30. (canceled)

31. The fusion polypeptide of claim 1 comprising:
a Wuhan coronavirus antigen and a B.1.617.2 coronavirus antigen;
a Wuhan coronavirus antigen and a BA.1 coronavirus antigen;
a B.1.351 coronavirus antigen, a Wuhan coronavirus antigen and a BA.1 coronavirus antigen;
a Wuhan coronavirus antigen, a Khosta2 sarbecovirus antigen, and a Bt SL-CoV-WIV1 antigen;
a BA.2 coronavirus antigen, a Kenya bat coronavirus BtKY72 antigen, and a BtSCoV-RsSHC014 antigen;
a Wuhan coronavirus antigen, a Pangolin-CoV GX/P2V antigen, and a Bt SL-CoV-WIV1 antigen;
a Wuhan coronavirus antigen, a Khosta2 sarbecovirus antigen, a BtSCoV-RsSHC014 antigen, and a Bt SL-CoV-WIV1 antigen;
a BA.2 coronavirus antigen, a bat SARS coronavirus HKU3 antigen, a Kenya bat coronavirus BtKY72 antigen, and a Bt SL-CoV-WIV1 antigen;
a Wuhan coronavirus antigen, a Khosta2 sarbecovirus antigen, a BtSCoV-RsSHC014 antigen, and a SARS-CoV-1 antigen; or
a Wuhan coronavirus antigen, a BtCoV-RaTG13 antigen, a Kenya bat coronavirus BtKY72 antigen, and a Bt SL-CoV-WIV1 antigen.

32-40. (canceled)

41. The fusion polypeptide of claim 1, wherein the first antigen comprises a receptor binding domain or an S1 domain.

42. The fusion polypeptide of claim 1, wherein the antigen comprises a sequence selected from the group consisting of SEQ ID NOs: 12-24 or a sequence with at least 90% identity, at least 91% identity, at least 92% identity, at least 93% identity, at least 94% identity, at least 95% identity, at least 96% identity, at least 97% identity, at least 98% identity, at least 99% identity to a sequence selected from the group consisting of SEQ ID NOs: 12-24.

43. A fusion polypeptide of any one of SEQ ID NOs: 1 to 11 or a sequence with at least 90% identity, at least 91% identity, at least 92% identity, at least 93% identity, at least 94% identity, at least 95% identity, at least 96% identity, at least 97% identity, at least 98% identity, at least 99% identity to any one of SEQ ID NOs: 1 to 11, respectively.

44-53. (canceled)

54. An immunogenic composition comprising the fusion polypeptide of claim 1, and a pharmaceutically acceptable salt, carrier, or adjuvant.

55-57. (canceled)

58. A method of treating or preventing a pathogenic infection comprising administering an effective amount of the fusion polypeptide of claim 1.

59. The method of claim 58, wherein the pathogenic infection comprises a viral infection, a bacterial infection, or a fungal infection; or
wherein the pathogenic infection comprises a viral infection.

60. (canceled)

61. The method of claim 59, wherein the viral infection comprises a coronavirus.

62. The method of claim 61, wherein the coronavirus comprises MERS-CoV, SARS-CoV-1, SARS-CoV-2, HCoV-HKU1, HCoV-OC43, BtCoV-RaTG13, Bt SL-CoV-WIV1, HKU4-CoV, HKU5-CoV, PDF-2108 COV, SHC014-CoV, BtSCoV-RsSHC014, BtSCoV-Rs3367, BANAL-52, BANAL-236, Pangolin-CoV GX/P2V, Bat coronavirus CDPHE15, Bat coronavirus HKU10, Rhinolophus ferrumequinum alphacoronavirus HuB-2013, Human coronavirus 229E, Lucheng Rn rat coronavirus, Mink coronavirus 1, Ferret coronavirus, Miniopterus bat coronavirus 1, Miniopterus bat coronavirus HKU8, Myotis ricketti alphacoronavirus Sax-2011, Nyctalus velutinus alphacoronavirus SC-2013, Pipistrellus kuhlii coronavirus 3398, Porcine epidemic diarrhea virus, Scotophilus bat coronavirus 512, Rhinolophus bat coronavirus HKU2, Human coronavirus NL63, NL63-related bat coronavirus strain BtKYNL63-9b, Sorex araneus coronavirus T14, Suncus murinus coronavirus X74, Alphacoronavirus 1, Canine coronavirus, Feline coronavirus, Human CCoV-HuPn-2018, Porcine transmissible gastroenteritis coronavirus, Swine acute diarrhea syndrome coronavirus (SADS-CoV), HCoV-NL63. NL63-related bat coronavirus strain BtKYNL63-9b, HCoV-229E, Khosta2 sarbecovirus, bat SARS-like coronavirus WIV1, Kenya bat coronavirus BtKY72, bat coronavirus SHC014, pangolin coronavirus Pang17, bat SARS coronavirus HKU3, SARS coronavirus Urbani, bat coronavirus RaTG13, Wuhan coronavirus, BA. 1 coronavirus, BA.2 coronavirus, B.1.351 coronavirus or any combination thereof; or virus with at least 60% nucleotide identity with MERS-CoV, SARS-CoV-1, SARS-CoV-2, HCoV-HKU1, HCoV-OC43, BtCoV-RaTG13, Bt SL-CoV-WIV1, HKU4-CoV, HKU5-CoV, PDF-2108 CoV, SHC014-CoV, BtSCoV-RsSHC014, BtSCoV-Rs3367, BANAL-52, BANAL-236, Pangolin-CoV GX/P2V, Bat coronavirus CDPHE15, Bat coronavirus HKU10, Rhinolophus ferrumequinum alphacoronavirus HuB-2013, Human coronavirus 229E, Lucheng Rn rat coronavirus, Mink coronavirus 1, Ferret coronavirus, Miniopterus bat coronavirus 1, Miniopterus bat coronavirus HKU8, Myotis ricketti alphacoronavirus Sax-2011, Nyctalus velutinus alphacoronavirus SC-2013, Pipistrellus kuhlii coronavirus 3398, Porcine epidemic diarrhea virus, Scotophilus bat coronavirus 512, Rhinolophus bat coronavirus HKU2, Human coronavirus NL63, NL63-related bat coronavirus strain BtKYNL63-9b, Sorex araneus coronavirus T14, Suncus murinus coronavirus X74, Alphacoronavirus 1, Canine coronavirus, Feline coronavirus, Human CCoV-HuPn-2018, Porcine transmissible gastroenteritis coronavirus, Swine acute diarrhea syndrome coronavirus (SADS-CoV), HCoV-NL63. NL63-related bat coronavirus strain BtKYNL63-9b, HCoV-229E, Khosta2 sarbecovirus, bat SARS-like coronavirus WIV1, Kenya bat coronavirus BtKY72, bat coronavirus SHC014, pangolin coronavirus Pang17, bat SARS coronavirus HKU3, SARS coronavirus Urbani, bat coronavirus RaTG13, Wuhan coronavirus, BA. 1 coronavirus, BA.2 coronavirus, B.1.351 coronavirus, or any combination thereof.

63. The method of claim 58, wherein the fusion polypeptide or the immunogenic composition:
is for administration one or more times; and/or
neutralizes two or more different pathogens, two or more different antigens, or both in the subject; and/or
reduces the quantity of the pathogen in the subject.

64. (canceled)

65. The method of claim 64, wherein the two or more different pathogens comprise two or more protein variants derived from a same parental or ancestral pathogen; or
wherein the two or more pathogens comprise two or more coronaviruses, two or more coronavirus variants, or any combination thereof.

66-67. (canceled)

68. A nucleic acid encoding the fusion polypeptide of claim 1; comprising a sequence selected from the group consisting of: SEQ ID NOs: 25-67; or encoding a fusion polypeptide with at least 90% identity, at least 91% identity, at least 92% identity, at least 93% identity, at least 94% identity, at least 95% identity, at least 96% identity, at least 97% identity, at least 98% identity, at least 99% identity to the fusion polypeptide of any one of SEQ ID Nos: 25-67.

69. A cell transformed with the nucleic acid of claim 68.

70. The cell of claim 69, wherein the cell comprises a mammalian cell, a yeast cell, a plant cell, a bacterial cell, or an insect/arthropod cell.

71. The cell of claim 70, wherein the mammalian cell comprises a Chinese Hamster Ovary (CHO) cell.
